(19) United States
(12) Patent Application Publication
(10) Pub. No.: US 2007/0185128 A1
(43) Pub. Date: Aug. 9, 2007

(54) Compounds and compositions for treating obesity

(76) Inventors: Kilian Waldemar Conde-Frieboes, Malov (DK); Michael Ankersen, Stenlose (DK); Ulrich Sensfuss, Valby (DK); Birgitte Schjellerup Wulff, Virum (DK); Henning Thogersen, Farum (DK); Philipp Lustenberger, Warthausen (DE); Klaus Rudolf, Warthausen (DE); Bernd Krist, Wien (AT); Stephan Muller, Warthausen (DE); Dirk Stenkamp, Biberach (DE); Marcus Schindler, Biberach (DE); Heike Wieland, Bad Soden (DE); Kirsten Arndt, Biberach (DE)

Correspondence Address: NOVO NORDISK, INC.;PATENT DEPARTMENT, 100 COLLEGE ROAD WEST, PRINCETON, NJ, 08540, US

(21) Appl. No.: 10/719,110

(22) Filed: Nov. 21, 2003

Related U.S. Application Data
(60) Provisional application No. 60/430,606, filed on Dec. 3, 2002.

(30) Foreign Application Priority Data
Nov. 22, 2002 (DK) PA 2002 01806

(51) Int. Cl. C07D 403/06; A61K 31/4965; A61K 31/497; C07D 241/04

(52) U.S. Cl. 514/252.13; 514/255.02; 544/359; 544/385

(57) ABSTRACT

Described herein are novel and useful compounds of the general formula 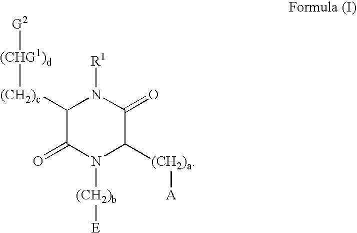 Compounds of this invention can advantageously exhibit melanocortin receptor agonist activity. New and useful compounds comprising such a chemical structure and methods of modulating melanocortin receptor activity in a subject (and promoting, inducing, and/or enhancing the treatment or prevention of related diseases) by administering such a compound or composition also are described.

### CROSS-REFERENCE TO RELATED APPLICATIONS

This application claims the benefit of priority under 35 U.S.C. 119 of Danish application no. PA 2002 01806 filed Nov. 22, 2002, and U.S. application No. 60/430,606, filed Dec. 3, 2002, the contents of which are both fully incorporated herein by reference.

### FIELD OF INVENTION

The present invention relates to novel compounds, pharmaceutical compositions containing them, and methods of regulating appetite (e.g., in the treatment of obesity and/or obesity-related disorders) using such compounds and compositions.

### BACKGROUND OF THE INVENTION

Obesity is a well known risk factor for the development of many very common diseases such as atherosclerosis, hypertension, type 2 diabetes (non-insulin dependent diabetes mellitus (NIDDM)), dyslipidaemia, coronary heart disease, osteoarthritis, and various other malignancies. Obesity also causes considerable problems through reduced motility and decreased quality of life. Even mild obesity increases the risk for premature death, diabetes, hypertension, atherosclerosis, gallbladder disease, and certain types of cancer. The incidence of obesity is increasing throughout the entire industrialised world. Yet, only a few pharmacological treatments for obesity are available to date.

Melanocortin peptides, including adrenocortiocotrophin (ACTH) and melanocyte stimulating hormone (MSH), are processed from the large polypeptide precursor, proopiomelanocortin (POMC). ACTH and MSH have long been recognised for their roles in the stress response and pigmentation, respectively. Melanocortin peptides are also recognized for having a diverse array of biological effects which include nerve regeneration, cardiovascular regulation, thermoregulation, fetal growth, immune functions, and behavioural responses. Melanocortin peptides, including ACTH and α-MSH, also have been demonstrated to have appetite suppressing activity when injected intracerebroventricularly in rats (Vergoni et al, European Journal of Pharmacology 179, 347-355 (1990)).

To date, a family of five melanocortin receptor subtypes has been identified (melanocortin receptors 1-5, also called MC1, MC2, MC3, MC4 and MC5). The MC1, MC2, and MC5 receptors are mainly expressed in peripheral tissues whereas MC3 and MC4 are mainly centrally expressed.

Results of recent research suggests a connection between the melanocortin receptors and obesity. MC4 knock out mice develop obesity (Huzar et al, Cell 88, 131-141 (1997)). Furthermore, studies of ectopic centrally expression of agouti (an MC1, MC3 and MC4 antagonist) or over-expression of an endogenously occurring MC3 and MC4 antagonist (agouti gene related peptide, AGRP) in the brain demonstrate that the over-expression of these two antagonists may be associated with the development of obesity (Kleibig et al, PNAS 92, 4728-4732 (1995)). Moreover, intracerebroventricular (“icv”) injection of a C-terminal fragment of AGRP was shown to be associated with increased feeding and antagonizes the inhibitory effect of α-MSH on food intake. In humans, several cases of families with obesity linked to frame shift mutations in the MC4 receptor have been described (Yeo et al, Nature Genetics 20, 111-112 (1998), Vaisse et al, Nature Genetics 20, 113-114). In view of these findings, molecules that can effectively modulate melanocortin receptor activity may serve as useful therapeutic agents in the treatment of obesity and obesity-related diseases. In view of the shortcomings attendant peptide drugs, there remains a need for improved and alternative compounds (particularly small molecule compounds) for modulating melanocortin receptor activity.

The invention described here provides such compounds as well as related compositions and methods of using such compositions and compounds in the induction, promotion, and/or enhancement of physiological responses associated with melanocortin receptor activity (including the induction, enhancement, and/or promotion of the treatment and/or prevention of obesity, obesity-related diseases, and several other conditions in subjects). These and other advantages of the invention, as well as additional inventive features, will be apparent from the description of the invention provided herein.

### SUMMARY OF THE INVENTION

The present invention relates to novel compounds of the general formula

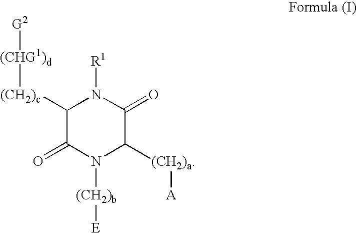

The invention also provides isomers, tautomers, diastereomers, and salts of such compounds. Additional aspects of the invention include compounds comprising a chemical structure according to Formula I (e.g., peptide-small molecule conjugates, larger small molecule structures comprising one or more of such structures).

In a preferred aspect, the inventive compound is characterized by one or more (preferably all) of the following characteristics:
A is —NR²R³ or guanidinyl, the last optionally substituted with C₁₋₆-alkyl, wherein
   R² and R³ independently of each other are hydrogen, C₁₋₆-alkyl,
   C₁₋₆-alkylene-N(R¹¹)(R¹²), C₁₋₆-alkylene-CN, C₁₋₆-alkylene-OH,
   C₁₋₆-alkylene-C(O)—N(R¹¹)(R¹²), (Z¹)ₑ-R¹³, or —CO—R¹⁴, wherein
      R¹¹ and R¹² independently of each other are hydrogen or C₁₋₆-alkyl;
      Z¹ is C₁₋₆-alkylene;
      e is an integer selected from 0 or 1;
      R¹³ is cycloalkyl, heterocyclyl, aryl, or heteroaryl; each of which may be optionally substituted with a substitutent selected from the group consisting of C₁₋₆-alkyl, amino, and —CO—O-Z⁴-R²³, wherein
         Z⁴ is C₁₋₆-alkylene; and
         R²³ is aryl; and
      R¹⁴ is hydrogen, C₁₋₆-alkyl, —N(R¹⁵)(R¹⁶), C₁₋₆-alkylene-N(R⁵)(R¹⁶),
      C(R¹⁷)(R¹⁸)—N(R¹⁹)(R²⁰), heterocyclyl, (Z²)_{f}-R²¹, heteroaryl, or C₁₋₆-alkoxy, wherein
         R¹⁵ and R¹⁶ independently of each other are hydrogen, or C₁₋₆-alkyl;
         R¹⁷ and R¹⁸ independently of each other are hydrogen,
         C₁₋₆-alkylene-NH₂ or (Z³)_{g}-R²²), wherein
            Z³ is C₁₋₆-alkylene;
            g is an integer selected from 0 or 1; and
            R²² is cycloalkyl, heterocyclyl, aryl or heteroaryl;
         R¹⁹ and R²⁰ independently of each other are hydrogen,
         C₂₋₆-alkylene-NH₂, C₁₋₆-alkylene-CF₃ or cycloalkyl; and
         Z² is C₁₋₆-alkylene;
         f is an integer selected from 0 or 1; and
         R²¹ is cycloalkyl, heterocyclyl, aryl or heteroaryl;
a is an integer selected from 1, 2, 3, 4, or 5;
E is cycloalkyl, heterocyclyl, aryl or heteroaryl; each of which may be optionally substituted with one or more substituents selected from the group consisting of halogen, hydroxy, cyano, nitro, —NR⁴R⁵, —CO—R⁶, C₁₋₆-alkyl, C₁₋₆-alkoxy, trifluoromethyl, trifluoromethoxy, and -L¹-Q¹, wherein
   R⁴ and R⁵ independently of each other are hydrogen, C₁₋₆-alkyl, —CO—R²⁴, or aryl, wherein
      R²⁴ is hydrogen, C₁₋₆-alkyl or C₁₋₆-alkoxy;
   R⁶ is C₁₋₆-alkyl or C₁₋₆-alkoxy;
   L¹ is a direct bond, —CH₂—, —O—, —CO—, —CH₂—O—, —O—CH₂— or —NR²⁵—, wherein
      R²⁵ is hydrogen or C₁₋₆-alkyl; and
   Q¹ is cycloalkyl, heterocyclyl, aryl or heteroaryl; each of which may be optionally substituted with one or more substituents selected from the group consisting of halogen, hydroxy, cyano, nitro, trifluoromethyl, trifluoromethoxy, —NR², R²⁷, —CO—R²⁸, —S(O)₂—R²⁹, C₁₋₆-alkyl, C₁₋₆-alkoxy, C₃₋₇-cycloalkyl and C₃₋₇-cycloalkoxy, wherein
      R²⁶ and R²⁷ independently of each other are hydrogen, C₁₋₆-alkyl, or —CO—R³⁰, wherein
         R³⁰ is hydrogen, C₁₋₆-alkyl or C₁₋₆-alkoxy;
      R²⁸ is C₁₋₆-alkyl or C₁₋₆-alkoxy; and
      R²⁹ is C₁₋₆-alkyl, —NH—C₁₋₆-alkyl, or —N(C₁₋₆-alkyl)₂;
   or
   Q¹ is L³-R³¹, wherein
      L³ is —CH₂—, —O—, —CO—, —CH₂—O—, —O—CH₂—, —CH₂—O—C(O)—, or —C(O)—O—CH₂—; and
      R³¹ is aryl or heteroaryl;
b is an integer selected from 0, 1, or 2;
G¹ is C₁₋₆-alkyl, C₁₋₆-alkoxy, cycloalkyl, C₃₋₇-cycloalkoxy, aryl or heteroaryl; each of which may be optionally substituted with halogen, hydroxy, cyano, nitro, trifluoromethyl, trifluoromethoxy, —NR⁷R⁸, C₁₋₆-alkyl, C₁₋₆-alkoxy, C₃₋₇-cycloalkyl, C₃₋₇-cycloalkoxy, wherein
   R⁷ and R⁸ independently of each other are hydrogen, C₁₋₆-alkyl, aryl, heteroaryl,
   —CO—R³² or —SO₂—R³³, wherein
      R³² is hydrogen, C₁₋₆-alkyl or C₁₋₆-alkoxy; and
      R³³ is C₁₋₆-alkyl, —NH—C₁₋₆-alkyl, —N(C₁₋₆-alkyl)₂;
G² is cycloalkyl, heterocyclyl, aryl, or heteroaryl; each of which may be optionally substituted with one or more substituents selected from the group consisting of halogen, hydroxy, cyano, nitro, difluoromethyl, trifluoromethyl, difluoromethoxy, trifluoromethoxy, —NR⁹R¹⁰, C₁₋₆-alkyl, C₁₋₆-alkoxy, C₃₋₇-cycloalkyl, C₃₋₇-cycloalkoxy or -L²-Q², wherein
   R⁹ and R¹⁰ are independently hydrogen, C₁₋₆-alkyl, aryl, heteroaryl, —CO—R³⁴ or
   —SO₂—R³⁵, wherein
      R³⁴ is hydrogen, C₁₋₆-alkyl or C₁₋₆-alkoxy; and
      R³⁵ is C₁₋₆-alkyl, —NH—C₁₋₆-alkyl, or —N(C₁₋₆-alkyl)₂;
   L² is a direct bond, —CH₂—, —O—, —CO—, —CH₂—O—, —O—CH₂— or —NR³⁶—, wherein
      R³⁶ is hydrogen or C₁₋₆-alkyl; and
   Q² is cycloalkyl, heterocyclyl, aryl or heteroaryl; each of which may be optionally substituted with halogen, hydroxy, cyano, nitro, trifluoromethyl, —NR³⁷R³⁸, —CO—R³⁹, —O—R⁴⁰, C₁₋₆-alkyl, C₁₋₆-hydroxyalkyl, C₃₋₇-cycloalkyl or C₃₋₇-cycloalkoxy, wherein
      R³⁷ and R³⁸ independently of each other are hydrogen, C₁₋₆-alkyl or —CO—R⁴¹, wherein
         R⁴¹ is hydrogen, C₁₋₆-alkyl or C₁₋₆-alkoxy;
      R³⁹ is hydrogen, C₁₋₆-alkyl or C₁₋₆-alkoxy; and
      R⁴⁰ is C₁₋₆-alkyl or trifluoromethyl;
c is an integer selected from 0, 1, or 2;
d is an integer selected from 0, or 1; and
R¹ is hydrogen, alkyl, alkenyl, or alkynyl.

The present invention also relates to compositions, including pharmaceutically acceptable compositions, containing compounds according to the present invention.

Another feature of the invention is the use of such compounds and compositions in the induction, enhancement, and/or promotion of physiological responses associated with binding and/or activating melanocortin receptors in a subject. In one exemplary aspect of this feature, the invention provides methods of administering compounds according to the present invention for the induction, promotion, and/or enhancement appetite regulation and/or for treating obesity and/or one or more obesity-related diseases including, without limitation, atherosclerosis, hypertension, diabetes, especially type 2 diabetes (NIDDM (non-insulin dependent diabetes mellitus)), impaired glucose tolerance, dyslipidaemia, coronary heart disease, gallbladder disease, osteoarthritis and various types of cancer such as endometrial, breast, prostate, and colon cancers and reducing the risk for premature death as well as promoting the treatment, reducing the severity, and/or preventing the onset of other conditions, such as diseases and disorders, which conditions are improved by activation and/or binding of melanocortin receptors, such as the MC4 receptor.

Additional aspects of the invention relate to use of compounds according to the present invention for preparing medicaments for increasing skin pigmentation, for protecting the skin against ultraviolet radiation (UVR) and for inhibiting the effects of UVR, for protecting the skin against local skin irritants (e.g. bacterial lipopolysaccharide), for modulating the inflammatory responses in the skin, for functionally antagonising the actions of proinflammatory cytokines produced in the skin after a local irritation, for regulating the immune response, for preventing contact dermatitis, and for inhibiting chronic inflammatory responses.

Further aspects of the invention relate to use of compounds according to the present invention for regulating glucocorticoid production, reducing blood pressure and heart rate, and for inducing natriuresis. Other aspects relate to use of compounds according to the present invention for regulating exocrine gland secretion, for regulating aldosterone secretion and thereby regulating blood pressure and natriuresis, for suppressing stress-induced alarm substances, and for stimulating exocrine glands, cardiac and testicular functions. The present invention also include aspects that relate to use of compounds according to the present invention for treating sexual dysfunction, increasing antipyretic activity, inducing lipolysis, and/or treating chronic pain.

### DESCRIPTION OF THE FIGURE

FIG. 1: Effect on food intake in schedule fed (8 h-13 h) male SPRD rat model as described in assay 1. The rats are dosed ip at 08.00 h with vehicle, sibutramine (3 mg/kg) and (S,S)-6-(4-amino-butyl)-1-biphenyl-4-ylmethyl-3-naphthalen-2-ylmethyl-piperazine-2,5-dione (example 11) (1, 3 or 10 mg/kg). Rat chow and water is available from just post dosing, and food intake is measured every hour from dosing to 3 hours post dosing.

The bars indicate the cumulated food intake over time.

### DETAILED DESCRIPTION OF THE INVENTION

To aid in understanding the invention described herein, the intended definitions of certain terms are provided here.

The term obesity implies an excess of adipose tissue. In this context obesity is best viewed as any degree of excess adiposity that imparts a health risk. The distinction between normal and obese individuals can only be approximated, but the health risk imparted by obesity is probably a continuum with increasing adiposity. However, in the context of the present invention, individuals with a body mass index (BMI=body weight in kilograms divided by the square of the height in meters) above 25 are to be regarded as obese. When energy intake exceeds energy expenditure, the excess calories are stored in adipose tissue, and if this net positive balance is prolonged, obesity results, i.e. there are two components to weight balance, and an abnormality on either side (intake or expenditure) can lead to obesity.

“Halogen” designates an atom selected from the group consisting of F, Cl, Br or I.

The use of prefixes of this structure: C_{x-y}-alkyl, C_{x-y}-alkenyl, C_{x-y}-alkynyl, C_{x-y}-cycloalyl or C_{x-y}-cycloalkyl-C_{x-y}-alkenyl- designates radical of the designated type having from x to y carbon atoms.

The term “alkyl” as used herein, alone or in combination, refers to a straight or branched chain saturated monovalent hydrocarbon radical having from one to ten carbon atoms, for example C₁₋₈-alkyl. Typical C₁₋₈-alkyl groups include, but are not limited to e.g. methyl, ethyl, n-propyl, isopropyl, n-butyl, sec-butyl, isobutyl, tert-butyl, n-pentyl, 2-methylbutyl, 3-methylbutyl, 4-methylpentyl, neopentyl, n-pentyl, n-hexyl, 1,2-dimethylpropyl, 2,2-dimethylpropyl, 1,2,2-trimethylpropyl and the like. The term “C₁₋₈-alkyl” as used herein also includes secondary C₃₋₈-alkyl and tertiary C₄₋₈-alkyl.

The term “C₁₋₆-alkyl” as used herein, alone or in combination, represents a straight or branched chain saturated monovalent hydrocarbon radical containing from 1 to 6 carbons atoms. Representative examples for “C₁₋₆-alkyl” include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like. Similarly the term “C₁₋₁₈-alkyl” represent a straight or branched carbon chain containing from 1 to 18 carbons atoms.

The term “alkylene” as used herein, alone or in combination, refers to a straight or branched chain saturated divalent hydrocarbon radical having from one to ten carbon atoms, for example C₁₋₈-alkylene. Examples of “alkylene” as used herein include, but are not limited to, methylene, ethylene, and the like.

The term “alkoxy” as used herein, alone or in combination, refers to the monovalent radical R^{a}O—, where R^{a} is alkyl as defined above, for example C₁₋₈-alkyl giving C₁₋₈-alkoxy. Typical C₁₋₈-alkoxy groups include, but are not limited to, methoxy, ethoxy, n-propoxy, isopropoxy, butoxy, sec-butoxy, tert-butoxy, pentoxy, isopentoxy, hexoxy, isohexoxy and the like.

The term “C₁₋₆-alkoxy” as used herein, alone or in combination, refers to the monovalent radical C₁₋₆-alkyl-O—, where C₁₋₆-alkyl is as defined above. Representative examples are methoxy, ethoxy, n-propoxy, isopropoxy, butoxy, sec-butoxy, tert-butoxy, pentoxy, isopentoxy, hexoxy, isohexoxy and the like.

The term “cycloalkyl” as used herein, alone or in combination, refers to a non-aromatic monovalent hydrocarbon radical having from three to twelve carbon atoms, and optionally with one or more degrees of unsaturation, for example C₃₋₈-cycloalkyl. Such a ring may be optionally fused to one or more benzene rings or to one or more of other cycloalkyl ring(s). Typical C₃₋₈-cycloalkyl groups include, but are not limited to, cyclopropyl, cyclobutyl, cyclopentyl, cyclohexyl, cyclohexenyl, cycloheptyl, cycloheptenyl, cyclooctyl and the like.

The term “C₃₋₆-cycloalkyl” as used herein, alone or in combination, refers to a non-aromatic monovalent hydrocarbon radical having from 3 to 6 carbon atoms. Representative examples are cyclopropyl, cyclobutyl, cyclopentyl, cyclohexyl and the like.

The term “heterocyclic” or the term “heterocyclyl” as used herein, alone or in combination, refers to a heterocyclic ring with for instance three to thirteen member atoms, for example C₃₋₁₀-heterocyclyl, such as C₃₋₈-heterocyclyl, having one or more degrees of unsaturation containing one or more heteroatomic substitutions selected from S, SO, SO₂, O, or N, for example selected from N, O, or S. Such a ring may be optionally fused to one or more of another “heterocyclic” ring(s) or cycloalkyl ring(s). Representative examples of C₃₋₁₀-heterocyclyl or C₃₋₈-heterocyclyl groups include, but are not limited to, pyrrolidinyl, piperidyl, piperazinyl, morpholinyl, thiomorpholinyl, aziridinyl, tetrahydrofuranyl and the like.

The term “aryl” as used herein, alone or in combination, refers to a carbocyclic aromatic ring radical or to a aromatic ring system radical with for instance six to thirteen member atoms, such as phenyl, biphenyl, naphthyl, anthracenyl, phenanthrenyl, fluorenyl, indenyl, pentalenyl, azulenyl, biphenylenyl, 5H-dibenzo[a,d]cyclohepten-5-yl, 10,11-dihydro-5H-dibenzo[a,d]cyclohepten-5-yl and the like. Aryl is also intended to include the partially hydrogenated derivatives of the carbocyclic systems enumerated above. Non-limiting examples of such partially hydrogenated derivatives are 1,2,3,4-tetrahydronaphthyl, 1,4-dihydronaphthyl and the like.

The term “aryloxy” as used herein denotes a group aryl-O—, wherein aryl is as defined above.

The term “heteroaryl”, as used herein, alone or in combination, refers to an aromatic ring radical with for instance 5 to 7 member atoms, or to an aromatic ring system radical with for instance from 7 to 18 member atoms, containing one or more heteroatoms selected from nitrogen, oxygen, or sulfur heteroatoms, wherein N-oxides and sulfur monoxides and sulfur dioxides are permissible heteroaromatic substitutions; such as e.g. furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5- triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, naphtothienyl, indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl, dibenzo[b,f]azepin-5-yl, 10,11-dihydro-dibenzo[b,f]azepin-5-yl and the like. Heteroaryl is also intended to include the partially hydrogenated derivatives of the heterocyclic systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like.

The term “hydroxyalkyl” as used herein, alone or in combination, represents an alkyl radical as described above, such as a C₁₋₆-alkyl, substituted with one or more hydroxy radicals. Examples of C₁₋₆-hydroxyalkyl radicals are 2-hydroxymethyl, 2-hydroxyethyl, 2-hydroxypropyl, 3-hydroxypropyl, 2,3-dihydroxypropyl and the like.

The term “optionally substituted” as used herein means that the groups in question are either unsubstituted or substituted with one or more of the substituents specified. When the groups in question are substituted with more than one substituent the substituents may be the same or different.

Certain of the above defined terms may occur more than once in the structural formulae, and upon such occurrence each term shall be defined independently of the other.

“Selective” or “selectivity” towards the MC1 receptor, when used herein with regard to a compound of the present invention being an agonist of said receptor, means that the compound does not bind or activate the other MC receptors, that is MC2, MC3, MC4 and MC5. Likewise, a compound being a selective agonist of the MC2 receptor means that the compound does not bind or activate the other MC receptors, that is MC1, MC3, MC4 and MC5. Likewise, a compound being a selective agonist of the MC3 receptor means that the compound does not bind or activate the other MC receptors, that is MC1, MC2, MC4 and MC5. Likewise, a compound being a selective agonist of the MC4 receptor means that the compound does not bind or activate the other MC receptors, that is MC1, MC2, MC3 and MC5. Likewise, a compound being a selective agonist of the MC5 receptor means that the compound does not bind or activate the other MC receptors, that is MC1, MC2, MC3 and MC4. A compound according to the present invention may also be said to be selective for two receptors, such as for instance the MC3 and MC4 receptor, meaning that the compound does not bind or activate the other MC receptors, in this case MC1, MC2, and MC5.

A “therapeutically effective amount” of a compound according to the present invention as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being.

The following is a list of abbreviations used in the description of the invention:
aq. aqueous
Boc tert-butyloxycarbonyl
Cbz benzyloxycarbonyl
CDI N,N′-carbonyldiimidazole
conc. concentrated
DCM dichloromethane
DIC N,N′-diisopropylcarbodiimide
DIPEA N,N-diisopropyl-ethyl-amine
DMF N,N-Dimethylformamide
equi equivalent
FMOC/fmoc 9-fluorenylmethyloxycarbonyl
h hour/hours
HOBt 1-hydroxybenzotriazole monohydrate
i No. intermediate number
LCMS liquid chromatography coupled with mass spectrometry
min. minutes
NMP N-methylpyrrolidone
TBDPS tert-butyl diphenylsilyl
TBTU 2-(1H-benzotriazol-1-yl)-1,1,3,3-tetramethyluronium tetrafluoroborate
TFA trifluoroacetic acid
TBDPS tert-butyl diphenylsilyl
THF tetrahydrofurane
org. organic
Rt or Rₜ retention time
RT room temperature
sat. saturated

As described elsewhere herein, the present invention relates to, among other things, novel compounds, pharmaceutical compositions containing the novel compounds, use of these compounds for preparing medicaments for appetite regulation or for treating obesity and obesity related diseases as well as to a method for treatment of obesity and, consequently, for the treatment of obesity related diseases and conditions such as atherosclerosis, hypertension, diabetes, especially type 2 diabetes (NIDDM (non-insulin dependent diabetes mellitus)), impaired glucose tolerance (IGT), dyslipidaemia, coronary heart disease, gallbladder disease, osteoarthritis and various types of cancer such as endometrial, breast, prostate and colon cancers and the risk for premature death as well as other conditions, such as diseases and disorders, which conditions are improved by activation of the melanocortin receptors. These and other aspects and features of the invention will be described in detail below.

Among other advantages, compounds according to the invention described herein can be effective in appetite regulation and/or in the treatment of obesity or obesity related diseases such as atherosclerosis, hypertension, diabetes, especially type 2 diabetes (NIDDM (non-insulin dependent diabetes mellitus)), dyslipidaemia, coronary heart disease, gallbladder disease, osteoarthritis and various types of cancer such as endometrial, breast, prostate and colon cancers and the risk for premature death as well as other conditions, such as diseases and disorders, which conditions are improved by activation of the MC4 receptor. Pharmaceutical compositions comprising the novel compounds of the invention also advantageously can be effective in the prevention or treatment of obesity or obesity related diseases as described herein as well as other conditions, such as diseases and disorders, which conditions are improved by binding and/or activation of the MC4 receptor.

As described above, in one aspect, the invention relates to compounds according to formula (I) 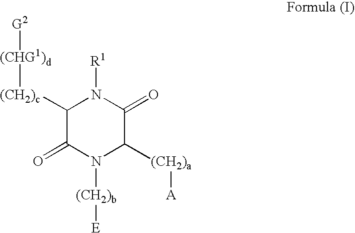
wherein
A is —NR²R³ or guanidinyl, the last optionally substituted with C₁₋₆-alkyl, wherein
   R² and R³ independently of each other are hydrogen, C₁₋₆-alkyl,
   C₁₋₆-alkylene-N(R¹¹)(R¹²), C₁₋₆-alkylene-CN, C₁₋₆-alkylene-OH,
   C₁₋₆-alkylene-C(O)—N(R¹¹)(R¹²), (Z¹)ₑ-R¹³, or —CO—R¹⁴, wherein
      R¹¹ and R¹² independently of each other are hydrogen or C₁₋₆-alkyl;
      Z¹ is C₁₋₆-alkylene;
      e is an integer selected from 0 or 1;
      R¹³ is cycloalkyl, heterocyclyl, aryl, or heteroaryl; each of which may be optionally substituted with a substitutent selected from the group consisting of C₁₋₆-alkyl, amino, and —CO—O-Z⁴-R²³, wherein
         Z⁴ is C₁₋₆-alkylene; and
         R²³ is aryl; and
      R¹⁴ is hydrogen, C₁₋₆-alkyl, —N(R¹⁵)(R¹⁶), C₁₋₆-alkylene-N(R¹⁵)(R¹⁶), C(R¹⁷)(R¹⁸)—N(R¹⁹)(R²⁰), heterocyclyl, (Z²)_{f}-R²¹, heteroaryl, or C₁₋₆-alkoxy, wherein
         R¹⁵ and R¹⁶ independently of each other are hydrogen, or C₁₋₆-alkyl;
         R¹⁷ and R¹⁸ independently of each other are hydrogen,
         C₁₋₆-alkylene-NH₂ or (Z³)_{g}-R²²), wherein
            Z³ is C₁₋₆-alkylene;
            g is an integer selected from 0 or 1; and
            R²² is cycloalkyl, heterocyclyl, aryl or heteroaryl;
         R¹⁹ and R²⁰ independently of each other are hydrogen,
         C₂₋₆-alkylene-NH₂, C₁₋₆-alkylene-CF₃ or cycloalkyl; and
         Z² is C₁₋₆-alkylene;
         f is an integer selected from 0 or 1; and
         R²¹ is cycloalkyl, heterocyclyl, aryl or heteroaryl;
a is an integer selected from 1, 2, 3, 4, or 5;
E is cycloalkyl, heterocyclyl, aryl or heteroaryl; each of which may be optionally substituted with one or more substituents selected from the group consisting of halogen, hydroxy, cyano, nitro, —NR⁴R⁵, —CO—R⁶, C₁₋₈-alkyl, C₁₋₆-alkoxy, trifluoromethyl, trifluoromethoxy, and -L¹-Q¹, wherein
   R⁴ and R⁵ independently of each other are hydrogen, C₁₋₆-alkyl, —CO—R²⁴, or aryl, wherein
      R²⁴ is hydrogen, C₁₋₆-alkyl or C₁₋₆-alkoxy;
   R⁶ is C₁₋₆-alkyl or C₁₋₆-alkoxy;
   L¹ is a direct bond, —CH₂—, —O—, —CO—, —CH₂—O—, —O—CH₂— or —NR²⁵—, wherein
      R²⁵ is hydrogen or C₁₋₆-alkyl; and
   Q¹ is cycloalkyl, heterocyclyl, aryl or heteroaryl; each of which may be optionally substituted with one or more substituents selected from the group consisting of halogen, hydroxy, cyano, nitro, trifluoromethyl, trifluoromethoxy, —NR²⁶R²⁷, —CO—R²⁸, —S(O)₂—R²⁹, C₁₋₆-alkyl, C₁₋₆-alkoxy, C₃₋₇-cycloalkyl and C₃₋₇-cycloalkoxy, wherein
      R²⁶ and R²⁷ independently of each other are hydrogen, C₁₋₆-alkyl, or —CO—R³⁰, wherein
         R³⁰ is hydrogen, C₁₋₆-alkyl or C₁₋₆-alkoxy;
      R²⁸ is C₁₋₆-alkyl or C₁₋₆-alkoxy; and
      R²⁹ is C₁₋₆-alkyl, —NH—C₁₋₆-alkyl, or —N(C₁₋₆-alkyl)₂;
   or
   Q¹ is L³-R³¹, wherein
      L³ is —CH₂—, —O—, —CO—, —CH₂—O—, —O—CH₂—, —CH₂—O—C(O)—, or —C(O)—O—CH₂—; and
      R³¹ is aryl or heteroaryl;
b is an integer selected from 0, 1, or 2;
G¹ is C₁₋₆-alkyl, C₁₋₆-alkoxy, cycloalkyl, C₃₋₇-cycloalkoxy, aryl or heteroaryl; each of which may be optionally substituted with halogen, hydroxy, cyano, nitro, trifluoromethyl, trifluoromethoxy, —NR⁷R⁸, C₁₋₆-alkyl, C₁₋₆-alkoxy, C₃₋₇-cycloalkyl, C₃₋₇-cycloalkoxy, wherein
   R⁷ and R⁸ independently of each other are hydrogen, C₁₋₆-alkyl, aryl, heteroaryl,
   —CO—R³² or —SO₂—R³³, wherein
      R³² is hydrogen, C₁₋₆-alkyl or C₁₋₆-alkoxy; and
      R³³ is C₁₋₆-alkyl, —NH—C₁₋₆-alkyl, —N(C₁₋₆-alkyl)₂;
G² is cycloalkyl, heterocyclyl, aryl, or heteroaryl; each of which may be optionally substituted with one or more substituents selected from the group consisting of halogen, hydroxy, cyano, nitro, difluoromethyl, trifluoromethyl, difluoromethoxy, trifluoromethoxy, —NR⁹R¹⁰, C₁₋₆-alkyl, C₁₋₆-alkoxy, C₃₋₇-cycloalkyl, C₃₋₇-cycloalkoxy or -L²-Q², wherein
   R⁹ and R¹⁰ are independently hydrogen, C₁₋₆-alkyl, aryl, heteroaryl, —CO—R³⁴ or
   —SO₂—R³⁵, wherein
      R³⁴ is hydrogen, C₁₋₆-alkyl or C₁₋₆-alkoxy; and
      R³⁵ is C₁₋₆-alkyl, —NH—C₁₋₆-alkyl, or —N(C₁₋₆-alkyl)₂;
   L² is a direct bond, —CH₂—, —O—, —CO—, —CH₂—O—, —O—CH₂— or —NR³⁶—, wherein
      R³⁶ is hydrogen or C₁₋₆-alkyl; and
   Q²is cycloalkyl, heterocyclyl, aryl or heteroaryl; each of which may be optionally substituted with halogen, hydroxy, cyano, nitro, trifluoromethyl, —NR³⁷R³⁸, —CO—R³⁹, —O—R⁴⁰, C₁₋₆-alkyl, C₁₋₆-hydroxyalkyl, C₃₋₇-cycloalkyl or C₃₋₇-cycloalkoxy, wherein
      R³⁷ and R³⁸ independently of each other are hydrogen, C₁₋₆-alkyl or —CO—R⁴¹, wherein
         R⁴¹ is hydrogen, C₁₋₆-alkyl or C₁₋₆-alkoxy;
      R³⁹ is hydrogen, C₁₋₆-alkyl or C₁₋₆-alkoxy; and
      R⁴⁰ is C₁₋₆-alkyl or trifluoromethyl;
c is an integer selected from 0, 1, or 2;
d is an integer selected from 0, or 1; and
R¹ is hydrogen, alkyl, alkenyl, or alkynyl;
   as well as any optical or geometric isomer or tautomer form thereof,
   or a pharmaceutically acceptable salt thereof.

Further aspects of the compounds of the present invention are clear from the exemplary aspects of the invention provided below and other sections of the description of the invention.

The compounds of the present invention may have one or more asymmetric centres and it is intended that stereoisomers (optical isomers), as separated, pure or partially purified stereoisomers or racemic mixtures thereof are included in the scope of the invention. Diastereomers, enantiomers, and tautomeric forms of compounds of general formula (I) including mixtures of these or pharmaceutically acceptable salts thereof also are provided by the invention.

Compounds of the invention are useful in a number of respects. One feature of the compounds of the invention is the ability to bind and preferably to activate one or more melanocortin receptors. For example, in one aspect, the invention provides a compound comprising, consisting essentially of, or having a structure of Formula (I), which is an agonist of a melanocortin receptor, such as the MC4 receptor. In a further aspect, the compound is selective for the MC4 receptor. In one aspect, the present invention relates to the use of a compound according to the present invention for increasing the activity of the MC4 receptor. In one aspect, the present invention relates to the use of a compound according to the present invention for treating a condition which is improved by the activation of the MC4 receptor. In another aspect, the present invention relates to the use of a compound according to the present invention for the preparation of a medicament for treating a condition which is improved by the activation of the MC4 receptor.

In one aspect, the present invention relates to a pharmaceutical composition comprising, as an active ingredient, at least one compound according to the present invention together with one or more pharmaceutically acceptable carriers or excipients. In one aspect, the present invention relates to a pharmaceutical composition comprising, as an active ingredient, at least one compound according to the present invention together with one or more pharmaceutically acceptable carriers, diluents or excipients in unit dosage form, comprising from about 0.05 mg to about 1000 mg, such as about 0.1 mg to about 500 mg, for example from about 0.5 mg to about 200 mg of a compound according to the present invention.

Compounds and compositions of the invention also are useful in promoting, enhancing, and/or inducing the treatment of disease and/or in preventing disease in a subject. In one exemplary aspect, the present invention relates to the use of a compound according to the present invention for the delaying or prevention of the progression from IGT to type 2 diabetes. In another exemplary aspect, the present invention relates to the use of a compound according to the present invention for the preparation of a medicament for the delaying or prevention of the progression from IGT to type 2 diabetes. In a further exemplary aspect, the present invention relates to the use of a compound according to the present invention for the delaying or prevention of the progression from non-insulin requiring type 2 diabetes to insulin requiring type 2 diabetes. In yet another aspect, the present invention relates to the use of a compound according to the present invention for the preparation of a medicament for the delaying or prevention of the progression from non-insulin requiring type 2 diabetes to insulin requiring type 2 diabetes. In still another aspect, the present invention relates to the use of a compound according to the present invention for appetite regulation. In a further aspect, the present invention relates to the use of a compound according to the present invention for treating a condition related to overweight or obesity. The invention also relates to the use of a compound according to the present invention for the preparation of a medicament for appetite regulation. In one aspect, the present invention provides a method of using a compound according to the present invention for the preparation of a medicament for treating a condition related to overweight or obesity.

In one aspect, the present invention relates to the use of a compound according to the present invention for treating a disease or condition selected from overweight or obesity, atherosclerosis, hypertension, diabetes, type 2 diabetes, impaired glucose tolerance, dyslipidaemia, coronary heart disease, gallbladder disease, osteoarthritis, cancer, sexual dysfunction and the risk for premature death in a patient in need thereof.

In one aspect, the present invention relates to the use of a compound according to the present invention for the preparation of a medicament for treating a disease or condition selected from overweight or obesity, atherosclerosis, hypertension, diabetes, type 2 diabetes, impaired glucose tolerance, dyslipidaemia, coronary heart disease, gallbladder disease, osteoarthritis, cancer, sexual dysfunction and the risk for premature death in a patient in need thereof.

Compounds of the invention can be used to promote, induce, and/or enhance the treatment of obesity (or being overweight), type 2 diabetes (for instance in obese patients), treating dyslipidemia (for instance in obese patients), sexual dysfunction. Compounds of the invention also can be used in a method of reducing the weight of a subject and/or the suppression of appetite or for satiety induction.

In another aspect, the present invention relates to the use of a compound according to the present invention for treatment of eating disorders such as bulimia and binge eating.

In a particular aspect, the invention relates to a method for the treatment of disorders related to melanocortin MC4 receptor, the method comprising administering to a subject in need thereof an effective amount of a compound according to formula (I)

In a still further aspect, the invention relates to a method for the treatment of obesity, the method comprising administering to a subject in need thereof an effective amount of a compound according to formula (I) or a pharmaceutically acceptable salt thereof, or of a composition according to any one of the preceding composition claims.

In still another aspect, the invention relates to a method for the treatment of diabetes, preferably type 2 diabetes, the method comprising administering to a subject in need thereof an effective amount of a compound according to formula (I) or a pharmaceutically acceptable salt thereof, or of a composition according to any one of the preceding composition claims.

In still another aspect, the invention relates to the use of a compound according to formula (I) or a pharmaceutically acceptable salt thereof for the preparation of a medicament.

Furthermore the invention relates to the use of a compound according to formula (I) or a pharmaceutically acceptable salt thereof for the preparation of a medicament for the treatment of disorders related to melanocortin MC4 receptor.

More particularly, the invention relates to the use of a compound according formula (I) or a pharmaceutically acceptable salt thereof for the preparation of a medicament having melanocortin MC4 agonist activity.

The invention relates furthermore to the use of a compound according to formula (I) or a pharmaceutically acceptable salt thereof for the preparation of a medicament for the treatment of obesity.

The invention relates furthermore to the use of a compound according to formula (I) or a pharmaceutically acceptable salt thereof for the preparation of a medicament for the treatment of diabetes preferably type 2 diabetes. Such treatment includes inter alia treatment for the purpose of delaying or prevention of the progression from IGT to type 2 diabetes as well as delaying or prevention of the progression from non-insulin requiring type 2 diabetes to insulin requiring type 2 diabetes.

The invention relates furthermore to the use of a compound according to formula (I) or a pharmaceutically acceptable salt thereof for the preparation of a medicament for the treatment of obesity and complications related to obesity and excessive food consumption and other eating disorders and specific complications related to obesity, such as hypertension, dyslipidemia, diabetes mellitus, coronary heart disease, congestive heart failure, stroke, gallstones, osteoarthritis, sleep apnea, cancer, women's health/reproduction (within this area is noted psychopathology of obesity, such as body image and binge eating).

Further more the compounds according to formula (I) may be useful for stimulation of melanin-production, skin darkening, inflammation, body temperature, pain perception, neuropathy, blood pressure, heart rate, vascular tone, natruresis, brain blood flow, nerve growth, placental development, aldosteron synthesis and release, thyroxin release, spermatogenesis, ovarian weight, prolactin, growth hormone and FSH secretion, uterine bleeding in women, sebum and pheromone secretion, blood glucose levels, intrauterine foetal growth, as well as other related to parturition, and to afford neuroprotective effects and for the treatment of eating disorders, improvement of libido activity in male and female and for stimulation of penile erection as well as psychiatric disorders, (such as depression, anxiety, motivational defects, cognitive disorders, memory loss and other psychiatric disorders as known by those skilled in the art), as well as addiction.

The present invention also provides pharmaceutical compositions comprising as an active ingredient, at least one compound, preferably in a pharmacologically effective amount, more preferably in a therapeutically effective amount, suitable for any of the uses according to the present invention together with one or more pharmaceutically acceptable carriers or excipients.

In a use or a method according to the present invention, a compound according to the present invention may also be administered in combination with one or more further active substances in any suitable ratios. Such further active agents may be selected from antidiabetic agents, antihyperlipidemic agents, antiobesity agents, antihypertensive agents and agents for the treatment of complications resulting from or associated with diabetes.

Suitable antidiabetic agents include insulin, GLP-1 (glucagon like peptide-1) derivatives such as those disclosed in WO 98/08871 (Novo Nordisk A/S), which is incorporated herein by reference, as well as orally active hypoglycemic agents.

Suitable orally active hypoglycemic agents include imidazolines, sulfonylureas, biguanides, meglitinides, oxadiazolidinediones, thiazolidinediones, insulin sensitizers, α-glucosidase inhibitors, agents acting on the ATP-dependent potassium channel of the pancreatic -cells eg potassium channel openers such as those disclosed in WO 97/26265, WO 99/03861 and WO 00/37474 (Novo Nordisk A/S) which are incorporated herein by reference, potassium channel openers, such as ormitiglinide, potassium channel blockers such as nateglinide or BTS-67582, glucagon antagonists such as those disclosed in WO 99/01423 and WO 00/39088 (Novo Nordisk A/S and Agouron Pharmaceuticals, Inc.), all of which are incorporated herein by reference, GLP-1 agonists such as those disclosed in WO 00/42026 (Novo Nordisk A/S and Agouron Pharmaceuticals, Inc.), which are incorporated herein by reference, DPP-IV (dipeptidyl peptidase-IV) inhibitors, PTPase (protein tyrosine phosphatase) inhibitors, glucokinase activators, such as those described in WO 02/08209 to Hoffmann La Roche, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antihyperlipidemic agents and antilipidemic agents, compounds lowering food intake, and PPAR (peroxisome proliferator-activated receptor) and RXR (retinoid X receptor) agonists such as ALRT-268, LG-1268 or LG-1069.

In one aspect of the uses and methods of the present invention, the compound involved may be administered in combination with insulin or insulin analogues.

In one aspect of the uses and methods of the present invention, the compound involved may be administered in combination with a sulphonylurea eg tolbutamide, chlorpropamide, tolazamide, glibenclamide, glipizide, glimepiride, glicazide or glyburide.

In one aspect of the uses and methods of the present invention, the compound involved may be administered in combination with a biguanide eg metformin.

In one aspect of the uses and methods of the present invention, the compound involved may be administered in combination with a meglitinide eg repaglinide or senaglinide/nateglinide.

In one aspect of the uses and methods of the present invention, the compound involved may be administered in combination with a thiazolidinedione insulin sensitizer eg troglitazone, ciglitazone, pioglitazone, rosiglitazone, isaglitazone, darglitazone, englitazone, CS-011/CI-1037 or T 174 or the compounds disclosed in WO 97/41097 (DRF-2344), WO 97/41119, WO 97/41120, WO 00/41121 and WO 98/45292 (Dr. Reddy's Research Foundation), which are incorporated herein by reference.

In one aspect of the uses and methods of the present invention, the compound involved may be administered in combination with an insulin sensitizer eg such as GI 262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313 (NN622/DRF-2725), WO 00/50414, WO 00/63191, WO 00/63192, WO 00/63193 (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), which are incorporated herein by reference.

In one aspect of the uses and methods of the present invention, the compound involved may be administered in combination with an α-glucosidase inhibitor eg voglibose, emiglitate, miglitol or acarbose.

In one aspect of the uses and methods of the present invention, the compound involved may be administered in combination with a glycogen phosphorylase inhibitor eg the compounds described in WO 97/09040 (Novo Nordisk A/S).

In one aspect of the uses and methods of the present invention, the compound involved may be administered in combination with a glucokinase activator.

In one aspect of the uses and methods of the present invention, the compound involved may be administered in combination with an agent acting on the ATP-dependent potassium channel of the pancreatic β-cells eg tolbutamide, glibenclamide, glipizide, glicazide, BTS-67582 or repaglinide.

In one aspect of the uses and methods of the present invention, the compound involved may be administered in combination with nateglinide.

In one aspect of the uses and methods of the present invention, the compound involved may be administered in combination with an antihyperlipidemic agent or a antilipidemic agent eg cholestyramine, colestipol, clofibrate, gemfibrozil, lovastatin, pravastatin, simvastatin, probucol or dextrothyroxine.

In one aspect of the uses and methods of the present invention, the compound involved may be administered in combination with more than one of the above-mentioned compounds eg in combination with metformin and a sulphonylurea such as glyburide; a sulphonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

In one aspect of the uses and methods of the present invention, the compound involved may be administered in combination with one or more antiobesity agents or appetite regulating agents.

Such agents may be selected from the group consisting of CART (cocaine amphetamine regulated transcript) agonists, NPY (neuropeptide Y) antagonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin reuptake inhibitors (fluoxetine, seroxat or citalopram), serotonin and norepinephrine reuptake inhibitors, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti related protein) inhibitors, H3 histamine antagonists such as those disclosed in WO 00/42023, WO 00/63208 and WO 00/64884, which are incorporated herein by reference, exendin-4, GLP-1 agonists and ciliary neurotrophic factor. Further antiobesity agents are bupropion (antidepressant), topiramate (anticonvulsant), ecopipam (dopamine D1/D5 antagonist), naltrexone (opioid antagonist), and peptide YY₃₋₃₆ (Batterham et al, Nature 418, 650-654 (2002)).

In one aspect, the antiobesity agent is leptin.

In one aspect, the antiobesity agent is peptide YY₃₋₃₆.

In one aspect, the antiobesity agent is a serotonin and norepinephrine reuptake inhibitor eg sibutramine.

In one aspect, the antiobesity agent is a lipase inhibitor eg orlistat.

In one aspect, the antiobesity agent is an adrenergic CNS stimulating agent eg dexamphetamine, amphetamine, phentermine, mazindol phendimetrazine, diethylpropion, fenfluramine or dexfenfluramine.

Furthermore, in the uses and methods of the present invention, the compound involved may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19th Edition, Gennaro, Ed., Mack Publishing Co., Easton, Pa., 1995.

It should be understood that any suitable combination of the compounds according to the invention with diet and/or exercise, one or more of the above-mentioned compounds and optionally one or more other active substances are considered to be within the scope of the present invention.

Other aspects of the present invention are clear from the appended claims.

Pharmaceutical Compositions

In another aspect, the present invention includes within its scope pharmaceutical compositions comprising, as an active ingredient, at least one of the compounds of the general formula (I) or a pharmaceutically acceptable salt thereof together with a pharmaceutically acceptable carrier or diluent.

Optionally, the pharmaceutical composition of the invention may comprise a compound of formula (I) combined with one or more compounds.

Pharmaceutical compositions containing a compound of the present invention may be prepared by conventional techniques, e.g. as described in Remington: The Science and Practise of Pharmacy, 19^{th} Ed., 1995. The compositions may appear in conventional forms, for example capsules, tablets, aerosols, solutions, suspensions or topical applications.

Typical compositions include a compound of formula (I) or a pharmaceutically acceptable acid addition salt thereof, associated with a pharmaceutically acceptable excipient which may be a carrier or a diluent or be diluted by a carrier, or enclosed within a carrier which can be in the form of a capsule, sachet, paper or other container. In making the compositions, conventional techniques for the preparation of pharmaceutical compositions may be used. For example, the active compound will usually be mixed with a carrier, or diluted by a carrier, or enclosed within a carrier which may be in the form of a ampoule, capsule, sachet, paper, or other container. When the carrier serves as a diluent, it may be solid, semi-solid, or liquid material which acts as a vehicle, excipient, or medium for the active compound. The active compound can be adsorbed on a granular solid container for example in a sachet. Some examples of suitable carriers are water, salt solutions, alcohols, polyethylene glycols, polyhydroxyethoxylated castor oil, peanut oil, olive oil, gelatine, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or colouring substances and the like, which do not deleteriously react with the active compounds.

The route of administration may be any route, which effectively transports the active compound to the appropriate or desired site of action, such as oral, nasal, pulmonary, buccal, subdermal, intradermal, transdermal or parenteral e.g. rectal, depot, subcutaneous, intravenous, intraurethral, intramuscular, intranasal, ophthalmic solution or an ointment, the oral route being preferred.

If a solid carrier is used for oral administration, the preparation may be tabletted, placed in a hard gelatin capsule in powder or pellet form or it can be in the form of a troche or lozenge. If a liquid carrier is used, the preparation may be in the form of a syrup, emulsion, soft gelatin capsule or sterile injectable liquid such as an aqueous or non-aqueous liquid suspension or solution.

For nasal administration, the preparation may contain a compound of formula (I) dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet which may be prepared by conventional tabletting techniques may contain:

| Core: | | |
|---|---|---|
| Active compound (as free compound or salt thereof) | 250 | mg |
| Colloidal silicon dioxide (Aerosil) ® | 1.5 | mg |
| Cellulose, microcryst. (Avicel) ® | 70 | mg |
| Modified cellulose gum (Ac-Di-Sol) ® | 7.5 | mg |
| Magnesium stearate | Ad. | |
| Coating: | | |
| HPMC approx. | 9 | mg |
| *Mywacett 9-40 T approx. | 0.9 | mg |

*Acylated monoglyceride used as plasticizer for film coating.

The compounds of the invention may be administered to a mammal, especially a human in need of such treatment, such as prevention, elimination, alleviation or amelioration of obesity. Such mammals include also animals, both domestic animals, e.g. household pets, and non-domestic animals such as wildlife.

The compounds of the invention may be effective over a wide dosage range. For example, in the treatment of adult humans, dosages from about 0.05 to about 1000 mg, for example from about 0.1 to about 500 mg, such as from about 0.5 mg to about 250 mg per day may be used. In choosing a regimen for patients it may frequently be necessary to begin with a higher dosage and when the condition is under control to reduce the dosage. The exact dosage will depend upon the mode of administration, on the therapy desired, form in which administered, the subject to be treated and the body weight of the subject to be treated, and the preference and experience of the physician or veterinarian in charge. A dosage of for instance from 1 to 100 mg/kg body weight, for example 10 mg/kg body weight pr day may be used.

Generally, the compounds of the present invention are dispensed in unit dosage form comprising from about 0.05 to about 1000 mg of active ingredient together with a pharmaceutically acceptable carrier per unit dosage.

Usually, dosage forms suitable for oral, nasal, pulmonal or transdermal administration comprise from about 0.05 mg to about 1000 mg, such as from about 0.5 mg to about 250 mg of the compounds of formula (I) admixed with a pharmaceutically acceptable carrier or diluent.

Any novel feature or combination of features described herein is contemplated within the scope of this invention.

### EXAMPLES

HPLC-MS (Method A)

The following instrumentation is used:
SciexAPI 100 Single quadropole mass spectrometer
Perkin Elmer Series 200 Quard pump
Perkin Elmer Series 200 autosampler
Applied Biosystems 785A UV detector
Sedex55 evaporative light scattering detector
   Valco column switch with a Valco actuator controlled by timed events from the pump.

The instrument control and data acquisition are done by the SciexSample control software running on a Macintosh PowerPC 7200 computer.

The HPLC pump is connected to four eluent reservoirs containing:

A: acetonitrile

B: water

C: 0.5% TFA in water

D: 0.02 M ammonium acetate

The requirements for samples are that they contain approximately 500 μg/ml of the compound to be analysed in an acceptable solvent such as methanol, ethanol, acetonitrile, THF, water and mixtures thereof. (High concentrations of strongly eluting solvents will interfere with the chromatography at low acetonitrile concentration.)

The analysis is performed at room temperature by injecting 20 μl of the sample solution on the column which is eluted with a gradient of acetonitrile in either 0.05% TFA or 0.002 M ammonium acetate. Depending on the analysis method varying elution conditions are used.

The eluate from the column is passed through a flow splitting T-connector which passed approximately 20 μl/min (1/50) through approx. 1 m. 75 μm fused silica capillary to the API interface of API 100 spectrometer.

The remaining 1.48 ml/min (49/50) is passed through the UV detector and to the ELS detector.

During the LC-analysis the detection data are acquired concurrently from mass spectrometer, UV detector and ELS detector.

The LC conditions, detector settings and mass spectrometer settings used for the different methods are given in the following tables.

| Column | Waters Symmetry C₁₈ 3 mm × 150 mm | | | |
|---|---|---|---|---|
| Gradient | 5%-90% acetonitrile in 0.05% TFA linearly | | | |
| | during 15 min at 1 ml/min | | | |
| Detec- | UV: 214 nm | | ELS: 40° C. | |
| tion | | | | |
| MS | Experiment: | Start: 100 amu | Stop: | Step: |
| | | | 800 amu | 0.2 amu |
| | Dwell: | 0.571 msec | | |
| | Method: | Scan 284 times = | | |
| | | 9.5 min | | |

HPLC-MS (Method B)

This method is identical to HPLC-MS (Method A) but using the following conditions and settings:

| Column | YMC ODS-A 120 Å s - 5μ 50 mm × 3 mm id | | | |
|---|---|---|---|---|
| Gradient | 5%-90% acetonitrile in 0.05% TFA linearly | | | |
| | during 7.5 min at 1.5 ml/min | | | |
| Detec- | UV: 214 nm | | ELS: 40° C. | |
| tion | | | | |
| MS | Experiment: | Start: 100 amu | Stop: | Step: |
| | | | 800 amu | 0.2 amu |
| | Dwell: | 0.571 msec | | |
| | Method: | Scan 284 times = | | |
| | | 9.5 min | | |

HPLC-MS (Method C)

The following instrumentation is used:
Hewlett Packard series 1100 G1312A Bin Pump
Hewlett Packard series 1100 Column compartment
Hewlett Packard series 1100 G13 15A DAD diode array detector
Hewlett Packard series 1100 MSD

The instrument is controlled by HP Chemstation software.

The HPLC pump is connected to two eluent reservoirs containing:

A: 0.01% TFA in water

B: 0.01% TFA in acetonitrile

The analysis is performed at 40° C. by injecting an appropriate volume af the sample (preferably 1 μl) onto the column which is eluted with a gradient of acetonitrile.

The HPLC conditions, detector settings and mass spectrometer settings used are giving in the following table.

| | |
|---|---|
| Column | Waters Xterra MS C-18 × 3 mm id |
| Gradient | 10%-100% acetonitrile lineary during 7.5 min at 1.0 ml/min |
| Detection | 210 nm (analog output from DAD) |
| MS | ionisation mode API-ES |
| | Scan 100-1000 amu step 0.1 amu |

HPLC-MS (Method D)

The following instrumentation is used:
Hewlett Packard series 1100 G1312A Bin Pump
Hewlett Packard series 1100 G13 15A DAD diode array detector
Sciex3000 triplequadropole mass spectrometer
Gilson 215 micro injector
Sedex55 evaporative light scattering detector

Pumps and detectors are controlled by MassChrom 1.1.1 software running on a Macintosh G3 computer. Gilson Unipoint Version 1.90 controls the auto-injector.

The HPLC pump is connected to two eluent reservoirs containing:

A: 0.01% TFA in water

B: 0.01% TFA in acetonitrile

The analysis is performed at room temperature by injecting an appropriate volume of the sample (preferably 10 μl) onto the column, which is eluted, with a gradient of acetonitrile. The eluate from the column passed through the UV detector to meet a flow splitter, which passed approximately 30 μl/min ( 1/50) through to the API Turbo ion-spray interface of API 3000 spectrometer. The remaining 1.48 ml/min ( 49/50) is passed through to the ELS detector.

The HPLC conditions, detector settings and mass spectrometer settings used are giving in the following table.

| | |
|---|---|
| Column | Waters X-Terra C18, 5μ, 50 mm × 3 mm id |
| Gradient | 5%-90% acetonitrile linearly during 7.5 min at 1.5 ml/min |
| Detection | 210 nm (analogue output from DAD) |
| MS | ionisation mode API Turbo ion-spray |
| | Scan 100-1000 amu step 0.1 amu |
| ELS | Gain 8 and 40° C. |

General Procedures
General procedure (A)—Synthesis on Solid Phase, Suitable for the Synthesis of Multiple Examples in Parallel
Step A:

Add 9 equi. of Fmoc protected amino acid, 1 equi. of DMAP and 4 equi. of DIPEA dissolved in 1000 μl DCM to 50 mg polystyrene resin loaded with the Wang-linker (1 mmol/g) in a suitable reaction vessel.

Add 4 equi. of DIC in 500 μl DCM, shake the vessel for 12 h and then wash the resin 6 times with 1800 μl DCM.

Step B:

Add 1500 μl of a 1:1 mixture of DMF and piperidine to the resin, shake for 30 min and wash 6 times with 1800 μl DMF.

Step C:

Add 5 equi. of aldehyde in 1000 μl NMP and 100 μl HOAc to the resin and shake the vessel for 12 h. Remove the liquid phase and add 1500 μl M NaCNBH₃ in MeOH/DCM 1:1. Shake the vessel for another 12 h. Wash the resin twice with 1800 μl MeOH, twice with 1700 μl DCM, 100 μl DIPEA and twice with 1800 μl DCM.

Step D:

Add 8 equi. of Boc-protected amino acid in 1000 μl THF and 4 equi. DIC in 500 μl to the resin and shake for 30 min. Add 50 μl DIPEA and shake for another 12 h. Remove the liquid phase by suction and repeat the procedure. Afterwards the resin is washed once with 1800 μl DMF and 10 times with 1800 μl DCM. The product is cleaved from the resin with 1500 μl TFA/DCM 1:1. After evaporation in vacuo of the solvent, the residual oil is taken up in 1 ml toluene and heated for 1 h to 60° C. The solvent is again removed in vacuo and the sample analysed by HPLC.

General Procedure (B) Solution Phase Synthesis

Step A:

10 mmol amino acid methyl ester hydrochloride, 1 equi. aldehyde and 1 equi. DIPEA are suspended in 100 ml THF and the resulting mixture is stirred overnight. Then 2.9 equi. NaCNBH₃, 10 ml MeOH and 5 ml HOAc are added and stirred for 3 h. The solvent is removed in vacuo and the residual oil is taken up in 100 ml ethyl acetate. The org. phase is washed once with 100 ml 1 M NaOH. The aq. phase is extracted once with 100 ml ethyl acetate and the combined org. phases are dried over sodium sulfate. The solvent is removed in vacuo and the crude product is used for the next step.

Step B:

18.4 mmol Boc-protected amino acid is dissolved in 50 ml THF, 0.5 equi. DIC is added and the resulting mixture is stirred for 20 min. Then the crude product of step A is added in 50 ml THF and stirred for 2 h. Another 0.25 equi. DIC is added and after 20 min 2 ml of DIPEA. The solvent is removed after 3 h of stirring and the residue is taken up in 100 ml ethyl acetate. The org. phase is washed with 100 ml 1 M HCl and 100 ml sat. NaHCO₃ and dried over sodium sulfate. The solvent is removed in vacuo and the residual oil is purified on silica with ethyl acetate/heptane.

Step C:

The purified product from step B is dissolved in 100 ml DCM and 100 ml TFA is added. The solvents are removed after 2 h. The residual oil is taken up in 100 ml toluene and the solvent is again removed in vacuo. The oil is taken up in 100 ml DCM and 1 ml DIPEA is added. The solvent is removed in vacuo and the product is purified on a C18 reverse phase column (Sep-Pak, Waters, 10 g) with 0.1% TFA in water and acetonitril.

General Procedure (C)—Synthesis on Solid Phase, Suitable for the Synthesis of Multiple Examples in Parallel

Step A:

A linker on polystyrene is prepared by stirring 20 g hydroxymethyl polystyrene (loading 0.87 mmol/g) with 11.3 g (4 equi.) carbonyldiimidazol (CDI) in 500 ml tetrahydrofuran (THF) for 17 h. Wash the resin then with dichloromethane and take it up in 500 ml N-methylpyrrolidone (NMP). Add 5.3 ml (4 equi) amino propanol and stir the mixture over night. Wash the resin once with methanol and once with dichloromethane. Repeat the two washing steps twice (3×(1×MeOH, 1×CH₂Cl₂)). Wash the resin once with diethylether and dry it in vacuo.

Step B:

Add 9 equi. of fmoc protected amino acid and 1 equi. of DMAP dissolved in 2000 μl THF to 50 mg polystyrene resin from step A in a suitable reaction vessel. Add 4 equi. of DIC, shake the vessel for 17 h and then wash the resin 2 times with 3000 PI NMP.

Step C:

Add 1000 μl NMP and 1000 μl piperidine and shake the mixture for 30 min. Wash the resin 5 times with 3000 μl NMP.

Step D:

Add 5 equi. aldehyde in 1500 μl NMP and 100 μl HOAc and shake the mixture for 6 h. Remove the liquid phase by suction and add 2000 μl of 0.5 M NaCNBH₃ in MeOH/DCM 1:1. Shake over night and wash the resin twice with 3000 μl MeOH, twice with 3000 μl DCM+100 μl DIPEA and twice with 3000 μl DCM.

Step E:

Add 8 equi. Boc-protected amino acid in 1500 μl THF and 4 equi. DIC. Shake the resin for 30 min and add 50 μl DIPEA. Shake for another 4 h and remove the liquid phase by suction. Add again 8 equi. Boc-protected amino acid in 1500 μl THF and 4 equi. DIC. Shake the resin for 30 min and add 50 μl DIPEA. Shake overnight and wash twice with 3000 μl NMP and 5 times with 3000 μl DCM. Add 1000 μl DCM and 1000 μl TFA and shake for 30 min. Wash the resin 5 times with 3000 μl DCM and twice with 3000 μl MeOH. Cleave the product from the resin with 1000 μl DCM and 1000 μl 33% MeNH₂ in ethanol for 1 h. The solvent is removed in a nitrogen stream and the samples are taken up in MeOH and analysed by HPLC-MS.

General Procedure (D) Mitsunobu Reaction on a Diketopiperazine

Step A:

A intermediate product is synthesised according to general procedure B using Boc-Tyr(t-bu)-OH for the acylation in step C.

Step B:

The product of step A (14 mmol) is dissolved in 100 ml DCM and 2 equi., 6.1 ml Boc-anhydrid and 1 equi., 2.4 ml DIPEA are added. The solvent is removed in vacuo and the product is purified and silica.

Step C:

The product of step B (1 mmol) is dissolved in 10 ml THF and 1 equi., 0.3 g triphenylphosphine and 1 equi. alcohol are added. Then 1 equi., 160 μl diethyl azadicarboxylate are added and the mixture is stirred over night. The solvent is removed in vacuo and the product is purified either on reverse phase or silica.

Step D:

The product of step C (0.5 mmol) is dissolved in 25 ml DCM and 25 ml TFA. The solvent is removed after 30 min and the solvents are removed in vacuo. The product is purified on a C18 reverse phase column (Sep-Pak, Waters, 10 g) with 0.1% TFA in water and acetonitril.

General Procedure (E): Ester Method

Step A:

To w-N-protected amino acid methyl ester hydrochloride (10 mmol) and aldehyde (10.3 mmol) in THF (80 ml) is added at room temperature sodium triacetoxyborohydride (11 mmol) and the mixture is stirred overnight. Sat. aq. potassium carbonate (100 ml) is added and the mixture is stirred for 1 h. The layers are separated and the aq. layer is extracted with ethyl acetate (3×100 ml). The combined org. layers are dried over sodium sulfate and evaporated in vacuo. Flash chromatography (silica, dichloromethane) afforded the product.

Examples of intermediate compounds of the formula below prepared according to the general procedure E, step A, are shown below.

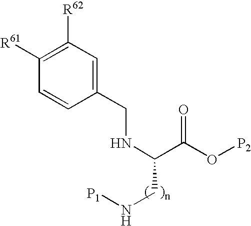

| | | | | | | | ESI-MS |
|---|---|---|---|---|---|---|---|
| i No. | R⁶¹ | R⁶² | P₁ | P₂ | n | Yield | (M + H)⁺ |
| i1 | Ph | H | Boc | Me | 1 | 80% | 385 |
| i2 | OPh | H | Boc | Me | 1 | 98% | 401 |
| i3 | H | OPh | Boc | Me | 1 | 59% | 401 |
| i4 | Ph | H | Cbz | Me | 1 | 77% | 419 |
| i5 | OPh | H | Cbz | Me | 1 | 70% | 435 |
| i6 | Ph | H | Boc | Me | 2 | 62% | 399 |
| i7 | OPh | H | Boc | Me | 2 | 63% | 415 |
| i8 | Ph | H | Cbz | Me | 3 | 48% | 447 |
| i9 | Br | H | Boc | Me | 4 | quant. | 430 |
| i10 | Ph | H | Boc | Me | 4 | 68% | 427 |
| i11 | OPh | H | Boc | Me | 4 | 37% | 443 |
| i12 | Bn | H | Boc | Me | 4 | 42% | 441 |
| i13 | OBn | H | Boc | Me | 4 | 38% | 457 |
| i14 | OBn | H | Boc | t-Bu | 4 | 85% | 499 |
| i15 | N(Me)Ph | H | Boc | Me | 4 | 61% | 456 |
| i16 | OCbz | H | Boc | Me | 4 | 56% | 501 |
| i17 | OTBDPS | H | Boc | Me | 4 | 45% | 605 |
| i18 | OPh | Me | Boc | Me | 4 | 43% | 457 |
| i19 | OPh | OMe | Boc | Me | 4 | 56% | 473 |
| i20 | O-4-pyridyl | H | Boc | Me | 4 | 31% | 444 |
| i21 | Ph | H | Cbz | Me | 4 | 65% | 461 |
| i22 | OPh | H | Cbz | Me | 4 | 19% | 477 |
| i23 | Ph | H | Cbz | Me | 5 | 45% | 475 |

Step B:

To ω-N-protected amino acid methyl ester hydrochloride (10 mmol) in MeOH/THF 1:1 (60 ml) is added at room temperature lithium hydroxide hydrate (40 mmol) in water (30 ml) and the mixture is stirred overnight. The mixture is diluted with water (100 ml) and acidified with sat. aq. potassium bisulfate (200 ml). The precipitate is collected by filtration, washed with water (3×50 ml), and dried at 60° C. to give the free acid product.

Examples of intermediate compounds of the formula below prepared according to the general procedure E, step B, are shown below:

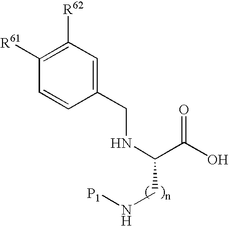

| | | | | | | ESI-MS |
|---|---|---|---|---|---|---|
| i No. | R⁶¹ | R⁶² | P₁ | n | Yield | (M + H)⁺ |
| i24 | Ph | H | Boc | 1 | 86% | 371 |
| i25 | OPh | H | Boc | 1 | 90% | 387 |
| i26 | H | OPh | Boc | 1 | 83% | 387 |
| i27 | Ph | H | Cbz | 1 | 91% | 405 |
| i28 | OPh | H | Cbz | 1 | 89% | 421 |
| i29 | Ph | H | Boc | 2 | 77% | 385 |
| i30 | OPh | H | Boc | 2 | 94% | 401 |
| i31 | Ph | H | Cbz | 3 | 70% | 433 |
| i32 | Ph | H | Boc | 4 | 94% | 413 |
| i33 | OPh | H | Boc | 4 | 96% | 429 |
| i34 | O-4-pyridyl | H | Boc | 4 | 60% | 430 |
| i35 | OPh | H | Cbz | 4 | 92% | 463 |

Step C:

To the product of general procedure E, step B (10 mmol), amino acid methyl ester (10 mmol), TBTU (10.4 mmol), and HOBt (10.2 mmol) in THF (150 ml) is added N-ethyldiisopropylamine (35 mmol) and the mixture is stirred at room temperature overnight. The mixture is concentrated in vacuo and diluted with ethyl acetate (150 ml). The org. layer is washed with sat. aq. sodium carbonate (3×) and water (2×), dried over sodium sulfate, and evaporated in vacuo. Flash chromatography (silica, petroleum ether/ethyl acetate 1:1→ethyl acetate/MeOH 49:1) afforded the product.

Examples of intermediate compounds of the formula below prepared according to the general procedure E, step C, are shown below:

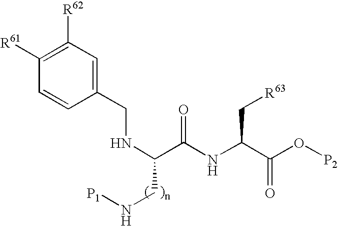

| | | | | | | | | ESI-MS |
|---|---|---|---|---|---|---|---|---|
| i No. | R⁶¹ | R⁶² | R⁶³ | P₁ | P₂ | n | Yield | (M + H)⁺ |
| i36 | Ph | H | 2-naphthyl | Boc | Me | 1 | 89% | 582 |
| i37 | OPh | H | 2-naphthyl | Boc | Me | 1 | quant | 598 |
| i38 | H | OPh | 2-naphthyl | Boc | Me | 1 | 86% | 598 |
| i39 | OPh | H | 3,4-Cl₂—C₆H₃ | Boc | Me | 1 | 91% | 617 |
| i40 | OPh | H | 4-CF₃—C₆H₄ | Boc | Me | 1 | quant | 616 |
| i41 | OPh | H | 4-OCF₃-3-Cl—C₆H₃ | Boc | Et | 1 | 78% | 681 |
| i42 | OPh | H | 4-OCF₃—C₆H₄ | Boc | Et | 1 | 74% | 646 |
| i43 | OPh | H | 4-OCF₂H—C₆H₄ | Boc | Et | 1 | 80% | 628 |
| i44 | Ph | H | 2-naphthyl | Cbz | Me | 1 | 92% | 616 |
| i45 | OPh | H | 2-naphthyl | Cbz | Me | 1 | 95% | 632 |
| i46 | Ph | H | 2-naphthyl | Boc | Me | 2 | 81% | 596 |
| i47 | OPh | H | 2-naphthyl | Boc | Me | 2 | 87% | 612 |
| i48 | Ph | H | 2-naphthyl | Cbz | Me | 3 | 87% | 644 |
| i49 | Ph | H | 2-naphthyl | Boc | Me | 4 | 86% | 624 |
| i50 | OPh | H | 5,6,7,8-tetrahydro- | Boc | Me | 4 | 70% | 644 |
| | | | naphthalen-2-yl | | | | | |
| i51 | OPh | H | 4-NH₂-3,5-Br₂—C₆H₂ | Boc | Me | 4 | 96% | 763 |
| i52 | OPh | H | 4-OH-3,5-Br₂—C₆H₂ | Boc | Me | 4 | 96% | 764 |
| i53 | OPh | H | 4-MeO-3-Cl-C₆H₃ | Boc | Me | 4 | 99% | 655 |
| i54 | O-4-pyridyl | H | 2-naphthyl | Boc | Me | 4 | 83% | 641 |
| i55 | Ph | H | 1-MeO-naphthalen-2-yl | Boc | Et | 4 | 45% | 668 |
| i56 | Ph | H | 6-Cl-naphthalen-2-yl | Boc | Et | 4 | 94% | 673 |
| i57 | OPh | H | 3,4-Et₂-C₆H₃ | Boc | Me | 4 | 99% | 646 |

Step D:

The product of general procedure E, step C (5 mmol) in toluene/n-butanol/glacial acetic acid 5:5:1 (200 ml) is heated to reflux for 24 h, concentrated in vacuo, and diluted with ethyl acetate (200 ml). The org. layer is washed with sat. aq. sodium carbonate (2×) and water (1×), dried over sodium sulfate, and evaporated in vacuo. Flash chromatography (silica, petroleum ether/ethyl acetate 3:1→1:3) afforded the product.

Examples of intermediate compounds of the formula below prepared according to the general procedure E, step D, are shown below:

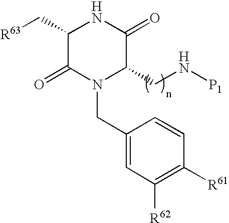

| i No. | R⁶¹ | R⁶² | R⁶³ | P₁ | n | Yield | ESI-MS |
|---|---|---|---|---|---|---|---|
| i58 | Ph | H | 2-naphthyl | Boc | 1 | 98% | (M − H)⁻ = 548 |
| i59 | Ph | H | 2-naphthyl | Cbz | 1 | quant | (M + H)⁺ = 584 |
| i60 | OPh | H | 2-naphthyl | Boc | 1 | crude | (M − H)⁻ = 564 |
| i61 | H | OPh | 2-naphthyl | Boc | 1 | quant | (M − H)⁻ = 564 |
| i62 | OPh | H | 4-OCF₃—C₆H₄ | Boc | 1 | 78% | (M − H)⁻ = 598 |
| i63 | OPh | H | 4-OCF₂H—C₆H₄ | Boc | 1 | 92% | (M − H)⁻ = 580 |
| i64 | OPh | H | 4-OCF₃-3-Cl—C₆H₃ | Boc | 1 | 40% | (M − H)⁻ = 632 |
| i65 | OPh | H | 3,4-Cl₂—C₆H₃ | Boc | 1 | 92% | (M + Na)⁺ = 606 |
| i66 | OPh | H | 4-CF₃—C₆H₄ | Boc | 1 | quant | (M + H)⁺ = 584 |
| i67 | Ph | H | 2-naphtyl | Boc | 2 | 83% | (M − H)⁻ = 562 |
| i68 | OPh | H | 2-naphthyl | Boc | 2 | 90% | (M + H)⁺ = 580 |
| i69 | Ph | H | 2-naphthyl | Cbz | 3 | 97% | (M − H)⁻ = 610 |
| i70 | Ph | H | 2-naphthyl | Boc | 4 | 50% | (M − H)⁻ = 646 |
| i71 | Ph | H | 1-MeO-naphthalen-2-yl | Boc | 4 | 69% | (M + H)⁺ = 622 |
| i72 | Ph | H | 6-Cl-naphthalen-2-yl | Boc | 4 | quant | (M + H)⁺ = 626 |
| i73 | OPh | H | 3,4-Et₂-C₆H₃ | Boc | 4 | quant | (M + H)⁺ = 614 |
| i74 | OPh | H | 4-MeO-3-Cl—C₆H₃ | Boc | 4 | 99% | (M + H)⁺ = 623 |
| i75 | OPh | H | 4-NH₂-3,5-Br₂—C₆H₂ | Boc | 4 | 64% | (M − H)⁻ = 727 |
| i76 | OPh | H | 4-OH-3,5-Br₂—C₆H₂ | Boc | 4 | 76% | (M + H)⁺ = 730 |
| i77 | O-4-pyridyl | H | 2-naphthyl | Boc | 4 | 76% | (M + H)⁺ = 609 |
| i78 | OPh | H | 5,6,7,8-tetrahydro- | Boc | 4 | 50% | (M + H)⁺ = 612 |
| | | | naphthalen-2-yl | | | | |

Step E:

To the N-Boc-protected product of general procedure E, step D (20 mmol) in dichloromethane (180 ml) is added dropwise trifluoroacetic acid (180 mmol) and the mixture is stirred overnight. The yellow solution is added dropwise to sat. aq. sodium carbonate (190 mmol) and the mixture is stirred for another 30 min. The org. layer is separated, dried over sodium sulfate, and concentrated in vacuo. Flash chromatography (silica, dichloromethane/MeOH 20:1) gave final products of formula (Ia), see table I (examples 1 to 8).

Step F:

To the O-TBDPS-protected product of general procedure F, step C (3.08 mmol) in THF (50 ml) is added N-ethyldiisopropylamine (6 mmol) followed by Boc anhydride (3.5 mmol) and the mixture is stirred at room temperature for 1 h. The org. layer is diluted with ethyl acetate (50 ml) and extracted with water (2×40 ml). The org. layer is separated and dried over sodium sulfate. Flash chromatography (silica, ethyl acetate/petroleum ether 3:1<ethyl acetate) afforded the Boc-protected intermediate.

To the N-Boc-O-TBDPS-protected intermediate (2 mmol) in THF (20 ml) is added at room temperature tetrabutylammonium fluoride (1 N in THF, 8 mmol), followed by glacial acetic acid (0.5 ml) and the mixture is stirred for 2 h. Water (30 ml) is added, the aq. layer is extracted with ethyl acetate (3×50 ml), and the combined org. layers are dried over sodium sulfate. Flash chromatography (silica, dichloromethane/MeOH 25:1) gave the crude phenolic intermediate.

To the crude phenol (0.5 mmol), the corresponding boronic acid (1.5 mmol), copper(II) acetate (0.5 mmol), and molecular sieves (4 A, powdered, 500 mg) in dichloromethane is added under argon at room temperature triethylamine (2.5 mmol) and the mixture is stirred overnight. Flash chromatography (silica, dichloromethane/MeOH 50:1→30:1) gave the product.

Examples of intermediates of the formula below prepared according to the general procedure E, step F, are shown below:

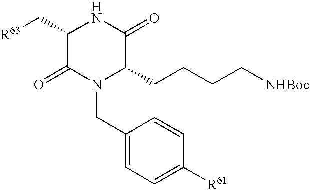

| i No. | R⁶¹ | R⁶³ | Yield | ESI-MS |
|---|---|---|---|---|
| i79 | O-(2-Me-C₆H₄) | 2-naphthyl | 41% | (M + H)⁺ = 622 |
| i80 | O-(3-Cl—C₆H₄) | 2-naphthyl | 72% | (M + H)⁺ = 642 |

N-Boc-Deprotection is achieved according to procedure E, step E to give the final products of formula (Ib), see table III, examples 9 to 10.

Step G:

N-Cbz-protected compound of general procedure E, step D (20 mmol) in MeOH (150 ml) is hydrogenated (50 psi) in the presence of Pd/C (10%) at 50° C. for 90 min. The catalyst is removed by filtration and the filtrate is concentrated in vacuo. Trituration (dichloromethane/ether/petroleum ether) and drying under high vacuum yields the final products of general formula (Id), see table VI, examples 21 to 25.

General Procedure (F): Anhydride Method

Step A:

To N-Boc-protected amino acid (20 mmol) in THF (150 ml) is added at room temperature N,N′-diisopropylcarbodiimide (10 mmol) and the mixture is stirred overnight. The mixture is concentrated in vacuo and the residue triturated (petroleum ether). The precipitate is collected by filtration and washed with petroleum ether and dried under high vacuum.

Examples of intermediate compounds of the formula below prepared according to the general procedure F, step F, are shown below:

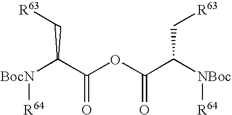

| i No. | R⁶³ | R⁶⁴ | Yield |
|---|---|---|---|
| i81 | 2-naphthyl | H | 90% |
| i82 | 2-naphthyl | Me | 99% |

Step B:

To the intermediate products from general procedure E, step A (2 mmol) and general procedure G, step A (4 mmol) in THF (60 ml) is added N-ethyldiisopropylamine (4.8 mmol) and the mixture is heated to reflux for 5 h and then stirred at room temperature overnight. The mixture is concentrated in vacuo, diluted with ethyl acetate (100 ml). The org. layer is washed with sat. aq. sodium bicarbonate, dried over sodium sulfate, and evaporated in vacuo. Flash chromatography (silica, ethyl acetate/petroleum ether 3:1→2:1) afforded the product.

Examples of intermediate compounds of the formula below prepared according to the general procedure F, step B, are shown below:

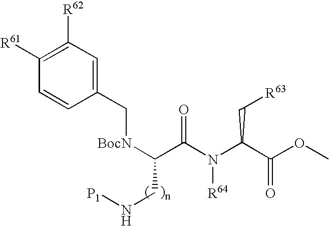

| i No. | R⁶¹ | R⁶² | R⁶³ | R⁶⁴ | P1 | n | Yield | ESI-MS |
|---|---|---|---|---|---|---|---|---|
| i83 | Ph | H | 2-naphthyl | H | Boc | 4 | 78% | (M + Na)⁺ = 746 |
| i84 | Ph | H | 2-naphthyl | H | Cbz | 4 | crude | (M + H)⁺ = 758 |
| i85 | Ph | H | 2-naphthyl | Me | Cbz | 4 | crude | (M + H)⁺ = 772 |
| i86 | OBn | H | 2-naphthyl | H | Boc | 4 | 81% | (M + Na)⁺ = 776 |
| i87 | CH2Ph | H | 2-naphthyl | H | Boc | 4 | 25% | (M + H)⁺ = 738 |
| i88 | N(Me)Ph | H | 2-naphthyl | H | Boc | 4 | 32% | (M + H)^{+ = 753} |
| i89 | OCbz | H | 2-naphthyl | H | Boc | 4 | 37% | (M + H)⁺ = 798 |
| i90 | OTBDPS | H | 2-naphthyl | H | Boc | 4 | crude | (M + H)⁺ = 902 |
| i91 | OPh | Me | 2-naphthyl | H | Boc | 4 | crude | (M + H)⁺ = 754 |
| i92 | OPh | OMe | 2-naphthyl | H | Boc | 4 | crude | (M + H)⁺ = 770 |
| i93 | Ph | H | 2-naphthyl | H | Cbz | 5 | crude | (M + H)⁺ = 772 |

Step C:

To the product of general procedure F, step B or the product of general procedure G, step B (2 mmol) in dichloromethane (50 ml) is added TFA (4.5 ml) and the mixture is stirred at room temperature for 3 h. The mixture is neutralized with sat. aq. sodium bicarbonate (250 ml) and the aq. layer is extracted with dichloromethane (4×150 ml). The combined org. layers are dried over sodium sulfate and evaporated in vacuo to give the final products of formula (Ic), see table IV, examples 11 to 20.

General Procedure (G): Acid Fluoride Method

Step A:

To N-Boc-protected amino acid (12 mmol) in dichloromethane (30 ml) is added at −15° C. pyridine (12 mmol) followed by slow addition of cyanuric fluoride (60 mmol). The mixture is stirred for 1 h at −15° C., diluted with ice water (60 ml) and dichloromethane (100 ml), and the precipitation removed by filtration. The layers are separated and the aq. layer is extracted with dichloromethane (30 ml). The combined org. layers are washed with ice water (50 ml), dried over sodium sulfate, and evaporated in vacuo at 20° C. to give the crude acid fluoride.

Examples of intermediate compounds of the formula below prepared according to the general procedure G, step A, are shown below:

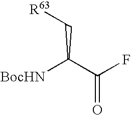

| i No: | R⁶³ | Yield |
|---|---|---|
| i94 | 2-naphthyl | 91%, crude |
| i95 | 3-MeO-naphthalen-2-yl | quant., crude |

Step B:

To the product of general procedure E, step A (4.5 mmol) and N-ethyldiisopropylamine (9 mmol) in dichloromethane (90 ml) is added at 0° C. the product of general procedure G, step D (6.75 mmol) and the mixture is stirred at 0° C. for 30 min., at room temperature for 2 h, and heated to reflux overnight. The mixture is diluted with dichloromethane (100 ml), the org. layer is washed with sat. aq. sodium bicarbonate, and dried over sodium sulfate. Flash chromatography (silica, ethyl acetate/petroleum ether 1:2) afforded the product.

Examples of intermediate compounds of the formula below prepared according to the general procedure G, step B, are shown below:

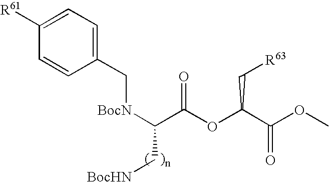

| i No | R⁶¹ | R⁶³ | n | Yield | ESI-MS |
|---|---|---|---|---|---|
| i96 | OPh | 2-naphthyl | 4 | 61% | (M + H)⁺ = 738 |
| i97 | OBn | 2-naphthyl | 4 | 28% | (M + Na)⁺ = 776 |
| i98 | Ph | 3-MeO-naphthalen-2-yl | 4 | crude | (M + H)⁺ = 754 |

Step C:

To the product of general procedure E, step E-G (3 mmol) and aldehyde (12 mmol) in THF (120 ml) is added p-toluenesulfonic acid hydrate (3.6 mmol) and sodium triacetoxyborohydride (12.5 mmol) and the mixture is stirred at room temperature for 48 h. Sat. aq. sodium bicarbonate (100 ml) is added, the mixture is stirred for another 30 min., and then extracted with ether (3×100 ml). The combined org. layers are dried over sodium sulfate and concentrated in vacuo. Flash chromatography (silica, ethyl acetate) and trituration (ether /petroleum ether)afforded the product.

Examples of intermediate compounds of the formula below prepared according to the general procedure G, step C, are shown below:

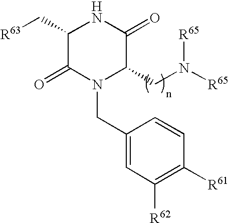

| i No. | R⁶¹ | R⁶² | R⁶³ | R⁶⁵ | n | Yield | ESI-MS |
|---|---|---|---|---|---|---|---|
| i99 | Ph | H | 2-naphthyl | N-Cbz-piperidin-4-yl-methyl | 1 | 96% | (M + H)⁺ = 913 |
| i100 | OPh | H | 2-naphthyl | N-Cbz-piperidin-4-yl-methyl | 1 | 85% | (M − H)⁺ = 928 |
| i101 | H | OPh | 2-naphthyl | N-Cbz-piperidin-4-yl-methyl | 1 | 93% | (M + H)⁺ = 928 |
| i102 | OPh | H | 2-naphthyl | 3-(N-Cbz-amino)-propyl | 1 | 60% | (M + H)⁺ = 848 |
| i103 | OPh | H | 2-naphthyl | 1H-imidazol-4-yl-methyl | 1 | 45% | (M + H)⁺ = 610 |
| i104 | OPh | H | 4-OCF₃—C₆H₄ | N-Boc-piperidin-4-yl-methyl | 1 | 59% | (M + H)⁺ = 894 |
| i105 | OPh | H | 4-OCF₂H—C₆H₄ | N-Boc-piperidin-4-yl-methyl | 1 | 74% | (M + H)⁺ = 876 |
| i106 | OPh | H | 4-CF₃—C₆H₄ | N-Cbz-piperidin-4-yl-methyl | 1 | 47% | (M + H)⁺ = 946 |
| i107 | OPh | H | 4-CF₃—C₆H₄ | N-Boc-piperidin-4-yl-methyl | 1 | 71% | (M + H)⁺ = 878 |
| i108 | OPh | H | 3,4-Cl₂—C₆H₃ | N-Boc-piperidin-4-yl-methyl | 1 | 82% | (M + H)⁺ = 878 |
| i109 | OPh | H | 2-naphthyl | N-Cbz-piperidin-4-yl-methyl | 2 | quant | (M + H)⁺ = 942 |

Step D:

To the product of general procedure E, step E-G (4 mmol) and aldehyde (4 mmol) in THF (100 ml) is added p-toluenesulfonic acid hydrate (4 mmol) and sodium triacetoxyborohydride (8.3 mmol) and the mixture is stirred at room temperature for 2 h. Sat. aq. sodium bicarbonate (100 ml) is added, the mixture is stirred for another 30 min., and then extracted with ether (4×100 ml). The combined org. layers are dried over sodium sulfate and concentrated in vacuo. Flash chromatography (silica, dichloromethane/MeOH 20:1) afforded the product.

Examples of intermediate compounds of the formula below prepared according to the general procedure G, step D, are shown below:

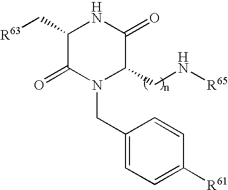

| i No. | R⁶¹ | R⁶³ | R⁶⁵ | n | Yield | ESI-MS |
|---|---|---|---|---|---|---|
| i110 | Ph | 2-naphthyl | N-Cbz-piperidin-4-yl-methyl | 1 | 81% | (M + H)⁺ = 681 |
| i111 | OPh | 2-naphthyl | N-Cbz-piperidin-4-yl-methyl | 1 | 78% | (M − H)⁺ = 697 |
| i112 | Ph | 2-naphthyl | 2-(N-Boc-amino)-ethyl | 1 | 67% | (M + H)⁺ = 593 |
| i113 | OPh | 4-CF₃—C₆H₄ | N-Cbz-piperidin-4-yl-methyl | 1 | 62% | (M + H)⁺ = 715 |
| i114 | OPh | 4-OCF₃-3-Cl— | N-Cbz-piperidin-4-yl-methyl | 1 | 42% | (M + H)⁺ = 765 |
| | | C₆H₃ | | | | |
| i115 | OPh | 3,4-Cl₂—C₆H₃ | N-Boc-piperidin-4-yl-methyl | 1 | 59% | (M + H)⁺ = 681 |
| i116 | Ph | 2-naphthyl | 1H-imidazol-4-yl-methyl | 1 | crude | (M + H)⁺ = 530 |
| i117 | Ph | 2-naphthyl | N-Boc-piperidin-4-yl | 1 | 45% | (M + H)⁺ = 633 |
| i118 | Ph | 2-naphthyl | 4-(N-Boc-amino)-cyclohexyl | 1 | 89% | (M + H)⁺ = 647 |
| i119 | Ph | 2-naphthyl | 4-pyridyl-methyl | 1 | crude | (M + H)⁺ = 541 |
| i120 | OPh | 2-naphthyl | N-Cbz-piperidin-4-yl-methyl | 2 | crude | (M + H)⁺ = 711 |

Step E: Acylation Using Free Carboxylic Acids

To the product of general procedure E, step E to G (0.6 mmol), the corresponding carboxylic acid (0.6 mmol), TBTU (0.615 mmol), and HOBt (0.615 mmol) in THF (15 ml) is added at room temperature N-ethyldiisopropylamine (2.1 mmol) and the mixture is stirred overnight. The mixture is diluted with ethyl acetate (100 ml), extracted with sat. aq. sodium bicarbonate (100 ml), and dried over sodium sulfate. Flash chromatography (silica, dichloromethane/MeOH 49:1→19:1) gave the product.

Examples of intermediate compounds of the formula below prepared according to the general procedure G, step E, are shown below:

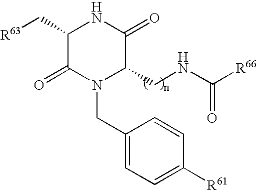

| i No. | R⁶¹ | R⁶² | R⁶⁶ | n | Yield | ESI-MS |
|---|---|---|---|---|---|---|
| i121 | Ph | 2-naphthyl | N,N-dimethyl-amino-methyl | 1 | 25% | (M + H)⁺ = 535 |
| i122 | Ph | 2-naphthyl | 3-(N,N-diethyl-amino)-ethyl | 1 | 62% | (M + H)⁺ = 577 |
| i123 | Ph | 2-naphthyl | N-Boc-piperidin-4-yl | 1 | 85% | (M − H)⁻ = 659 |
| i124 | Ph | 2-naphthyl | N-Boc-piperidin-3-yl | 1 | 97% | (M + H)⁺ = 661 |

Step F: Acylation Using Acid Chlorides

To the product of general procedure E, step E to G or general procedure G, step C (0.7 mmol) and the corresponding acid chloride (0.77 mmol) in dichloromethane (20 ml) is added at room temperature N-ethyldiisopropylamine (2.1 mmol) and the mixture is stirred overnight. The mixture is diluted with dichloromethane (100 ml), extracted with sat. aq. sodium bicarbonate (100 ml), and dried over sodium sulfate. Flash chromatography (silica, dichloromethane/MeOH 49:1→19:1) gave the product.

Examples of intermediate compounds of the formula below prepared according to the general procedure G, step F, are shown below:

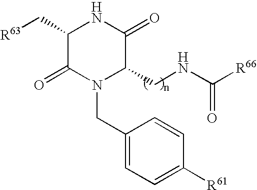

| i No. | R⁶¹ | R⁶³ | R⁶⁶ | n | Yield | ESI-MS |
|---|---|---|---|---|---|---|
| i125 | Ph | 2-naphthyl | 4-cyanophenyl | 1 | 97% | (M + H)⁺ =579 |
| i126 | Ph | 2-naphthyl | 4-methyl- | 1 | 96% | (M + H)⁺ =576 |
| | | | piperazyl | | | |
| i127 | Ph | 2-naphthyl | 4-pyridyl | 1 | 79% | (M + H)⁺ = 555 |

Step G: Cbz-Deprotection

N-Cbz-Protected product from general procedure G, step C (3 mmol) in MeOH (150 ml) is hydrogenated (50 psi) in the presence of Pd/C (10%) at 50° C. for 90 min. The catalyst is removed by filtration and the filtrate is concentrated in vacuo. Purification by HPLC (ZorbaxSB-C18 (5 μm) column, gradient of water/ MeCN +0.1% formic acid, detection at 254 nm and 230 nm) and lyophilization afforded the product.

Examples of intermediate compounds of the formula below prepared according to the general procedure G, step G, are shown below:

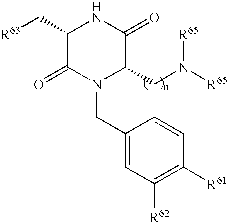

| i No. | R⁶¹ | R⁶² | R⁶³ | R⁶⁵ | n | Yield | ESI-MS |
|---|---|---|---|---|---|---|---|
| i128 | Ph | H | 2-naphthyl | piperidin-4-yl-methyl | 1 | 99% | (M + H)⁺ = 644 |
| i129 | OPh | H | 2-naphthyl | piperidin-4-yl-methyl | 1 | 89% | (M + H)⁺ = 660 |
| i130 | H | OPh | 2-naphthyl | piperidin-4-yl-methyl | 1 | 90% | (M + H)⁺ = 660 |
| i131 | OPh | H | 2-naphthyl | piperidin-4-yl-methyl | 1 | 78% | (M + H)⁺ = 580 |
| i132 | OPh | H | 4-CF₃—C₆H₄ | piperidin-4-yl-methyl | 1 | 91% | (M + H)⁺ = 678 |
| i133 | OPh | H | 2-naphthyl | piperidin-4-yl-methyl | 2 | 93% | (M + H)⁺ = 674 |

Step H: Cbz-Deprotection

N-Cbz-Protected product from general procedure G, step D to F (3 mmol) in MeOH (150 ml) is hydrogenated (50 psi) in the presence of Pd/C (10%) at 50° C. for 90 min. The catalyst is removed by filtration and the filtrate is concentrated in vacuo. Trituration (dichloromethane /ether/petroleum ether) and drying under high vacuum yielded the product.

Examples of intermediate compounds of the formula below prepared according to the general procedure G, step H, are shown below:

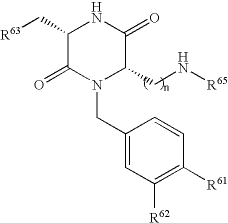

| i No. | R⁶¹ | R⁶² | R⁶³ | R⁶⁵ | n | Yield | ESI-MS |
|---|---|---|---|---|---|---|---|
| i134 | Ph | H | 2-naphthyl | piperidin-4-yl-methyl | 1 | 75% | (M + H)⁺ = 547 |

Step I: Boc-Deprotection

To the N-Boc-protected product of general procedure G, step C (2 mmol) in dichloromethane (18 ml) is added dropwise trifluoroacetic acid (18 mmol) and the mixture is stirred overnight. The yellow solution is added dropwise to sat. aq. sodium carbonate (19 mmol) and the mixture is stirred for another 30 min. The org. layer is separated, dried over sodium sulfate, and concentrated in vacuo Purification by HPLC (ZorbaxSB-C18 (5 pm) column, gradient of water/MeCN +0.1% formic acid, detection at 254 nm and 230 nm) and lyophilization afforded the product.

Examples of intermediate compounds of the formula below prepared according to the general procedure G, step I, are shown below:

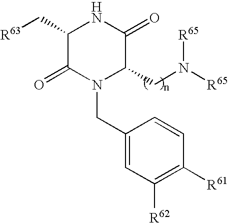

| i No. | R⁶¹ | R⁶² | R⁶³ | R⁶⁵ | n | Yield | ESI-MS |
|---|---|---|---|---|---|---|---|
| i135 | OPh | H | 4-OCF₃—C₆H₄ | piperidin-4-yl-methyl | 1 | quant. | (M + H)⁺ = 694 |
| i136 | OPh | H | 4-CF₂H—C₆H₄ | piperidin-4-yl-methyl | 1 | 59% | (M + H)⁺ = 676 |
| i137 | OPh | H | 3,4-Cl₂—C₆H₃ | piperidin-4-yl-methyl | 1 | 97% | (M + H)⁺ = 679 |
| i138 | OPh | H | 4-CF₃—C₆H₄ | piperidin-4-yl-methyl | 1 | 33% | (M + H)⁺ = 678 |

Step J: Boc-Deprotection

To the N-Boc-protected product of general procedure G, step D to F (2 mmol) in dichloromethane (18 ml) is added dropwise trifluoroacetic acid (18 mmol) and the mixture is stirred overnight. The yellow solution is added dropwise to sat. aq. sodium carbonate (19 mmol) and the mixture is stirred for another 30 min. The org. layer is separated, dried over sodium sulfate, and concentrated in vacuo. Purification by HPLC (ZorbaxSB-C18 (5 pm) column, gradient of water/MeCN +0.1% formic acid, detection at 254 nm and 230 nm) and lyophilization afforded the product.

Intermediate compounds prepared according to the general procedure G, step J:

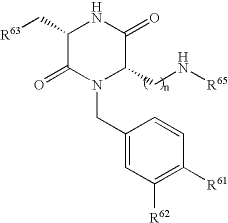

| i No. | R⁶¹ | R⁶² | R⁶³ | R⁶⁵ | n | Yield | ESI-MS |
|---|---|---|---|---|---|---|---|
| i139 | Ph | H | 2-naphthyl | 2-amino-ethyl | 1 | 16% | (M + H)⁺ = 493 |
| i140 | Ph | H | 2-naphthyl | piperidin-4-yl | 1 | 52% | (M + H)⁺ = 533 |
| i141 | Ph | H | 2-naphthyl | 4-amino-cyclo-hexyl | 1 | 61% | (M + H)⁺ = 547 |
| i142 | Ph | H | 2-naphthyl | 3-amino-propionyl | 1 | 89% | (M + H)⁺ = 521 |
| i143 | Ph | H | 2-naphthyl | piperidin-4-yl-carbonyl | 1 | 99% | (M + H)⁺ = 561 |
| i144 | Ph | H | 2-naphthyl | piperidin-3-yl-carbonyl | 1 | 87% | (M + H)⁺ = 561 |

Examples of compounds according to the present invention are shown below. These examples are provided for illustrative purposes only and shall not be construed as limiting the scope of the present invention as defined by the appended claims.

### Examples 1 to 8

Intermediate compounds prepared according to the general procedure E, step E of the general formula (Ia) are shown in Table I, and compounds prepared according to the general procedure E, step E of the general formula (Ia) are shown in Table II

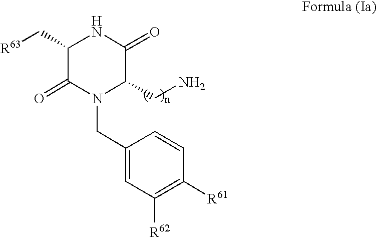

The number of the intermediate used as starting compound in general procedure E, step E are stated under i No.

TABLE I

| Intermediate | | | | | | | ESI-MS |
|---|---|---|---|---|---|---|---|
| no | i No. | R⁶¹ | R⁶² | R⁶³ | n | Yield | (M + H)⁺ |
| i145 | i58 | Ph | H | 2-naphthyl | 1 | 93% | 450 |
| i146 | i60 | OPh | H | 2-naphthyl | 1 | 81% | 466 |
| i147 | i61 | H | OPh | 2-naphthyl | 1 | 90% | 466 |
| i148 | i66 | OPh | H | 4-CF₃—C₆H₄ | 1 | quant. | 484 |
| i149 | i62 | OPh | H | 4-OCF₃—C₆H₄ | 1 | 98% | 500 |
| i150 | i63 | OPh | H | 4-OCF₂H—C₆H₄ | 1 | 98% | 482 |
| i151 | i64 | OPh | H | 4-CF₃-3-Cl—C₆H₃ | 1 | 89% | 534 |
| i152 | i65 | OPh | H | 3,4-Cl₂—C₆H₃ | 1 | 93% | 485 |
| i153 | i67 | Ph | H | 2-naphthyl | 2 | 51% | 464 |
| i154 | i68 | OPh | H | 2-naphthyl | 2 | 57% | 480 |

TABLE II

| Example | | | | | | | ESI-MS |
|---|---|---|---|---|---|---|---|
| No | i No. | R⁶¹ | R⁶² | R⁶³ | n | Yield | (M + H)⁺ |
| 1 | i70 | Ph | H | 2-naphthyl | 4 | 60% | 492 |
| 2 | i74 | OPh | H | 4-MeO-3-Cl—C₆H₃ | 4 | 97% | 522 |
| 3 | i71 | Ph | H | 1-MeO-naphthalen-2-yl | 4 | 48% | 522 |
| 4 | i72 | Ph | H | 6-Cl-naph-thalen-2-yl | 4 | 30% | 526 |
| 5 | i75 | OPh | H | 4-NH₂-3,5-Br₂—C₆H₂ | 4 | 98% | 629 |
| 6 | i76 | OPh | H | 4-OH-3,5-Br₂—C₆H₂ | 4 | 99% | 630 |
| 7 | i77 | O-4-pyridyl | H | 2-naphthyl | 4 | 70% | 509 |
| 8 | i78 | OPh | H | 5,6,7,8-tetrahydronaphthalen-2-yl | 4 | 52% | 512 |

### Examples 9 to 10

Compounds prepared according to the general procedure E, step E of the general formula (Ib) are shown in Table III:

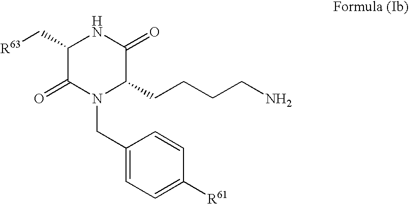

The number of the intermediate used as starting compound in general procedure E, step E are stated under i No.

TABLE III

| Example | | | | | ESI-MS |
|---|---|---|---|---|---|
| no | i No. | R⁶¹ | R⁶³ | Yield | (M + H)⁺ |
| 9 | i79 | O-(2-Me—C₆H₄) | 2-naphthyl | 99% | 522 |
| 10 | i80 | O-(3-Cl—C₆H₄) | 2-naphthyl | 81% | 542 |

### Examples 11 to 20

Intermediate compounds prepared according to the general procedure F, step C of general formula (Ic) are shown in Table IV, and compounds prepared according to the general procedure F, step C of general formula (Ic) are shown in Table V:

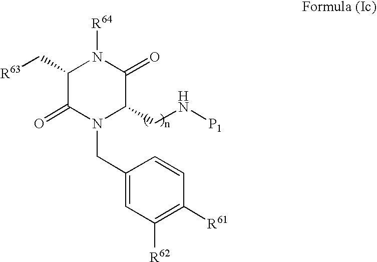

The number of the intermediate used as starting compound in general procedure F, step C are stated under i No.

TABLE IV

| Intermediate | | | | | | | | | ESI-MS |
|---|---|---|---|---|---|---|---|---|---|
| no | i No. | R⁶¹ | R⁶² | R⁶³ | R⁶⁴ | P₁ | n | Yield | (M + H)⁺ |
| i155 | i84 | Ph | H | 2-naphthyl | H | Cbz | 4 | 99% | 626 |
| i156 | i85 | Ph | H | 2-naphthyl | Me | Cbz | 4 | 99% | 640 |
| i157 | i93 | Ph | H | 2-naphthyl | H | Cbz | 5 | 99% | 640 |

TABLE V

| Example | | | | | | | | | ESI-MS |
|---|---|---|---|---|---|---|---|---|---|
| no | i No. | R⁶¹ | R⁶² | R⁶³ | R⁶⁴ | P₁ | n | Yield | (M + H)⁺ |
| 11 | i83 | Ph | H | 2-naphthyl | H | H | 4 | 97% | 492 |
| 12 | i98 | Ph | H | 3-MeO-naphthalen-2-yl | H | H | 4 | 99% | 522 |
| 13 | i96 | OPh | H | 2-naphthyl | H | H | 4 | 97% | 508 |
| 14 | i86 | OBn | H | 2-naphthyl | H | H | 4 | 96% | 522 |
| 15 | i90 | OTBDPS | H | 2-naphthyl | H | H | 4 | quant. | 670 |
| 16 | i89 | OCbz | H | 2-naphthyl | H | H | 4 | 99% | 566 |
| 17 | i88 | N(Me)Ph | H | 2-naphthyl | H | H | 4 | 99% | 521 |
| 18 | i87 | CH₂Ph | H | 2-naphthyl | H | H | 4 | 99% | 506 |
| 19 | i91 | OPh | Me | 2-naphthyl | H | H | 4 | 99% | 522 |
| 20 | i92 | OPh | OMe | 2-naphthyl | H | H | 4 | 99% | 538 |

### Examples 21 to 25

Compounds prepared according to the general procedure E, step G of the general formula (Id) are shown in Table VI:

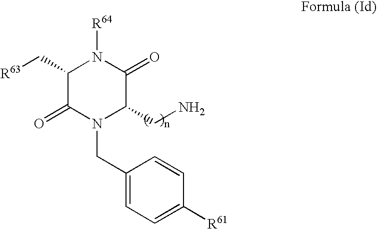

The number of the intermediate used as starting compound in general procedure E, step G are stated under i No.

TABLE VI

| Example | | | | | | | ESI-MS |
|---|---|---|---|---|---|---|---|
| no | i No. | R⁶¹ | R⁶³ | R⁶⁴ | n | Yield | (M + H)⁺ |
| 21 | i59 | Ph | 2-naphthyl | H | 1 | 75% | 450 |
| 22 | i69 | Ph | 2-naphthyl | H | 3 | 35% | 478 |
| 23 | i155 | Ph | 2-naphthyl | H | 4 | quant. | 492 |
| 24 | i156 | Ph | 2-naphthyl | Me | 4 | 66% | 506 |
| 25 | i157 | Ph | 2-naphthyl | H | 5 | 97% | 506 |

### Examples 26 and 27

Compounds of general formula (Ie) is synthesised on an ACT 440×T MOS robot according to general procedure A using as first building block (step A) Fmoc-D-Lys(Boc)-OH, Fmoc-D-Arg(Pbf)-OH, Fmoc-L-Lys(Boc)-OH or Fmoc L-Arg(Pbf)-OH. Benzaldehyde, 2-naphthylaldehyde, biphenyl-4-carbaldehyde or 4-benzyloxy-benzaldehyde is used as second building block (step C). The third building block (step D) is covered by Boc-D-Phe-OH, Boc-β-(2-naphthyl)-L-Ala-OH, Boc-D-Ser(Bzl)-OH, Boc-β-(2-naphthyl)-D-Ala-OH, Boc-L-Phe-OH, or Boc-L-Ser(Bzl)-OH. 24 random samples are analysed using HPLC-MS method B.

Examples of compounds prepared according to said procedure of the general formula (Ie) are shown in Table VII:

TABLE VII

| | |
|---|---|
| | Formula (Ie) |
| 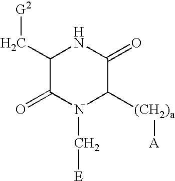 | |

| Example | | | | | Stereo | Stereo |
|---|---|---|---|---|---|---|
| No | a | A | E | G² | pos 3 | pos 6 |
| 26 | 4 | —NH₂ | -4-Biph | -2-Np | S | S |
| 27 | 4 | —NH₂ | -Ph(4-OBzl) | -2-Np | S | S |

Stereo pos 3 and 6 = Absolute stereochemistry at the position 3 and 6, respectively, of the diketopiperazin ring system

### Example 28 (General Procedure (B))

### (S,S)-6-(4-Amino-butyl)-1-biphenyl-4-ylmethyl-3-naphthalen-1-ylmethyl-piperazine-2,5-dione

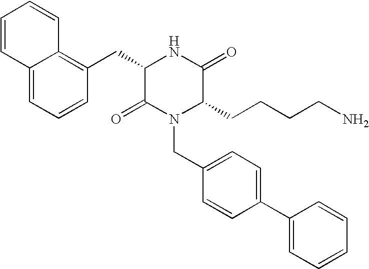 Step A:

30 mmol, 8.9 g H-Lys(Boc)-OMe hydrochloride, 1 equi., 5.5 g biphenyl-4-carbaldehyde and 1 equi., 5.1 ml DIPEA are suspended in 300 ml THF and the resulting mixture is stirred overnight. Then 2.9 equi., 5.4 g NaCNBH₃, 30 ml MeOH and 5 ml HOAc are added and the mixture is stirred for 7 h. The solvent is removed in vacuo and the residual oil is taken up in 300 ml ethyl acetate. The org. phase is washed once with 300 ml 1 M NaOH. The aq. phase is extracted once with 300 ml ethyl acetate and the combined org. phases are dried over sodium sulfate. The solvent is removed in vacuo and the crude product is used for the next step.

Step B:

20.0 mmol, 6.3 g Boc-1-NaI-OH is dissolved in 50 ml THF, 0.5 equi., 1.6 ml DIC is added and the resulting mixture is stirred for 20 min. Then 10 mmol of the crude product of step A is added in 100 ml THF and stirred overnight. Another 0.25 equi., 0.7 ml DIC is added and after 10 min 1 ml of DIPEA. The solvent is removed after 3 h of stirring and the residue is taken up in 100 ml ethyl acetate. The org. phase is washed with 100 ml 1 M HCl and twice with 100 ml 1 M NaOH and dried over sodium sulfate. The solvent is removed in vacuo and the residual oil is purified on silica with ethyl acetate/heptane (1:1).

Step C:

The purified product from step B is dissolved in 50 ml DCM and 50 ml TFA is added. The solvents are removed after 1 h. The residual oil is taken up in 50 ml DCM and 1 ml DIPEA is added. Another 1 ml of DIPEA is added after 1 h and again after an additional 90 min. The solvent is removed in vacuo and the product is purified on a C18 reverse phase column (Sep-Pak, Waters, 10 g) with 0.1% TFA in water and acetonitril.

The product is freeze dried from 0.1 N HCl in water.

¹H NMR (400.13 MHz, DMSO-d₆): δ 0.69 (m, 3H), 1.22 (m, 3H), 2.43 (m, 2H), 3.47 (m, 1H), 3.61 (m, 2H), 4.10 (m, 1H), 4.45 (m, 1H), 4.94 (1H), 7.30 (m, 2H), 7.47 (m, 7H), 7.65 (m, 2H), 7.88 (m, 1H), 7.93 (m, 2H), 7.98 (m, 1H), 8.23 (m, 1H), 8.39 (m, 1H); HPLC-MS (Method C): m/z=492 (M+1), 983 (2M+1); Rₜ=3.43 min.

### Example 29 (General Procedure (B))

### (S,S)-6-(4-Amino-butyl)-3-(4-benzyloxy-benzyl)-1-biphenyl-4-ylmethyl-piperazine-2,5-dione

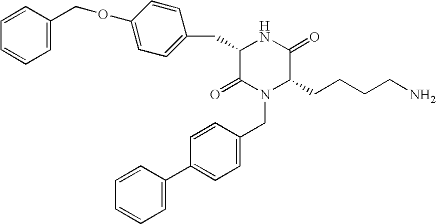 Step A:

The intermediate from example 28, Step A is used.

Step B:

20.0 mmol, 7.5 g Boc-1-Tyr(bzl)-OH is dissolved in 50 ml THF, 0.5 equi., 1.6 ml DIC is added and the resulting mixture is stirred for 40 min. Then 10 mmol of the crude product of step A is added in 50 ml THF. After 6 h 1.7 ml DIPEA is added and stirred overnight. The solvent is removed in vacuo and the residue is taken up in 100 ml ethyl acetate. The org. phase is washed twice with 100 ml 1 M HCl and twice with 100 ml sat. NaHCO₃ and dried over sodium sulfate. The solvent is removed in vacuo and the residual oil is purified on silica with ethyl acetate/heptane (2:3).

Step C:

The purified product from step B is dissolved in 100 ml DCM and 100 ml TFA is added. The solvents are removed in vacuo after 30 min. The residual oil is taken up in 100 ml DCM and 3 ml DIPEA is added. After 1 h the solvent is removed in vacuo and the oil is taken up in 100 ml DCM and 3 ml DIPEA. The solvent is removed in vacuo after 2 h and the product is purified on a C18 reverse phase column (Sep-Pak, Waters, 10 g) with 0.1% TFA in water and acetonitril. HPLC-MS (Method C): m/z=548 (M+1); Rₜ=3.23 min.

### Example 30 (General procedure (B))

### (S,S)-6-(4-Amino-butyl)-1,3-bis-biphenyl-4-ylmethyl-piperazine-2,5-dione

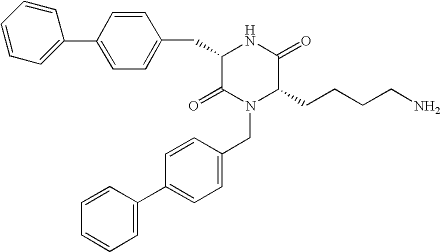 Step A:

The intermediate from example 28, Step A is used.

Step B:

20.0 mmol, 6.8 g Boc-p-phenyl-Phe-OH is dissolved in 50 ml THF, 0.5 equi., 1.6 ml DIC is added and the resulting mixture is stirred for 30 min. Then 10 mmol of the crude product of step A is added in 50 ml THF. After 4.5 h 1.7 ml DIPEA is added and the mixture is stirred overnight. The solvent is removed in vacuo and the residue is taken up in 100 ml ethyl acetate. The org. phase is washed twice with 100 ml 1 M HCl and twice with 100 ml sat. NaHCO₃ and dried over sodium sulfate. The solvent is removed in vacuo and the residual oil is purified on silica with ethyl acetate/heptane (2:3).

Step C:

The purified product from step B is dissolved in 80 ml DCM and 80 ml TFA is added. The solvents are removed in vacuo after 70 min. The residual oil is taken up in 100 ml DCM and 1.8 ml DIPEA is added. After 1 h 1 ml DIPEA is added. The solvent is removed in vacuo after 3 h. The oil is again taken up in 100 ml DCM and 2 ml DIPEA are added. The solvent is removed in vacuo after stirring for 1.5 h and the product is purified on a C18 reverse phase column (Sep-Pak, Waters, 10 g) with 0.1% TFA in water and acetonitril. HPLC-MS (Method C): m/z=518 (M+1); Rₜ=3.14 min.

### Example 31

### (S,S)-6-(4-Amino-butyl)-3-naphthalen-2-ylmethyl-1-(4-phenoxy-benzyl)-piperazine-2,5-dione

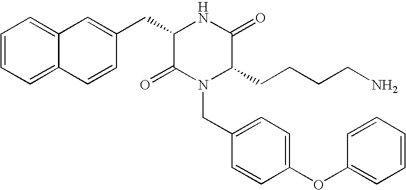 Step A: 2-tert-Butoxycarbonylamino-3-(2-naphtyl)propionic acid (5.00 g, 15.85 mmol) is dissolved in 100 ml of tetrahydrofuran in a 500 ml flask equipped with a magnetic stirrer. O-(1H-Benzotriazol-1-yl)-N,N,N′,N′-tetramethyluronium hexafluorophosphate (6.31 g, 16.64 mmol), 1-hydroxybenzotriazole (2.43 g, 15.85 mmol) and N-ethyldiisopropylamine (3.80 ml, 22.19 mmol) are added, and the mixture is stirred for 30 min, after which 20 ml of methanol is added. Stirred overnight at room temperature to give a clear yellow solution. Evaporated to a crude mixture, which is taken up in 150 ml of ethyl acetate and washed with 25 ml of aqueous sodium hydrogen sulfate (10%), 25 ml of aqueous sodium hydrogen carbonate (saturated), 25 ml of water, and 25 ml of brine, dried over magnesium sulfate and filtered. Concentrated in vacuo to give a crude oil, which is purified by flash chromatography (150 g of SiO₂, heptane:ethyl acetate (8:2)) to afford 5.52 g (quantitative yield) of (2S)-2-tert-butoxycarbonylamino-3-(2-naphthyl)propionic acid methyl ester as a yellow crystalline oil.

HPLC-MS: Rₜ=6.57 min., (M+1)=330, % Area by ELS=100

Step B:

(2S)-2-tert-Butoxycarbonylamino-3-(2-naphthyl)propionic acid methyl ester (5.52 g, theoretically 15.85 mmol) is dissolved in 20 ml of ethyl acetate in a 500 ml flask equipped with a magnetic stirrer. To the stirred solution is added 80 ml of 2.8 M hydrogen chloride in ethyl acetate and the reaction is stirred for 2.5 hours under nitrogen. The clear mixture is concentrated in vacuo to give a solid, which is taken up in ethyl acetate, stirred for 10 min and filtered. The solid is dried in vacuo at 40° C. to give 3.91 g (93%) of (2S)-2-Amino-3-(2-naphthyl)propionic acid methyl ester hydrochloride as a white solid.

HPLC-MS: Rt=3.70 min., (M+1)=230, % Area by ELS=100

Step C:

2- tert-Butoxycarbonylamino-6-(9H-fluoren-9-ylmethoxycarbonylamino)hexanoic acid (4.87 g, 10.39 mmol) is dissolved in 60 ml of dimethylformamide in a 250 ml flask equipped with a magnetic stirrer. 1-Hydroxybenzotriazole (1.59 g, 10.39 mmol) and N-ethyl-N′-(3-dimethylaminopropyl)-carbodiimide hydrochloride (1.99 g, 10.39 mmol) are added and the mixture is stirred for 30 min, after which (2S)-2-amino-3-(2-naphthyl)propionic acid methyl ester (2.76 g, 10.39 mmol) and N-ethyldiisopropylamin (3.6 ml, 20.78 mmol) are added. Stirred for 3 days to give a clear orange solution. The reaction is added to 200 ml of ethyl acetate and washed with a mixture of 25 ml of water and 25 ml of aqueous sodium hydrogen carbonate (saturated). The aqueous phase is extracted with 100 ml of ethyl acetate. The combined organic phases are then washed with 50 ml of aqueous sodium hydrogen sulfate (10%), 50 ml of brine, dried over magnesium sulfate and filtered. This solution of (2S)-2-[2-tert-butoxycarbonylamino-6-(9H-fluoren-9-ylmethoxycarbonylamino)hexanoyl amino]-(2S)-3-(2-naphthyl)propionic acid methyl ester is used directly in the next step without further purification.

HPLC-MS: Rt=7.73 min., (M+1)=680, % Area by ELS=99

Step D: To (2S)-2-[2-tert-butoxycarbonylamino-6-(9H-fluoren-9-ylmethoxycarbonylamino)hexanoylamino]-(2S)-3-(2-naphthyl)propionic acid methyl ester (theoretically 10.39 mmol in 250 ml of ethyl acetate) is added 250 ml of 2.8 M hydrogen chloride in ethyl acetate. The reaction is stirred for 2 hours under nitrogen. Concentrated in vacuo to afford 5.86 g (92%) of (2S)-2-[2-amino-6-(9H-fluoren-9-ylmethoxycarbonylamino)hexanoylamino]-(2S)-3-(2-naphthyl)propionic acid methyl ester hydrochloride as orange oil.

HPLC-MS: Rt=5.43 min., (M+1)=580, % Area by ELS=74

Step E:

To a solution of 2S)-2-[2-amino-6-(9H-fluoren-9-ylmethoxycarbonylamino)hexanoylamino]-(2S)-3-(2-naphthyl)propionic acid methyl ester (0.50 g, 0.81 mmol) in a mixture of 5 ml of tetrahydrofuran and 5 ml of methanol is added sodium acetate (0.27 g, 3.24 mmol), 4-phenoxybenzaldehyde (0.14 ml, 0.81 mmol), molecular sieves (4A) and 1.0 M sodium cyanoborohydride (0.81 ml, 0.81 mmol) in tetrahydrofuran. Stirred overnight and then filtered through Hyflo Super Cel®. Concentrated in vacuo to afford (2S)-2-[6-(9H-fluoren-9-ylmethoxycarbonylamino)-2-(4-phenoxybenzylamino)hexanoylamino]-(2S)-3-(2-naphthyl)propionic acid methyl ester (theoretically 0.81 mmol) as a solid, which is used without further purification.

HPLC-MS: Rt=7.53 min., (M+1)=762, % Area by ELS=100

Step F:

A solution of (2S)-2-[6-(9H-fluoren-9-ylmethoxycarbonylamino)-2-(4-phenoxybenzylamino)hexanoylamino]-(2S)-3-(2-naphthyl)propionic acid methyl ester (theoretically 0.81 mmol) in 15 ml of toluene, 15 ml of 1-butanol and 3 ml of acetic acid is stirred for 12 hours at 100° C. in a 250 ml flask equipped with a condenser. Concentrated in vacuo, dissolved in 100 ml of dichloromethane and washed with 20 ml of aqueous sodium hydrogen carbonate (saturated), 20 ml of aqueous sodium hydrogen sulfate (10%), 20 ml of brine, dried over magnesium sulfate, filtered and concentrated in vacuo to afford {(2S,5S)-4-[5-(2-naphthyl)methyl-3,6-dioxo-1-(4-phenoxybenzyl)piperazin-2-yl]butyl}carbamic acid (9H-fluoren-9-ylmethyl)ester (theoretically 0.81 mmol) as an oil. Used without further purification.

HPLC-MS: Rt=8.28 min., (M+1)=730, % Area by ELS=100

Step G:

To a solution of {(2S,5S)-4-[5-(2-naphthyl)methyl-3,6-dioxo-1-(4-phenoxybenzyl) piperazin-2-yl]butyl}carbamic acid (9H-fluoren-9-ylmethyl)ester (theoretically 0.81 mmol) in 10 ml of dichloromethane is added 10 ml of tris(2-aminoethyl)amine. Stirred for 2 hours under nitrogen. The reaction is added to 100 ml of dichloromethane and washed with 30 ml of brine, 3×50 ml of aqueous phosphate buffer (pH 6.6), 50 ml of brine, dried over magnesium sulfate, filtered and concentrated in vacuo to afford a crude oil which is purified by preparative HPLC (20-40% CH₃CN in water/0.1% trifluoroacetic acid, 40 min). The obtained pure fractions are combined and 1 ml of 1N aqueous hydrogen chloride is added. The compound is lyophilized to give 60.1 mg (14%) of the title compound as a hydrochloride-salt.

HPLC (A1): Rt=33.22 min., 100% (214 nm); HPLC (B1): Rt=35.14 min., 100% (214 nm); HPLC-MS: Rt=4.83 min., (M+1)=508, % Area by ELS=100

### Example 32 (General Procedure (B))

### (S,S)-6-(4-Amino-butyl)-3-benzo[b]thiophen-3-ylmethyl-1-biphenyl-4-ylmethyl-piperazine-2,5-dione

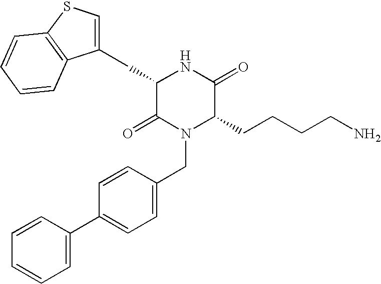 Step A:

The intermediate from example 28, Step A is used.

Step B:

10.0 mmol, 3.21 g Boc-β-(3-benzothienyl)-Ala-OH is dissolved in 30 ml THF, 0.5 equi., 775 μl DIC is added and the resulting mixture is stirred for 30 min. Then 5 mmol of the crude product of step A is added in 30 ml THF. After 2.5 h 855 μl DIPEA is added and the mixture is stirred overnight. The solvent is removed in vacuo and the residue is taken up in 100 ml ethyl acetate. The org. phase is washed twice with 50 ml 1 M HCl and twice with 50 ml sat. NaHCO₃ and dried over sodium sulfate. The solvent is removed in vacuo and the residual oil is purified on silica with ethyl acetate/heptane (1:2).

Step C:

The purified product from step B is dissolved in 50 ml DCM and 50 ml TFA is added. The solvents are removed in vacuo after 40 min. The residual oil is taken up in 50 ml DCM and 2.0 ml DIPEA is added. The solvent is removed in vacuo after 3 h. The oil is again taken up in 50 ml DCM and 1.5 ml DIPEA are added. The solvent is removed in vacuo after stirring for 1.5 h and the product is purified on a C18 reverse phase column (Sep-Pak, Waters, 10 g) with 0.1% TFA in water and acetonitril.

HPLC-MS (Method C): m/z=498 (M+1); Rₜ=2.86 min.

### Example 33 (General Procedure (B))

### (S,S)-6-(4-Amino-butyl)-3-(4-benzoyl-benzyl)-1-biphenyl-4-ylmethyl-piperazine-2,5-dione

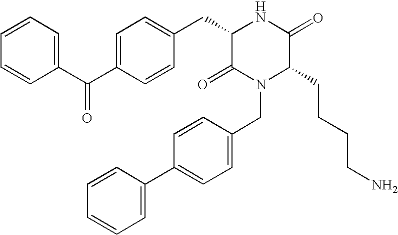 Step A:

The intermediate from example 28, Step A is used.

Step B:

10.0 mmol, 3.7 g Boc-p-Bz-Phe-OH is dissolved in 30 ml THF, 0.5 equi., 755 μl DIC is added and the resulting mixture is stirred for 30 min. Then 5 mmol of the crude product of step A is added in 30 ml THF. After 2.5 h 855 μl DIPEA is added and the mixture is stirred overnight. The solvent is removed in vacuo and the residue is taken up in 70 ml ethyl acetate. The org. phase is washed twice with 50 ml 1 M HCl and twice with 50 ml sat. NaHCO₃ and dried over sodium sulfate. The solvent is removed in vacuo and the residual oil is purified on silica with ethyl acetate/heptane (2:3).

Step C:

The purified product from step B is dissolved in 50 ml DCM and 50 ml TFA is added. The solvents are removed in vacuo after 45 min. The residual oil is taken up in 50 ml DCM and 3.0 ml DIPEA is added. The solvent is removed in vacuo after 2.3 h and the product is purified on a C18 reverse phase column (Sep-Pak, Waters, 10 g) with 0.1% TFA in water and acetonitril.

HPLC-MS (Method C): m/z=546 (M+1); Rₜ=2.98 min.

### Example 34

### (S,S)-6-(4-Amino-butyl)-1-(4′-methoxy-biphenyl-4-ylmethyl)-3-naphthalen-2-ylmethyl-piperazine-2,5-dione

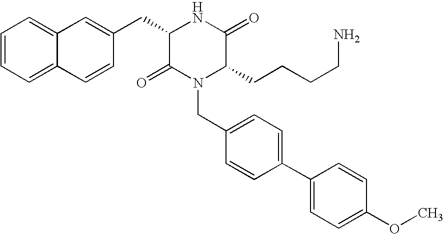

35 mg of the title compound is synthesized as described for example 31 using 4′-methoxy-biphenyl-4-carbaldehyde instead of 4-phenoxybenzaldehyde.

The title compound is purified by Sep-Pak® using 70% acetonitrile in water/0.1 M hydrogen chloride as a mobile phase. The mobile phase is removed by lyophilization.

HPLC (A): Rt=32.92 min., 90% (214 nm); HPLC (B): Rt=34.51 min., 89% (214 nm); HPLC-MS: Rt=4.67 min., (M+1)=522, % Area by ELS=97

### Example 35

### (S,S)-6-(4-Amino-butyl)-3-naphthalen-2-ylmethyl-1-(4′-trifluoromethyl-biphenyl-4-ylmethyl)piperazine-2,5-dione

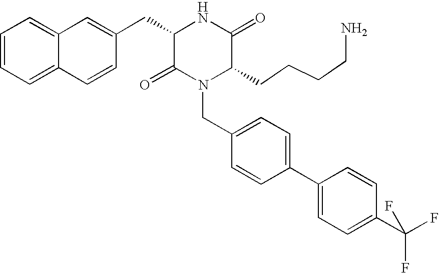

24 mg of the title compound is synthesized as described for example 31 using 4′-trifluoromethyl-biphenyl-4-carbaldehyde instead of 4-phenoxybenzaldehyde.

The title compound is purified by Sep-Pak® using 70% acetonitrile in water/0,1 M hydrogen chloride as a mobile phase. The mobile phase is removed by lyophilization.

HPLC (A1): Rₜ=38.26 min., 94% (214 nm); HPLC (B1): Rₜ=40.39 min., 94% (214 nm); HPLC-MS: Rt=5.22 min, (M+1)=560, % Area by ELS=100.

### Example 36

### (S,S)-6-(4-Amino-butyl)-1-(4′-chloro-biphenyl-4-ylmethyl)-3-naphthalen-2-ylmethyl-piperazine-2,5-dione

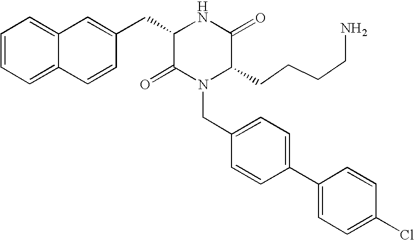

35 mg of the title compound is synthesized as described for example 31 using 4′-chloro-biphenyl-4-carbaldehyde instead of 4-phenoxybenzaldehyde.

The title compound is purified by Sep-Pak® using 70% acetonitrile in water /0,1 M hydrogen chloride as a mobile phase. The mobile phase is removed by lyophilization.

HPLC (A1): Rt=38.93 min., 87% (214 nm); HPLC (B1): Rₜ=38.64 min., 90% (214 nm); HPLC-MS: Rt=4.88 min, (M+1)=526, % Area by ELS=70.

### Example 37

### (S,S)-6-(4-Amino-butyl)-1-(9H-fluoren-2-ylmethyl)-3-naphthalen-2-ylmethyl-piperazine-2,5-dione

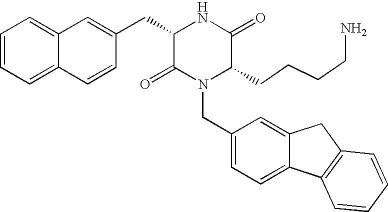

16.6 mg of the title compound is synthesized as described for example 31 using 9H-fluorene-carbaldehyde instead of 4-phenoxybenzaldehyde.

The title compound is purified by preparative HPLC (25-45% acetonitrile in water /0.1% trifluoroacetic acid, 40 min). The obtained pure fractions are combined and 1 N aqueous hydrogen chloride is added. The mobile phase is removed by lyophilization.

HPLC (A1): Rₜ=33.34 min., 98% (214 nm); HPLC (B1): Rₜ=35.38 min., 98% (214 nm); HPLC-MS: Rt=4.93 min, (M+1)=504, % Area by ELS=66

### Example 38

### (S,S)-4′-[2-(4-Amino-butyl)-5-naphthalen-2-ylmethyl-3,6-dioxo-piperazin-1-ylmethyl]-biphenyl-2-carboxylic acid methyl

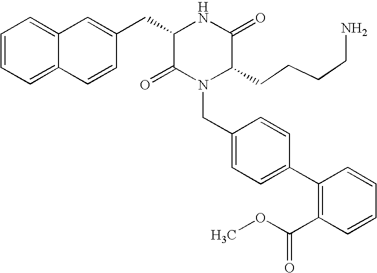

10.5 mg of the title compound is synthesized as described for example 31 using 9H-fluorene-carbaldehyde instead of 4-phenoxybenzaldehyde.

The title compound is purified by preparative HPLC (23-43% acetonitrile in water /0.1% trifluoroacetic acid, 40 min). The obtained pure fractions are combined and 1 N aqueous hydrogen chloride is added. The mobile phase is removed by lyophilization.

HPLC (A1): Rt=33.03 min., 100% (214 nm); HPLC (B1): Rt=34.98 min., 100% (214 nm); HPLC-MS: Rt=4.42 min, (M+1)=550, % Area by ELS=100

### Example 39 (General Procedure (B))

### (S,S)-6-(4-Amino-butyl)-3-(4-benzoyl-benzyl)-1-(4-phenoxy-benzyl)-piperazine-2,5-dione

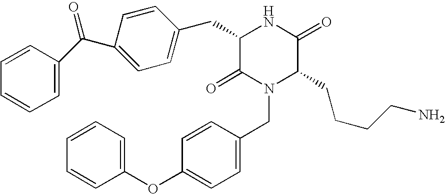 Step A:

20 mmol, 5.9 g H-Lys(Boc)-OMe hydrochloride, 1 equi., 3.6 ml 4-phenoxy benzaldehyde and 1 equi., 3.5 ml DIPEA are suspended in 200 ml THF and the resulting mixture is stirred overnight. Then 2.9 equi., 3.7 g NaCNBH₃, 20 ml MeOH and 10 ml HOAc are added and the mixture is stirred for 3 h. The solvent is removed in vacuo and the residual oil is taken up in 200 ml ethyl acetate. The org. phase is washed twice with 100 ml 1 M NaOH. and dried over sodium sulfate. The solvent is removed in vacuo and the crude product is used for the next step.

Step B:

5.0 mmol, 1.9 g Boc-p-Bz-Phe-OH is dissolved in 15 ml THF, 0.5 equi. and 390 μl DIC is added and the resulting mixture is stirred for 30 min. Then 2.5 mmol of the crude product of step A is added in 15 ml THF. After 2.5 h 430 μl DIPEA is added and the mixture is stirred overnight. The solvent is removed in vacuo and the residue is taken up in 40 ml ethyl acetate. The org. phase is washed twice with 30 ml 1 M HCl and twice with 30 ml sat. NaHCO₃ and dried over sodium sulfate. The solvent is removed in vacuo and the residual oil is purified on silica with ethyl acetate/heptane (2:3).

Step C:

The purified product from step B is dissolved in 30 ml DCM and 30 ml TFA is added. The solvents are removed in vacuo after 30 min. The residual oil is taken up in 30 ml DCM and 2.0 ml DIPEA is added. The solvent is removed in vacuo after 2 h and the product is purified on a C18 reverse phase column (Sep-Pak, Waters, 10 g) with 0.1% TFA in water and acetonitril.

HPLC-MS (Method C): m/z=562 (M+1); Rₜ=3.02 min.

### Example 40 (General Procedure (B))

### (S,S)-6-(4-Amino-butyl)-3-(4-methoxy-benzyl)-1-(4-phenoxy-benzyl)-piperazine-2,5-dione

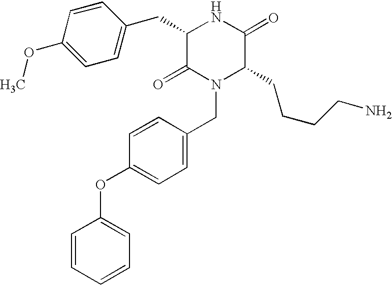 Step A:

The intermediate from example 39, Step A is used.

Step B:

5.0 mmol, 1.5 g Boc-p-methoxy-Phe-OH is dissolved in 15 ml THF, 0.5 equi., 390 μl DIC is added and the resulting mixture is stirred for 30 min. Then 2.5 mmol of the crude product of step A is added in 15 ml THF. After 3.5 h 430 μl DIPEA is added and the mixture is stirred overnight. The solvent is removed in vacuo and the residue is taken up in 40 ml ethyl acetate. The org. phase is washed twice with 25 ml 1 M HCl and twice with 25 ml sat. NaHCO₃ and dried over sodium sulfate. The solvent is removed in vacuo and the residual oil is purified on silica with ethyl acetate/heptane (2:3).

Step C:

The purified product from step B is dissolved in 30 ml DCM and 30 ml TFA is added. The solvents are removed in vacuo after 30 min. The residual oil is taken up in 30 ml DCM and 2.0 ml DIPEA is added. The solvent is removed in vacuo after 2.3 h and the product is purified on a C18 reverse phase column (Sep-Pak, Waters, 10 g) with 0.1% TFA in water and acetonitril. HPLC-MS (Method C): m/z=488 (M+1); Rₜ=2.92 min.

### Example 41 (General Procedure (B))

### (S,S)-6-(4-Amino-butyl)-3-(4-chloro-benzyl)-1-(4-phenoxy-benzyl)-piperazine-2,5-dione

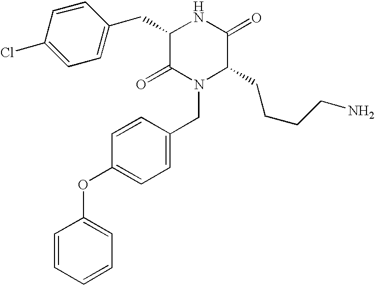 Step A:

The intermediate from example 39, Step A is used.

Step B:

5.0 mmol, 1.5 g Boc-p-chloro-Phe-OH is dissolved in 15 ml THF, 0.5 equi., 390 μl DIC is added and the resulting mixture is stirred for 30 min. Then 2.5 mmol of the crude product of step A is added in 15 ml THF. After 2.5 h 430 μl DIPEA is added and the mixture is stirred overnight. The solvent is removed in vacuo and the residue is taken up in 40 ml ethyl acetate. The org. phase is washed twice with 25 ml 1 M HCl and twice with 25 ml sat. NaHCO₃ and dried over sodium sulfate. The solvent is removed in vacuo and the residual oil is purified on silica with ethyl acetate/heptane (2:3).

Step C:

The purified product from step B is dissolved in 25 ml DCM and 25 ml TFA is added. The solvents are removed in vacuo after 30 min. The residual oil is taken up in 40 ml DCM and 2.0 ml DIPEA is added. The solvent is removed in vacuo after 1.5 h and the product is purified on a C18 reverse phase column (Sep-Pak, Waters, 10 g) with 0.1% TFA in water and acetonitril.

HPLC-MS (Method C): m/z =492 (M+1); Rₜ=2.75 min.

### Example 42 (General Procedure (B))

### (S,S)-6-(4-Amino-butyl)-3-(4-methyl-benzyl)-1-(4-phenoxy-benzyl)-piperazine-2,5-dione

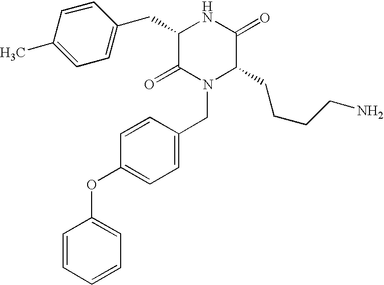 Step A:

The intermediate from example 39, Step A is used.

Step B:

5.0 mmol, 1.4 g Boc-p-chloro-Phe-OH is dissolved in 15 ml THF, 0.5 equi., 390 μl DIC is added and the resulting mixture is stirred for 30 min. Then 2.5 mmol of the crude product of step A is added in 15 ml THF. After 2 h 430 μl DIPEA is added and the mixture is stirred overnight. The solvent is removed in vacuo and the residue is taken up in 40 ml ethyl acetate. The org. phase is washed twice with 25 ml 1 M HCl and twice with 25 ml sat. NaHCO₃ and dried over sodium sulfate. The solvent is removed in vacuo and the residual oil is purified on silica with ethyl acetate/heptane (2:3).

Step C:

The purified product from step B is dissolved in 25 ml DCM and 25 ml TFA is added. The solvents are removed in vacuo after 30 min. The residual oil is taken up in 25 ml DCM and 2.0 ml DIPEA is added. The solvent is removed in vacuo after 1 h and the product is purified on a C18 reverse phase column (Sep-Pak, Waters, 10 g) with 0.1% TFA in water and acetonitril.

HPLC-MS (Method C): m/z=472 (M+1); Rₜ=2.80 min.

### Example 43

### (S,S)-4′-[2-(4-Amino-butyl)-5-naphthalen-2-ylmethyl-3,6-dioxo-piperazin-1-ylmethyl]-biphenyl-2-carbonitrile

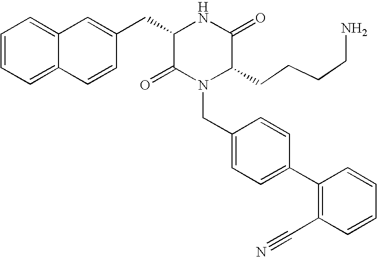

26 mg of the title compound is synthesized as described for example 31 using 4′-formyl-biphenyl-2-carbonitrile instead of 4-phenoxybenzaldehyde.

The title compound is purified by preparative HPLC (23-43% acetonitrile in water /0.1% trifluoroacetic acid, 40 min). The obtained pure fractions are combined and 1 N aqueous hydrogen chloride is added. The mobile phase is removed by lyophilization.

HPLC-MS: Rₜ=4.32 min, (M+1)=517, % Area by ELS=100

### Example 44

### (S,S)-6-(4-Amino-butyl)-1-(4-cyclohexyloxy-benzyl)-3-naphthalen-2-ylmethyl-piperazine-2,5-dione

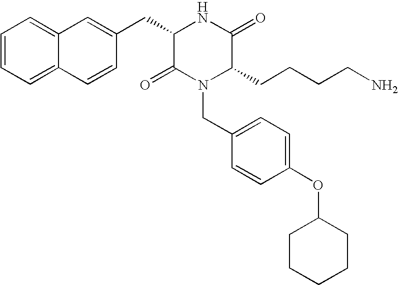 Step A:

To a solution of H-Lys(Boc)-OMe HCl (3.0 g, 10 mmol) in THF (120 ml) is added 4-hydroxy-benzaldehyde (1.23 g, 10 mmol) and N,N-diisopropylethylamine (1.76 ml, 10 mmol), and the mixture is stirred for 5 h at room temperature. Then methanol (10 ml), acetic acid (4.8 ml) and sodium cyanoborohydride (1.9 g, 30 mmol) is added and the mixture is stirred overnight at room temperature. Then the mixture is evaporated in vacuo and the residue is taken up in ethyl acetate (150 ml) and filtered. The filtrate is washed with 1 N sodium hydroxide (75 ml). The aqueous phase is extracted with ethyl acetate (75 ml) and the combined organic phases are dried over sodium sulfate and evaporated to dryness to give the crude product, which is used in the next step without further purification.

HPLC-MS (Method C): m/z=367 (M+1); Rₜ=2.23 min.

Step B:

To a solution of Boc-O-2-naphthyl-Ala-OH (4.4 g, 14 mmol) in THF (30 ml) is added N,N′-diisopropylcarbodiimide (1.1 ml, 7.1 mmol) and the mixture is stirred for 30 min at room temperature. A solution of crude product from step A (2.60 g, 7.1 mmol) in THF (20 ml) is added and the mixture is stirred for 7 h at room temperature. Then triethylamine (2.0 ml, 14 mmol) is added and stirring is continued overnight. The mixture is evaporated in vacuo and the residue is taken up in ethyl acetate (75 ml) and washed successively with 1 N HCl (50 ml) and saturated aqueous sodium hydrogen carbonate (50 ml), dried over sodium sulfate and evaporated to dryness. Column chromatography on silica (ethyl acetate/heptane (1:3 to 1:1)) afforded the intermediate in a yield of 2.4 g (50%).

HPLC-MS (Method C): m/z=686 (M+23); Rₜ=5.14 min.

Step C:

Triphenylphosphine (356 mg, 1.4 mmol) and cyclohexanol (136 mg, 1.4 mmol) is added to a solution of the product from step B (300 mg, 0.45 mmol) in THF (20 ml) with stirring at room temperature under nitrogen. A solution of diethyl azodicarboxylate (214 ml, 1.4 mmol) in THF (5 ml) is added dropwise during 30 min while the temperature is kept below 30° C. with cooling on an ice-bath. After stirring at room temperature for about 3 days the mixture is evaporated to dryness and purified on silica with ethyl acetate/heptane (1:3) affording a crude product, which is used in the next step without further purification.

Step D:

The product from step C (50 mg, 0.067 mmol) is dissolved in DCM (10 ml) and TFA (5 ml) is added. The solution is stirred for 2 h at room temperature. After evaporation in vacuo the residue is taken up in toluene (10 ml) and the solvent is again removed in vacuo. The residue is now dissolved in DCM (10 ml) and N,N-diisopropylethylamine (100 μl, 0.57 mmol) is added. After stirring overnight, the mixture is evaporated in vacuo and the residue is purified on a C18 reverse phase column (Sep-Pak, Waters, 10 g) with 0.1% TFA in water and acetonitrile. The product is dissolved in a mixture of 1 N HCl and methanol and evaporated in vacuo affording 25 mg of the title compound as the hydrochloride.

HPLC-MS (Method C): m/z=514 (M+1); Rₜ=3.3 min.

### Example 45

### (S,S)-6-(4-Amino-butyl)-3-naphthalen-2-ylmethyl-1-[4-(3-trifluoromethyl-cyclohexyloxy)-benzyl]-piperazine-2,5-dione

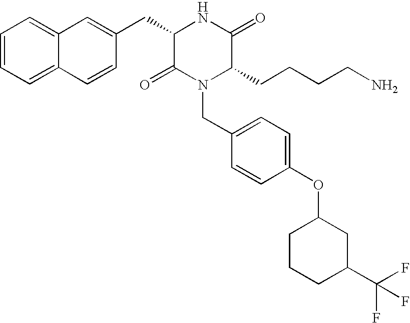 Step A:

Triphenylphosphine (356 mg, 1.4 mmol) and 3-trifluoromethylcyclohexanol (235 mg, 1.4 mmol) is added to a solution of the product from step B in example 44 (312 mg, 0.456 mmol) in THF (20 ml) with stirring at room temperature under nitrogen. A solution of diethyl azodicarboxylate (214 ml, 1.4 mmol) in THF (5 ml) is added dropwise during 30 min while the temperature is kept below 30° C. with cooling on an ice-bath. After stirring at room temperature for 2 days the mixture is evaporated to dryness and purified on silica with ethyl acetate/heptane (1:4) affording 400 mg of the product, which is used in the next step without further purification.

HPLC-MS (Method C): m/z=836 (M+23); Rₜ=6.5 min.

Step B:

The product from step A (202 mg, 0.24 mmol) is dissolved in DCM (15 ml) and TFA (15 ml) is added. The solution is stirred for 6 h at room temperature. After evaporation in vacuo the residue is taken up in toluene (10 ml) and the solvent is again removed in vacuo. The residue is now dissolved in DCM (20 ml) and N,N-diisopropylethylamine (83 μl, 0.48 mmol) is added. After stirring overnight, the mixture is evaporated in vacuo and the residue is purified on a C18 reverse phase column (Sep-Pak, Waters, 10 g) with 0.1% TFA in water and. The product is dissolved in a mixture of 1 N HCl and methanol and evaporated in vacuo affording 100 mg of the title compound as the hydrochloride

HPLC-MS (Method C): m/z=582 (M+1); Rₜ=3.4 min.

### Example 46

### (S,S)-6-(4-Amino-butyl)-1-(4-cyclohexyl-benzyl)-3-naphthalen-2-ylmethyl-piperazine-2,5-dione

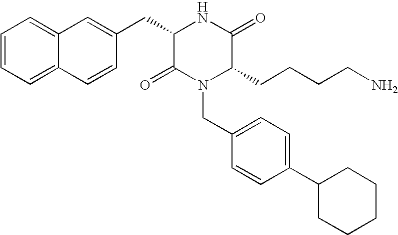 Step A:

To a solution of H-Lys(Boc)-OMe HCl (1.04 g, 3.5 mmol) in THF (40 ml) is added 4-cyclohexyl-benzaldehyde (0.67 g, 3.5 mmol) and N,N-diisopropylethylamine (0.5 ml, 3 mmol), and the mixture is stirred for 4 h at room temperature. Then methanol (3.4 ml), acetic acid (1.6 ml) and sodium cyanoborohydride (0.65 g, 1 0 mmol) is added and the mixture is stirred overnight at room temperature. Then the mixture is evaporated in vacuo and the residue is taken up in ethyl acetate (150 ml) and washed with saturated aqueous sodium chloride (30 ml) and 1N sodium hydroxide (30 ml). The organic phase is dried over sodium sulfate and evaporated to dryness to give the crude product, which is used in the next step without further purification.

HPLC-MS (Method C): m/z=433 (M+1); Rₜ=3.7 min.

Step B:

To a solution of Boc-β-2-naphthyl-Ala-OH (1.8 g, 5.6 mmol) in THF (15 ml) is added N,N′-diisopropylcarbodiimide (0.35 ml, 2.8 mmol) and the mixture is stirred for 30 min at room temperature. A solution of crude product from step A (1.20 g, 2.8 mmol) in THF (15 ml) is added and the mixture is stirred for 7 h at room temperature. Then N,N-diisopropylethylamine (0.72 ml, 5.6 mmol) is added and stirring is continued overnight. The mixture is evaporated in vacuo and the residue is taken up in ethyl acetate (75 ml) and washed successively with 1 N HCl (50 ml) and saturated aqueous sodium hydrogen carbonate (50 ml), dried over sodium sulfate and evaporated to dryness. Column chromatography on silica with ethyl acetate/heptane (1:3 to 1:1) afforded the intermediate in a yield of 420 mg (20%).

HPLC-MS (Method C): m/z=752 (M+23), 730 (M+1); Rₜ=6.7 min.

Step C:

The product from step B is dissolved in DCM (30 ml) followed by the addition of TFA (10 ml). The solution is stirred for 2 h at room temperature. After evaporation in vacuo the residue is taken up in toluene (30 ml) and the solvent is again removed in vacuo. The residual oil is now dissolved in DCM (20 ml) and N,N-diisopropylethylamine (0.98 ml, 5.6 mmol) is added. After stirring overnight, the mixture is evaporated in vacuo and the residue is purified on a C18 reverse phase column (Sep-Pak, Waters, 10 g) with 0.1% TFA in water and acetonitrile The product is dissolved in a mixture of 1 N HCl and methanol and evaporated in vacuo affording 150 mg of the title compound as the hydrochloride

HPLC-MS (Method C): m/z=498 (M+1); Rₜ=3.4 min.

### Example 47

### (S,S)-1-Biphenyl-4-ylmethyl-6-(4-dimethylamino-butyl)-3-naphthalen-2-ylmethyl-piperazine-2,5-dione

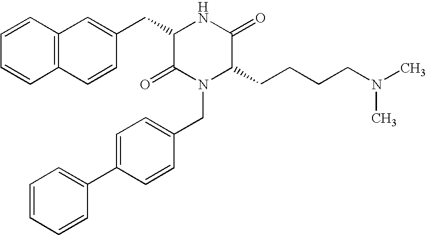 Step A:

0.123 g (0.25 mmol) of (S,S)-6-(4-amino-butyl)-1-biphenyl-4-ylmethyl-3-naphthalen-2-ylmethyl-piperazine-2,5-dione (example 11) is mixed with 1 ml of tetrahydrofuran, 0.288 ml (3.8 mmol) of 37% formalin solution, and 0.045 ml of acetic acid. The mixture is stirred for 30 minutes. 0.027 g (0.425 mmol) of sodium cyanoborohydride is added, followed by 1 ml of tetrahydrofuran and 1 ml of methanol. The mixture is stirred for 24 hours and then poured into 50 ml of 37% aqueous hydrochloric acid. After filtration, the resulting filtrate is cooled and treated with sodium hydroxide until pH =14. The resulting precipitate is collected by filtration, washed with water and dried in vacuo to give 54 mg of the product.

HPLC-MS (Method B): m/z=520 (M+1); Rₜ=4.43 min.

### Example 48

### (S,S)-1-Biphenyl-4-ylmethyl-6-(4-methylamino-butyl)-3-naphthalen-2-ylmethyl-piperazine-2,5-dione

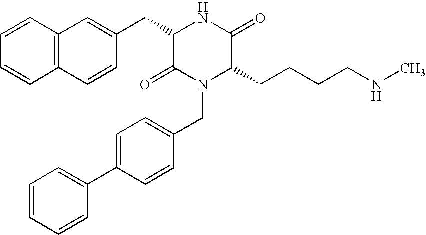 Step A:

0.295 g (0.60 mmol) of (S,S)-6-(4-amino-butyl)-1-biphenyl-4-ylmethyl-3-naphthalen-2-ylmethyl-piperazine-2,5-dione (product from example 11) is mixed with 11 ml of dichloromethane and 0.185 ml (1.32 mmol) of triethylamine. A solution of 0.133 g (0.60 mmol) 2-nitrobenzenesulfonylchloride in 5 ml of dichloromethane is added. The mixture is stirred for 30 minutes. After addition of 25 ml of dichloromethane, the mixture is washed with 1 M aqueous hydrochloric acid (4×40 ml), water (50 ml), and aqueous sodium hydrogencarbonate (40 ml). The organic phase afforded, after drying with Na₂SO₄, filtration, and evaporation, 0.385 g (0.57 mmol) of (S,S)-N-[4-(1-biphenyl-4-ylmethyl-5-naphthalen-2-ylmethyl-3,6-dioxo-piperazin-2-yl)-butyl]-2-nitrobenzenesulfonamide.

Step B:

0.169 g (0.250 mmol) of the sulfonamide obtained by step A is mixed with 0.021 g (0.150 mmol) of potassium carbonate, 0.6 ml of dimethylformamide, and 0.036 ml (0.575 mmol) of methyl iodide. The mixture is stirred for 22 hours. The methyl iodide is evaporated off.

Step C:

The suspension obtained by step B is treated with 0.069 g (0.50 mmol) of potassium carbonate, 0.30 ml of dimethylformamide, and 0.070 ml (1.0 mmol) of 2-mercaptoethanol and stirred for four hours. The mixture is partitioned between 40 ml of ethyl acetate and 20 ml of 0.2 M aqueous sodium hydroxide. The organic phase is washed with 0.2 M aqueous sodium hydroxide (2×20 ml) and water (30 ml). Drying over sodium sulfate, filtration and evaporation afforded 0.141 g of a tough yellow residue. This is dissolved in 1.5 ml of dichloromethane and eluted through a silicagel column (30 ml of “Kieselgel 60”, 230-400 mesh, Macherey-Nagel GmbH & Co. KG) with ethyl acetate/methanol/aq.NH₃ (10:10:1; aq. NH₃=25% aqueous ammonia). The eluate is collected as 10-ml-fractions. One fraction (Rf=0.33 with methanol/aq.NH₃ 95:5 on silicagel-TLC) is evaporated to give 9 mg of the product.

¹H NMR (400 MHz, CDCl₃): δ1.05-1.45 ppm (m, 6H), 2.33 ppm (m_{c}, 2H) overlapped with 2.36 ppm (s, 3H), 3.23 ppm (dd, J=13.7 Hz and 8.6 Hz, 1H), 3.53 ppm (dd, J=13.7 Hz and 3.6 Hz, 1H), 3.82 ppm (dd, J=7.2 Hz and 3.3 Hz, 1H), 4.03 ppm (d, J=14.8 Hz, 1H), 4.43 ppm (dd, J=8.6 Hz and 3.6 Hz, 1H), 5.34 ppm (d, J=14.8 Hz, 1H), 6.03 ppm (broad s, 1H), 7.30-7.57 ppm (m, 12H), 7.70 ppm (s, 1H), 7.80-7.85 ppm (m, 3H); HPLC-MS (Method B): m/z=506 (M+1); Rₜ=4.38 min.

### Example 49 (General Procedure (B))

### (S,S)-6-(4-Amino-butyl)-3-(4-ethoxy-benzyl)-1-(4-phenoxy-benzyl)-piperazine-2,5-dione

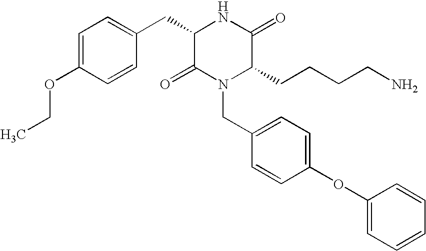 Step A:

The intermediate from example 39, Step A is used.

Step B:

5.0 mmol, 1.6 g Boc-Tyr(Et)-OH is dissolved in 15 ml THF, 0.5 equi., 390 μl DIC is added and the resulting mixture is stirred for 35 min. Then 2.5 mmol of the crude product of step A is added in 15 ml THF. After 4 h 430 μl DIPEA is added and the mixture is stirred overnight. The solvent is removed in vacuo and the residue is taken up in 60 ml ethyl acetate. The org. phase is washed twice with 30 ml 1 M HCl and twice with 30 ml sat. NaHCO₃ and dried over sodium sulfate. The solvent is removed in vacuo and the residual oil is purified on silica with ethyl acetate/heptane (2:3).

Step C:

The purified product from step B is dissolved in 30 ml DCM and 30 ml TFA is added. The solvents are removed in vacuo after 30 min. The residual oil is taken up in 30 ml DCM and 2.0 ml DIPEA is added. The solvent is removed in vacuo after 1.5 h and the product is purified on a C18 reverse phase column (Sep-Pak, Waters, 10 g) with 0.1% TFA in water and acetonitril.

HPLC-MS (Method C): m/z=502 (M+1); Rₜ=2.81 min.

### Example 50 (General Procedure (D))

### (S,S)-6-(4-Amino-butyl)-1-biphenyl-4-ylmethyl-3-(4-propoxy-benzyl)-piperazine-2,5-dione

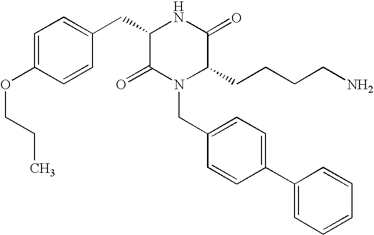 Step A:

50 mmol, 16.9 g Boc-Tyr(t-bu)-OH is dissolved in 150 ml THF and 0.5 equi, 3.9 ml DIC is added. The mixture is stirred for 30 min and the intermediate of example 28, step A, 25 mmol, 10.7 g in 100 ml THF is added. After 2 h 1 equi., 4.2 ml DIPEA is added and the mixture is stirred overnight. The solvent is removed in vacuo and the oil is taken up in 250 ml ethyl acetate. The org. phase is washed twice with 150 ml 1 M HCl and twice with 150 ml sat. NaHCO₃, the org. phase is dried over sodium sulfate and the solvent is removed in vacuo. The residual oil is purified on silica with ethyl acetate:heptane 2:3.

The pure product is dissolved in 100 ml DCM and 100 ml TFA. The solvent is removed after 15 min and the oil taken up in 150 ml DCM and 5 ml DIPEA is added and the mixture stirred at room temperature. After 20 min another 5 ml DIPEA are added and again after 3 h and 5 h. The solvent is removed in vacuo after 6 h and used for unpurified for the next step.

Step B:

The product of step A (14 mmol) is dissolved in 100 ml DCM and 2 equi, 6.1 ml Boc-anhydrid and 1 equi., 2.45 ml DIPEA are added. The solvent is removed in vacuo and the product is purified on silica using ethyl acetate.

Step C:

1 mmol, 0.6 g of the product of step B is dissolved in 50 ml THF. 1 equi., 0.3 g triphenylphosphine and 1 equi., 75 μl 1-propanol are added. The reaction is started by adding 1 equi., 160 μl diethyl azadicarboxylate. The mixture is stirred over night at room temperature. The product is purified on a C18 reverse phase column (Sep-Pak, Waters, 10 g) with 0.1% TFA in water and acetonitril.

Step D:

0.5 mmol, 0.3 g of the product of step C is dissolved in 25 ml DCM and 25 ml TFA. The solvent is removed in vacuo after 20 min and the product is purified on a C18 reverse phase column (Sep-Pak, Waters, 10 g) with 0.1% TFA in water and acetonitril.

HPLC-MS (Method C): m/z=500 (M+1); Rₜ=3.00 min.

### Example 51 (General Procedure (D))

### (S,S)-6-(4-Amino-butyl)-1-biphenyl-4-ylmethyl-3-(4-isopropoxy-benzyl)-piperazine-2,5-dione

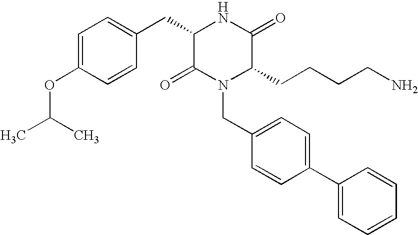 Step A and B:

The intermediate of example 50 is used.

Step C:

0.5 mmol, 0.3 g of the product of step B is dissolved in 5 ml THF. 1.5 equi., 0.2 g triphenylphosphine and 1.5 equi., 58 μl 2-propanol are added. The reaction is started by adding 1.5 equi., 120 μl diethyl azadicarboxylate. The mixture is stirred over night at room temperature. The product is purified on a C18 reverse phase column (Sep-Pak, Waters, 10 g) with 0.1% TFA in water and acetonitril.

Step D:

0.5 mmol, 0.3 g of the product of step C is dissolved in 25 ml DCM and 25 ml TFA. The solvent is removed in vacuo after 20 min and the product is purified on a C18 reverse phase column (Sep-Pak, Waters, 10 g) with 0.1% TFA in water and acetonitril.

HPLC-MS (Method C): m/z=500 (M+1); Rₜ=2.91 min.

### Example 52 (General Procedure (B))

### (S,S)-6-(4-Amino-butyl)-1-(4-phenoxy-benzyl)-3-(4-pyrrol-1-yl-benzyl)-piperazine-2,5-dione

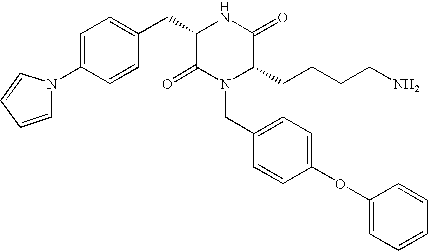 Step A:

The intermediate from example 39, Step A is used.

Step B:

5.0 mmol, 1.7 g Boc-4-(1-pyrrolyl)-Phe-OH is dissolved in 15 ml THF, 0.5 equi., 390 μl DIC is added and the resulting mixture is stirred for 40 min. Then 2.5 mmol of the crude product of step A is added in 15 ml THF. After 4 h 430 μl DIPEA is added and the mixture is stirred overnight. The solvent is removed in vacuo and the residue is taken up in 50 ml ethyl acetate. The org. phase is washed twice with 30 ml 1 M HCl and twice with 30 ml sat. NaHCO₃ and dried over sodium sulfate. The solvent is removed in vacuo and the residual oil is purified on silica with ethyl acetate/heptane (2:3).

Step C:

The purified product from step B is dissolved in 25 ml DCM and 25 ml TFA is added. The solvents are removed in vacuo after 15 min. The residual oil is taken up in 40 ml DCM and 2.0 ml DIPEA is added. The solvent is removed in vacuo after 40 min and the product is purified on a C18 reverse phase column (Sep-Pak, Waters, 10 g) with 0.1% TFA in water and acetonitril.

HPLC-MS (Method C): m/z=523 (M+1); Rₜ=3.15 min.

### Example 53 (General Procedure (D))

### (S,S)-6-(4-Amino-butyl)-1-biphenyl-4-ylmethyl-3-(4-cyclopropylmethoxy-benzyl)-piperazine-2,5-dione

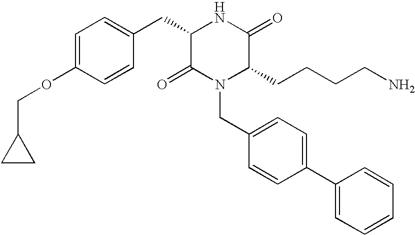 Step A and B:

The intermediate of example 50 is used.

Step C:

0.5 mmol, 0.3 g of the product of step B is dissolved in 4 ml THF. 1.5 equi., 0.2 g triphenylphosphine in 1 ml THF and 1.5 equi., 61 μl cyclopropyl methanol are added. The reaction is started by adding 1.5 equi., 120 μl diethyl azadicarboxylate. The mixture is stirred over night at room temperature. The product is purified on a C18 reverse phase column (Sep-Pak, Waters, 10 g) with 0.1% TFA in water and acetonitril.

Step D:

0.4 mmol, 0.2 g of the product of step C is dissolved in 20 ml DCM and 20 ml TFA. The solvent is removed in vacuo after 15 min and the product is purified on a C18 reverse phase column (Sep-Pak, Waters, 10 g) with 0.1% TFA in water and acetonitril.

HPLC-MS (Method C): m/z=512 (M+1); Rₜ=3.22 min.

### Example 54 (General Procedure (D))

### (S,S)-6-(4-Amino-butyl)-1-biphenyl-4-ylmethyl-3-(4-cyclohexyloxy-benzyl)-piperazine-2,5-dione

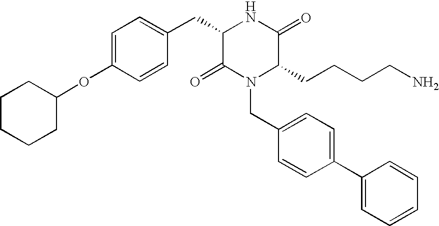 Step A and B:

The intermediate of example 50 is used.

Step C:

0.5 mmol, 0.3 g of the product of step B is dissolved in 5 ml THF. 1.5 equi., 0.2 g triphenylphosphine in 1 ml THF and 1.5 equi., 85 mg cyclohexanol in 1 ml THF are added. The reaction is started by adding 1.5 equi., 120 μl diethyl azadicarboxylate. The mixture is stirred over night at room temperature. The product is purified on a C18 reverse phase column (Sep-Pak, Waters, 10 g) with 0.1% TFA in water and acetonitril.

Step D:

0.2 mmol, 0.1 g of the product of step C is dissolved in 10 ml DCM and 10 ml TFA. The solvent is removed in vacuo after 30 min and the product is purified on a C18 reverse phase column (Sep-Pak, Waters, 10 g) with 0.1% TFA in water and acetonitril.

HPLC-MS (Method C): m/z=540 (M+1); Rₜ=3.56 min.

### Example 55

### (S,S)-1-Biphenyl-4-ylmethyl-6-(4-isopropylamino-butyl)-3-naphthalen-2-ylmethyl-piperazine-2,5-dione

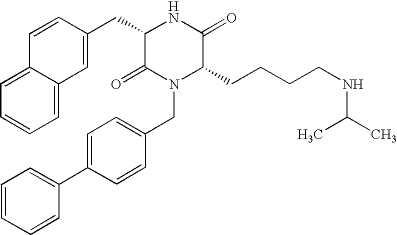 Step A:

0.143 g (0.29 mmol) of (S,S)-6-(4-amino-butyl)-1-biphenyl-4-ylmethyl-3-naphthalen-2-ylmethyl-piperazine-2,5-dione (product from example 11) is mixed with 1.5 ml of dichloromethane, 0.107 ml (1.45 mmol) of acetone, 0.045 ml of acetic acid, and 0.1 g of sodium sulfate. The mixture is stirred for 5 hours. Dichloromethane and acetone are evaporated off in vacuo.

Step B:

To the residue obtained from step A, a solution of 0.031 g (0.493 mmol) of sodium cyanoborohydride in 1.5 ml of tetrahydrofuran and 0.4 ml of methanol is added, followed by 1.0 ml of dichloromethane. The mixture is stirred for two hours. The liquids are evaporated. The residue is treated with 1 ml of tetrahydrofuran and 13 ml of aqueous 37% hydrochloric acid. The resulting suspension is filtered and the filtrate is treated with solid and aqueous sodium hydroxide until pH =14. Filtration and washing with water afforded 0.065 g of the crude product. This is dissolved in 2 ml of methanol, and 1 ml of water is added dropwise. After ice-cooling, the resulting precipitate is collected by filtration and washed with methanol/water (1:1) to give 15 mg of the product.

¹H NMR (400 MHz, CDCl₃): δ 1.02 ppm (s, 6H), 1.22-1.81 ppm (m, 6H), 2.40 ppm (m_{c}, 2H), 2.74 ppm (m_{c}, 1H), 3.21 ppm (dd, J=14 Hz and 9 Hz, 1H), 3.56 ppm (d, J=14 Hz, 1H), 3.85 ppm (m_{c}, 1H), 4.05 ppm (d, J=15 Hz, 1H), 4.43 ppm (m_{c}, 1H), 5.35 ppm (d, J=15 Hz, 1H), 5.90 (s, 1H), 7.30-7.58 ppm (m, 12H), 7.70 (s, 1H), 7.78-7.85 ppm (m, 3H); HPLC-MS (Method B): m/z=534 (M+1); Rₜ=5.10 min.

### Example 56

### (S,S)-6-(4-Amino-butyl)-1-biphenyl-4-ylmethyl-3-(4-phenoxy-benzyl)-piperazine-2,5-dione

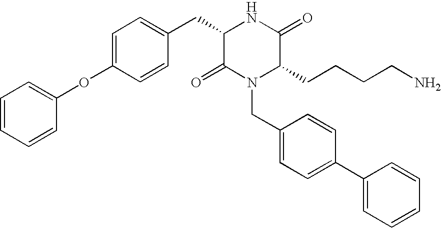 Step A:

The intermediate of example 50, step B is used.

Step B:

A mixture of the product of step A, Cu(OAc)₂ (1 equi), phenylboronic acid (2 equi), pyridine (5 equi) and crushed molecular sieves (4 Å) in dichloromethane are stirred under an air atmosphere for 72 h, filtered through a plug of silica and purified by flash chromatography (eluant ethyl acetate/heptane). Addition of 20 ml dichloromethane and 3 ml trifluoroacetic acid per 500 mg compound removed the Boc protecting group and after purification by reverse phase HPLC (water, actetonitrile, trifluoroactetic acid eluant) afforded the title compound.

¹H NMR (CDCl₃): δ0.8-1.7 (6H, m), 2.7-3.2 (4H, m), 3.65 (1H, d), 4.05 (1H, d), 4.80 (1H, s), 5.05 (1H, d), 6.8-7.5 (18H, m), 7.8-8.1 (2H, bs).

LCMS: 534 (M+); HPLC-MS (Method C): m/z=534 (M+1); Rₜ=3.22 min.

### Example 57

### (S,S)-6-(4-Amino-butyl)-1-biphenyl-4-ylmethyl-3-(4-m-tolyloxy-benzyl)-piperazine-2,5-dione

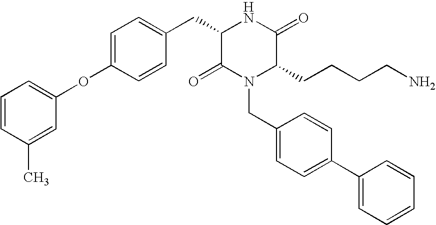 Step A:

The intermediate from example 56, Step A, is used

Step B:

A mixture of the product of step A, Cu(OAc)₂ (1 equi), 3-methylphenylboronic acid (2 equi), pyridine (5 equi) and crushed molecular sieves (4 Å) in dichloromethane are stirred under an air atmosphere for 72 h, filtered through a plug of silica and purified by flash chromatography (eluant ethyl acetate/heptane). Addition of 20 ml dichloromethane and 3 ml trifluoroacetic acid per 500 mg compound removed the Boc protecting group and after purification by reverse phase HPLC (water, actetonitrile, trifluoroactetic acid eluant) afforded the title compound.

¹H NMR (CDCl₃): δ 0.9-1.7 (6H, m), 2.30 (3H, s), 2.7-3.2 (4H, m), 3.6-3.7 (1H, m), 4.05 (1H, d), 42-4.3 (1H, m), 5.10 (1H, d), 6.6-7.5 (17H, m), 7.8-8.1 (2H, bs).

LCMS: 548 (M+); HPLC-MS (Method C): m/z=548 (M+1); Rₜ=3.40 min.

### Example 58

### (S,S)-6-(4-Amino-butyl)-1-biphenyl-4-ylmethyl-3-[4-(4-methoxy-phenoxy)-benzyl]-piperazine-2,5-dione

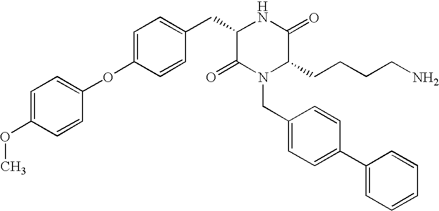 Step A:

The intermediate from example 56, Step A, is used

Step B:

A mixture of the product of step A, Cu(OAc)₂ (1 equi), 4-methoxyphenylboronic acid (2 equi), pyridine (5 equi) and crushed molecular sieves (4 Å) in dichloromethane are stirred under an air atmosphere for 72 h, filtered through a plug of silica and purified by flash chromatography (eluant ethyl acetate/heptane). Addition of 20 ml dichloromethane and 3 ml trifluoroacetic acid per 500 mg compound removed the Boc protecting group and after purification by reverse phase HPLC (water, actetonitrile, trifluoroactetic acid eluant) afforded the title compound.

HPLC-MS (Method C): m/z=564 (M+1); Rₜ=3.44 min.

### Example 59

### (S,S)-6-(4-Amino-butyl)-1-[4-(4-dimethylamino-phenoxy)-benzyl]-3-naphthalen-2-ylmethyl-piperazine-2,5-dione

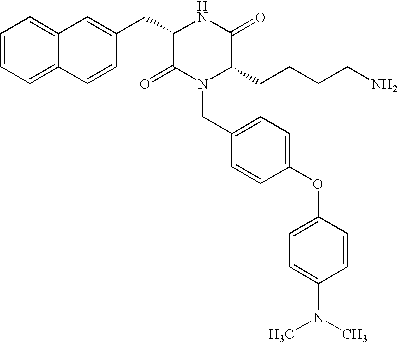 Step A:

A slurry of the compound obtained in example 44 step B (140 mg, 0.21 mmol), 4-(dimethylamino)phenylboronic acid (104 mg, 0.63 mmol), copper(II) acetate (76 mg, 0.42 mmol), triethylamine (146 μl, 1.05 mmol) and powdered molecular sieves (4 Å) in THF is stirred at room temperature for about two days. The mixture is filtered and the filtrate is evaporated in vacuo. The product is isolated from the residue by column chromatography on silica with ethyl acetate/heptane (1:2) and used directly in the following step

Step B:

A solution of the product from step A and TFA (3 ml) in DCM (10 ml) is stirred at room temperature overnight. After evaporation in vacuo the residue is taken up in toluene and the solvent is again removed in vacuo. The residual oil is now dissolved in DCM, and N,N-diisopropylethylamine (0.5 ml, 2.8 mmol) is added. After stirring overnight, the mixture is evaporated in vacuo and the residue is purified on preparative LC-MS. The product is dissolved in a mixture of 1 N HCl and methanol and evaporated in vacuo affording 30 mg of the title compound as the hydrochloride.

HPLC-MS (Method C): m/z=551 (M+1); Rₜ=2.2 min.

### Example 60

### (S,S)-6-(4-Amino-butyl)-1-[4-(4-methoxy-phenoxy)-benzyl]-3-naphthalen-2-ylmethyl-piperazine-2,5-dione

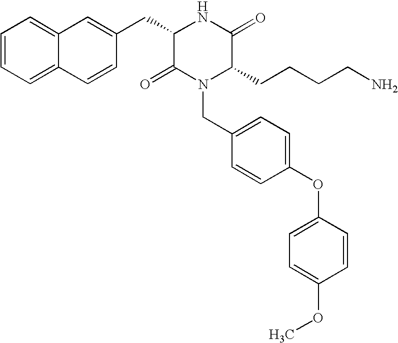 Step A:

A slurry of the compound obtained in example 44 step B (140 mg, 0.21 mmol), 4-methoxyphenylboronic acid (96 mg, 0.63 mmol), copper(II) acetate (76 mg, 0.42 mmol), triethylamine (146 μl, 1.05 mmol) and powdered molecular sieves (4 Å) in THF is stirred at room temperature for about two days. The mixture is filtered and the filtrate is evaporated in vacuo. The product is isolated from the residue by column chromatography on silica with ethyl acetate/heptane (1:2) and used directly in the following step

Step B:

A solution of the product from step A and TFA (3 ml) in DCM (10 ml) is stirred at room temperature overnight. After evaporation in vacuo the residue is taken up in toluene and the solvent is again removed in vacuo. The residual oil is now dissolved in DCM, and N,N-diisopropylethylamine (0.5 ml, 2.8 mmol) is added. After stirring overnight, the mixture is evaporated in vacuo and the residue is purified on preparative LC-MS. The product is dissolved in a mixture of 1 N HCl and methanol and evaporated in vacuo affording 85 mg of the title compound as the hydrochloride.

HPLC-MS (Method ): m/z=538 (M+1); Rₜ=3.2 min.

### Example 61

### (S,S)-1-[4-(3-Acetyl-phenoxy)-benzyl]-6-(4-amino-butyl)-3-naphthalen-2-ylmethyl-piperazine-2,5-dione

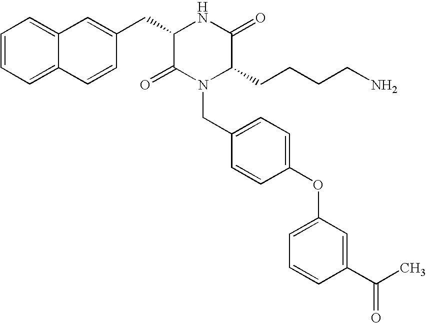 Step A:

A slurry of the compound obtained in example 44 step B (140 mg, 0.21 mmol), 3-acetyl-phenylboronic acid (103 mg, 0.63 mmol), copper(II) acetate (76 mg, 0.42 mmol), triethylamine (146 μl, 1.05 mmol) and powdered molecular sieves (4 Å) in THF is stirred at room temperature for about two days. The mixture is filtered and the filtrate is evaporated in vacuo. The product is isolated from the residue by column chromatography on silica with ethyl acetate/heptane (1:2) and used directly in the following step

Step B:

A solution of the product from step A and TFA (3 ml) in DCM (10 ml) is stirred at room temperature overnight. After evaporation in vacuo the residue is taken up in toluene and the solvent is again removed in vacuo. The residual oil is now dissolved in DCM, and N,N-diisopropylethylamine (0.5 ml, 2.8 mmol) is added. After stirring overnight, the mixture is evaporated in vacuo and the residue is purified on preparative LC-MS. The product is dissolved in a mixture of 1 N HCl and methanol and evaporated in vacuo affording 85 mg of the title compound as the hydrochloride.

HPLC-MS (Method): m/z=550 (M+1); Rₜ=2.8 min.

### Example 62

### (S,S)-6-(4-Amino-butyl)-1-[4-(4-ethanesulfonyl-phenoxy)-benzyl]-3-naphthalen-2-ylmethyl-piperazine-2,5-dione

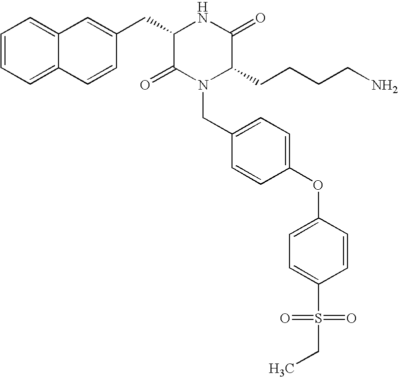 Step A:

A slurry of the compound obtained in example 44 step B (140 mg, 0.21 mmol), 4-(ethylsulfonyl)phenylboronic acid (135 mg, 0.63 mmol), copper(II) acetate (76 mg, 0.42 mmol), triethylamine (146 μl, 1.05 mmol) and powdered molecular sieves (4 Å) in THF is stirred at room temperature for about two days. The mixture is filtered and the filtrate is evaporated in vacuo. The product is isolated from the residue by column chromatography on silica with ethyl acetate/heptane (1:2) and used directly in the following step

Step B:

A solution of the product from step A and TFA (3 ml) in DCM (10 ml) is stirred at room temperature overnight. After evaporation in vacuo the residue is taken up in toluene and the solvent is again removed in vacuo. The residual oil is now dissolved in DCM, and N,N-diisopropylethylamine (0.5 ml, 2.8 mmol) is added. After stirring overnight, the mixture is evaporated in vacuo and the residue is purified on preparative LC-MS. The product is dissolved in a mixture of 1 N HCl and methanol and evaporated in vacuo affording 50 mg of the title compound as the hydrochloride.

HPLC-MS (Method C): m/z=600 (M+1); Rₜ=2.9 min.

### Example 63

### (S,S)-6-(4-Amino-butyl)-1-biphenyl-4-ylmethyl-3-[4-(4-chloro-phenoxy)-benzyl]-piperazine-2,5-dione

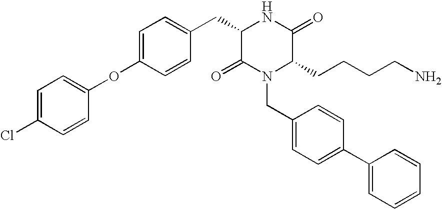 Step A:

The intermediate from example 56, Step A, is used

Step B:

A mixture of the product of step A, Cu(OAc)₂ (1 equi), 4-chlorophenylboronic acid (2 equi), pyridine (5 equi) and crushed molecular sieves (4 Å) in dichloromethane are stirred under an air atmosphere for 72 h, filtered through a plug of silica and purified by flash chromatography (eluant ethyl acetate/heptane). Addition of 20 ml dichloromethane and 3 ml trifluoroacetic acid per 500 mg compound removed the Boc protecting group and after purification by reverse phase HPLC (water, actetonitrile, trifluoroactetic acid eluant) afforded the title compound.

¹H NMR (CDCl₃): δ0.9-1.7 (6H, m), 2.7-3.2 (4H, m), 3.6-3.7 (1H, m), 4.05 (1H, d), 4.3-4.4 (1H, m), 5.10 (1H, d), 6.7-7.5 (17H, m), 7.7-7.9 (2H, bs).

LCMS: 568 (M+); HPLC-MS (Method C): m/z=568 (M+1); Rₜ=3.72 min.

### Example 64

### (S,S)-3-[4-(4-Acetyl-phenoxy)-benzyl]-6-(4-amino-butyl)-1-biphenyl-4-ylmethyl-piperazine-2,5-dione

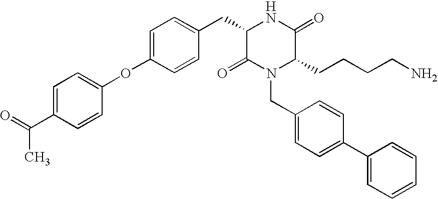 Step A:

The intermediate from example 56, Step A, is used

Step B:

A mixture of the product of step A, Cu(OAc)₂ (1 equi), 4-acetylphenylboronic acid (2 equi), pyridine (5 equi) and crushed molecular sieves (4 Å) in dichloromethane are stirred under an air atmosphere for 72 h, filtered through a plug of silica and purified by flash chromatography (eluant ethyl acetate/heptane). Addition of 20 ml dichloromethane and 3 ml trifluoroacetic acid per 500 mg compound removed the Boc protecting group and after purification by reverse phase HPLC (water, actetonitrile, trifluoroactetic acid eluant) afforded the title compound.

¹H NMR (CDCl₃): δ 0.9-1.7 (6H, m), 2.50 (3H, s), 2.7-3.2 (4H, m), 3.6-3.7 (1H, m), 4.05 (1H, d), 4.3-4.4 (1H, m), 5.05 (1H, d), 6.8-7.9 (17H, m), 7.9-8.1 (2H, bs).

LCMS: 576 (M+); HPLC-MS (Method C): m/z=568 (M+1); Rₜ=3.72 min.

### Example 65

### (S,S)-3-[4-(3-Acetyl-phenoxy)-benzyl]-6-(4-amino-butyl)-1-biphenyl-4-ylmethyl-piperazine-2,5-dione

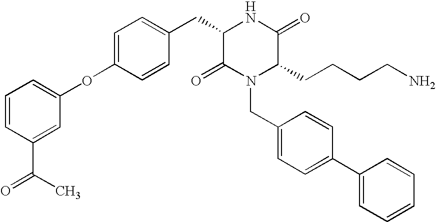 Step A:

The intermediate from example 50, Step B, is used

Step B:

A mixture of the product of step A, Cu(OAc)₂ (1 equi), 3-acetylphenylboronic acid (2 equi), pyridine (5 equi) and crushed molecular sieves (4 Å) in dichloromethane are stirred under an air atmosphere for 72 h, filtered through a plug of silica and purified by flash chromatography (eluant ethyl acetate/heptane). Addition of 20 ml dichloromethane and 3 ml trifluoroacetic acid per 500 mg compound removed the Boc protecting group and after purification by reverse phase HPLC (water, actetonitrile, trifluoroactetic acid eluant) afforded the title compound.

¹H NMR (CDCl₃): δ 0.9-1.7 (6H, m), 2.50 (3H, s), 2.7-3.3 (4H, m), 3.6-3.7 (1H, m), 4.0 (1H, d), 4.3 (1H, bs), 5.05 (1H, d), 6.8-7.6 (17H, m), 7.8-8.0 (2H, bs).

LCMS: 576 (M+); HPLC-MS (Method C): m/z=576 (M+1); Rₜ=3.31 min.

### Example 66 (General procedure (D))

### (S,S)-6-(4-Amino-butyl)-1-biphenyl-4-ylmethyl-3-(4-methoxy-benzyl)-piperazine-2,5-dione

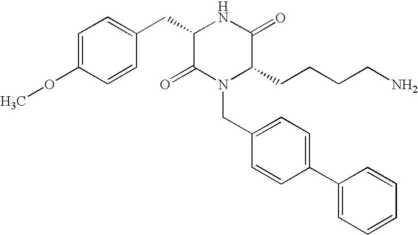

Step A and B:

The intermediate of example 50 is used.

Step C:

0.5 mmol, 0.3 g of the product of step B is dissolved in 5 ml THF. 1.5 equi., 0.2 g triphenylphosphine and 1.5 equi., 31 μl methanol are added. The reaction is started by adding 1.5 equi., 120 μl diethyl azadicarboxylate. The mixture is stirred over night at room temperature. The product is purified on a C18 reverse phase column (Sep-Pak, Waters, 10 g) with 0.1% TFA in water and acetonitril.

Step D:

0.4 mmol, 0.2 g of the product of step C is dissolved in 10 ml DCM and 10 ml TFA. The solvent is removed in vacuo after 15 min and the product is purified on a C18 reverse phase column (Sep-Pak, Waters, 10 g) with 0.1% TFA in water and acetonitril.

HPLC-MS (Method C): m/z=472 (M+1); Rₜ=2.61 min.

### Example 67 (General Procedure (D))

### (S,S)-6-(4-Amino-butyl)-1-biphenyl-4-ylmethyl-3-(4-ethoxy-benzyl)-piperazine-2,5-dione

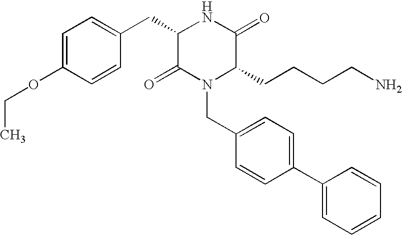 Step A and B:

The intermediate of example 50 is used.

Step C:

0.5 mmol, 0.3 g of the product of step B is dissolved in 5 ml THF. 1.5 equi., 0.2 g triphenylphosphine in 1 ml THF and 1.5 equi., 44 μl ethanol are added. The reaction is started by adding 1.5 equi., 120 μl diethyl azadicarboxylate. The mixture is stirred over night at room temperature. The product is purified on a C18 reverse phase column (Sep-Pak, Waters, 10 g) with 0.1% TFA in water and acetonitril.

Step D:

0.4 mmol, 0.2 g of the product of step C is dissolved in 10 ml DCM and 10 ml TFA. The solvent is removed in vacuo after 20 min and the product is purified on a C18 reverse phase column (Sep-Pak, Waters, 10 g) with 0.1% TFA in water and acetonitril.

HPLC-MS (Method C): m/z=486 (M+1); Rₜ=2.75 min.

### Example 68

### (S,S)-6-(4-Amino-butyl)-1-biphenyl-4-ylmethyl-3-[4-(3-trifluoromethoxy-phenoxy)-benzyl]-piperazine-2,5-dione

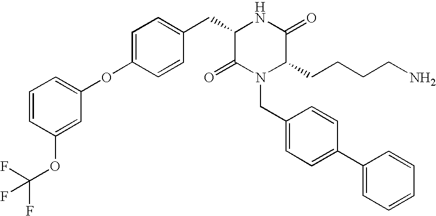 Step A:

The intermediate from example 50, Step B, is used

Step B:

A mixture of the product of step A, Cu(OAc)₂ (1 equi), 3-(trifluoromethoxy) benzeneboronic acid (2 equi), pyridine (5 equi) and crushed molecular sieves (4 Å) in dichloromethane are stirred under an air atmosphere for 72 h, filtered through a plug of silica and purified by flash chromatography (eluant ethyl acetate/heptane). Addition of 20 ml dichloromethane and 3 ml trifluoroacetic acid per 500 mg compound removed the Boc protecting group and after purification by reverse phase HPLC (water, actetonitrile, trifluoroactetic acid eluant) afforded the title compound.

¹H NMR (CDCl₃): δ0.9-1.7 (6H, m), 2.8-3.2 (4H, m), 3.6-3.7 (1H, m), 4.0 (1H, d), 4.3-4.4 (1H, m), 5.05 (1H, d), 6.7-7.5 (17H, m), 7.8-8.0 (2H, bs).

LCMS: 618 (M+); HPLC-MS (Method C): m/z=618 (M+1); Rₜ=3.93 min.

### Example 69

### (S,S)-6-(4-Amino-butyl)-1-biphenyl-4-ylmethyl-3-[4-(4-fluoro-phenoxy)-benzyl]-piperazine-2,5-dione

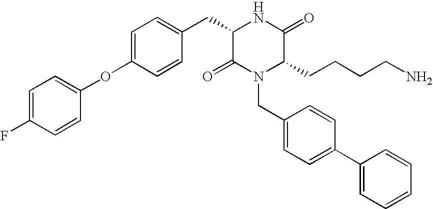 Step A:

The intermediate from example 50, Step B, is used

Step B:

A mixture of the product of step A, Cu(OAc)₂ (1 equi), 4-fluorophenylboronic acid (2 equi), pyridine (5 equi) and crushed molecular sieves (4 Å) in dichloromethane are stirred under an air atmosphere for 72 h, filtered through a plug of silica and purified by flash chromatography (eluant ethyl acetate/heptane). Addition of 20 ml dichloromethane and 3 ml trifluoroacetic acid per 500 mg compound removed the Boc protecting group and after purification by reverse phase HPLC (water, actetonitrile, trifluoroactetic acid eluant) afforded the title compound.

¹H NMR (CDCl₃): δ0.7-1.7 (6H, m), 2.7-3.2 (4H, m), 3.6-3.7 (1H, m), 4.0 (1H, d), 4.2-4.3 (1H, m), 5.05 (1H, d), 6.7-7.5 (17H, m), 7.8-8.0 (2H, bs).

### Example 70

### (S,S)-6-(4-Amino-butyl)-1-biphenyl-4-ylmethyl-3-[4-(3-nitro-phenoxy)-benzyl]-piperazine-2,5-dione

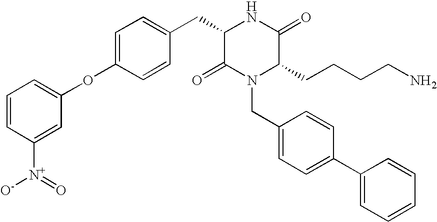 Step A:

The intermediate from example 50, Step B, is used

Step B:

A mixture of the product of step A, Cu(OAc)₂ (1 equi), 3-nitrophenylboronic acid (2 equi), pyridine (5 equi) and crushed molecular sieves (4 Å) in dichloromethane are stirred under an air atmosphere for 72 h, filtered through a plug of silica and purified by flash chromatography (eluant ethyl acetate/heptane). Addition of 20 ml dichloromethane and 3 ml trifluoroacetic acid per 500 mg compound removed the Boc protecting group and after purification by reverse phase HPLC (water, actetonitrile, trifluoroactetic acid eluant) afforded the title compound.

¹H NMR (CDCl₃): δ0.7-1.7 (6H, m), 2.7-3.3 (4H, m), 3.6-3.7 (1H, d), 4.0 (1H, d), 4.3-4.4 (1H, m), 5.1 (1H, d), 6.9-7.9 (17H, m), 7.9-8.1 (2H, bs).

LCMS: 579 (M+); HPLC-MS (Method C): m/z=579 (M+1); Rₜ=3.45 min.

### Example 71 (General Procedure (D))

### (S,S)-6-(4-Amino-butyl)-1-(4-phenoxy-benzyl)-3-(4-propoxy-benzyl)-piperazine-2,5-dione

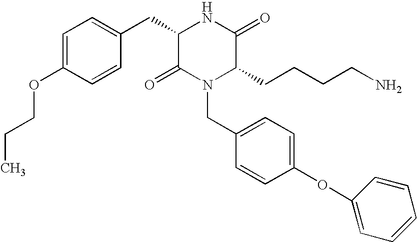 Step A:

45 mmol, 15.5 g Boc-Tyr(t-bu)-OH is dissolved in 100 ml THF and 0.5 equi, 3.5 ml DIC is added. The mixture is stirred for 30 min and the intermediate of example 39, step A, 22 mmol, 9.9 g in 40 ml THF is added. After 2.5 h1 equi., 2.8 ml DIPEA is added and the mixture is stirred overnight. The solvent is removed in vacuo and the oil is taken up in 200 ml ethyl acetate. The org. phase is washed twice with 130 ml 1 M HCl and twice with 130 ml sat. NaHCO₃, the org. phase is dried over sodium sulfate and the solvent is removed in vacuo. The residual oil is purified on silica with ethyl acetate:heptane 2:3.

The pure product is dissolved in 100 ml DCM and 100 ml TFA. The solvent is removed after 20 min and the oil taken up in 100 ml DCM and 5 ml DIPEA is added and the mixture stirred at room temperature. After 45 min another 5 ml DIPEA are added and again after 2 h. The solvent is removed in vacuo after 3 h and the crude product used in the next step.

Step B:

The product of step A (13 mmol) is dissolved in 100 ml DCM and 2 equi, 5.5 ml Boc-anhydrid and 1 equi., 2.2 ml DIPEA are added. The solvent is removed in vacuo after 2 hand the product is purified on silica using ethyl acetate.

Step C:

1 mmol, 0.6 g of the product of step B is dissolved in 50 ml THF. 1 equi., 0.3 g triphenylphosphine and 1 equi., 75 μl 1-propanol are added. The reaction is started by adding 1 equi., 160 μl diethyl azadicarboxylate. The mixture is stirred over night at room temperature. The product is purified on a C18 reverse phase column (Sep-Pak, Waters, 10 g) with 0.1% TFA in water and acetonitril.

Step D:

0.5 mmol, 0.3 g of the product of step C is dissolved in 25 ml DCM and 25 ml TFA. The solvent is removed in vacuo after 20 min and the product is purified on a C18 reverse phase column (Sep-Pak, Waters, 10 g) with 0.1% TFA in water and acetonitril.

HPLC-MS (Method C): m/z=516 (M+1); Rₜ=2.90 min.

### Example 72

### (S,S)-6-(4-Amino-butyl)-1-biphenyl-4-ylmethyl-3-[4-(pyridin-3-yloxy)-benzyl]-piperazine-2,5-dione

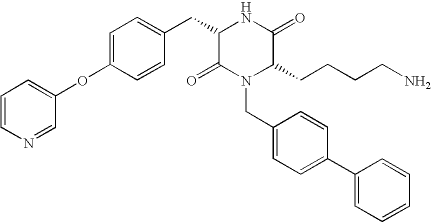 Step A:

The intermediate from example 50, Step B, is used

Step B:

A mixture of the product of step A, Cu(OAc)₂ (1 equi), pyridine-3-boronic acid (2 equi), pyridine (5 equi) and crushed molecular sieves (4 Å) in dichloromethane are stirred under an air atmosphere for 72 h, filtered through a plug of silica and purified by flash chromatography (eluant ethyl acetate/heptane). Addition of 20 ml dichloromethane and 3 ml trifluoroacetic acid per 500 mg compound removed the Boc protecting group and after purification by reverse phase HPLC (water, actetonitrile, trifluoroactetic acid eluant) afforded the title compound.

¹H NMR (CDCl₃): δ0.7-1.7 (6H, m), 2.7-3.3 (4H, m), 3.6-3.7 (1H, d), 3.95 (1H, d), 4.4-4.5 (1H, m), 5.2 (1H, d), 7.0-8.5 (19H, m).

LCMS: 535 (M+); HPLC-MS (Method C): m/z=535 (M+1); Rₜ=2.69 min.

### Example 73

### (S,S)-6-(4-Amino-butyl)-1-biphenyl-4-ylmethyl-3-[4-(4-dimethylamino-phenoxy)-benzyl]-piperazine-2,5-dione

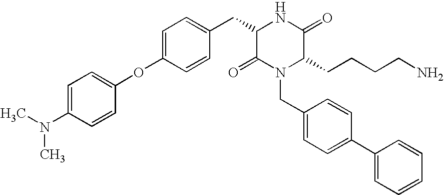 Step A:

The intermediate from example 50, Step B, is used

Step B:

A mixture of the product of step A, Cu(OAc)₂ (1 equi), 4-dimethylamino phenylboronic acid (2 equi), pyridine (5 equi) and crushed molecular sieves (4 Å) in dichloromethane are stirred under an air atmosphere for 72 h, filtered through a plug of silica and purified by flash chromatography (eluant ethyl acetate/heptane). Addition of 20 ml dichloromethane and 3 ml trifluoroacetic acid per 500 mg compound removed the Boc protecting group and after purification by reverse phase HPLC (water, actetonitrile, trifluoroactetic acid eluant) afforded the title compound.

¹H NMR (CDCl₃): δ 0.7-1.7 (6H, m), 2.7-3.3 (4H, m), 3.05 (6H, s), 3.6-3.7 (1H, d), 4.0 (1H, d), 4.3-4.4 (1H, m), 5.1 (1H, d), 6.8-7.5 (17H, m), 7.8-8.0 (2H, bs).

LCMS: 577 (M+); HPLC-MS (Method C): m/z=577 (M+1); Rₜ=2.46 min.

### Example 74

### (S,S)-6-(4-Amino-butyl)-3-naphthalen-2-ylmethyl-1-(6-phenyl-pyridin-3-ylmethyl)-piperazine-2,5-dione

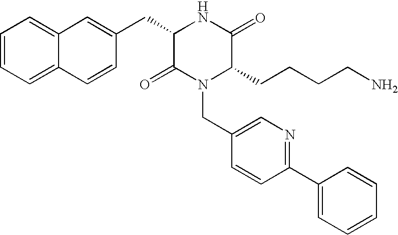 Step A:

To a solution of H-Lys(Boc)-OMe HCl (0.47 g, 1.57 mmol) in THF (15 ml) is added 6-phenyl-nicotinaldehyde (289 mg, 1.58 mmol) and N,N-diisopropylethylamine (0.30 ml, 1.73 mmol), and the mixture is stirred in the presence of powdered molecular sieves (4 Å) overnight at room temperature. Then methanol (1.6 ml), acetic acid (0.8 ml) and sodium cyanoborohydride (0.30 g, 4.7 mmol) is added and the mixture is stirred for 5 h at room temperature. The mixture is evaporated in vacuo and the residue is taken up in ethyl acetate (30 ml) and filtered. The filtrate is washed with 1 N sodium hydroxide (25 ml), dried over sodium sulfate and evaporated to dryness to give 645 mg (97%) of the crude product, which is used in the next step without further purification.

HPLC-MS (Method C): m/z=428 (M+1); Rₜ=2.8 min.

Step B:

To a solution of Boc-β-2-naphthyl-Ala-OH (693 mg, 2.2 mmol) in THF (8 ml) is added N,N′-diisopropylcarbodiimide (1.1 ml, 7.1 mmol) and the mixture is stirred for 35 min at room temperature. A solution of the crude product from step A in THF (10 ml) is added and the mixture is stirred overnight at room temperature. Then N,N-diisopropylethylamine (0.38 ml, 2.2 mmol) is added and stirring is continued overnight. The mixture is evaporated in vacuo and the residue is taken up in ethyl acetate (50 ml) and washed successively with 1 N HCl (30 ml) and saturated aqueous sodium hydrogen carbonate (30 ml), dried over sodium sulfate and evaporated to dryness. Column chromatography on silica with ethyl acetate/heptane (1:2) afforded the intermediate in a yield of 0.61 g (55%).

HPLC-MS (Method C): m/z=725 (M+1); Rₜ=5.4 min.

Step C:

A solution of the product from step B (534 mg, 0.74 mmol) and TFA (10 ml) in DCM (20 ml) is stirred for 2 h at room temperature. After evaporation in vacuo the residue is taken up in toluene (10 ml) and the solvent is again removed in vacuo. The residue is now dissolved in DCM (20 ml) and N,N-diisopropylethylamine (0.40 ml, 2.3 mmol) is added. After stirring overnight, the mixture is evaporated in vacuo and the residue is purified on a C1 8 reverse phase column (Sep-Pak, Waters, 10 g) with 0.1% TFA in water and acetonitrile. The product is dissolved in a mixture of 1 N HCl and methanol and evaporated in vacuo affording 200 mg of the title compound as the hydrochloride

HPLC-MS (Method C): m/z=493 (M+1); Rₜ=2.5 min.

### Example 75

### (S,S)-3-{4-[5-(4-Amino-butyl)-4-biphenyl-4-ylmethyl-3,6-dioxo-piperazin-2-ylmethyl]-phenoxy}-benzaldehyde

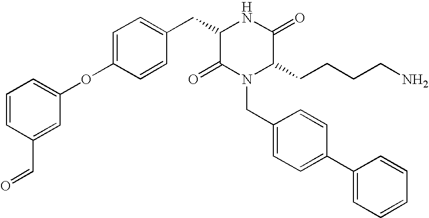 Step A:

The intermediate from example 50, Step B, is used

Step B:

A mixture of the product of step A, Cu(OAc)₂ (1 equip, 3-formylphenylboronic acid (2 equi), pyridine (5 equi) and crushed molecular sieves (4 Å) in dichloromethane are stirred under an air atmosphere for 72 h, filtered through a plug of silica and purified by flash chromatography (eluant ethyl acetate/heptane). Addition of 20 ml dichloromethane and 3 ml trifluoroacetic acid per 500 mg compound removed the Boc protecting group and after purification by reverse phase HPLC (water, actetonitrile, trifluoroactetic acid eluant) afforded the title compound.

¹H NMR (DMSO-d₆): δ 0.7-1.7 (6H, m), 2.7-3.3 (4H, m), 3.6-3.7 (1H, d), 4.0 (1H, d), 4.35 (1H, s), 5.1 (1H, d), 6.7-7.5 (17H, m), 7.9-8.1 (2H, bs), 9.9 (1H, s).

LCMS: 562 (M+);

### Example 76

### (S,S)-6-(4-Amino-butyl)-1-(4-bromo-benzyl)-3-naphthalen-2-ylmethyl-piperazine-2,5-dione

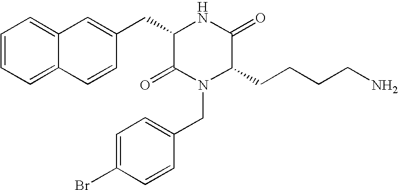 Step A:

To a solution of H-Lys(Boc)-OMe HCl (5.0 g, 16.7 mmol) in THF (150 ml) is added 4-bromobenzaldehyde (3.1 g, 16.7 mmol) and N,N-diisopropylethylamine (3.0 ml, 16.7 mmol), and the mixture is stirred in the presence of powdered molecular sieves (4 Å) for 4 h at room temperature. Then methanol (17 ml), acetic acid (8.0 ml) and sodium cyanoborohydride (3.1 g, 50 mmol) is added and the mixture is stirred for two days at room temperature. The mixture is evaporated in vacuo and the residue is taken up in ethyl acetate (100 ml) and filtered. The filtrate is washed with 1 N sodium hydroxide (75 ml), dried over sodium sulfate and evaporated to dryness in vacuo to give the crude product, which is used in the next step without further purification.

HPLC-MS (Method C): m/z=429/431 (M+1); Rₜ=2.8 min.

Step B:

To a solution of Boc-β-2-naphthyl-Ala-OH (8.52 g, 27 mmol) in THF (100 ml) is added N,N′-diisopropylcarbodiimide (2.1 ml, 13.5 mmol) and the mixture is stirred for 30 min at room temperature. A solution of the crude product from step A (5.7 g, 13.5 mmol) in THF (100 ml) is added and the mixture is stirred overnight at room temperature. Then N,N-diisopropylethylamine (5.0 ml, 27 mmol) is added and stirring is continued overnight. The mixture is evaporated in vacuo and the residue is taken up in ethyl acetate (100 ml) and washed successively with 1 N HCl (50 ml) and saturated aqueous sodium hydrogen carbonate (50 ml), dried over sodium sulfate and evaporated to dryness. Column chromatography on silica with ethyl acetate/heptane (1:2) afforded the intermediate in a yield of 0.30 g (3%).

HPLC-MS (Method C): m/z=748/750 (M+23); Rₜ=6.1 min.

Step C:

A solution of the product from step B and TFA (10 ml) in DCM (20 ml) is stirred for 2 h at room temperature. After evaporation in vacuo the residue is taken up in toluene (20 ml) and the solvent is again removed in vacuo. The residue is now dissolved in DCM (20 ml) and N,N-diisopropylethylamine (0.8 ml, 4.6 mmol) is added. After stirring overnight, the mixture is evaporated in vacuo and the residue is purified on a C18 reverse phase column (Sep-Pak, Waters, 10 g) with 0.1% TFA in water and acetonitrile affording. The product is dissolved in a mixture of 1 N HCl and methanol and evaporated in vacuo affording 123 mg (60%) of the title compound as the hydrochloride.

HPLC-MS (Method C): m/z=494/496 (M+1); Rₜ=2.7 min.

### Example 77

### (S,S)-6-(4-Amino-butyl)-3-(4-isopropoxy-benzyl)-1-(4-phenoxy-benzyl)-piperazine-2,5-dione

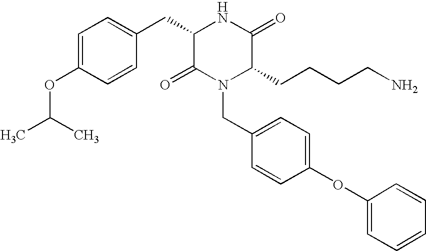 Step A and B:

The intermediate of example 71, step B is used.

Step C:

0.5 mmol, 0.3 g of the product of step B is dissolved in 5 ml THF. 1.5 equi., 0.2 g triphenylphosphine and 1.5 equi., 58 μl 2-propanol are added. The reaction is started by adding 1.5 equi., 120 μl diethyl azadicarboxylate. The mixture is stirred for 6 h at room temperature. The product is purified on a C18 reverse phase column (Sep-Pak, Waters, 10 g) with 0.1% TFA in water and acetonitril.

Step D:

0.3 mmol, 0.2 g of the product of step C is dissolved in 15 ml DCM and 15 ml TFA. The solvent is removed in vacuo after 20 min and the product is purified on a C18 reverse phase column (Sep-Pak, Waters, 10 g) with 0.1% TFA in water and acetonitril.

HPLC-MS (Method C): m/z=516 (M+1); Rₜ=2.90 min.

### Example 78

### (S,S)-6-[4-(2-Amino-ethylamino)-butyl]-1-(4-phenoxy-benzyl)-3-(4-propoxy-benzyl)-piperazine-2,5-dione

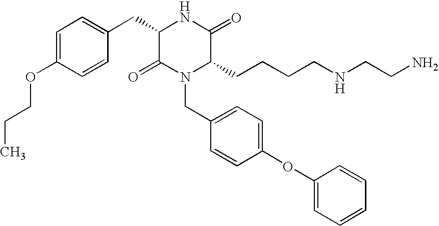 Step A:

1.35 g (2.6 mmol) of example 71 is dissolved in 40 ml acetonitril. 0.6 g (1 equi.) 2-(Boc-amino)-ethylbromide, 0.2 g (0.5 equi.) potassium iodide and 1.15 ml 1,8-diazabicyclo[5.4]undec-7-ene (DBU) are added. The mixture is stirred for 4 days and then the solvent is removed in vacuo and the product is purified on a C18 reverse phase column (Sep-Pak, Waters, 10 g) with 0.1% TFA in water and acetonitril.

Step B:

0.7 g (1.1 mmol) of the product from step A is dissolved in 30 ml dichlormethane and 30 ml TFA. The solvent is removed in vacuo after 20 min and the product is purified on a C18 reverse phase column (Sep-Pak, Waters, 10 g) with 0.1% TFA in water and acetonitril.

HPLC-MS (Method C): m/z=559 (M+1); Rₜ=2.62 min.

### Example 79

### (S,S)-3-Amino-N-(1-biphenyl-4-ylmethyl-5-naphthalen-2-ylmethyl-3,6-dioxo-piperazin-2-ylmethyl)-3-methyl-N-piperidin-4-ylmethyl-butyramide

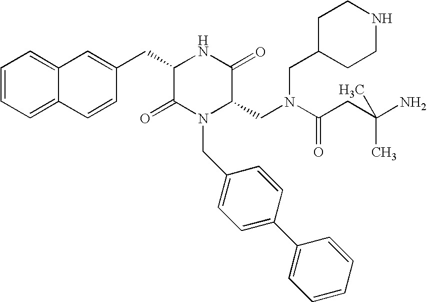 Step A:

To a solution of 2-tert-butoxycarbonylamino-3-(9H-fluoren-9-ylmethoxy carbonylamino)propionic acid (5.00 g, 11.72 mmol) in 100 ml of tetrahydrofuran are added 1-hydroxybenzotriazole (3.59 g, 23.44 mmol), N-ethyl-N′-(3-dimethylaminopropyl)-carbodiimide hydrochloride (2.36 g, 12.31 mmol) and N-ethyldiisopropylamin (2.81 ml, 16.41 mmol). Stirred for 30 min, then 20 ml of methanol is added. Stirred overnight to give a clear yellow mixture. The mixture is concentrated in vacuo, dissolved in 150 ml of ethyl acetate and washed with 25 ml of aqueous sodium hydrogen sulfate (10%), 25 ml of aqueous sodium hydrogen carbonate (saturated), 25 ml of water, and 25 ml of brine, dried over magnesium sulfate and filtered. Concentrated in vacuo to give 4.80 g (93%) of (S)-2-tert-butoxycarbonylamino-3-(9H-fluoren-9-ylmethoxycarbonylamino)propionic acid methyl ester as colorless crystalline oil.

HPLC-MS: Rₜ=6.80 min., (M+1)=441, % Area by ELS=95

Step B:

(S)-2-tert-Butoxycarbonylamino-3-(9H-fluoren-9-ylmethoxycarbonylamino)propionic acid methyl ester (4.80 g, 10.90 mmol) is dissolved in 20 ml of ethyl acetate in a 250 ml flask equipped with a magnetic stirrer. To the stirred solution is added 80 ml of 2.8 M hydrogen chloride in ethyl acetate and the reaction is stirred for 2 hours under nitrogen. Concentrated in vacuo to give a white solid, which is taken up in ethyl acetate, stirred and filtered. The solid is dried in vacuo at 40° C. to give 3.00 g (73%) of ((S)-2-amino-3-(9H-fluoren-9-ylmethoxycarbonylamino)propionic acid methyl ester hydrochloride as a white solid.

HPLC-MS: Rₜ=4.43 min., (M+1)=341, % Area by ELS=94

Step C:

((S)-2-Amino-3-(9H-fluoren-9-ylmethoxycarbonylamino)propionic acid methyl ester hydrochloride (2.98 g, 7.91 mmol) is dissolved in a mixture of 40 ml of tetrahydrofuran and 40 ml of methanol. Sodium acetate (2.59 g, 31.6 mmol), biphenyl-4-carbaldehyde (1.44 g, 7.91 mmol), molecular sieves (4 Å) and sodium cyanoborohydride (8.7 ml, 8.7 mmol) is added. Stirred overnight under nitrogen. Filtered through Hyflo Super Cel® to give a clear solution which is concentrated in vacuo, dissolved in 100 ml of ethyl acetate and washed with 25 ml of aqueous sodium hydrogen carbonate (saturated), 25 ml of water, 25 ml of brine, dried over magnesium sulfate and filtered. Addition of 5 ml 2.8 M hydrogen chloride in ethyl acetate resulted in precipitation of a white solid, which is isolated by filtration and dried to afford 4.01 g (93%) of (2S)-2-[(biphenyl-4-ylmethyl)amino]-3-(9H-fluoren-9-ylmethoxycarbonylamino)propionic acid methyl ester hydrochloride.

Step D:

To a solution of (S)-2-tert-butoxycarbonylamino-3-(2-naphtyl)propionic acid (4.65 g, 14.7 mmol) in 30 ml of tetrahydrofuran is added N-ethyl-N′-(3-dimethylaminopropyl)-carbodiimide hydrochloride (1.41 g, 7.37 mmol). Stirred for 30 min, then (2S)-2-[(biphenyl-4-ylmethyl)amino]-3-(9H-fluoren-9-ylmethoxycarbonylamino)propionic acid methyl ester hydrochloride (4.00 g, 7.37 mmol) and N-ethyldiisopropylamin (2.52 ml, 14.7 mmol) are added. Stirred for 3 days. The mixture is concentrated in vacuo, dissolved in 100 ml of ethyl acetate and 25 ml of aqueous sodium hydrogen sulfate (10%), mixed and separated. The aqueous phase is extracted with 50 ml of ethyl acetate and the combined organic phases are washed with 25 ml of water, 25 ml of brine, dried over magnesium sulfate and filtered. Concentrated in vacuo to give 7.62 g of yellow foam, which is analyzed by LC-MS, indicating only 16% of (2S)-2-[biphenyl-4-ylmethyl-(2S)-(2-tert-butoxycarbonylamino-3-(2-naphthyl)propionyl)amino]-3-(9H-fluoren-9-ylmethoxycarbonylamino)propionic acid methyl ester. The crude product is dissolved in 50 ml of tetrahydrofuran and 2-tert-butoxycarbonylamino-3-(2-naphtyl)propionic acid (2.32 g, 7.37 mmol), bromo-tris-pyrrolidino-phosphonium hexafluorophosphate (3.44 g, 7.37 mmol) and N-ethyldiisopropylamin (1.26 ml, 7.37 mmol) are added. Stirred overnight and concentrated in vacuo. Taken up in 150 ml of dichloromethane and filtered through Hyflo Super Cel®. The clear filtrate is washed with 50 ml of aqueous sodium hydrogen sulfate (10%), 50 ml of aqueous sodium hydrogen carbonate (saturated), 50 ml of water, and 50 ml of brine, dried over magnesium sulfate and filtered. Purified by flash chromatography (400 g of SiO₂, (heptane:ethyl acetate (1:1)) to give 1.25 g (21%) of (2S)-2-[biphenyl-4-ylmethyl-(2S)-(2-tert-butoxycarbonylamino-3-(2-naphthyl)propionyl)amino]-3-(9H-fluoren-9-ylmethoxycarbonylamino)propionic acid methyl ester.

HPLC-MS: Rₜ=8.83 min., (M+1)=804, % Area by ELS=45

Step E:

To a solution of (2S)-2-[biphenyl-4-ylmethyl-(2S)-(2-tert-butoxycarbonylamino-3-(2-naphthyl)propionyl)amino]-3-(9H-fluoren-9-ylmethoxycarbonylamino)propionic acid methyl ester (1.20 g, 1.49 mmol) in 20 ml of dichloromethane is added 20 ml of trifluoroacetic acid. Stirred for 2 hours, after which the solvent is removed in vacuo. Stripped 2 times from dichloromethane to afford 1.26 g (theoretically 1.49 mmol) of (2S)-2-[((2S)-2-amino-3-(2-naphthyl)propionyl)biphenyl-4-ylmethylamino]-3-(9H-fluoren-9-ylmethoxy carbonylamino)propionic acid methyl ester trifluoroacetic acetate as yellow foam.

HPLC-MS: Rt=6.93 min., (M+1)=705, % Area by ELS=40

Step F:

To a solution of (2S)-2-[((2S)-2-amino-3-(2-naphthyl)propionyl)biphenyl-4-ylmethylamino]-3-(9H-fluoren-9-ylmethoxycarbonylamino)propionic acid methyl ester trifluoroacetic acetate in 20 ml of dichloromethane is added 2 ml of N-ethyldiisopropylamin. Stirred for 4 hours, then 80 ml of dichloromethane is added, and the mixture is washed with 20 ml of aqueous sodium hydrogen sulfate (10%), 20 ml of aqueous sodium hydrogen carbonate (saturated), 20 ml of brine, dried over magnesium sulfate, filtered and concentrated in vacuo to afford 1.02 g (theoretically 1.49 mmol) of ((2S,5S)-1-Biphenyl-4-ylmethyl-5-(2-naphthyl)methyl-3,6-dioxo-piperazin-2-ylmethyl)carbamic acid 9H-fluoren-9-ylmethyl ester as yellow foam.

HPLC-MS: Rt=7.90 min., (M+1)=672, % Area by ELS=96

Step G:

To a solution of ((2S,5S)-1-biphenyl-4-ylmethyl-5-(2-naphthyl)methyl-3,6-dioxo-piperazin-2-ylmethyl)carbamic acid 9H-fluoren-9-ylmethyl ester (theoretically 1.49 mmol) in 10 ml of dichloromethane is added 10 ml of tris(2-aminoethyl)amine. Stirred for 2 hours under nitrogen. The mixture is added 30 ml of dichloromethane and 30 ml of brine, mixed and separated. The aqueous phase is extracted 2 times with 20 of dichloromethane, and the combined organic phases are washed with 3 times of 30 ml of aqueous phosphate buffer (pH: 6.6), 20 ml of brine, dried over magnesium sulfate, filtered and concentrated in vacuo to afford 0.50 g (74%) of (3S,6S)-6-Aminomethyl-1-biphenyl-4-ylmethyl-3-(2-naphthyl)methylpiperazine-2,5-dione as yellow foam.

HPLC-MS: Rt=4.86 min., (M+1)=450, % Area by ELS=100

Step H:

To a solution of (3S,6S)-6-aminomethyl-1-biphenyl-4-ylmethyl-3-(2-naphthyl)methylpiperazine-2,5-dione (0.15 g, 0.33 mmol) in 5 ml of tetrahydrofuran and 5 ml of methanol is added sodium acetate (0.11 g, 1.32 mmol), 4-formyl-piperidine-1-carboxylic acid tert-butyl ester (0.070 g, 0.33 mmol), molecular sieves (4A) and 1.0 M sodium cyanoborohydride (0.33 ml, 0.33 mmol) in tetrahydrofuran. Stirred overnight and then filtered through Hyflo Super Cel®. Concentrated in vacuo, dissolved in 50 ml of dichloromethane and washed with 10 ml of aqueous sodium hydrogen carbonate (saturated), 10 ml of brine, dried over magnesium sulfate, filtered and concentrated in vacuo to afford 0.21 g (100%) of 4-{[((2S,5S)-1-biphenyl-4-ylmethyl-5-(2-naphthyl)methyl-3,6-dioxo-piperazin-2-ylmethyl)amino]methyl}piperidine-1-carboxylic acid tert-butyl ester as orange oil.

HPLC-MS: Rt=5.57 min., (M+1)=647, % Area by ELS=87

Step I

To a solution of 3-tert-butoxycarbonylamino-3-methyl-butyric acid (0.034 g, 0.16 mmol) in 5 ml of dichloromethane is added 1-hydroxy-7-azabenzotriazole (0.021 g, 0.16 mmol) and N-ethyl-N′-(3-dimethylaminopropyl)-carbodiimide hydrochloride (0.030 g, 0.16 mmol). Stirred for 30 min after which 4-{[((2S,5S)-1-biphenyl-4-ylmethyl-5-(2-naphthyl)methyl-3,6-dioxo-piperazin-2-ylmethyl)-amino]methyl}piperidine-1-carboxylic acid tert-butyl ester (0.10 g, 0.16 mmol) and N-ethyldiisopropylamin (0.035 ml, 0.20 mmol) are added. Stirred overnight to give a clear yellow solution. The mixture is added to 10 ml of dichloromethane and 5 ml of aqueous sodium hydrogen sulfate (10%), mixed and separated. The aqueous phase is extracted with 5 ml of dichloromethane, and the combined organic phases are washed with 5 ml of aqueous sodium hydrogen carbonate (saturated), 5 ml of brine, dried over magnesium sulfate, filtered and concentrated in vacuo to afford 0.15 g (theoretically 0.16 mmol) of 4-{[((2S,5S)-1-biphenyl-4-ylmethyl-5-(2-naphthyl)methyl-3,6-dioxo-piperazin-2-ylmethyl)-(3-tert-butoxycarbonylamino-3-methyl-butyryl)amino]methyl}piperidine-1-carboxylic acid tert-butyl ester as yellow oil.

HPLC-MS: Rt=8.20 min., (M+1)=847, % Area by ELS=89

Step J:

To a solution of 4-{[((2S,5S)-1-biphenyl-4-ylmethyl-5-(2-naphthyl)methyl-3,6-dioxo-piperazin-2-ylmethyl)-(3-tert-butoxycarbonylamino-3-methyl-butyryl)amino]methyl}piperidine-1-carboxylic acid tert-butyl ester in 5 ml of dichloromethane is added 5 ml of trifluoroacetic acid. Stirred for 2 hours, concentrated in vacuo, stripped 2 times from dichloromethane. Purified by preparative HPLC (20-40% acetonitrile in water /0.1% trifluoroacetic acid, 40 min). The obtained pure fractions are combined and 1 ml of 1 N aqueous hydrogen chloride is added. The compound is lyophilized to give 55 mg (52%) of the title compound as a hydrochloride salt.

HPLC (A): Rₜ=30.17 min., 99% (214 nm); HPLC (B): Rₜ=32.87 min., 99% (214 nm); HPLC-MS: Rₜ=4.30 min., (M+1)=646, % Area by ELS=100

### Example 80

### (S,S)-3-Amino-N-(1-biphenyl-4-ylmethyl-5-naphthalen-2-ylmethyl-3,6-dioxo-piperazin-2-ylmethyl)-N-pyridin-4-ylmethyl-propionamide

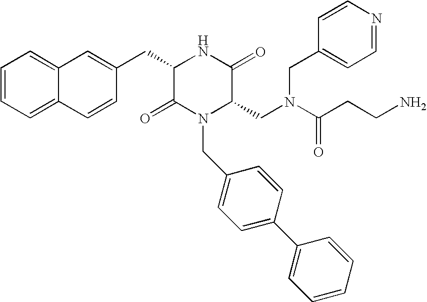

21.5 mg of the title compound is synthesized as described for (S,S)-3-amino-N-(1-biphenyl-4-ylmethyl-5-naphthalen-2-ylmethyl-3,6-dioxo-piperazin-2-ylmethyl)-3-methyl-N-piperidin-4-ylmethyl-butyramide using pyridine-4-carbaldehyde instead of 4-formyl-piperidine-1-carboxylic acid tert-butyl ester and 3-tert-butoxycarbonylamino-propionic acid instead of 3-tert-butoxycarbonylamino-3-methyl-butyric acid.

The title compound is purified by preparative HPLC (20-40% acetonitrile in water /0.1% trifluoroacetic acid, 40 min). To the combined pure fractions are added 1 ml of 1 M aqueous hydrogen chloride and the mobile phase is removed by lyophilisation.

HPLC (A1): Rₜ=28.77 min., 100% (214 nm); HPLC (B1): Rₜ=30.66 min., 100% (214 nm); HPLC-MS: Rₜ=4.23 min., (M+1)=612, % Area by ELS=100

### Example 81

### (S,S)-3-Amino-N-[5-(4-ethoxy-benzyl)-3,6-dioxo-1-(4-phenoxy-benzyl)-piperazin-2-ylmethyl]-3-methyl-N-piperidin-4-ylmethyl-butyramide

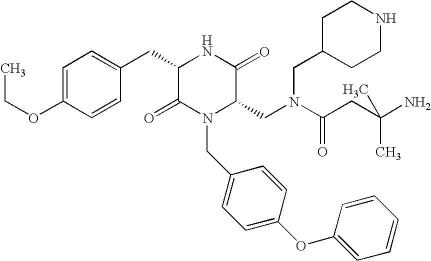

55 mg of the title compound is synthesized as described for (S,S)-3-amino-N-(1-biphenyl-4-ylmethyl-5-naphthalen-2-ylmethyl-3,6-dioxo-piperazin-2-ylmethyl)-3-methyl-N-piperidin-4-ylmethyl-butyramide using 4-phenoxybenzaldehyde instead of biphenyl-4-carbidehyde and (S)-2-tert-butoxycarbonylamino-3-(4-ethoxyphenyl)propionic acid instead of (S)-2-tert-butoxycarbonylamino-3-(2-naphtyl)propionic acid.

The title compound is purified by preparative HPLC (23-43% acetonitrile in water/0.1% trifluoroacetic acid, 40 min). To the combined pure fractions are added 1 ml of 1 M aqueous hydrogen chloride and the mobile phase is removed by lyophilisation.

HPLC (A1): Rt=29.14 min., 99% (214 nm); HPLC (B1): Rt=31.59 min., 100% (214 nm); HPLC-MS: Rt=4.37 min., (M+1)=656, % Area by ELS=100

### Example 82

### (S,S)-3-Amino-N-[5-(4-ethoxy-benzyl)-3,6-dioxo-1-(4-phenoxy-benzyl)-piperazin-2-ylmethyl]-N-piperidin-4-ylmethyl-propionamide

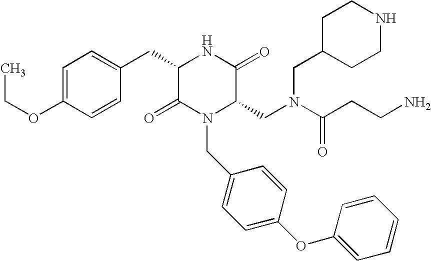

55 mg of the title compound is synthesized as described for (S,S)-3-amino-N-(1-biphenyl-4-ylmethyl-5-naphthalen-2-ylmethyl-3,6-dioxo-piperazin-2-ylmethyl)-3-methyl-N-piperidin-4-ylmethyl-butyramide using 4-phenoxybenzaldehyde instead of biphenyl-4-carbaldehyde and (S)-2-tert-butoxycarbonylamino-3-(4-ethoxyphenyl)propionic acid instead of (S)-2-tert-butoxycarbonylamino-3-(2-naphtyl)propionic acid.

The title compound is purified by preparative HPLC (23-43% acetonitrile in water /0.1% trifluoroacetic acid, 40 min). To the combined pure fractions are added 1 ml of 1 M aqueous hydrogen chloride and the mobile phase is removed by lyophilisation.

HPLC (A1): Rt=29.14 min., 99% (214 nm); HPLC (B1): Rt=31.59 min., 100% (214 nm); HPLC-MS: Rt=4.37 min., (M+1)=656, % Area by ELS=100

### Example 83

### (S,S)-6-{[Bis-(3H-imidazol-4-ylmethyl)-amino]-methyl}-3-(4-ethoxy-benzyl)-1-(4-phenoxy-benzyl)piperazine-2,5-dione

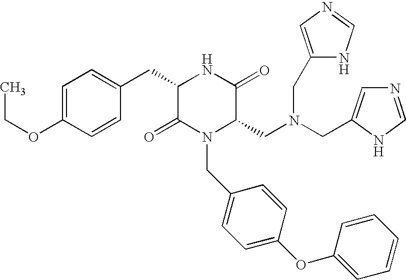 Step A:

6.27 g of (S)-3-tert-butoxycarbonylamino-2-(4-phenoxybenzylamino)propionic acid methyl ester is synthesized as described for (2S)-2-[(biphenyl-4-ylmethyl)amino]-3-(9H-fluoren-9-ylmethoxycarbonylamino)propionic acid methyl ester using (S)-2-amino-3-tert-butoxycarbonylamino-propionic acid methyl ester instead of (S)-2-Amino-3-(9H-fluoren-9-ylmethoxycarbonylamino)propionic acid methyl ester.

HPLC-MS: Rt=4.87 min., (M+1)=401, % Area by ELS=99

Step B:

To a solution of (S)-2-tert-butoxycarbonylamino-3-(4-ethoxyphenyl)propionic acid in 10 ml of dichloromethane is added O-(7-azabenzotriazol-1-yl)-N,N,N′,N′-tetramethyluronium hexafluorophosphate (5.70 g, 15.0 mmol), 1-hydroxybenzotriazole (2.04 g, 15.0 mmol) and N-ethyldiisopropylamin (2.57 ml, 15.0 mmol). Stirred for 20 min, after which a solution of (S)-3-tert-butoxycarbonylamino-2-(4-phenoxybenzylamino)propionic acid methyl ester (3.00 g, 7.49 mmol) in 10 ml of dichloromethane is added. Stirred overnight to give a yellow slurry. The mixture is diluted with 100 ml of dichloromethane and washed with 20 ml of aqueous sodium hydrogen sulfate (10%), 20 ml of aqueous sodium hydrogen carbonate (saturated), 20 ml of brine, dried over magnesium sulfate and filtered. Concentrated in vacuo to give a crude oil, which is purified by flash chromatography (100 g of SiO₂, heptane:ethyl acetate (7:3)) to afford 6.08 g (theoretically 7.49 mmol) of (2S)-3-tert-butoxycarbonylamino-2-[[(2S)-2-tert-butoxycarbonylamino-3-(4-ethoxyphenyl)propionyl]-(4-phenoxybenzyl)amino]propionic acid methyl ester as colorless oil.

Step C:

(2S)-3-tert-butoxycarbonylamino-2-[[(2S)-2-tert-butoxycarbonylamino-3-(4-ethoxyphenyl)propionyl]-(4-phenoxybenzyl)amino]propionic acid methyl ester (6.08 g, theoretically 7.49 mmol) is dissolved in 100 ml of dichloromethane and 100 ml of trifluoroacetic acid. Stirred for 2 hours, concentrated in vacuo, stripped 2 times from dichloromethane to give a thin orange oil. Dissolved in 100 ml of dichloromethane, 10 ml of N-ethyldiisopropylamin is added and the resulting mixture is stirred for 2 hours. Diluted with 100 ml of dichloromethane and 20 ml of aqueous sodium hydrogen carbonate (saturated), mixed and separated. The aqueous phase is extracted with 100 ml of dichloromethane, and the combined organic phases are washed with 20 ml of brine, dried over magnesium sulfate and filtered. Concentrated in vacuo to afford 5.19 g (theoretically 7.49 mmol) of (3S,6S)-(6-Aminomethyl-3-(4-ethoxybenzyl)-1-(4-phenoxybenzyl)piperazine-2,5-dione as yellow oil.

HPLC-MS: Rₜ=4.80 min., (M+1)=460, % Area by ELS=89

Step D:

To a solution of 3S,6S)-(6-aminomethyl-3-(4-ethoxybenzyl)-1-(4-phenoxybenzyl)piperazine-2,5-dione (0.24 g, 0.35 mmol) in 10 ml of tetrahydrofuran and 10 ml of methanol is added 4(5)-imidazolecarboxaldehyde (0.10 g, 1.1 mmol), molecular sieves (4A), acetic acid (42 μl, 0.20 mmol) and sodium cyanoborohydride (1.1 ml, 1.1 mmol). Stirred for 5 days. Filtered through Hyflo Super Cel®, concentrated in vacuo and purified by preparative HPLC (25-45% acetonitrile in water /0.1% trifluoroacetic acid, 40 min). The obtained pure fractions are combined and 2 ml of 1 N aqueous hydrogen chloride is added. The compound is lyophilized to give 132 mg (52%) of the title compound as a hydrochloride-salt.

HPLC (A1): Rₜ=29.27 min., 99% (214 nm); HPLC (B1): Rₜ=31.47 min., 98% (214 nm); HPLC-MS: Rₜ=4.40 min., (M+1)=620, % Area by ELS=100

### Example 84

### (S,S)-3-Amino-N-(2-amino-2-methyl-propyl)-N-[5-(4-ethoxy-benzyl)-3,6-dioxo- -(4-phenoxy-benzyl)-piperazin-2-ylmethyl]-3-methyl-butyramide

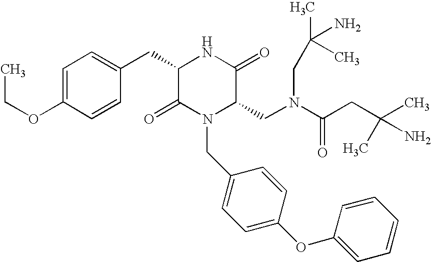

2.4 mg of the title compound is synthesized as described for (S,S)-3-amino-N-(1-biphenyl-4-ylmethyl-5-naphthalen-2-ylmethyl-3,6-dioxo-piperazin-2-ylmethyl)-3-methyl-N-piperidin-4-ylmethyl-butyramide using 4-phenoxybenzaldehyde instead of biphenyl-4-carbaldehyde, (S)-2-tert-butoxycarbonylamino-3-(4-ethoxyphenyl)propionic acid instead of (S)-2-tert-butoxycarbonylamino-3-(2-naphtyl)propionic acid and (1,1-dimethyl-2-oxo-ethyl)carbamic acid tert-butyl ester instead of 4-formyl-piperidine-1-carboxylic acid tert-butyl ester. The title compound is purified by preparative HPLC (23-43% acetonitrile in water/0.1% trifluoroacetic acid, 40 min). To the combined pure fractions are added 1 ml of 1 M aqueous hydrogen chloride and the mobile phase is removed by lyophilisation.

HPLC (h8): Rt=9.09 min., 84% (214 nm); HPLC-MS: Rt=4.60 min., (M+1)=630, % Area by ELS=100

### Example 85

### (S,S)-1-[4-(4-Acetyl-phenoxy)-benzyl]-6-(4-amino-butyl)-3-naphthalen-2-ylmethyl-piperazine-2,5-dione

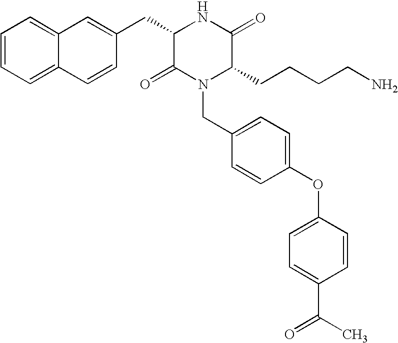 Step A:

To a solution of 4-hydroxybenzaldehyde (2.44 g, 20 mmol), triethylamine (3.37 ml, 24.2 mmol) and a catalytic amount of 4-dimethylaminopyridine in DCM (50 ml) is added a solution of tert-butyidimethylsilyl chloride in DCM (25 ml) dropwise during 30 min at 0° C. The mixture is allowed to reach room temperature and stirred overnight. The mixture is evaporated in vacuo and the residue is purified on silica with ethyl acetate/heptane (1:4) to give the product, which is used in the next step.

HPLC-MS (Method C): m/z=237 (M+1); Rₜ=5.5 min.

Step B:

To a solution of H-Lys(Boc)-OMe HCl (3.0 g, 11.3 mmol) in THF (80 ml) is added the protected aldehyde from step A (2.66 g, 16.7 mmol) and N,N-diisopropylethylamine (2.0 ml, 11.3 mmol), and the mixture is stirred in the presence of powdered molecular sieves (4 Å) overnight at room temperature. Then methanol (10 ml), acetic acid (4.8 ml) and sodium cyanoborohydride (2.1 g, 34 mmol) is added and the mixture is stirred for 7 h at room temperature. The mixture is evaporated in vacuo and the residue is taken up in ethyl acetate (80 ml) and filtered. The filtrate is washed with 1 N sodium hydroxide (60 ml), dried over sodium sulfate and evaporated to dryness in vacuo to give the crude product (5.97 g), which is used in the next step without further purification.

HPLC-MS (Method C): m/z=481 (M+1); Rₜ=3.6 min.

Step C:

To a solution of Boc-O-2-naphthyl-Ala-OH (2.0 g, 6.35 mmol) in THF (15 ml) is added N,N′-diisopropylcarbodiimide (0.49 ml, 3.17 mmol) and the mixture is stirred for 30 min at room temperature. A solution of the crude product from step B (1.52 g, ca. 3.1 mmol) in THF is added and the mixture is stirred for 4 h at room temperature. Then N,N-diisopropylethylamine (1.1 ml, 6.4 mmol) is added and stirring is continued overnight. The mixture is evaporated in vacuo and the residue is taken up in ethyl acetate (50 ml) and washed successively with 1 N HCl (30 ml) and saturated aqueous sodium hydrogen carbonate (30 ml), dried over sodium sulfate and evaporated to dryness. Column chromatography on silica with ethyl acetate/heptane (1:2) afforded the intermediate in a yield of 1.10 g.

HPLC-MS (Method C): m/z=800 (M+23); Rₜ=7.0 min.

Step D:

A solution of the product from step C (1.1 g, 1.45 mmol) and TFA (10 ml) in DCM (25 ml) is stirred for 2 h at room temperature. After evaporation in vacuo the residue is taken up in toluene (20 ml) and the solvent is again removed in vacuo. The residue is now dissolved in DCM (25 ml) and N,N-diisopropylethylamine (1.0 ml, 5.8 mmol) is added. After stirring overnight, the mixture is evaporated in vacuo and the residue is taken up in ethyl acetate and stirred with 1 N HCl (20 ml) for 5 h at room temperature. After evaporation in vacuo, the residue is purified on a C18 reverse phase column (Sep-Pak, Waters, 10 g) with 0.1% TFA in water and acetonitrile affording 364 mg of the ring-closed deprotected product.

HPLC-MS (Method C): m/z=432 (M+1); Rₜ=1.8 min.

Step E:

A solution of di-tert-butyl dicarbonate (203 mg, 0.93 mmol) and N,N-diisopropylethylamine (161 μl, 0.93 mmol) in DCM is added dropwise to a solution of the product from step D, and the mixture is stirred overnight at room temperature. The mixture is evaporated in vacuo and the residue is purified on silica with ethyl acetate to give 365 mg of the Boc-protected product, which is used in the next step.

HPLC-MS (Method C): m/z=554 (M+23); Rₜ=3.8 min.

Step F:

A slurry of the product from step E (115 mg, 0.216 mmol), 4-acetylphenylboronic acid (177 mg, 1.08 mmol), copper(II) acetate (196 mg, 1.08 mmol), triethylamine (150 μl, 1.05 mmol) and powdered molecular sieves (4 Å) in THF is stirred at room temperature for about two days. The mixture is filtered and the filtrate is evaporated in vacuo. The product is isolated from the residue by column chromatography on silica with ethyl acetate/heptane (1:2) and used directly in the following step

HPLC-MS (Method C): m/z=672 (M+23), 550 (M−100+1); Rₜ=4.7 min.

Step G:

The Boc-protected product from step F (100 mg, 0.15 mmol) is stirred with TFA (3 ml) in DCM (10 ml) for 1 h at room temperature. After evaporation in vacuo, the residual oil is purified on a C18 reverse phase column (Sep-Pak, Waters, 10 g) with 0.1% TFA in water and acetonitrile. The product is dissolved in a mixture of 1 N HCl and methanol and evaporated in vacuo affording 364 mg of the title compound as the hydrochloride

HPLC-MS (Method C): m/z=550 (M+1); Rₜ=2.8 min

### Example 86

### (S,S)-6-(4-Amino-butyl)-1-biphenyl-4-ylmethyl-3-[4-(3-hydroxymethyl-phenoxy)-benzyl]-piperazine-2,5-dione

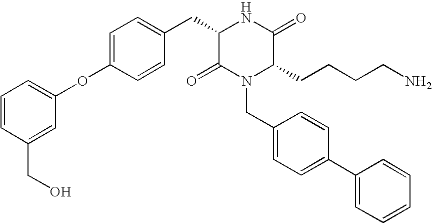 Step A:

288 mg of the boc-protected product of example 75 is dissolved in 10 ml ethanol and 17 mg sodium borohydride is added. After a few hours (TLC control) the product is formed and the solvent is removed in vacuo. Water is added and the mixture is extracted with ethyl acetate. The organic phase is dried over sodium sulfate. The solvent is removed in vacuo and the residual is purified on silica with ethyl acetate.

Step B:

72 mg of the product from step A is dissolved in 10 ml dichlormethane and 1 ml trifluoroacetic acid is added. The mixture is stirred over night. The solvent is removed in vacuo and the residual is purified on a C18 reverse phase column.

¹H NMR (CDCl₃): δ 0.7-1.7 (6H, m), 2.7-3.3 (4H, m), 3.6-3.7 (1H, m), 3.85 (1H, d), 4.3-4.4 (1H, m), 4.5 (2H, s), 5.25 (1H, d), 6.7-7.6 (17H, m), 7.8-8.1 (2H, bs).

HPLC-MS (Method C): m/z=564 (M+1); Rₜ=2.96 min.

### Example 87

### (S,S)-6-{4-[(1H-imidazol-2-ylmethyl)-amino]-butyl}-3-(4-methoxy-benzyl)-1-(4-phenoxy-benzyl)piperazine-2,5-dione

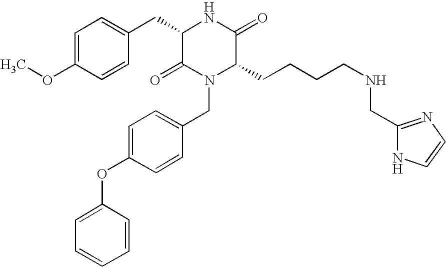 Step A:

The product resin from general procedure C, step A, is used. To 0.18 g of this resin are added sequentially a solution of 0.468 mmol Boc-Lys(Fmoc)-OH in 1.6 ml of 1,2-dichloropropane/tetrahydrofuran (1:1), 0.045 ml (0.288 mmol) of diisopropylcarbodiimide, and a solution of 0.036 mmol of 4-dimethylamino pyridine in 0.2 ml of 1,2-dichloropropane. The mixture is shaken for 15 hours. The liquids are filtered off and the resin is washed with dimethylformamide (2×2 ml), tetrahydrofuran (2×2 ml), and dichloromethane (2×2 ml).

Step B:

The resin obtained by step A is shaken with a mixture of 2.5 ml trifluoroacetic acid/dichloromethane 1:1 for one hour. The liquids are filtered off and the resin is washed with tetrahydrofuran (2×2 ml), tetrahydrofuran/ethyldiisopropylamine 3:1 (3×2 ml), methanol (2 ml) and tetrahydrofuran (2 ml).

Step C:

To the resin obtained by step B, a solution of 0.36 mmol of 4-phenoxybenzaldehyde in 1.7 ml of 1-methyl-2-pyrrolidone and 0.1 ml of acetic acid are added. The mixture is shaken for three hours. The liquids are filtered off. The resin is shaken with a solution of 0.9 mmol of sodium cyanoborohydride in 1.7 ml of dichloromethane/methanol 1:1 for one hour. The liquids are filtered off. The resin is washed with methanol (2×2 ml), dichloromethane/methanol 1:1 (2 ml), dichloromethane/ethyldiisopropylamine 19:1 (2×2 ml), and tetrahydrofuran (2×2.5 ml).

Step D:

To the resin obtained by step C, a solution of 0.468 mmol of Boc-Tyr(Me)-OH in 1.6 ml of 1,2-dichloropropane/tetrahydrofuran 1:1 is added, followed by a solution of 0.288 mmol of diisopropylcarbodiimide in 0.2 ml of 1,2-dichloropropane. The mixture is shaken for 30 minutes. 0.043 ml (0.252 mmol) of ethyldiisopropylamine is added, and shaking is continued for 14 hours. The liquids are filtered off and the resin is washed with tetrahydrofuran (2×2.5 ml). The same amounts of Boc-Tyr(Me)-OH and diisopropylcarbodiimide as described above are added and the mixture is shaken for 45 minutes. 0.043 ml (0.252 mmol) of ethyldiisopropylamine is added, and shaking is continued for 7 hours. The liquids are filtered off and the resin is washed with dimethylformamide (2×2 ml) and tetrahydrofuran (2×3 ml).

Step E:

The resin obtained by step D is shaken with a mixture of 1.5 ml of dimethylformamide and 0.5 ml of piperidine for 30 min. The liquids are filtered off and the resin is washed with dimethylformamide (2×2 ml) and tetrahydrofuran (2×3 ml).

Step F:

To the resin obtained by step E, a suspension of 0.36 mmol of imidazole-2-carbaldehyde in 1.8 ml of 1-methyl-2-pyrrolidone/tetrahydrofuran 17:1 is added, followed by 0.1 ml of acetic acid. The mixture is shaken for 2.5 hours. The liquids are filtered off and the resin is washed with dichloromethane (4×2 ml). A solution of 0.90 mmol of sodium cyanoborohydride in 1.7 ml of dichloromethane/methanol 1:1 and 0.05 ml of acetic acid are added and the mixture is shaken for one hour. The liquids are filtered off and the resin is washed with methanol (2×2 ml), dichloromethane/methanol 1:1 (2 ml), tetrahydrofuran (2×3 ml), dichloromethane /ethyldiisopropylamine 8:1 (2×1.8 ml), and dichloromethane (5×2 ml).

Step G:

The resin obtained by step F is shaken with 2.5 ml of dichloromethane/trifluoroacetic acid 1:1 for 30 minutes. The liquids are filtered off and the resin is washed with dichloromethane (2×2 ml), tetrahydrofuran (2×2.5 ml), and methanol (2×2.5 ml).

Step H:

To the resin obtained by step G, 2.0 ml of dichloromethane and 1.0 ml of 40% methylamine in methanol are added. The mixture is shaken for 3.5 hours. The mixture is filtered and the filtrate is collected. The resin is washed with 3.5 ml of dichloromethane/methanol 6:1 and the washing filtrate is collected. Both filtrates are mixed and evaporated to give a residue.

Step I:

The residue obtained by step H is dissolved in a mixture of 4.8 ml of water, 3.2 ml of acetonitrile and 0.8 ml of 1 M aqueous hydrochloric acid and purified by HPLC. Addition of dilute aqueous hydrochloric acid and freeze-drying afforded 12.2 mg of the product.

HPLC-MS (Method B): m/z=568 (M+1); Rₜ=4.67 min.

### Example 88

### (S,S)-3-(4-Methoxy-benzyl)-1-(4-phenoxy-benzyl)-6-{4-[(pyridin-2-ylmethyl)-amino]-butyl}-piperazine-2,5-dione

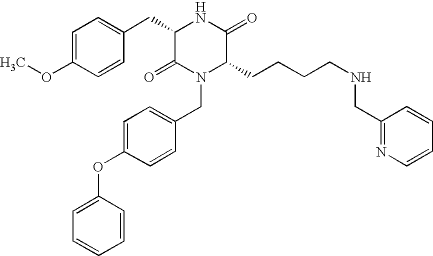 Steps A-H:

The same procedure as described for example 87, steps A-H, is performed. In step F, a solution of pyridine-2-carbaldehyde is used instead of the imidazole-2-carbaldehyde suspension.

Step I:

The residue obtained by step H is dissolved in a mixture of 4.8 ml water, 3.2 ml of acetonitrile and 0.8 ml of 1 M aqueous hydrochloric acid and purified by HPLC. Addition of dilute aqueous hydrochloric acid and freeze-drying afforded 20.6 mg of the product.

HPLC-MS (Method B): m/z=579 (M+1); Rₜ=4.97 min.

### Example 89

### (2R,2′S,5′S)-2-Amino-N-[5-(4-ethoxy-benzyl)-3,6-dioxo-1-(4-phenoxy-benzyl)-piperazin-2-ylmethyl]-3-(1H-imidazol-4-yl)-propionamide

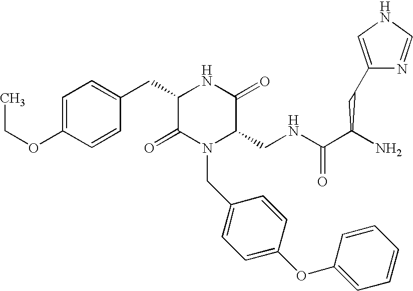 Step A:

To a solution of (1R)-4-(2-tert-butoxycarbonylamino-2-carboxyethyl)imidazole-1-carboxylic acid tert-butyl ester (0.35 mmol) in 1 ml of dichloromethane is added O-(7-azabenzotriazol-1-yl)-N,N,N′,N′-tetrametyluronium hexafluorophosphate (0.13, 0.35 mmol, 1-hydroxybenzotriazole (0.048 g, 0.35 mmol) and N-ethyldiisopropylamin (120 μl, 0.70 mmol). Stirred for 20 min, after which a solution of (S3S,6S)-(6-aminomethyl-3-(4-ethoxybenzyl)-1-(4-phenoxybenzyl)piperazine-2,5-dione (0.24 g, 0.35 mmol) in 1 ml of dichloromethane is added. Stirred overnight to give a yellow solution. Diluted with 15 ml of dichloromethane, washed with 2.5 ml of aqueous sodium hydrogen sulfate (10%), 2.5 ml of aqueous sodium hydrogen carbonate (saturated), 2.5 ml of brine, dried over magnesium sulfate and filtered. Concentrated in vacuo to afford 0.33 g (theoretically 0.35 mmol) of 4-(2-tert-butoxycarbonylamino-2-(1R)-{[(2S,5S)-5-(4-ethoxybenzyl)-3,6-dioxo-1-(4-phenoxybenzyl)piperazin-2-ylmethyl]carbamoyl}ethyl)imidazole-1-carboxylic acid tert-butyl ester as yellow oil.

HPLC-MS: Rt=6.53 min., (M+1)=797, % Area by ELS=70

Step B:

To a solution of -(1R)-{[(2S,5S)-5-(4-ethoxybenzyl)-3,6-dioxo-1-(4-phenoxybenzyl)piperazin-2-ylmethyl]carbamoyl}ethyl)imidazole-1-carboxylic acid tert-butyl ester (theoretically 0.35 mmol) in 5 ml of dichloromethane is added 5 ml of trifluoroacetic acid. Stirred for 2 hours, concentrated in vacuo and purified by preparative HPLC (23-43% acetonitrile in water /0.1% trifluoroacetic acid, 40 min). To the combined pure fractions are added 2 ml of 1 M aqueous hydrogen chloride and the mobile phase is removed by lyophilisation to afford 88.1 mg of the title compound. HPLC (A1): Rₜ=29.68 min., 100% (214 nm); HPLC (B1): Rt=31.84 min., 100% (214 nm); HPLC-MS: Rt=4.33 min., (M+1)=597, % Area by ELS=100

### Example 90

### (S,S)-2-(3-Amino-propylamino)-N-[1-[4-(methyl-phenyl-amino)-benzyl]-3,6-dioxo-5-(4-propoxy-benzyl)-piperazin-2-ylmethyl]-acetamide

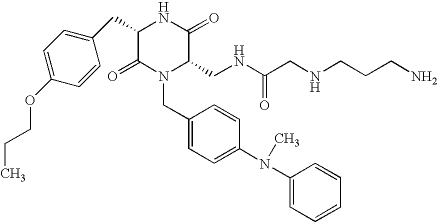 Step A:

0.5 g (2.0 mmol) H-Dap(Boc)-OMe hydrochloride and 0.4 g 4-(methyl-phenyl-amino)benzaldehyde are taken up in 20 ml THF. 340 μl DIPEA is added and the mixture is stirred over night. 0.4 g NaCNBH₃, 2 ml methanol and 1 ml HOAc are added and the mixture is stirred for 5 h. The solvent is removed in vacuo and the residual oil is taken up in 75 ml ethyl acetate. The org. phase is washed twice with 50 ml 1 N NaOH and dried over sodium sulfate. The solvent is removed in vacuo and the crude material is used for the next step.

Step B:

1.4 g (3.9 mmol) Boc-Tyr(tBu)-OH are dissolved in 20 ml THF. 300 μl diisopropylcarbodiimide are added and the mixture is stirred for 1 h. The crude product from step A is added in 10 ml THF. After 2.5 h 320 μl DIPEA is added and the reaction is stirred over night. Another 320 μl DIPEA are added and after 1 h the solvent is removed in vacuo. The residual oil is taken up in 50 ml ethyl acetate. The org. phase is washed twice with 50 ml 1 N HCl, twice with 50 ml sat. sodium hydrogen carbonate and dried over sodium sulfate. The solvent is removed in vacuo and the residual oil is purified on silica using ethyl acetate/heptane 2:3.

Step C:

0.5 g (0.8 mmol) of the product from step B is dissolved in 15 ml dichlormethane and 15 ml TFA. The solvent is removed after 30 min and the residual oil is dissolved in 25 ml dichlormethane and 2 ml DIPEA. The solvent is removed after 45 min and the oil is purified on a C18 reverse phase column (Sep-Pak, Waters, 10 g) with 0.1% TFA in water and acetonitril.

Step D:

0.3 g (0.7 mmol) of the product from step C is dissolved in 15 ml dichlormethane. 290 μl Boc-anhydrid and 115 μl DIPEA are added. The mixture is stirred over night. The solvent is removed in vacuo and the oil is purified on silica with ethyl acetate.

Step E:

0.2 g (0.4 mmol) of the product from step D is dissolved in 5 ml THF. 0.14 g (1.5 equi.) triphenylphosphine and 41 μl 1-propanol are added. 87 μl diethylazadicarboxylate is added and the mixture is stirred over night. 0.10 g (1 equi) triphenylphosphine, 28 μl 1-propanol and 58 μl diethylazadicarboxylate are added again and the reaction is stirred for a second night. The solvent is removed in vacuo and the oil is purified on a C18 reverse phase column (Sep-Pak, Waters, 10 g) with 0.1% TFA in water and acetonitril.

Step F:

0.22 g (0.4 mmol) of the product from step E is dissolved in 10 ml dichlormethane and 10 ml TFA. The solvent is removed in vacuo after 25 min.

HPLC-MS (Method C): m/z=601 (M+1); Rₜ=2.77 min.

### Examples 91 to 102

Compounds of general formula (If) is synthesised on a small shaker according to general procedure C using as first building block (step B) Fmoc-L-Lys(Boc)-OH. 3-Phenoxybenzaldehyde, biphenyl-4-carbaldehyde, benzaldehyde or 4-benzyloxy-benzaldehyde is used as second building block (step D). The third building block (step E) is covered by Boc-β-(2-naphthyl)-L-Ala-OH, Boc-L-Tyr(bz)-OH, Boc-L-Trp(Boc)-OH, Boc-β-(1-naphthyl)-L-Ala-OH, Boc-L-Bip-OH or Boc-L-Phe-OH, samples are analysed using HPLC-MS method D.

Examples of compounds prepared according to said procedure of the general formula (If) are shown in Table VIII.

TABLE VIII

| | | | | | | Formula (If) | |
|---|---|---|---|---|---|---|---|
| | | | | | | | Pur- |
| Exam- | | | | | Ster- | Ster- | ity |
| ple | | | | | eo | eo | by |
| # | a | A | E | G² | pos 3 | pos 6 | ELS |
| 91 | 4 | —NH₂ | -3-PhOPh | -2-Np | S | S | 76% |
| 92 | 4 | —NH₂ | -3-PhOPh | -Ph(4-OBzl) | S | S | 67% |
| 93 | 4 | —NH₂ | -3-PhOPh | -1-Np | S | S | 74% |
| 94 | 4 | —NH₂ | -3-PhOPh | -4-Biph | S | S | 76% |
| 95 | 4 | —NH₂ | -4-Biph | -2-Np | S | S | 85% |
| 96 | 4 | —NH₂ | -4-Biph | -Ph(4-OBzl) | S | S | 75% |
| 97 | 4 | —NH₂ | -4-Biph | -1-Np | S | S | 80% |
| 98 | 4 | —NH₂ | -4-Biph | -4-Biph | S | S | 73% |
| 99 | 4 | —NH₂ | -Ph(4-OBzl) | -2-Np | S | S | 76% |
| 100 | 4 | —NH₂ | -Ph(4-OBzl) | -Ph(4-OBzl) | S | S | 73% |
| 101 | 4 | —NH₂ | -Ph(4-OBzl) | -1-Np | S | S | 76% |
| 102 | 4 | —NH₂ | -Ph(4-OBzl) | -4-Biph | S | S | 75% |

Stereo pos 3 and 6 = Absolute stereochemistry at the position 3 and 6, respectively, of the diketopiperazin ring system

### Example 103

### N-[4-((2S,5S)-1-Biphenyl-4-ylmethyl-5-naphthalen-2-ylmethyl-3,6-dioxo-piperazin-2-yl)-butyl]-acetamide

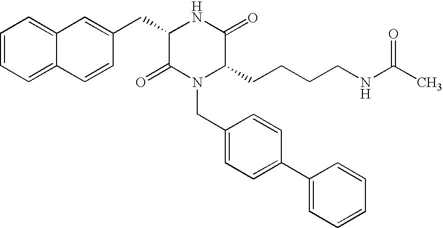

To the corresponding product of general procedure E, step G (196 mg, 0.4 mmol) and acetic anhydride (0.042 ml, 0.44 mmol) in dichloromethane (5 ml) is added at room temperature N-ethyldiisopropylamine (0.1 ml, 0.8 mmol) and the mixture is stirred for 1 h. Flash chromatography (silica, dichloromethane/MeOH 30:1 <20:1) gave the product (188 mg, 88%). ESI-MS: (M+H)⁺=534.

### Example 104

### (3S,6S)-1-Biphenyl-4-ylmethyl-6-(4-dimethylamino-butyl)-3-naphthalen-2-ylmethyl-piperazine-2,5-dione

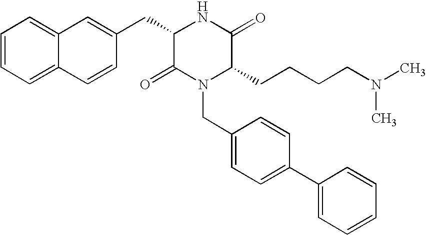

To the corresponding product of general procedure E, step G (245 mg, 0.5 mmol) and formaldehyde (37% in water, 0.67 ml, 8.9 mmol) in MeOH (10 ml) is added in portions sodium borohydride (151 mg, 4 mmol) and the mixture is stirred at room temperature overnight. Sat aq. sodium bicarbonate (40 ml) is added and the mixture is extracted with dichloromethane (3×70 ml). The combined org. layers are dried over sodium sulfate and purified by flash chromatography (silica, dichloromethane/MeOH 20:1+1% conc. aq. ammonia) to give the product (72 mg, 28%). ESI-MS: (M+H)⁺=520.

### Example 105

### N-[4-((2S,5S)-1-Biphenyl-4-ylmethyl-5-naphthalen-2-ylmethyl-3,6-dioxo-piperazin-2-yl)-butyl]-guanidine hydrochloride

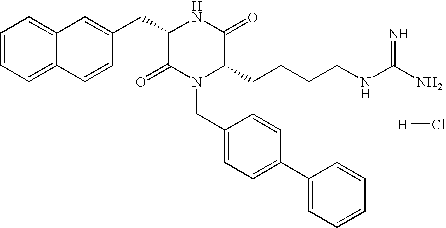

To the corresponding product of general procedure E, step G (196 mg, 0.4 mmol) in DMF (5 ml) is added pyrazole-1-carboxamidine hydrochloride (60 mg, 0.41 mmol) and the mixture is stirred at room temperature overnight. Ether is added and the white precipitate collected by filtration. The precipitate is washed repeatedly with ether and dried under high vacuum to give the product (186 mg, 82%). ESI-MS: (M+Cl)⁻=570.

### Example 106

### (3S,6S)-6-[4-(3-Amino-pyridin-2-ylamino)-butyl]-3-naphthalen-2-ylmethyl-1-(4-phenoxy-benzyl)piperazine-2,5-dione

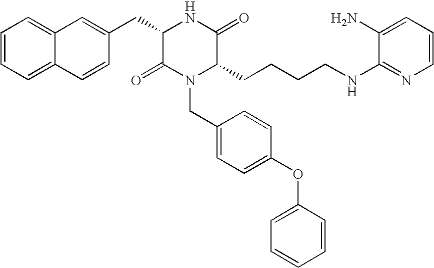 Step 1:

To the corresponding product of general procedure E, step G (100 mg, 0.2 mmol) and 2-chloro-3-nitro-pyridine (40 mg, 0.25 mmol) in DMF (1 ml) is added N-ethyldiisopropylamine (0.07 ml, 0.4 mmol) and the mixture is stirred at room temperature for 72 h. The mixture is diluted with ice water (50 ml) and the precipitate is collected by filtration. Flash chromatography (silica, dichloromethane/MeOH 30:1) gave the corresponding 3-nitropyridyl intermediate (90 mg, 73%). ESI-MS: (M+H)⁺=630

Step 2:

The 3-nitropyridyl intermediate (0.14 mmol) is hydrogenated (50 psi) in MeOH (15 ml) in the presence of Raney nickel (100 mg) at room temperature for 1 h. The catalyst is removed by filtration and the filtrate concentrated in vacuo. The residue is dissolved in ether (10 ml) and the product precipitated by addition of HCl (6-7 N in isopropanol) to give the product (38%). ESI-MS: (M+H)⁺=600.

### Example 107

### {4-[(2S,5S)-5-Naphthalen-2-ylmethyl-3,6-dioxo-1-(4-phenoxy-benzyl)-piperazin-2-yl]-butylamino}-acetonitrile

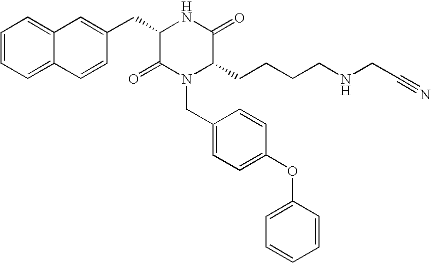

The corresponding product of general procedure E, step G (150 mg, 0.295 mmol) and chloroacetonitrile (0.02 ml, 0.313 mmol) in EtOH (1.5 ml) is heated to reflux for 3 h. Chloroacetonitril (0.01 ml) is added and the mixture is heated for another 2 h. The mixture is concentrated in vacuo and the residue purified by flash chromatography (silica, dichloromethane/MeOH 20:1) to give the product (80 mg, 50%). ESI-MS: (M+H)⁺=547

### Example 108

### N-((2S,5S)-1-Biphenyl-4-ylmethyl-5-naphthalen-2-ylmethyl-3,6-dioxo-piperazin-2-ylmethyl)-N-piperidin-4-ylmethyl-acetamide

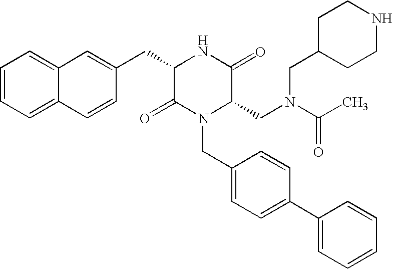 Step 1:

To the Cbz-protected corresponding product from general procedure G, step D (300 mg, 0.44 mmol) and acetic anhydride (0.085 ml, 0.90 mmol) is added at room temperature N-ethyldiisopropylamine (0.25 ml) and the mixture is stirred for 4 h. Sat. aq. sodium bicarbonate is added and the mixture is extracted with dichloromethane (3×50 ml). The combined org. layer are dried over sodium sulfate and purified by flash chromatography (silica, dichloromethane/MeOH 20:1) to give the acylated intermediate (300 mg, 94%). ESI-MS: (M+H)⁺=723.

Step 2:

The acylated intermediate (290 mg, 0.40 mmol) in MeOH (30 ml) is hydrogenated (50 psi) in the presence of Pd/C (10%) at 50° C. for 1 h. The catalyst is removed by filtration and the filtrate concentrated in vacuo. The residue is triturated (dichloromethane/ether) to give the product (112 mg, 47%). ESI-MS: (M+H)⁺=589.

### Example 109

### (3S,6S)-1-Biphenyl-4-ylmethyl-6-[(cyclohexylmethyl-piperidin-4-ylmethyl-amino)-methyl]-3-naphthalen-2-ylmethyl-piperazine-2,5-dione

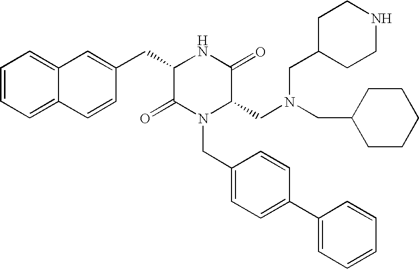 Step 1:

To the corresponding Cbz-protected product from general procedure G, step D (300 mg, 0.44 mmol) and cyclohexanecarboxaldehyde (0.12 ml, 0.99 mmol) in THF (20 ml) is added glacial acetic acid (0.06 ml) and the mixture is stirred for 1 h. Sodium triacetoxyborohydride (240 mg, 1.076 mmol) is added and the mixture is stirred for 3 days. Sat. aq. sodium bicarbonate is added, the mixture stirred for 30 min., and then extracted with ether (3×70 ml). The combined org. layers are dried over sodium sulfate, concentrated in vacuo, and the residue purified by flash chromatography (silica, dichloromethane/MeOH 30:1) to give the Cbz-protected intermediate (260 mg, 76%). ESI-MS: (M+H)⁺=777.

Step 2:

The Cbz-protected intermediate (250 mg, 0.322 mmol) in MeOH (30 ml) is hydrogenated (50 psi) in the presence of Pd/C (10%) at 50° C. for 1 h. The catalyst is removed by filtration and the filtrate is concentrated in vacuo. The residue is triturated (dichloromethane/ether) to give the product (125 mg, 60%). ESI-MS: (M+H)⁺=643.

### Example 110

### (3S,6S)-1-Biphenyl-4-ylmethyl-6-[(ethyl-piperidin-4-ylmethyl-amino)-methyl]-3-naphthalen-2-ylmethyl-piperazine-2,5-dione

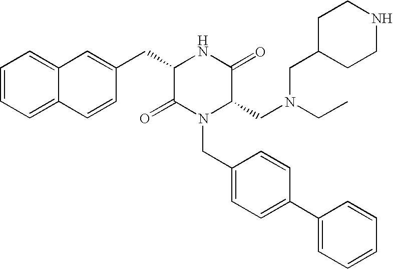 Step 1:

To the corresponding Cbz-protected product from general procedure G, step D (300 mg, 0.44 mmol) and acetaldehyde (100 mg, 2.27 mmol) in THF (20 ml) is added glacial acetic acid (0.06 ml) and the mixture is stirred for 1 h. Sodium triacetoxyborohydride (240 mg, 1.076 mmol) is added and the mixture is stirred for 3 days. Sat. aq. sodium bicarbonate is added, the mixture stirred for 30 min., and then extracted with ether (3×70 ml). The combined org. layers are dried over sodium sulfate, concentrated in vacuo, and the residue purified by flash chromatography (silica, dichloromethane/MeOH 30:1) to give the Cbz-protected intermediate (180 mg, 58%). ESI-MS: (M+H)⁺=709.

Step 2:

The Cbz-protected intermediate (250 mg, 0.322 mmol) in MeOH (30 ml) is hydrogenated (50 psi) in the presence of Pd/C (10%) at 50° C. for 1 h. The catalyst is removed by filtration and the filtrate is concentrated in vacuo. The residue is triturated (dichloromethane ether) to give the product (56 mg, 41%). ESI-MS: (M+H)⁺=575.

### Example 111

### (3S,6S)-1-Biphenyl-4-ylmethyl-3-naphthalen-2-ylmethyl-6-[(piperidin-4-ylmethyl-pyridin-4-ylmethyl-amino)-methyl]-piperazine-2,5-dione

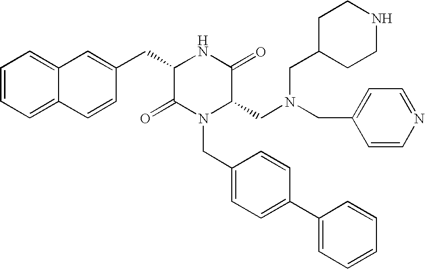 Step 1:

To the corresponding Cbz-protected product from general procedure G, step D (300 mg, 0.44 mmol) and 4-pyridylcarbaldehyde (100 mg, 0.934 mmol) in THF (20 ml) is added glacial acetic acid (0.06 ml) and the mixture is stirred for 1 h. Sodium triacetoxyborohydride (240 mg, 1.076 mmol) is added and the mixture is stirred for 3 days. Another portion of 4-pyridinecarboxaldehyde (100 mg, 0.934 mmol), glacial acetic acid (0.06 ml), and sodium triacetoxyborohydride (240 mg, 1.076 mmol) is added and the mixture is stirred for another 2 days. Sat. aq. sodium bicarbonate is added, the mixture stirred for 30 min., and then extracted with ethyl acetate (3×70 ml). The combined org. layers are dried over sodium sulfate, concentrated in vacuo, and the residue purified by flash chromatography (silica, dichloromethane/MeOH 20:1) to give the Cbz-protected intermediate (260 mg, 76%). ESI-MS: (M+H)⁺=772.

Step 2:

The Cbz-protected intermediate (230 mg, 0.298 mmol) in MeOH (30 ml) is hydrogenated (50 psi) in the presence of Pd/C (10%) at 50° C. for 2 h. The catalyst is removed by filtration and the filtrate is concentrated in vacuo. Flash chromatography (alumina (activity II-III), dichloromethane/MeOH 10:1→5:1) gave the product (85 mg, 45%). ESI-MS: (M+H)⁺=638.

### Example 112

### 3-Amino-N-((2S,5S)-1-biphenyl-4-ylmethyl-5-naphthalen-2-ylmethyl-3,6-dioxo-piperazin-2-ylmethyl)-N-piperidin-4-ylmethyl-propionamide

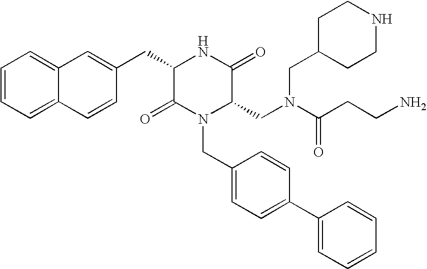 Step 1:

To the corresponding Cbz-protected product from general procedure G, step D (240 mg, 0.353 mmol), 3-N-Cbz-aminopropionic acid (240 mg, 1.075 mmol), HOBt (140 mg, 1.034 mmol), and TBTU (340 mg, 1.06 mmol) in THF (15 ml) is added at room temperature N-ethyldiisopropylamine (0.2 ml, 1.148 mmol) and the mixture is stirred overnight. Sat. aq. sodium bicarbonate (40 ml) is added, the mixture stirred for 30 min., and then extracted with dichloromethane (3×70 ml). The combined org. layers are dried over sodium sulfate, concentrated in vacuo, and the residue purified by flash chromatography (silica, dichloromethane/MeOH 20:1) to give the bis-Cbz-protected intermediate (287 mg, 92%). ESI-MS: (M+H)⁺=886.

Step 2:

The Cbz-protected intermediate (272 mg, 0.308 mmol) in MeOH (40 ml) is hydrogenated (50 psi) in the presence of Pd/C (10%) at 50° C. for 2 h. The catalyst is removed by filtration and the filtrate is concentrated in vacuo. Trituration (dichloromethane/ether) of the residue gave the product (115 mg, 60%). ESI-MS: (M+H)⁺=618.

### Example 113

### 4-{[((2S,5S)-1-Biphenyl-4-ylmethyl-5-naphthalen-2-ylmethyl-3,6-dioxo-piperazin-2-ylmethyl)(piperidine-4-carbonyl)-amino]-methyl}-piperidine-1-carboxylic acid benzyl ester

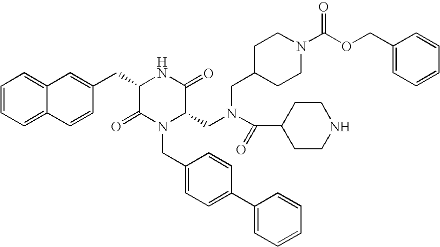 Step 1:

To the corresponding Cbz-protected product from general procedure G, step D (300 mg, 0.441 mmol), N-Boc-piperidin-4-yl carboxylic acid (125 mg, 0.545 mmol), HOBT (70 mg, 0.517 mmol), and TBTU (170 mg, 0.529 mmol) in THF (15 ml) is added at room temperature N-ethyldiisopropylamine (0.1 ml, 0.57 mmol) and the mixture is stirred overnight. Sat. aq. sodium bicarbonate (40 ml) is added, the mixture stirred for 15 min., and then extracted with dichloromethane (3×70 ml). the combined org. layers are dried over sodium sulfate, concentrated in vacuo, and the residue purified by flash chromatography (silica, dichloromethane/MeOH 20:1) to give the bis-protected intermediate (120 mg, 31%). ESI-MS: (M+H)⁺=892.

Step 2:

To the bis-protected intermediate (270 mg, 0.303 mmol) in dichloromethane (5 ml) is added at room temperature TFA (0.5 ml) and the mixture is stirred for 2.5 h. Sat. aq. sodium bicarbonate is added, the mixture is stirred for 15 min., and then extracted with dichloromethane (3×80 ml). The combined org. layers are dried over sodium sulfate, concentrated in vacuo, and the residue purified by flash chromatography (alumina (activity II-III), dichloromethane/MeOH 20:1→10:1) to give the Cbz-protected product (185 mg, 77%). ESI-MS: (M+H)⁺=792.

### Example 114

### 4-{[((2S,5S)-1-Biphenyl-4-ylmethyl-5-naphthalen-2-ylmethyl-3,6-dioxo-piperazin-2-ylmethyl)((RS)-piperidine-3-carbonyl)-amino]-methyl}-piperidine-1-carboxylic acid benzyl ester

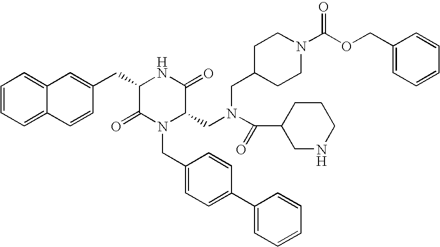 Step 1:

To the corresponding Cbz-protected product from general procedure G, step D (300 mg, 0.441 mmol), N-Boc-piperidin-3-yl carboxylic acid (125 mg, 0.545 mmol), HOBT (70 mg, 0.517 mmol), and TBTU (170 mg, 0.529 mmol) in THF (15 ml) is added at room temperature N-ethyldiisopropylamine (0.1 ml, 0.57 mmol) and the mixture is stirred for 3 days. Sat. aq. sodium bicarbonate (40 ml) is added, the mixture stirred for 15 min., and then extracted with dichloromethane (3×70 ml). The combined org. layers are dried over sodium sulfate, concentrated in vacuo, and the residue purified by flash chromatography (silica, dichloromethane/MeOH 20:1) to give the bis-protected intermediate (184 mg, 47%). ESI-MS: (M+H)⁺=892.

Step 2:

To the bis-protected intermediate (176 mg, 0.197 mmol) in dichloromethane (5 ml) is added at room temperature TFA (0.5 ml) and the mixture is stirred for 3 h. Sat. aq. sodium bicarbonate is added, the mixture is stirred for 15 min., and then extracted with dichloromethane (3×80 ml). The combined org. layers are dried over sodium sulfate and concentrated in vacuo. Trituration (dichloromethane/ether) gave the Cbz-protected product (88 mg, 56%). ESI-MS: (M+H)⁺=792.

### Example 115

### Piperidine-4-carboxylic acid ((2S,5S)-1-biphenyl-4-ylmethyl-5-naphthalen-2-ylmethyl-3,6-dioxo-piperazin-2-ylmethyl)-piperidin-4-ylmethyl-amide

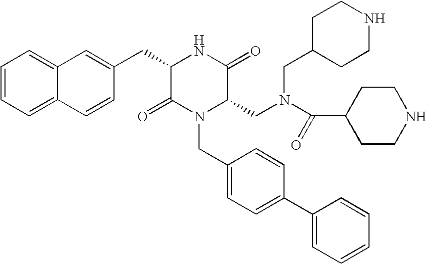

The Cbz-protected precursor from example 113 (170 mg, 0.215 mmol) in MeOH (30 ml) is hydrogenated (50 psi) in the presence of Pd/C (10%) at 50° C. for 1.5 h. The catalyst is removed by filtration and the filtrate concentrated in vacuo. The residue is triturated (dichloromethane/ether) to give the product (110 mg, 78%). ESI-MS: (M+H)⁺=658.

### Example 116

### (RS)-Piperidine-3-carboxylic acid ((2S,5S)-1-biphenyl-4-ylmethyl-5-naphthalen-2-ylmethyl-3,6-dioxo-piperazin-2-ylmethyl)-piperidin-4-ylmethyl-amide

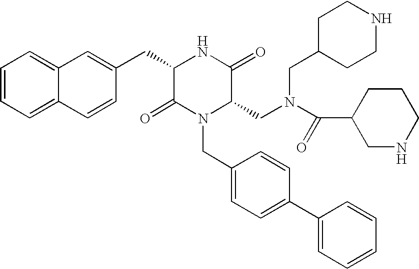

The Cbz-protected precursor from example 114 (77 mg, 0.097 mmol) in MeOH (20 ml) is hydrogenated (50 psi) in the presence of Pd/C (10%) at 50° C. for 1.5 h. The catalyst is removed by filtration and the filtrate concentrated in vacuo. The residue is triturated (dichloromethane/ether) to give the product (44 mg, 69%). ESI-MS: (M+H)⁺=658.

### Example 117

### 4-Amino-N-((2S,5S)-1-biphenyl-4-ylmethyl-5-naphthalen-2-ylmethyl-3,6-dioxo-piperazin-2-ylmethyl)-N-piperidin-4-ylmethyl-butyramide

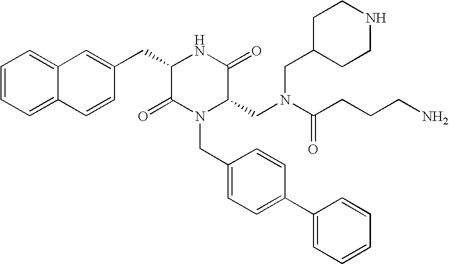 Step 1:

To the corresponding Cbz-protected product from general procedure G, step D (240 mg, 0.353 mmol), 4-N-Cbz-aminobutyric acid (240 mg, 1.012 mmol), HOBt (140 mg, 1.034 mmol), and TBTU (340 mg, 1.034 mmol) in THF (20 ml) is added at room temperature N-ethyldiisopropylamine (0.1 ml, 0.57 mmol) and the mixture is heated to reflux for 6 h. Sat. aq. sodium bicarbonate (40 ml) is added, the mixture stirred for 15 min., and then extracted with dichloromethane (3×70 ml). The combined org. layers are dried over sodium sulfate, concentrated in vacuo, and the residue purified by flash chromatography (silica, dichloromethane/MeOH 20:1) to give the bis-Cbz-protected intermediate (160 mg, 50%). ESI-MS: (M+H)⁺=900.

Step 2:

The bis-Cbz-protected intermediate (160 mg, 0.178 mmol) in MeOH (30 ml) is hydrogenated (50 psi) in the presence of Pd/C (10%) at 50° C. for 1.5 h. The catalyst is removed by filtration and the filtrate concentrated in vacuo. The residue is triturated (dichloromethane ether) to give the product (80 mg, 71%). ESI-MS: (M+H)⁺=632.

### Example 118

### (3S,6S)-6-{[(3-Amino-propyl)-piperidin-4-ylmethyl-amino]-methyl}-1-biphenyl-4-ylmethyl-3-naphthalen-2-ylmethyl-piperazine-2,5-dione

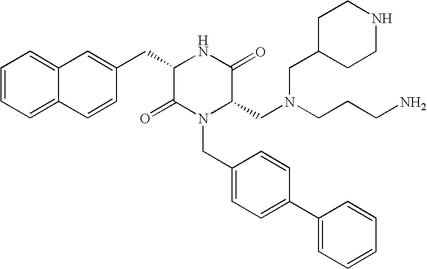 Step 1:

To the corresponding Cbz-protected product from general procedure G, step D (400 mg, 0.588 mmol) and 3-N-Cbz-propionaldehyde (250 mg, 1.146 mmol) in THF (20 ml) is added glacial acetic acid (0.06 ml) and the mixture is stirred for 1 h. Sodium triacetoxyborohydride (300 mg, 1.345 mmol) is added and the mixture is stirred for 3 days. Sat. aq. sodium bicarbonate is added, the mixture stirred for 30 min., and then extracted with ether (3×70 ml). The combined org. layers are dried over sodium sulfate, concentrated in vacuo, and the residue purified by flash chromatography (silica, dichloromethane/MeOH 30:1) to give the bis-Cbz-protected intermediate (450 mg, 88%). ESI-MS: (M+H)⁺=872.

Step 2:

The Cbz-protected intermediate (440 mg, 0.505 mmol) in MeOH (40 ml) is hydrogenated (50 psi) in the presence of Pd/C (10%) at 50° C. for 1.5 h. The catalyst is removed by filtration and the filtrate is concentrated in vacuo. The residue is triturated (dichloromethane/ether) to give the product (154 mg, 51%). ESI-MS: (M+H)⁺=604.

### Example 119

### 1H-Imidazole-4-carboxylic acid [(2S,5S)-5-naphthalen-2-ylmethyl-3,6-dioxo-1-(4-phenoxybenzyl)-piperazin-2-ylmethyl]-piperidin-4-ylmethyl-amide

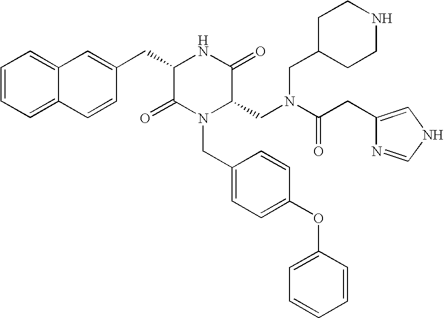 Step 1:

To the corresponding Cbz-protected product from general procedure G, step D (225 mg, 0.323 mmol), 4-imidazole-acetic acid (170 mg, 1.046 mmol), HOBT (140 mg, 1.034 mmol), and TBTU (340 mg, 1.06 mmol) in THF (15 ml) is added at room temperature N-ethyldiisopropylamine (0.4 ml, 2.296 mmol) and the mixture is stirred for 6 h. Sat. aq. sodium bicarbonate (40 ml) is added, the mixture stirred for 30 min., and then extracted with ethyl acetate (3×70 ml). The combined org. layers are dried over sodium sulfate, concentrated in vacuo, and the residue purified by flash chromatography (alumina (activity II-III), dichloromethane/MeOH 20:1→8:1) to give the Cbz-protected intermediate (100 mg, 38%). ESI-MS: (M+H)⁺=805.

Step 2:

The Cbz-protected intermediate (100 mg, 0.124 mmol) in MeOH (20 ml) is hydrogenated (50 psi) in the presence of Pd/C (10%) at 50° C. for 1 h. The catalyst is removed by filtration and the filtrate is concentrated in vacuo. Trituration (dichloromethane/ether) of the residue gave the product (46 mg, 55%). ESI-MS: (M+H)⁺=671.

### Example 120

### 2-Amino-N-[(2S,5S)-5-naphthalen-2-ylmethyl-3,6-dioxo-1-(4-phenoxy-benzyl)-piperazin-2-ylmethyl]-N-piperidin-4-ylmethyl-acetamide

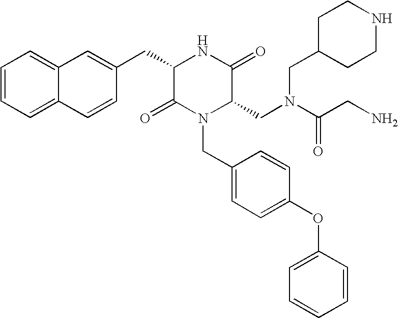 Step 1:

To the corresponding Cbz-protected product from general procedure G, step D (225 mg, 0.323 mmol), N-Cbz-glycine (220 mg, 1.052 mmol), HOBT (140 mg, 1.034 mmol), and TBTU (340 mg, 1.06 mmol) in THF (15 ml) is added at room temperature N-ethyldiisopropylamine (0.2 ml, 1.148 mmol) and the mixture is stirred overnight. Sat. aq. sodium bicarbonate (40 ml) is added, the mixture stirred for 30 min., and then extracted with ether (3×70 ml). The combined org. layers are dried over sodium sulfate, concentrated in vacuo, and the residue purified by flash chromatography (silica, dichloromethane/MeOH 20:1) to give the Cbz-protected intermediate (184 mg, 64%). ESI-MS: (M+H)⁺=888.

Step 2:

The Cbz-protected intermediate (176 mg, 0.0.1.98 mmol) in MeOH (25 ml) is hydrogenated (50 psi) in the presence of Pd/C (10%) at 50° C. for 1.5 h. The catalyst is removed by filtration and the filtrate is concentrated in vacuo. Trituration (dichloromethane/ether) of the residue gave the product (50 mg, 41%). ESI-MS: (M+H)⁺=620.

### Example 121

### 3-Amino-N-[(2S,5S)-5-naphthalen-2-ylmethyl-3,6-dioxo-1-(4-phenoxy-benzyl)-piperazin-2-ylmethyl]-N-piperidin-4-ylmethyl-propionamide

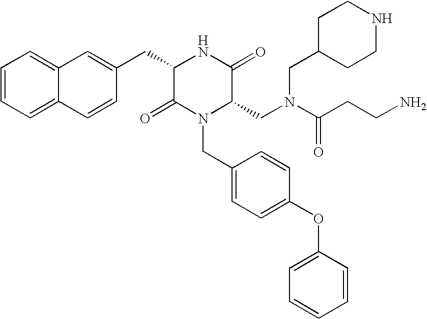 Step 1:

To the corresponding Cbz-protected product from general procedure G, step D (246 mg, 0.353 mmol), 3-N-Cbz-aminopropionic acid (236 mg, 1.06 mmol), HOBT (140 mg, 1.03 mmol), and TBTU (340 mg, 1.06 mmol) in THF (15 ml) is added at room temperature N-ethyldiisopropylamine (0.2 ml, 1.14 mmol) and the mixture is stirred overnight. Sat. aq. sodium bicarbonate (40 ml) is added, the mixture stirred for 30 min., and then extracted with dichloromethane (3×70 ml). The combined org. layers are dried over sodium sulfate, concentrated in vacuo, and the residue purified by flash chromatography (silica, dichloromethane/MeOH 19:1) to give the bis-Cbz-protected intermediate (280 mg, 88%). ESI-MS: (M+H)⁺=902.

Step 2:

The Cbz-protected intermediate (280 mg, 0.31 mmol) in MeOH (20 ml) is hydrogenated (50 psi) in the presence of Pd/C (10%) at 50° C. for 2 h. The catalyst is removed by filtration and the filtrate is concentrated in vacuo to give the product (180 mg, 92%). ESI-MS: (M+H)⁺=634.

### Example 122

### N-[(2S,5S)-5-Naphthalen-2-ylmethyl-3,6-dioxo-1-(4-phenoxy-benzyl)-piperazin-2-ylmethyl]-2-piperidin-4-yl-N-piperidin-4-ylmethyl-acetamide

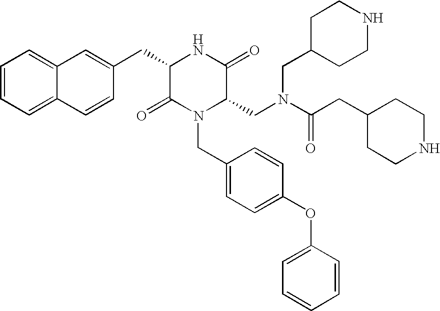 Step 1:

To the corresponding Cbz-protected product from general procedure G, step D (225 mg, 0.323 mmol), N-Boc-piperidin-4-yl-acetic acid (270 mg, 1.054 mmol), HOBT (140 mg, 1.03 mmol), and TBTU (340 mg, 1.06 mmol) in THF (20 ml) is added at room temperature N-ethyldiisopropylamine (0.2 ml, 1.14 mmol) and the mixture is stirred overnight. Sat. aq. sodium bicarbonate (40 ml) is added, the mixture stirred for 30 min., and then extracted with dichloromethane (3×70 ml). The combined org. layers are dried over sodium sulfate, concentrated in vacuo, and the residue purified by flash chromatography (silica, dichloromethane/MeOH 20:1) to give the bis-protected intermediate (162 mg, 54%). ESI-MS: (M+HCOO)⁻=966.

Step 2:

To the bis-protected intermediate (162 mg, 0.176 mmol) in dichloromethane.(5 ml) is added at room temperature TFA (0.36 ml) and the mixture is stirred for 2.5 h. Sat. aq. sodium bicarbonate is added, the mixture is stirred for 15 min., and then extracted with dichloromethane (3×70 ml). The combined org. layers are dried over sodium sulfate and concentrated in vacuo to give the crude Cbz-protected intermediate (135 mg, 93%). ESI-MS: (M+H)⁺=822.

Step 3:

The crude Cbz-protected intermediate (135 mg, 0.164 mmol) in MeOH (25 ml) is hydrogenated (50 psi) in the presence of Pd/C (10%) at 50° C. for 1.5 h. The catalyst is removed by filtration and the filtrate is concentrated in vacuo to give the product (48 mg, 42%). ESI-MS: (M+H)⁺=688.

### Example 123

### (RS)-2,5-Diamino-pentanoic acid [(2S,5S)-5-naphthalen-2-ylmethyl-3,6-dioxo-1-(4-phenoxybenzyl)-piperazin-2-ylmethyl]-piperidin-4-ylmethyl-amide

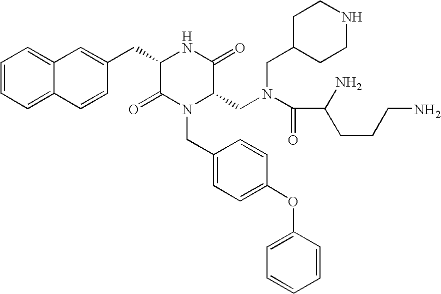 Step 1:

To the corresponding Cbz-protected product from general procedure G, step D (209 mg, 0.300 mmol), N,N′-di-Cbz-DL-ornithine (420 mg, 1.049 mmol), HOBT (140 mg, 1.03 mmol), and TBTU (340 mg, 1.06 mmol) in THF (15 ml) is added at room temperature N-ethyldiisopropylamine (0.2 ml, 1.14 mmol) and the mixture is stirred for 20 h. Sat. aq. sodium bicarbonate (40 ml) is added, the mixture stirred for 30 min., and then extracted with ether (3×70 ml). The combined org. layers are dried over sodium sulfate, concentrated in vacuo, and the residue purified by flash chromatography (silica, dichloromethane/MeOH 20:1) to give the Cbz-protected intermediate (175 mg, 54%). ESI-MS: (M+H)⁺=1078.

Step 2:

The Cbz-protected intermediate (160 mg, 0.148 mmol) in MeOH (25 ml) is hydrogenated (50 psi) in the presence of Pd/C (10%) at 50° C. for 110 min. The catalyst is removed by filtration and the filtrate is concentrated in vacuo to give the product (75 mg, 75%). ESI-MS: (M+H)⁺=677.

### Example 124

### (3S,6S)-6-{[(3-Dimethylamino-propyl)-piperidin-4-ylmethyl-amino]-methyl}-3-naphthalen-2-ylmethyl-1-(4-phenoxy-benzyl)-piperazine-2,5-dione

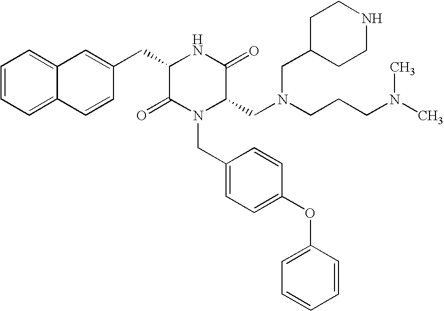 Step 1:

To the corresponding Cbz-protected product from general procedure G, step D (712 mg, 1.023 mmol) and 1,3-dibromopropane (0.5 ml, 4.904 mmol) in DMF (2.5 ml) is added at room temperature N-ethyldiisopropylamine (0.25 ml, 1.435 mmol) and the mixture is heated to 50° C. for 6 h and then stirred at room temperature overnight. The mixture is diluted with water (50 ml) and extracted with ether (3×80 ml). The combined org. layers are dried over sodium sulfate, concentrated in vacuo, and the residue is purified by flash chromatography (silica, dichloromethane/MeOH 20:1) to give the intermediate bromide (495 mg, 59%). ESI-MS: (M+H)⁺=818.

Step 2:

To the intermediate bromide (585 mg, 0.715 mmol) is added dimethylamine (2N in THF, 10 ml, 20 mmol) and the mixture is stirred at room temperature for 20 h. The formed precipitate is removed by filtration and the filtrate is concentrated in vacuo. Flash chromatography (alumina (activity II-III), dichloromethane/MeOH 40:1) afforded the Cbz-protected intermediate (432 mg, 77%). ESI-MS: (M+H)⁺=783.

Step 3:

The Cbz-protected intermediate (432 mg, 0.552 mmol) in MeOH (30 ml) is hydrogenated (50 psi) in the presence of Pd/C (10%) at 50° C. for 1 h. The catalyst is removed by filtration and the filtrate is concentrated in vacuo to give the product (333 mg, 93%). ESI-MS: (M+H)⁺=648.

### Example 125

### 3-Amino-N-(1-methyl-piperidin-4-ylmethyl)-N-[(2S,5S)-5-naphthalen-2-ylmethyl-3,6-dioxo-1-(4-phenoxy-benzyl)-piperazin-2-ylmethyl]-propionamide

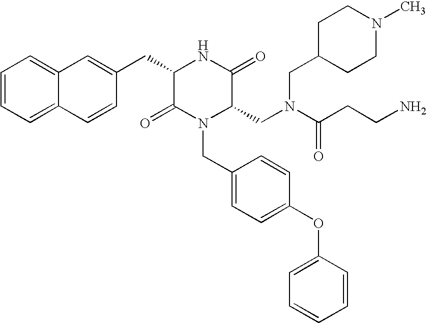 Step 1:

To the corresponding Cbz-protected product from general procedure G, step D (238 mg, 0.341 mmol), 3-N-Boc-propionic acid (200 mg, 1.06 mmol), HOBT (140 mg, 1.03 mmol), and TBTU (340 mg, 1.06 mmol) in THF (15 ml) is added at room temperature N-ethyldiisopropylamine (0.2 ml, 1.14 mmol) and the mixture is stirred overnight. Sat. aq. sodium bicarbonate (40 ml) is added, the mixture stirred for 30 min., and then extracted with ether (3×70 ml). The combined org. layers are dried over sodium sulfate, concentrated in vacuo, and the residue purified by flash chromatography (silica, dichloromethane/MeOH 20:1) to give the bis -protected intermediate (252 mg, 85%). ESI-MS: (M+H)⁺=902.

Step 2:

The bis-protected intermediate (243 mg, 0.28 mmol) in MeOH (25 ml) and formaldehyde (37% in water, 0.5 ml) is hydrogenated (50 psi) in the presence of Pd/C (10%) at 50° C. for 1 h. The catalyst is removed by filtration and the filtrate is concentrated in vacuo Flash chromatography (alumina (activity II-III), dichloromethane/MeOH 30:1) gave the deprotected-alkylated intermediate (171 mg, 70%). ESI-MS: (M+H)⁺=748.

Step 3:

To the Boc-protected intermediate (81 mg, 0.108 mmol) in dichloromethane (5 ml) is added at 0° C. TFA (0.17 ml) and the mixture is stirred for 4 h. Sat. aq. sodium bicarbonate is added, the mixture is stirred for 15 min., and then extracted with dichloromethane (3×70 ml). The combined org. layers are dried over sodium sulfate and concentrated in vacuo Trituration (ether/petroleum ether) gave the product (30 mg, 42%). ESI-MS: (M+H)⁺=648

### Example 126

### Piperidine-3-carboxylic acid [(2S,5S)-5-naphthalen-2-ylmethyl-3,6-dioxo-1-(4-phenoxy-benzyl)piperazin-2-ylmethyl]-piperidin-4-ylmethyl-amide

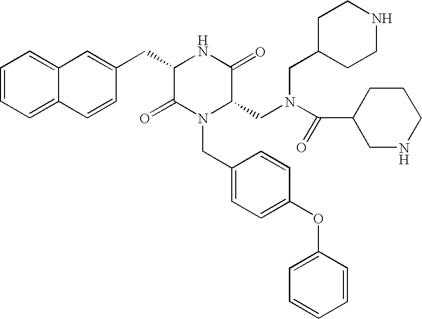 Step 1:

To the corresponding Cbz-protected product from general procedure G, step D (323 mg, 0.441 mmol), N-Cbz-piperidin-3-yl carboxylic acid (370 mg, 1.405 mmol), and TBTU (450 mg, 1.400 mmol) in THF (15 ml) is added at room temperature N-ethyldiisopropylamine (0.3 ml, 1.718 mmol) and the mixture is stirred at room temperature overnight and then heated to reflux for 8 h. Sat. aq. sodium bicarbonate (40 ml) is added, the mixture stirred for 15 min., and then extracted with ether (3×70 ml). The combined org. layers are dried over sodium sulfate, concentrated in vacuo, and the residue purified by flash chromatography (silica, dichloromethane/MeOH 20:1) to give a mixture of the 2 diastereoisomeric bis-Cbz-protected intermediates. Flash chromatography (silica, ethyl acetate/MeOH 100:0→50:1) afforded the 2 diastereoisomers (23% and 16%). ESI-MS: (M+H)⁺=942.

Step 2:

The 2 Cbz-protected diastereoisomers are submitted separately to hydrogenation in MeOH (20 ml) at 50 psi in the presence of Pd/C (10%) at 50° C. for 2 h. The catalyst is removed by filtration and the filtrate is concentrated in vacuo to give the product (41% and 35%). ESI-MS: (M+H)⁺=674.

### Example 127

### (3S,6S)-1-Biphenyl-4-ylmethyl-6-{[bis-(1-methyl-piperidin-4-ylmethyl)-amino]-methyl}-3-naphthalen-2-ylmethyl-piperazine-2,5-dione

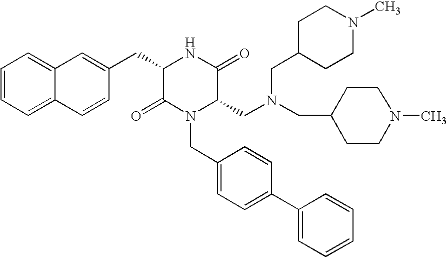

To the corresponding product of general procedure G, step G (171 mg, 0.266 mmol) and formaldehyde (37% in water, 0.1 ml, 1.232 mmol) in THF (10 ml) is added at room temperature sodium triacetoxyborohydride (200 mg, 0.944 mmol) and the mixture is stirred for 3 days. Sat. aq. sodium bicarbonate is added, the mixture is stirred for 15 min., and then extracted with ethyl acetate (3×80 ml). The combined org. layers are dried over sodium sulfate, concentrated in vacuo, and the residue is purified by flash chromatography (alumina (activity II-III), dichloromethane/MeOH 30:1 <20:1) to give the product (54 mg, 30%). ESI-MS: (M+H)⁺=672.

### Example 128

### (3S,6S)-6-{[(3-Amino-propyl)-piperidin-4-ylmethyl-amino]-methyl}-1-(4-phenoxy-benzyl)-3-(4-trifluoromethyl-benzyl)-piperazine-2,5-dione

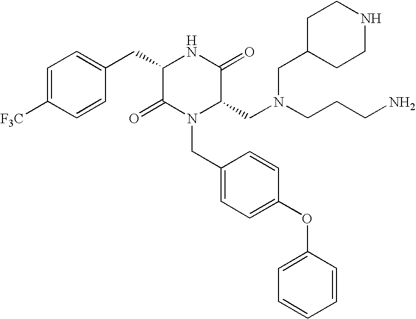 Step 1:

To the corresponding Cbz-protected product from general procedure G, step D (543 mg, 0.760 mmol) and 3-N-Cbz-propionaldehyde (480 mg, 2.320 mmol) in THF (35 ml) is added p-toluenesulfonic acid hydrate (144 mg, 0.760 mmol) and the mixture is stirred for 1 h. Sodium triacetoxyborohydride (600 mg, 2.690 mmol) is added and the mixture is stirred 6 h. Sat. aq. sodium bicarbonate is added, the mixture stirred for 30 min., and then extracted with ethyl acetate (3×70 ml). The combined org. layers are dried over sodium sulfate, concentrated in vacuo, and the residue purified by flash chromatography (silica, dichloromethane/MeOH 33:1) to give the bis-Cbz-protected intermediate (690 mg, quant. yield). ESI-MS: (M+H)⁺=906.

Step 2:

The bis-Cbz-protected intermediate (690 mg, 0.760 mmol) in MeOH (30 ml) is hydrogenated (50 psi) in the presence of Pd/C (10%) at 50° C. for 4 h. The catalyst is removed by filtration and the filtrate is concentrated in vacuo. The residue purified by HPLC (ZorbaxSB-C18 (5 μm) column, gradient of water/MeCN +0.1% formic acid, detection at 254 nm and 230 nm) to give the product (470 mg, 97%). ESI-MS: (M+H)⁺=638.

### Example 129

### (3S,6S)-6-{[(3-Hydroxy-propyl)-piperidin-4-ylmethyl-amino]-methyl}-1-(4-phenoxy-benzyl)-3-(4-trifluoromethyl-benzyl)-piperazine-2,5-dione

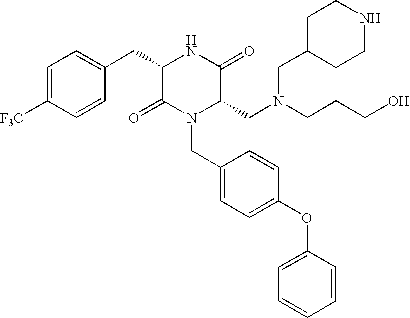 Step 1:

To the corresponding Cbz-protected product from general procedure G, step D (225 mg, 0.315 mmol) and 3-bromo-propanol (0.1 ml, 1.073 mmol) in DMF (2 ml) is added at room temperature N-ethyldiisopropylamine (0.2 ml, 1.148 mmol) and the mixture is heated to 120° C. for 3 h. 3-bromo-propanol (0.1 ml, 1.073 mmol) is added and the mixture heated to 120° C. for another 3 h. Water (50 ml) is added and the aq. layer is extracted with ether (3×70 ml). The combined org. layers are dried over sodium sulfate, concentrated in vacuo, and the residue is purified by flash chromatography (alumina (activity II-III), dichloromethane/MeOH 30:1→20:1) to give the Cbz-protected intermediate (70 mg, 29%). ESI-MS: (M+H)⁺=773.

Step 2:

The Cbz-protected intermediate (70 mg, 0.091 mmol) in MeOH (25 ml) is hydrogenated (50 psi) in the presence of Pd/C (10%) at 50° C. for 1.5 h. The catalyst is removed by filtration and the filtrate is concentrated in vacuo. The triturated (dichloromethane/ether) to give the product (29 mg, 50%). ESI-MS: (M+H)⁺=639.

### Example 130

### 3-Amino-N-[(2S,5S)-3,6-dioxo-1-(4-phenoxy-benzyl)-5-(4-trifluoromethyl-benzyl)-piperazin-2-ylmethyl]-N-piperidin-4-ylmethyl-propionamide

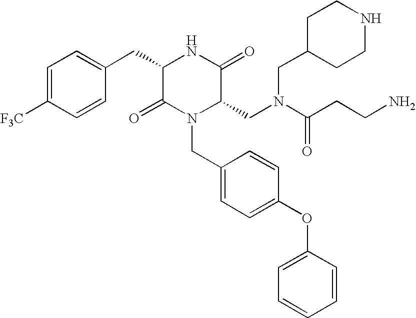 Step 1:

To the corresponding Cbz-protected product from general procedure G, step D (460 mg, 0.644 mmol), 3-N-Cbz-aminopropionic acid (425 mg, 1.904 mmol), HOBT (283 mg, 1.841 mmol), and TBTU (614 mg, 1.891 mmol) in THF (30 ml) is added at room temperature N-ethyldiisopropylamine (0.47 ml, 2.689 mmol) and the mixture is stirred for 4 days. Sat. aq. sodium bicarbonate (40 ml) is added, the mixture stirred for 30 min., and then extracted with dichloromethane (3×70 ml). The combined org. layers are dried over sodium sulfate, concentrated in vacuo, and the residue purified by flash chromatography (silica, dichloromethane/MeOH 49:1→19:1) to give the bis-Cbz-protected intermediate (400 mg, 67%). ESI-MS: (M+H)⁺=920.

Step 2:

The Cbz-protected intermediate (266 mg, 0.0.289 mmol) in MeOH (40 ml) is hydrogenated (50 psi) in the presence of Pd/C (10%) at 50° C. for 75 min. The catalyst is removed by filtration and the filtrate is concentrated in vacuo. Trituration (dichloromethane/ether) of the residue gave the product (120 mg, 64%). ESI-MS: (M+H)⁺=652.

### Example 131

### N-((2S,5S)-1-Biphenyl-4-ylmethy-5-naphthalen-2-ylmethyl-3,6-dioxo-piperazin-2-ylmethyl)-2-(RS)-morpholin-2-yl-N-piperidin-4-ylmethyl-acetamide

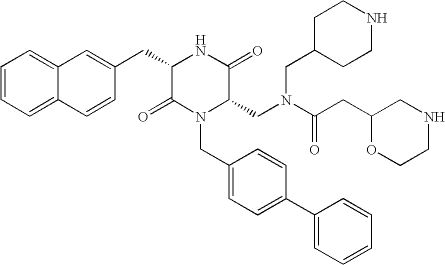 Step 1:

To the corresponding Cbz-protected product from general procedure G, step D (237 mg, 0.349 mmol), N-Cbz-2carboxymorpholine (300 mg, 1.074 mmol), HOBT (130 mg, 0.960 mmol), and TBTU (320 mg, 0.997 mmol) in THF (15 ml) is added at room temperature N-ethyldiisopropylamine (0.2 ml, 1.139 mmol) and the mixture is stirred for 20 h. Sat. aq. sodium bicarbonate (40 ml) is added, the mixture stirred for 30 min., and then extracted with ether (3×70 ml). The combined org. layers are dried over sodium sulfate, concentrated in vacuo, and the residue purified by flash chromatography (silica, dichloromethane/MeOH 30:1→20:1) to give the bis-Cbz-protected intermediate (198 mg, 60%). ESI-MS: (M+H)⁺=942.

Step 2:

The Cbz-protected intermediate (189 mg, 0.201 mmol) in MeOH (30 ml) is hydrogenated (50 psi) in the presence of Pd/C (10%) at 50° C. for 1.5 h. The catalyst is removed by filtration and the filtrate is concentrated in vacuo. Trituration (dichloromethane/ether) of the residue gave the product (126 mg, 93%). ESI-MS: (M+H)⁺=674.

### Example 132

### (3S,6S)-1-Biphenyl-4-ylmethyl-3-naphthalen-2-ylmethyl-6-[(piperidin-4-ylmethyl-pyridin-3-ylmethyl-amino)-methyl]-piperazine-2,5-dione

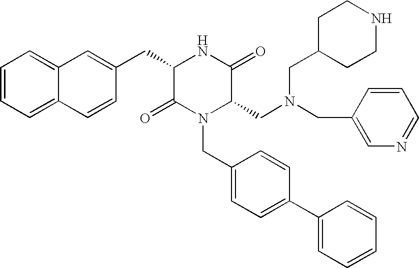 Step 1:

To the corresponding Cbz-protected product from general procedure G, step D (238 mg, 0.35 mmol) and 3-pyridylcarbaldehyde (120 mg, 1.12 mmol) in THF (15 ml) is added sodium triacetoxyborohydride (250 mg, 1.121 mmol) is added and the mixture is stirred for 20 h. Sat. aq. sodium bicarbonate is added, the mixture stirred for 30 min., and then extracted with ethyl acetate (3×70 ml). The combined org. layers are dried over sodium sulfate, concentrated in vacuo, and the residue purified by flash chromatography (silica, dichloromethane/MeOH 20:1 <15:1) to give the Cbz-protected intermediate (257 mg, 95%). ESI-MS: (M+H)⁺=772.

Step 2:

The Cbz-protected intermediate (260 mg, 0.32 mmol) in MeOH (30 ml) is hydrogenated (50 psi) in the presence of Pd/C (10%) at 50° C. for 2 h. The catalyst is removed by filtration and the filtrate is concentrated in vacuo to give the product (180 mg, 75%). ESI-MS: (M+H)⁺=638.

### Example 133

### (3S,6S)-1-(4-Phenoxy-benzyl)-6-[(piperidin-4-ylmethyl-pyridin-3-ylmethyl-amino)-methyl]-3-(4-trifluoromethyl-benzyl)-piperazine-2,5-dione

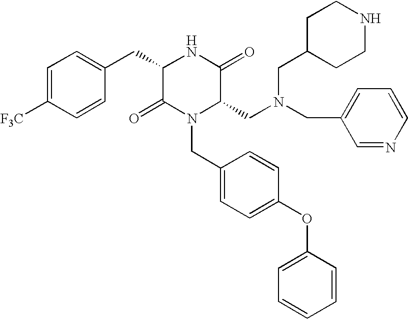 Step 1:

To the corresponding Cbz-protected product from general procedure G, step D (250 mg, 0.25 mmol) and 3-pyridylcarbaldehyde (120 mg, 1.12 mmol) in THF (15 ml) is added sodium triacetoxyborohydride (250 mg, 1.12 mmol) is added and the mixture is stirred overnight. Sat. aq. sodium bicarbonate is added, the mixture stirred for 30 min., and then extracted with ethyl acetate (3×70 ml). The combined org. layers are dried over sodium sulfate, concentrated in vacuo, and the residue purified by flash chromatography (silica, dichloromethane/MeOH 30:1) to give the Cbz-protected intermediate (230 mg, 81%). ESI-MS: (M+H)⁺=806.

Step 2:

The Cbz-protected intermediate (220 mg, 0.273 mmol) in MeOH (50 ml) is hydrogenated (50 psi) in the presence of Pd/C (10%) at 50° C. for 1 h. The catalyst is removed by filtration and the filtrate is concentrated in vacuo. Trituration (dichloromethane/ether) gave the product (173 mg, 94%). ESI-MS: (M+H)⁺=672.

### Example 134

### N-((2S,5S)-1-Biphenyl-4-ylmethyl-5-naphthalen-2-ylmethyl-3,6-dioxo-piperazin-2-ylmethyl)-2-cyclopropylamino-N-piperidin-4-ylmethyl-acetamide

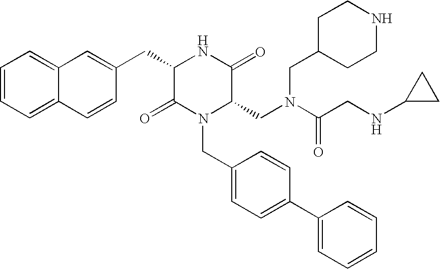 Step 1:

To the corresponding Cbz-protected product from general procedure G, step D (570 mg, 0.837 mmol) and bromoacetyl bromide (0.075 ml, 0.862 mmol) in dichloromethane (20 ml) is added at 0° C. N-ethyldiisopropylamine (0.2 ml, 1.148 mmol) and the mixture is stirred at 0° C. for 30 min. Sat. aq. sodium bicarbonate is added, the mixture is stirred for 15 min., and then extracted with dichloromethane (3×80 ml). The combined org. layers are dried over sodium sulfate, concentrated in vacuo, and the residue is purified by trituration (dichloromethane/ether) to afford the intermediate bromide (580 mg, 86%). ESI-MS: (M+H)⁺=801.

Step 2:

The intermediate bromide (285 mg, 0.355 mmol) and cyclopropylamine (150 mg, 2.627 mmol) in THF (5 ml) is stirred at room temperature for 3 days. The mixture is concentrated in vacuo and the residue purified by flash chromatography (alumina (activity II-III), dichloromethane/MeOH 40:1→20:1) to give the Cbz-protected intermediate (260 mg, 94%). ESI-MS: (M+H)⁺=778.

Step 3:

The Cbz-protected intermediate (250 mg, 0.321 mmol) in MeOH (30 ml) is hydrogenated (50 psi) in the presence of Pd/C (10%) at 50° C. for 2 h. The catalyst is removed by filtration and the filtrate is concentrated in vacuo to give the product (1350 mg, 65%). ESI-MS: (M+H)⁺=644.

### Example 135

### N-((2S,5S)-1-Biphenyl-4-ylmethyl-5-naphthalen-2-ylmethyl-3,6-dioxo-piperazin-2-ylmethyl)-N-piperidin-4-ylmethyl-2-(2,2,2-trifluoro-ethylamino)-acetamide

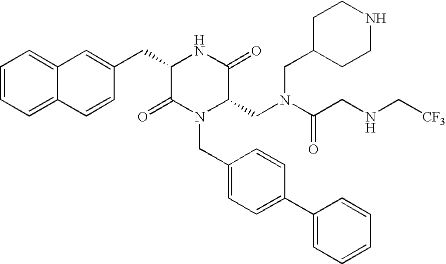 Step 1:

To the corresponding Cbz-protected product from general procedure G, step D (570 mg, 0.837 mmol) and bromoacetyl bromide (0.075 ml, 0.862 mmol) in dichloromethane (20 ml) is added at 0° C. N-ethyldiisopropylamine (0.2 ml, 1.148 mmol) and the mixture is stirred at 0° C. for 30 min. Sat. aq. sodium bicarbonate is added, the mixture is stirred for 15 min., and then extracted with dichloromethane (3×80 ml). The combined org. layers are dried over sodium sulfate, concentrated in vacuo, and the residue is purified by trituration (dichloromethane/ether) to afford the intermediate bromide (580 mg, 86%). ESI-MS: (M+H)⁺=801.

Step 2:

The intermediate bromide (285 mg, 0.355 mmol) and 2,2,2-trifluoroethylamine (300 mg, 3.028 mmol) in THF (5 ml) is stirred at room temperature for 3 days. The mixture is concentrated in vacuo and the residue purified by flash chromatography (silica, dichloromethane/MeOH 20:1→15:1) to give the Cbz-protected intermediate (290 mg, 99%). ESI-MS: (M+H)⁺=820.

Step 3:

The Cbz-protected intermediate (280 mg, 0.341 mmol) in MeOH (30 ml) is hydrogenated (50 psi) in the presence of Pd/C (10%) at 50° C. for 2 h. The catalyst is removed by filtration and the filtrate is concentrated in vacuo to give the product (1710 mg, 73%). ESI-MS: (M+H)⁺=686.

### Example 136

### N-((2S,5S)-1-Biphenyl-4-ylmethyl-5-naphthalen-2-ylmethyl-3,6-dioxo-piperazin-2-ylmethyl)-2-imidazol-1-yl-N-piperidin-4-ylmethyl-acetamide

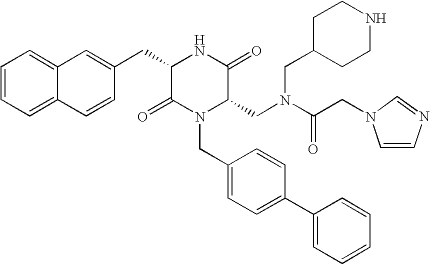 Step 1:

To the corresponding Cbz-protected product from general procedure G, step D (570 mg, 0.837 mmol) and bromoacetyl bromide (0.075 ml, 0.862 mmol) in dichloromethane (20 ml) is added at 0° C. N-ethyldiisopropylamine (0.2 ml, 1.148 mmol) and the mixture is stirred at 0° C. for 30 min. Sat. aq. sodium bicarbonate is added, the mixture is stirred for 15 min., and then extracted with dichloromethane (3×80 ml). The combined org. layers are dried over sodium sulfate, concentrated in vacuo, and the residue is purified by trituration (dichloromethane/ether) to afford the intermediate bromide (580 mg, 86%). ESI-MS: (M+H)⁺=801.

Step 2:

The intermediate bromide (306 mg, 0.382 mmol) and imidazole (100 mg, 1.469 mmol) in THF (10 ml) is heated to reflux for 3 h. Water (50 ml) is added and the aq. layer is extracted with ethyl acetate (3×70 ml). The combined org. layers are dried over sodium sulfate, concentrated in vacuo, and the residue purified by flash chromatography (alumina (activity II-III), dichloromethane/MeOH 30:1→20:1) to give the Cbz-protected intermediate (180 mg, 60%). ESI-MS: (M+H)⁺=789.

Step 3:

The Cbz-protected intermediate (181 mg, 0.217 mmol) in MeOH (30 ml) is hydrogenated (50 psi) in the presence of Pd/C (10%) at 50° C. for 1 h. The catalyst is removed by filtration, the filtrate is concentrated in vacuo, and the residue is triturated (dichloromethane/ether) to give the product (123 mg, 87%). ESI-MS: (M+H)⁺=655.

### Example 137

### 2-[((2S,5S)-1-Biphenyl-4-ylmethyl-5-naphthalen-2-ylmethyl-3,6-dioxo-piperazin-2-ylmethyl)piperidin-4-ylmethyl-amino]-acetamide

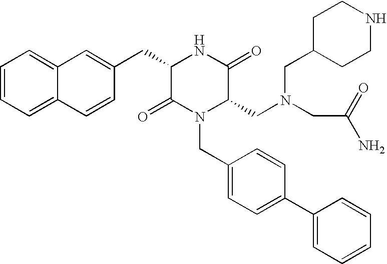 Step 1:

To the corresponding Cbz-protected product of general procedure G, step D (680 mg, 1.0 mmol) and 2-bromoactamid (155 mg, 1.1 mmol) in DMF (10 ml) is added sodium bicarbonate (233 mg, 2.2 mmol) and the mixture is stirred at 80° C. for 5 h. The reaction mixture is poured into ice water (200 ml), the white precipitate is collected by filtration and washed with water to give the intermediate (730 mg, 99%). ESI-MS: (M+H)⁺=738.

Step 2:

The Cbz-protected intermediate (730 mg, 0.989 mmol) in MeOH (50 ml) is hydrogenated (50 psi) in the presence of Pd/C (10%) at 50° C. for 25 min. The catalyst is removed by filtration. The filtrate is concentrated in vacuo and washed with ether to give the product (400 mg, 67%). ESI-MS: (M+H)⁺=604.

### Example 138

### N-((2S,5S)-1-Biphenyl-4-ylmethyl-5-naphthalen-2-ylmethyl-3,6-dioxo-piperazin-2-ylmethyl)-N-piperidin-4-ylmethyl-2-pyridin-3-yl-acetamide

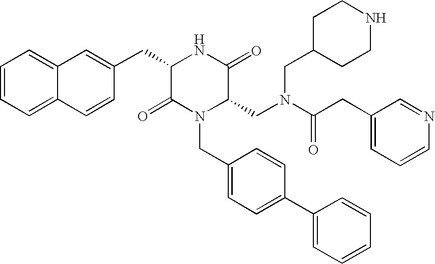 Step 1:

To the corresponding Cbz-protected product from general procedure G, step D (238 mg, 0.349 mmol), 3-pyridylacetic acid (150 mg, 1.094 mmol), HOBT (130 mg, 0.960 mmol), and TBTU (320 mg, 0.997 mmol) in THF (15 ml) is added at room temperature N-ethyldiisopropylamine (0.25 ml, 1.424 mmol) and the mixture is heated to reflux for 5 h. Sat. aq. sodium bicarbonate (40 ml) is added, the mixture stirred for 10 min., and then extracted with ethyl acetate (3×70 ml). The combined org. layers are dried over sodium sulfate, concentrated in vacuo, and the residue purified by flash chromatography (silica, dichloromethane/MeOH 20:1→10:1) to give the Cbz-protected intermediate (257 mg, 92%). ESI-MS: (M+H)⁺=800.

Step 2:

The Cbz-protected intermediate (247 mg, 0.309 mmol) in MeOH (30 ml) is hydrogenated (50 psi) in the presence of Pd/C (10%) at 50° C. for 1.5 h. The catalyst is removed by filtration and the filtrate is concentrated in vacuo. Trituration (dichloromethane/ether) of the residue gave the product (158 mg, 77%). ESI-MS: (M+H)⁺=666.

### Example 139

### N-((2S,5S)-1-Biphenyl-4-ylmethyl-5-naphthalen-2-ylmethyl-3,6-dioxo-piperazin-2-ylmethyl)-N-piperidin-4-ylmethyl-nicotinamide

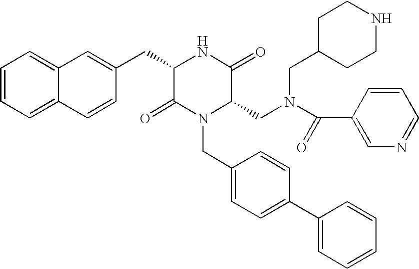 Step 1:

To the corresponding Cbz-protected product from general procedure G, step D (238 mg, 0.349 mmol) and nicotinic acid chloride (100 mg, 0.545 mmol) in dichloromethane (10 ml) is added at 0° C. N-ethyldiisopropylamine (0.25 ml, 1.424 mmol) and the mixture is stirred at 0° C. for 1 h. Sat. aq. sodium bicarbonate (40 ml) is added, the mixture stirred for 10 min., and then extracted with dichloromethane (3×70 ml). The combined org. layers are dried over sodium sulfate, concentrated in vacuo, and the residue purified by flash chromatography (silica, dichloromethane/MeOH 20:1→15:1) to give the Cbz-protected intermediate (257 mg, 93%). ESI-MS: (M+H)⁺=786.

Step 2:

The Cbz-protected intermediate (247 mg, 0.314 mmol) in MeOH (30 ml) is hydrogenated (50 psi) in the presence of Pd/C (10%) at 50° C. for 1.5 h. The catalyst is removed by filtration and the filtrate is concentrated in vacuo. Trituration (dichloromethane/ether) of the residue gave the product (150 mg, 73%). ESI-MS: (M+H)⁺=652.

### Example 140

### N-((2S,5S)-1-Biphenyl-4-ylmethyl-5-naphthalen-2-ylmethyl-3,6-dioxo-piperazin-2-ylmethyl)-N-piperidin-4-ylmethyl-2-pyrrolidin-1-yl-acetamide

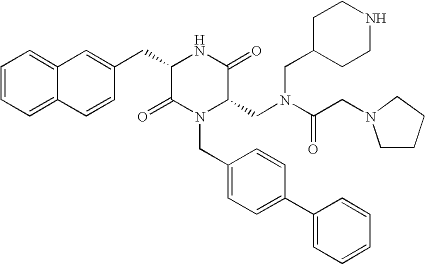 Step 1:

To the corresponding Cbz-protected product from general procedure G, step D (570 mg, 0.837 mmol) and bromoacetyl bromide (0.075 ml, 0.862 mmol) in dichloromethane (20 ml) is added at 0° C. N-ethyldiisopropylamine (0.2 ml, 1.148 mmol) and the mixture is stirred at 0° C. for 30 min. Sat. aq. sodium bicarbonate is added, the mixture is stirred for 15 min., and then extracted with dichloromethane (3×80 ml). The combined org. layers are dried over sodium sulfate, concentrated in vacuo, and the residue is purified by trituration (dichloromethane/ether) to afford the intermediate bromide (580 mg, 86%). ESI-MS: (M+H)⁺=801.

Step 2:

The intermediate bromide (323 mg, 0.382 mmol) and pyrrolidine (100 mg, 1.406 mmol) in THF (10 ml) is stirred at room temperature for 1 h. Sat. aq. sodium bicarbonate is added and the aq. layer is extracted with ethyl acetate (3×80 ml). The combined org. layers are dried over sodium sulfate and the residue is purified by flash chromatography (alumina (activity II-III), dichloromethane/MeOH 10:1) to give the Cbz-protected intermediate (200 mg, 66%). ESI-MS: (M+H)⁺=792.

Step 3:

The Cbz-protected intermediate (190 mg, 0.240 mmol) in MeOH (30 ml) is hydrogenated (50 psi) in the presence of Pd/C (10%) at 50° C. for 1 h. The catalyst is removed by filtration and the filtrate is concentrated in vacuo to give the product (160 mg, quant.). ES-MS: (M+H)⁺=658.

### Example 141

### 3-Amino-N-((2S,5S)-1-biphenyl-4-ylmethyl-5-naphthalen-2-ylmethyl-3,6-dioxo-piperazin-2-ylmethyl)-N-pyridin-3-ylmethyl-propionamide

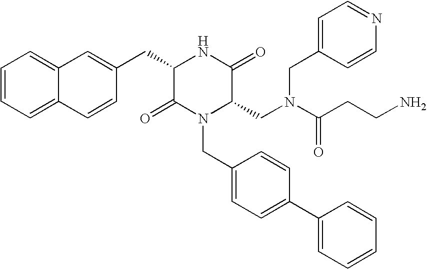 Step 1:

The corresponding product of general procedure E, step E (1.0 g, 2.224 mmol), 3-pyridylcarbaldehyde (300 mg, 2.801 mmol), and a trace of p-toluenesulfonic acid is heated to reflux for 2 h. The precipitate is collected by filtration and dried under high vacuum (1.080 g, 90%).

Step 2:

The Schiff's base (270 mg, 0.501 mmol) in MeOH (30 ml) is hydrogenated (50 psi) in the presence of Raney nickel (130 mg) at 50° C. for 2 h. The catalyst is removed by filtration and the filtrate is concentrated in vacuo. Trituration (dichloromethane/ether) afforded the intermediate (216 mg, 80%). ESI-MS: (M+H)⁺=541.

Step 3:

To the intermediate from the previous step (220 mg, 0.407 mmol), 3-N-Cbz-propionic acid (280 mg, 1.254 mmol), HOBT (160 mg, 1.182 mmol), and TBTU (370 mg, 1.152 mmol) in THF (15 ml) is added at room temperature N-ethyldiisopropylamine (0.3 ml, 1.709 mmol) and the mixture is stirred for 1 day. Sat. aq. sodium bicarbonate (40 ml) is added, the mixture stirred for 10 min., and then extracted with ether (3×70 ml). The combined org. layers are dried over sodium sulfate, concentrated in vacuo, and the residue purified by flash chromatography (alumina (activity II-III), dichloromethane/MeOH 30:1) to give the Cbz-protected intermediate (115 mg, 38%). ESI-MS: (M+H)⁺=746.

Step 4: The Cbz-protected intermediate (110 mg, 0.140 mmol) in MeOH (30 ml) is hydrogenated (50 psi) in the presence of Pd/C (10%) at 50° C. for 1.5 h. The catalyst is removed by filtration and the filtrate is concentrated in vacuo. Trituration (dichloromethane/ether) of the residue gave the product (75 mg, 88%). ESI-MS: (M+H)⁺=612.

### Examples 142 to 148

Active compounds prepared according to the general procedure C of general formula (Ig) are shown in Table IX:

TABLE IX

| | |
|---|---|
| | Formula (Ig) |
| 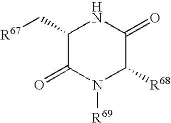 | |

| Example | | | | |
|---|---|---|---|---|
| No | R⁶⁷ | R⁶⁸ | R⁶⁹ | ESI-MS |
| 142 | 2-naphthyl | 4-amino-butyl | biphenyl-4-ylmethyl | (M + H)⁺ = 492 |
| 143 | 2-naphthyl | 4-amino-butyl | 4-phenoxy-benzyl | (M + H)⁺ = 508 |
| 144 | 3,4-Cl₂—C₆H₃ | 4-amino-butyl | 4-phenoxy-benzyl | (M + H)⁺ = 526 |
| 145 | 2-naphthyl | 4-amino-butyl | 9H-fluoren-2-ylmethyl | (M + H)⁺ = 504 |
| 146 | 1-naphthyl | 4-amino-butyl | 4-phenoxy-benzyl | (M + H)⁺ = 508 |
| 147 | 1-naphthyl | 4-amino-butyl | 9H-fluoren-2-ylmethyl | (M + H)⁺ = 504 |
| 148 | 1-naphthyl | 4-amino-butyl | 4-benzyloxy-benzyl | (M + H)⁺ = 522 |

Biological Methods
Melanocortin Receptor 1 (MC1) Binding Assay

The MC1 receptor binding assay is performed on HEK293 cell membranes stably expressing the MC1 receptor. The assay is performed in a total volume of 250 μl; 25 μl ¹²⁵NDP-α-MSH (≈33 pM in final concentration) 25 μl test compound/control and 200 μl cell membrane (35 μg/ml). The samples are incubated at 30° C. for 90 min in the Greiner microtitter plates and separated on GF/B filters that are pre-wetted for 60 min in 0.5% PEI, and washed 2-3 times with NaCl (0.9%) before separation of bound from unbound radio ligand by filtration. After filtration the filters are washed with ice-cold 0.9% NaCl 10 times. The filters are dried at 50° C. for 30 min, sealed and 30 μl Microscint 0 (Packard, cat no. 6013616) are added to each well and the plates are counted in a Topcounter 1 min/well.

The data are analysed by a non-linear regression analysis of binding curves, using a windows program GraphPad Prism, GraphPad software, USA.

Melanocortin Receptor 1, 3 and 5 (MC1, MC3 and MC5) cAMP Functional Assay

The cAMP assays for MC1, MC3 and MC5 receptors are performed on cells stably expressing the MC1, MC3 and MC5 receptors respectively. The receptors were cloned from cDNA by PCR and inserted into the pcDNA 3 expression vector. Stable clones were selected using 1 mg /ml G418.

Cells at app. 80-90% confluence are washed 3× with PBS, lifted from the plates with Versene and diluted in PBS. Centrifuged 2 min at 1300 rpm, and the supernatant removed. The cells are washed twice with stimulation buffer, and resuspended in stimulation buffer to a final concentration of 1×10⁶ cells/ml. (Use 7 ml/96 well plate). 50 μl cell suspension is added to the FlashPlate containing 50 μl of test-compound or reference compound (all diluted in H₂O). The plates are incubated for 30 minutes at room temperature (RT) on a plate-shaker that shakes at low rate. The reaction is stopped with 100 μl Detection Mixpro well (Detection Mix=11 ml Detection Buffer+100 μl (˜2 μCi) cAMP [¹²⁵I] Tracer). The plates are then sealed with plastic, shaken for 30 minutes, and allowed to stand overnight (or for 2 hours), and counted in the Topcounter 1 min/well. In general the assay procedure described in the kit-protocol (Flash Plate® cAMP assay (NEN™ Life Science Products cat no SMP004)) is followed, however the cAMP standards are diluted in H₂O and not in stimulation buffer.

Melanocortin Receptor 4 (MC4) Binding Assay

In Vitro ¹²⁵NDP-α-MSH Binding to Recombinant SF9 Cells Expressing Human MC4 Receptor (Filtration Assay).

The assay is performed in 5 ml minisorb vials, (Sarstedt No. 55.526) or in 96 well filterplate, Millipore MADVN 6550 and using SF9 cells expressing the human MC4 receptor (obtained from Professer Wikberg, Uppsala, Sweden). The SF9 cells are kept at −80° C. until assay, and the assays is run directly on a dilution of this cell suspension, without further preparation. The suspension is diluted to give maximal 10% specific binding, app 50-100 fold dilution. The assay is performed in a total volume of 200 μl; 50 μl cell suspension, 50 μl ¹²⁵NDP-α-MSH (≈79 pM in final concentration), 50 μl test-peptide and 50 μl binding buffer pH 7 is mixed and incubated for 2 h at 25° C. (Binding buffer; 25 mM HEPES pH 7.0, 1 mM CaCl₂, 1 mM MgSO₄, 1 mM EGTA, 0.02% Bacitracin and 0.2% BSA). Peptides are dissolved in H₂O and diluted in binding buffer. Radioligand and membranes are diluted in binding buffer. The incubation is stopped by dilution with 5 ml ice-cold 0.9% NaCl, followed by rapid filtration through Whatman GF/C filters pre-treated for 1 hour with 0.5% polyethyleneimine. The filters are washed with 3×5 ml ice-cold NaCl. The radioactivity retained on the filters is counted using a Cobra II auto gamma counter.

The data are analysed by a non-linear regression analysis of binding curves, using a windows program GraphPad Prism, GraphPad software, USA.

Melanocortin Receptor 4 (MC4) cAMP Assay

BHK cells expressing the MC4 receptor are stimulated with potential MC4 agonists, and the degree of stimulation of cAMP is measured using the Flash Plate® cAMP assay (NEN™ Life Science Products cat no SMP004).

The MC4 receptor expressing BHK cells were made by transfecting the cDNA encoding MC4 receptor into BHK570/KZ10-20-48, and selecting for stable clones expressing the MC4 receptor. The MC4 receptor cDNA is bought from Euroscreen in addition to a CHO cell line expressing the MC4 receptor. The cells are grown in DMEM, 10% FCS, 1 mg/ml G418, 250 nM MTX and 1% penicillin/streptomycin.

Cells at app. 80-90% confluence are washed 3× with PBS, lifted from the plates with Versene and diluted in PBS. Centrifuged 2 min at 1300 rpm, and the supernatant removed. The cells are washed twice with stimulation buffer, and resuspended in stimulation buffer to a final concentration of 0.75×10⁶ cells/ml. (Use 7 ml/96 well plate). 50 μl cell suspension is added to the Flashplate containing 50 μl of test-compound or reference compound (all diluted in H₂O). The mixture is shaken for 5 minutes, and allowed to stand for 25 minutes at RT. The reaction is stopped with 100 μl Detection Mixpro well (Detection Mix=11 ml Detection Buffer+100 μl (˜2 μCi) cAMP [¹²⁵I] Tracer). The plates are then sealed with plastic, shaken for 30 minutes, and allowed to stand overnight (or for 2 hours), and counted in the Topcounter 2 min/well. In general the assay procedure described in the kit-protocol (Flash Plate® cAMP assay (NEN™ Life Science Products cat no SMP004)) is followed, however the cAMP standards are diluted in H₂O and not in stimulation buffer.

EC₅₀ values is calculated by non-linear regression analysis of dose response curves (6 points minimum) using the windows program GraphPad Prism, GraphPad software, USA. All results are expressed in nM.

Pharmacological Methods

Assay (I) Experimental Protocol for Efficacy Testing on Appetite with MC4 Analogues, Using a Schedule—Fed Rat Model.

TAC:SPRD @mol rats or Wistar rats from M&B Breeding and Research Centre A/S, Denmark are used for the experiments. The rats have a bodyweight 200-250 g at the start of experiment. The rats arrive at least 10-14 days before start of experiment with a bodyweight of 180-200 g. Each dose of compound is tested in a group of 8 rats. A vehicle group of 8 rats is included in each set of testing.

When the animals arrive they are housed individually. After a habituating period of 4-7 days with free access to food and water, the schedule feeding is initiated. The rats are allowed to eat from 08 am to 01 pm each day. In the remaining period only access to water is allowed. They are kept on this feeding schedule for 8 days before start of experiment. In this period the animals are handled and dosed in the relevant way (ip, po, sc.) with saline at least 3 times. The experiment is conducted in the rat home cages. Immediately before dosing the rats are randomised to the different treatment groups (n=8) by bodyweight. They are dosed according to bodyweight at 08 am, with a 1 ml/kg solution either, ip, po or sc. The dosing time is recorded for each group. Following dosing the rats are returned to their home cages, where they now have access to food and water. The food consumption is recorded individually, each hour for 3 hours. At the end of the experimental session, the animals are euthanised.

The individual data are recorded in Microsoft excel sheets. Outliers are excluded after using the Grubbs statistical evaluation test for outliers and the result presented graphically by using the GraphPad Prism program.

Drug:

Vehicle 1 ml/kg i.p

Diet Consumed, Grams

| | Raw Data | | | | | | | | Weight |
|---|---|---|---|---|---|---|---|---|---|
| Rat | Diet | Time | | | | Diet Consumed | | | of RAT, |
| # | in | in | 1 hr | 2 hr | 3 hr | 1 hr | 2 hr | 3 hr | g |
| 33 | 150.7 | 8.13 | 148.7 | 144.3 | 143.6 | 2 | 6.4 | 7.1 | 232.4 |
| 29 | 150.4 | | 147.4 | 143.0 | 140.3 | 3.0 | 7.4 | 10.1 | 226.8 |
| 15 | 143.7 | | 140.7 | 136.9 | 133.3 | 3.0 | 6.8 | 10.4 | 198.7 |
| 20 | 126.7 | | 124.7 | 121.4 | 117.4 | 2.0 | 5.3 | 9.3 | 234.1 |
| 11 | 113.5 | | 111.2 | 105.6 | 101.3 | 2.3 | 7.9 | 12.2 | 215 |
| 13 | 99.1 | | 95.5 | 91.5 | 89.4 | 3.6 | 7.6 | 9.7 | 235.3 |
| 2 | 116.7 | | 115.3 | 111.2 | 108.9 | 1.4 | 5.5 | 7.8 | 202.2 |
| 40 | 147.0 | | 144.0 | 138.8 | 137.1 | 3.0 | 8.2 | 9.9 | 207.1 |
| X | | | | | | 2.5 | 6.9 | 9.6 | 219.0 |
| sd | | | | | | .7 | 1.1 | 1.6 | 15.1 |

Drug:

Sibutramine, 3 mg/kg i.p.

Diet Consumed. Grams

| | Raw Data | | | | | | | | Weight |
|---|---|---|---|---|---|---|---|---|---|
| Rat | Diet | Time | | | | Diet Consumed | | | of RAT, |
| # | in | in | 1 hr | 2 hr | 3 hr | 1 hr | 2 hr | 3 hr | g |
| 17 | 131.2 | 8.27 | 128.8 | 125.6 | 123 | 2.4 | 5.6 | 8.2 | 218.2 |
| 4 | 122.0 | | 121.1 | 118.0 | 116.0 | 0.9 | 4.0 | 6.0 | 238.3 |
| 14 | 146.8 | | 142.7 | 139.4 | 137.9 | 4.1 | 7.4 | 8.9 | 186 |
| 7 | 144.1 | | 141.5 | 137.2 | 134.6 | 2.6 | 6.9 | 9.5 | 222 |
| 22 | 134.9 | | 130.8 | 127.2 | 123.7 | 4.1 | 7.7 | 11.2 | 233.9 |
| 30 | 121.2 | | 119.3 | 114.2 | 112.2 | 1.9 | 7.0 | 9.0 | 202.8 |
| 35 | 128.6 | | 123.7 | 121.4 | 118.6 | 4.9 | 7.2 | 10.0 | 211.5 |
| 31 | 147.4 | | 140.1 | 136.9 | 135.1 | 7.3 | 10.5 | 12.3 | 217.1 |
| X | | | | | | 3.5 | 7.0 | 9.4 | 216.2 |
| sd | | | | | | 2.0 | 1.8 | 1.9 | 16.7 |

Drug:

### (S,S)-6-(4-Amino-butyl)-1-biphenyl-4-ylmethyl-3-naphthalen-2-ylmethyl-piperazine-2,5-dione (example 23) 1 mg/kg i.p.

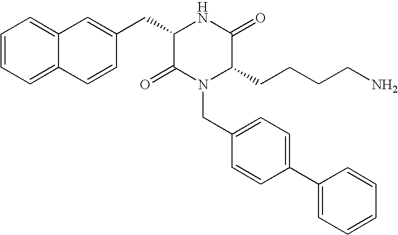

Diet Consumed, Grams

| | Raw Data | | | | | | | | Weight |
|---|---|---|---|---|---|---|---|---|---|
| Rat | Diet | Time | | | | Diet Consumed | | | of RAT, |
| # | in | in | 1 hr | 2 hr | 3 hr | 1 hr | 2 hr | 3 hr | g |
| 10 | 117.7 | 8.40 | 113.4 | 110.1 | 108 | 4.3 | 7.6 | 9.7 | 253.5 |
| 16 | 140.7 | | 139.3 | 136.2 | 133.7 | 1.4 | 4.5 | 7.0 | 203.6 |
| 32 | 137.0 | | 135.0 | 132.3 | 128.6 | 2.0 | 4.7 | 8.4 | 211.2 |
| 28 | 162.4 | | 160.3 | 153.4 | 153.4 | 2.1 | 9.0 | 9.0 | 228.3 |
| 21 | 140.0 | | 138.4 | 134.2 | 134.2 | 1.6 | 5.8 | 5.8 | 228.6 |
| 9 | 129.0 | | 127.6 | 123.4 | 120.9 | 1.4 | 5.6 | 8.1 | 239.8 |
| 5 | 152.8 | | 149.4 | 146.4 | 142.2 | 3.4 | 6.4 | 10.6 | 204.8 |
| 38 | 140.3 | | 137.5 | 133.5 | 131.6 | 2.8 | 6.8 | 8.7 | 217.5 |
| X | | | | | | 2.4 | 6.3 | 8.4 | 223.4 |
| sd | | | | | | 1.0 | 1.5 | 1.5 | 17.5 |

Drug:

### (S,S)-6-(4-Amino-butyl)-1-biphenyl-4-ylmethyl-3-naphthalen-2-ylmethyl-piperazine-2,5-dione (example 23) 3 mg/kg i.p.

Diet Consumed, Grams

| | Raw Data | | | | | | | | Weight |
|---|---|---|---|---|---|---|---|---|---|
| Rat | Diet | Time | | | | Diet Consumed | | | of RAT, |
| # | in | in | 1 hr | 2 hr | 3 hr | 1 hr | 2 hr | 3 hr | g |
| 39 | 139.4 | 8.17 | 135.4 | 132.2 | 128.3 | 4 | 7.2 | 11.1 | 202.4 |
| 37 | 124.0 | | 119.8 | 115.6 | 114.2 | 4.2 | 8.4 | 9.8 | 203.4 |
| 19 | 155.1 | | 151.4 | 150.4 | 149.2 | 3.7 | 4.7 | 5.9 | 222.3 |
| 6 | 158.1 | | 153.1 | 149.5 | 148.0 | 5.0 | 8.6 | 10.1 | 200.1 |
| 25 | 146.7 | | 144.2 | 138.3 | 135.8 | 2.5 | 8.4 | 10.9 | 235.9 |
| 24 | 103.5 | | 102.5 | 98.5 | 98.2 | 1.0 | 5.0 | 5.3 | 211.2 |
| 3 | 99.9 | | 98.4 | 94.3 | 92.4 | 1.5 | 5.6 | 7.5 | 222.3 |
| 26 | 141.0 | | 135.3 | 132.0 | 131.3 | 5.7 | 9.0 | 9.7 | 218 |
| X | | | | | | 3.4 | 7.1 | 8.5 | 216.2 |
| sd | | | | | | 1.8 | 1.9 | 2.2 | 12.3 |

Drug:

### (S,S)-6-(4-Amino-butyl)-1-biphenyl-4-ylmethyl-3-naphthalen-2-ylmethyl-piperazine-2,5-dione (example 23) 10 mg/kg i.p.

Diet Consumed, Grams

Raw Data Diet Consumed Weight

| | Raw Data | | | | | | | | Weight |
|---|---|---|---|---|---|---|---|---|---|
| Rat | Diet | Time | | | | Diet Consumed | | | of RAT, |
| # | in | in | 1 hr | 2 hr | 3 hr | 1 hr | 2 hr | 3 hr | g |
| 27 | 111.5 | 8.35 | 104.8 | 103.6 | 100.8 | 6.7 | 7.9 | 10.7 | 234.7 |
| 36 | 151.2 | | 148.7 | 144.9 | 144.9 | 2.5 | 6.3 | 6.3 | 234.7 |
| 34 | 153.6 | | 153.4 | 149.0 | 147.8 | .2 | 4.6 | 5.8 | 226.7 |
| 23 | 154.1 | | 150.9 | 149.0 | 149.0 | 3.2 | 5.1 | 5.1 | 228 |
| 8 | 117.2 | | 115.1 | 113.4 | 111.8 | 2.1 | 3.8 | 5.4 | 180.3 |
| 18 | 122.8 | | 119.8 | 117.0 | 117.0 | 3.0 | 5.8 | 5.8 | 211.8 |
| 12 | 155.0 | | 153.5 | 150.8 | 149.9 | 1.5 | 4.2 | 5.1 | 197.4 |
| 1 | 143.6 | | 142.6 | 139.8 | 136.9 | 1.0 | 3.8 | 6.7 | 228 |
| X | | | | | | 2.7 | 5.4 | 6.3 | 216.2 |
| sd | | | | | | 2.0 | 1.4 | 2.0 | 20.8 |

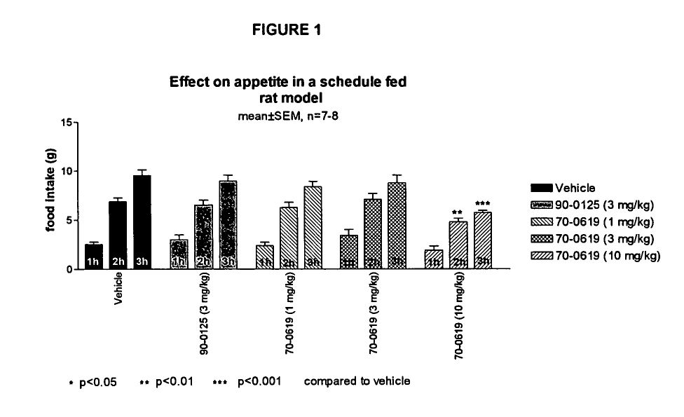

These results are graphically represented in FIG. 1.

### EXEMPLARY ASPECTS OF THE INVENTION

The following is a nonlimiting list of some of the exemplary aspects and features of the invention:
1. A compound of the general formula (I) 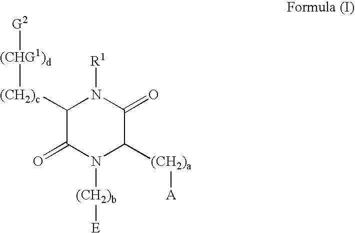
wherein
A is —NR²R³ or guanidinyl, the last optionally substituted with C₁₋₆-alkyl, wherein
   R² and R³ independently of each other are hydrogen, C₁₋₆-alkyl,
   C₁₋₆-alkylene-N(R¹¹)(R¹²), C₁₋₆-alkylene-CN, C₁₋₆-alkylene-OH,
   C₁₋₆-alkylene-C(O)—N(R¹¹)(R¹²), (Z¹)ₑ-R¹³, or —CO—R¹⁴, wherein
      R¹¹ and R¹² independently of each other are hydrogen or C₁₋₆-alkyl;
      Z¹ is C₁₋₆-alkylene;
      e is an integer selected from 0 or 1;
      R¹³ is cycloalkyl, heterocyclyl, aryl, or heteroaryl; each of which may be optionally substituted with a substitutent selected from the group consisting of C₁₋₆-alkyl, amino, and —CO—O-Z⁴-R²³, wherein
         Z⁴ is C₁₋₆-alkylene; and
         R²³ is aryl; and
      R¹⁴ is hydrogen, C₁₋₆-alkyl, —N(R¹⁵)(R¹⁶), C₁₋₆-alkylene-N(R¹⁵)(R¹⁶),
      C(R¹⁷)(R¹⁸)—N(R¹⁹)(R²⁰), heterocyclyl, (Z²)_{f}-R²¹, heteroaryl, or C₁₋₆-alkoxy, wherein
         R¹⁵ and R¹⁶ independently of each other are hydrogen, or C₁₋₆-alkyl;
         R¹⁷ and R¹⁸ independently of each other are hydrogen,
         C₁₋₆-alkylene-NH₂ or (Z³)_{g}-R²²), wherein
            Z³ is C₁₋₆-alkylene;
            g is an integer selected from 0 or 1; and
            R²² is cycloalkyl, heterocyclyl, aryl or heteroaryl;
         R¹⁹ and R²⁰ independently of each other are hydrogen,
         C₂₋₆-alkylene-NH₂, C₁₋₆-alkylene-CF₃ or cycloalkyl; and
         Z² is C₁₋₆-alkylene;
         f is an integer selected from 0 or 1; and
         R²¹ is cycloalkyl, heterocyclyl, aryl or heteroaryl;
a is an integer selected from 1, 2, 3, 4, or 5;
E is cycloalkyl, heterocyclyl, aryl or heteroaryl; each of which may be optionally substituted with one or more substituents selected from the group consisting of halogen, hydroxy, cyano, nitro, —NR⁴R⁵, —CO-R⁶, C₁₋₆-alkyl, C₁₋₆-alkoxy, trifluoromethyl, trifluoromethoxy, and -L¹-Q¹, wherein
   R⁴ and R⁵ independently of each other are hydrogen, C₁₋₆-alkyl, —CO—R²⁴, or aryl, wherein
      R²⁴ is hydrogen, C₁₋₆-alkyl or C₁₋₆-alkoxy;
   R⁶ is C₁₋₆-alkyl or C₁₋₆-alkoxy;
   L¹ is a direct bond, —CH₂—, —O—, —CO—, —CH₂—O—, —O—CH₂— or —NR²⁵—, wherein
      R²⁵ is hydrogen or C₁₋₆-alkyl; and
   Q¹ is cycloalkyl, heterocyclyl, aryl or heteroaryl; each of which may be optionally substituted with one or more substituents selected from the group consisting of halogen, hydroxy, cyano, nitro, trifluoromethyl, trifluoromethoxy, —NR²⁶R²⁷, —CO—R²⁸, —S(O)₂—R²⁹, C₁₋₆-alkyl, C₁₋₆-alkoxy, C₃₋₇-cycloalkyl and C₃₋₇-cycloalkoxy, wherein
      R²⁶ and R²⁷ independently of each other are hydrogen, C₁₋₆-alkyl, or —CO—R³⁰, wherein
         R³⁰ is hydrogen, C₁₋₆-alkyl or C₁₋₆-alkoxy;
      R²⁸ is C₁₋₆-alkyl or C₁₋₆-alkoxy; and
      R²⁹ is C₁₋₆-alkyl, —NH—C₁₋₆-alkyl, or —N(C₁₋₆-alkyl)₂;
   or
   Q¹ is L³-R³¹, wherein
      L³ is —CH₂—, —O—, —CO—, —CH₂—O—, —O—CH₂—, —CH₂—O—C(O)—, or —C(O)—O—CH₂—; and
      R³¹ is aryl or heteroaryl;
b is an integer selected from 0, 1, or 2;
G¹ is C₁₋₆-alkyl, C₁₋₆-alkoxy, cycloalkyl, C₃₋₇-cycloalkoxy, aryl or heteroaryl; each of which may be optionally substituted with halogen, hydroxy, cyano, nitro, trifluoromethyl, trifluoromethoxy, —NR⁷R⁸, C₁₋₆-alkyl, C₁₋₆-alkoxy, C₃₋₇-cycloalkyl, C₃₋₇-cycloalkoxy, wherein
   R⁷ and R⁸ independently of each other are hydrogen, C₁₋₆-alkyl, aryl, heteroaryl,
   —CO—R³² or —SO₂—R³³, wherein
      R³² is hydrogen, C₁₋₆-alkyl or C₁₋₆-alkoxy; and
      R³³ is C₁₋₆-alkyl, —NH—C₁₋₆-alkyl, —N(C₁₋₆-alkyl)₂;
G² is cycloalkyl, heterocyclyl, aryl, or heteroaryl; each of which may be optionally substituted with one or more substituents selected from the group consisting of halogen, hydroxy, cyano, nitro, difluoromethyl, trifluoromethyl, difluoromethoxy, trifluoromethoxy, —NR⁹R¹⁰, C₁₋₆-alkyl, C₁₋₆-alkoxy, C₃₋₇-cycloalkyl, C₃₋₇-cycloalkoxy or -L²-Q², wherein
   R⁹ and R¹⁰ are independently hydrogen, C₁₋₆-alkyl, aryl, heteroaryl, —CO—R³⁴ or
   —SO₂—R³⁵, wherein
      R³⁴ is hydrogen, C₁₋₆-alkyl or C₁₋₆-alkoxy; and
      R³⁵ is C₁₋₆-alkyl, —NH—C₁₋₆-alkyl, or —N(C₁₋₆-alkyl)₂;
   L² is a direct bond, —CH₂—, —O—, —CO—, —CH₂—O—, —O—CH₂— or —NR³⁶—, wherein
      R³⁶ is hydrogen or C₁₋₆-alkyl; and
   Q² is cycloalkyl, heterocyclyl, aryl or heteroaryl; each of which may be optionally substituted with halogen, hydroxy, cyano, nitro, trifluoromethyl, —NR³⁷R³⁸, —CO—R³⁹, —O—R⁴⁰, C₁₋₆-alkyl, C₁₋₆-hydroxyalkyl, C₃₋₇-cycloalkyl or C₃₋₇-cycloalkoxy, wherein
      R³⁷ and R³⁸ independently of each other are hydrogen, C₁₋₆-alkyl or —CO—R⁴¹, wherein
         R⁴¹ is hydrogen, C₁₋₆-alkyl or C₁₋₆-alkoxy;
      R³⁹ is hydrogen, C₁₋₆-alkyl or C₁₋₆-alkoxy; and
      R⁴⁰ is C₁₋₆-alkyl or trifluoromethyl;
c is an integer selected from 0, 1, or 2;
d is an integer selected from 0, or 1; and
R¹ is hydrogen, alkyl, alkenyl, or alkynyl;
   as well as any optical or geometric isomer or tautomer form thereof,
   or a pharmaceutically acceptable salt thereof.
   2. A compound according to aspect 1, wherein A is —NR²R³, wherein
   R² and R³ are as defined in aspect 1.
      3. A compound according to aspect 1 or aspect 2, wherein
   R² is hydrogen, C₁₋₆-alkyl, C₁₋₆-alkylene-N(R¹¹)(R¹²), C₁₋₆-alkylene-CN, C₁₋₆-alkylene-OH,
   C₁₋₆-alkylene-C(O)—N(R¹¹)(R¹²), (Z¹)ₑ-R¹³, or —CO—R¹⁴; and
   R³ is hydrogen, C₁₋₆-alkyl, C₁₋₆-alkylene-N(R¹¹)(R¹²), (Z¹)ₑ-R¹³, or —CO—R¹⁴;
   wherein
      R¹¹, R¹², Z¹, e, R³, and R¹⁴ in each case are as defined in aspect 1.
         4. A compound according to aspect 1 or aspect 2, wherein
   R² and R³ independently of each other are hydrogen, C₁₋₆-alkyl,
   C₁₋₆-alkylene-N(R¹¹)(R¹²), (Z¹)ₑ-R¹³, or —CO—R¹⁴, wherein
      R¹¹, R¹², Z¹, e, R³, and R¹⁴ is as defined in aspect 1.
         5. A compound according to any of aspects 1 to 4, wherein
   R¹¹ and R¹² are hydrogen.
      6. A compound according to aspect 1 or aspect 2, wherein
   R² and R³ independently of each other are hydrogen, C₁₋₆-alkyl, C₁₋₆-alkylene-CN,
   C₁₋₆-alkylene-OH, C₁₋₆-alkylene-C(O)—NH₂, (Z¹)ₑ-R³, or —CO—R¹⁴, wherein
      Z¹, e, R¹³, and R¹⁴ are as defined in aspect 1.
         7. A compound according to any of aspects 1 to 6, wherein
   e is 1; and
   Z¹ is —CH₂—.
      8. A compound according to any of aspects 1 to 7, wherein
   R¹³ is cycloalkyl, or aryl; each of which may be optionally substituted with a substitutent selected from the group consisting of C₁₋₆-alkyl, amino, and —CO—O-Z⁴-R²³, wherein
      Z⁴ and R²³ is as defined in aspect 1.
         9. A compound according to aspect 8, wherein
   R¹³ is C₃₋₇-cycloalkyl, or C₆₋₁₃-aryl; each of which may be optionally substituted with a substitutent selected from the group consisting of C₁₋₆-alkyl, amino, and —CO—O-Z⁴-R²³, wherein
      Z⁴ and R²³ is as defined in aspect 1.
         10. A compound according to any of aspects 1 to 7, wherein
   R¹³ is heterocyclyl, or heteroaryl; each of which may be optionally substituted with a substitutent selected from the group consisting of C₁₋₆-alkyl, amino, and —CO—O-Z⁴-R²³, wherein
      Z⁴ and R²³ is as defined in aspect 1.
         11. A compound according to aspect 10, wherein
   R¹³ is C₃₋₁₀-heterocyclyl or C₅₋₁₄-heteroaryl; each of which may be optionally substituted with a substitutent selected from the group consisting of C₁₋₆-alkyl, amino, and
   —CO—O-Z⁴-R²³, wherein
      Z⁴ and R²³ is as defined in aspect 1.
         12. A compound according to any of aspects 1 to 11, wherein R²³ is C₆₋₁₃-aryl.
         13. A compound according to aspect 12, wherein R²³ is phenyl.
         14. A compound according to any of aspects 1 to 13, wherein
   R² and R³ independently of each other are hydrogen, C₁₋₆-alkyl, or —CO—R¹⁴, wherein
      R¹⁴ is as defined in aspect 1.
         15. A compound according to any of aspects 1 to 14, wherein
   R¹⁴ is hydrogen, C₁₋₆-alkyl, —NR¹⁵R¹⁶, C₁₋₆-alkylene-N(R¹⁵)(R¹⁶), C(R¹⁷)(R¹⁸)—N(R¹⁹)(R²⁰),
   C₃₋₁₀-heterocyclyl, (Z²)_{f}-R²¹, C₅₋₁₄-heteroaryl, or C₁₋₆-alkoxy, wherein
      R¹⁵, R¹⁶, R¹⁷, R¹⁸, R¹⁹, R²⁰, Z², f, and R²¹ are as defined in aspect 1.
         16. A compound according to any of aspects 1 to 15, wherein R¹⁵ and R¹⁶ are hydrogen.
         17. A compound according to any of aspects 1 to 16, wherein
   R¹⁴ is hydrogen, C₁₋₆-alkyl, C₁₋₆-alkylene-NH₂, C(R¹⁷)(R¹⁸)—N(R¹⁹)(R²⁰),
   C₃₋₁₀-heterocyclyl, (Z²)_{f}-R²¹, or C₅₋₁₄-heteroaryl, wherein
      R¹⁷, R¹⁸, R¹⁹, R²⁰, Z², f, and R²¹ are as defined in aspect 1.
         18. A compound according to aspect 17, wherein
   R¹⁴ is hydrogen, C₁₋₆-alkyl, C₁₋₆-alkylene-NH₂, C(R¹⁷)(R¹⁸)—N(R¹⁹)(R²⁰), C₅₋₆-heterocyclyl,
   (Z²)_{f}-R²¹, or C₅₋₆-heteroaryl, wherein
      R¹⁷, R¹⁸, R¹⁹, R²⁰, Z², f, and R²¹ are as defined in aspect 1.
         19. A compound according to aspect 18, wherein
   R¹⁴ is hydrogen, C₁₋₆-alkyl, C₁₋₆-alkylene-NH₂, C(R¹⁷)(R¹⁸)—N(R¹⁹)(R²⁰), piperidinyl,
   (Z²)_{f}-R²¹, or pyridinyl, wherein
      R¹⁷, R¹⁸, R¹⁹, R²⁰, Z², f, and R²¹ are as defined in aspect 1
         20. A compound according to any of aspects 1 to 19, wherein
   R¹⁷ and R¹⁸ independently of each other are hydrogen, C₁₋₆-alkylene-NH₂ or (Z³)_{g}-R²²), wherein
      Z³ is —CH₂—; and
      g is 1; and
      R²² is as defined in aspect 1.
         21. A compound according to any of aspects 1 to 20, wherein
   R²² is C₃₋₁₂-cycloalkyl, C₃₋₁₀-heterocyclyl, C₆₋₁₃-aryl or C₅₋₁₄-heteroaryl.
      22. A compound according to aspect 21, wherein
   R²² is C₃₋₇-cycloalkyl, C₅₋₆-heterocyclyl, C₆₋₁₃-aryl or C₅₋₆-heteroaryl.
      23. A compound according to aspect 22, wherein
   R²² is C₅₋₆-heterocyclyl.
      24. A compound according to any of aspects 1 to 23, wherein
   R¹⁷ and R¹⁸ are hydrogen.
      25. A compound according to any of aspects 1 to 24, wherein
   R¹⁹ and R²⁰ independently of each other are hydrogen, C₂₋₆-alkylene-NH₂,
   C₁₋₆-alkylene-CF₃ or C₃₋₇-cycloalkyl.
      26. A compound according to aspect 25, wherein
   R¹⁹ and R²⁰ are hydrogen.
      27. A compound according to any of aspects 1 to 26, wherein
   f is 1;
   Z² is —CH₂—; and
   R²¹ is as defined in aspect 1.
      28. A compound according to any of aspects 1 to 27, wherein
   R²¹ is heterocyclyl or heteroaryl.
      29. A compound according to aspect 28, wherein
   R²¹ is C₃₋₁₀-heterocyclyl or C₅₋₁₄-heteroaryl.
      30. A compound according to aspect 29, wherein
   R²¹ is C₅₋₆-heterocyclyl or C₅₋₆-heteroaryl.
      31. A compound according to aspect 30, wherein
   R²¹ is piperidinyl, morpholinyl, imidazolyl, pyrrolidinyl, or pyridinyl.
      32. A compound according to aspect 15, wherein
   R¹⁴ is hydrogen, C₁₋₆-alkyl, —NR¹⁵R¹⁶, or C₁₋₆-alkoxy, wherein
      R¹⁵ and R¹⁶ are as defined in aspect 1.
         33. A compound according to any of aspects 1 to 32, wherein R¹⁵ and R¹⁶ are hydrogen.
         34. A compound according to any of aspects 1 to 33, wherein
   R² and R³ independently of each other are hydrogen or C₁₋₆-alkyl.
      35. A compound according to aspect 34, wherein
   R² and R³ are hydrogen.
      36. A compound according to aspect 1, wherein A is guanidinyl optionally substituted with C₁₋₆-alkyl.
      37. A compound according to any of aspects 1 to 36, wherein a is 1.
      38. A compound according to any of aspects 1 to 35 with the proviso that when A is —NR²R³ and R² and R³ are hydrogen, then a is 4 or 5.
      39. A compound according to any of aspects 1 to 38, wherein the sum of the carbon and nitrogen atoms in the —(CH₂)ₐ-A group is at least 4.
      40. A compound according to aspect 39, wherein the sum of the carbon and nitrogen atoms in the —(CH₂)ₐ-A group is at least 5.
      41. A compound according to any of aspects 1 to 40, wherein a is 4.
      42. A compound according to any of aspects 1 to 40, wherein a is 5.
      43. A compound according to aspect 1 or aspect 2, wherein
   R² is C₃₋₆-alkyl, C₃₋₆-alkylene-N(R¹¹)(R¹²), C₃₋₆-alkylene-CN, C₃₋₆-alkylene-OH,
   C₃₋₆-alkylene-C(O)—N(R¹¹)(R¹²), (Z¹)ₑ-R¹³, or —CO—R¹⁴; and
   R³ is C₃₋₆-alkyl, C₃₋₆-alkylene-N(R¹¹)(R¹²), (Z¹)ₑ-R¹³, or —CO—R¹⁴;
   wherein
      R¹¹, R¹², Z¹, e, and R¹³ in each case are as defined in aspect 1, and
      R¹⁴ is C₂₋₆-alkyl, C₂₋₆-alkylene-N(R¹⁵)(R¹⁶), C(R¹⁷)(R¹⁸)—N(R¹⁹)(R²⁰), heterocyclyl,
      (Z²)_{f}-R²¹, heteroaryl, C₂₋₆-alkoxy, or —N(R⁴²)(R⁴³), wherein
         R¹⁵ and R¹⁶ independently of each other are hydrogen, or C₁₋₆-alkyl;
         R¹⁷ and R¹³ independently of each other are hydrogen,
         C₁₋₆-alkylene-NH₂ or (Z³)₉—R²²), wherein
            Z³ is C₁₋₆-alkylene;
            g is an integer selected from 0 or 1; and
            R²² is cycloalkyl, heterocyclyl, aryl or heteroaryl;
         R¹⁹ and R²⁰ independently of each other are hydrogen,
         C₂₋₆-alkylene-NH₂, C₁₋₆-alkylene-CF₃ or cycloalkyl; and
         Z² is C₁₋₆-alkylene;
         f is an integer selected from 0 or 1; and
         R²¹ is cycloalkyl, heterocyclyl, aryl or heteroaryl; and
         R⁴² and R⁴³ independently of each other are C₁₋₆-alkyl.
            44. A compound according to aspect 1 or aspect 2, wherein
   R² and R³ independently of each other are C₃₋₆-alkyl, C₃₋₆-alkylene-N(R¹¹)(R¹²),
   (Z¹)ₑ-R¹³, or —CO—R¹⁴, wherein
      R¹¹, R¹², Z¹, e, and R¹³ in each case are as defined in aspect 1, and
      R¹⁴ is C₂₋₆-alkyl, C₂₋₆-alkylene-N(R¹⁵)(R¹⁶), C(R¹⁷)(R¹⁸)—N(R¹⁹)(R²⁰), heterocyclyl,
      (Z²)_{f}-R²¹, heteroaryl, C₂₋₆-alkoxy, or —N(R⁴²)(R⁴³), wherein
         R¹⁵ and R¹⁶ independently of each other are hydrogen, or C₁₋₆-alkyl;
         R¹⁷ and R¹⁸ independently of each other are hydrogen,
         C₁₋₆-alkylene-NH₂ or (Z³)_{g}-R²²), wherein
            Z³ is C₁₋₆-alkylene;
            g is an integer selected from 0 or 1; and
            R²² is cycloalkyl, heterocyclyl, aryl or heteroaryl;
         R¹⁹ and R²⁰ independently of each other are hydrogen,
         C₂₋₆-alkylene-NH₂, C₁₋₆-alkylene-CF₃ or cycloalkyl; and
         Z² is C₁₋₆-alkylene;
         f is an integer selected from 0 or 1; and
         R²¹ is cycloalkyl, heterocyclyl, aryl or heteroaryl; and
         R⁴² and R⁴³ independently of each other are C₁₋₆-alkyl.
            45. A compound according to aspect 43 or aspect 44, wherein
   R¹¹ and R¹² are hydrogen.
      46. A compound according to aspect 1 or aspect 2, wherein
   R² and R³ independently of each other are C₃₋₆-alkyl, C₃₋₆-alkylene-CN,
   C₃₋₆-alkylene-OH, C₃₋₆-alkylene-C(O)—NH₂, (Z¹)ₑ-R¹³, or —CO—R¹⁴, wherein
      Z¹, e, and R¹³ are as defined in aspect 1, and
      R¹⁴ is C₂₋₆-alkyl, C₂₋₆-alkylene-N(R¹⁵)(R¹⁶), C(R¹⁷)(R¹⁸)—N(R¹⁹)(R²⁰), heterocyclyl,
      (Z²)_{f}-R²¹, heteroaryl, C₂₋₆-alkoxy, or —N(R⁴²)(R⁴³), wherein
         R¹⁵ and R¹⁶ independently of each other are hydrogen, or C₁₋₆-alkyl;
         R¹⁷ and R¹⁸ independently of each other are hydrogen,
         C₁₋₆-alkylene-NH₂ or (Z³)_{g}-R²²), wherein
            Z³ is C₁₋₆-alkylene;
            g is an integer selected from 0 or 1; and
            R²² is cycloalkyl, heterocyclyl, aryl or heteroaryl;
         R¹⁹ and R²⁰ independently of each other are hydrogen,
         C₂₋₆-alkylene-NH₂, C₁₋₆-alkylene-CF₃ or cycloalkyl; and
         Z² is C₁₋₆-alkylene;
         f is an integer selected from 0 or 1; and
         R²¹ is cycloalkyl, heterocyclyl, aryl or heteroaryl; and
         R⁴² and R⁴³ independently of each other are C₁₋₆-alkyl.
            47. A compound according to any of aspects 43 to 46, wherein
   e is 1; and
   Z¹ is —CH₂—.
      48. A compound according to any of aspects 43 to 47, wherein
   R¹³ is cycloalkyl, or aryl; each of which may be optionally substituted with a substitutent selected from the group consisting of C₁₋₆-alkyl, amino, and —CO—O-Z⁴-R²³, wherein
      Z⁴ and R²³ is as defined in aspect 1.
         49. A compound according to aspect 48, wherein
   R¹³ is C₃₋₇-cycloalkyl, or C₆₋₁₃-aryl; each of which may be optionally substituted with a substitutent selected from the group consisting of C₁₋₆-alkyl, amino, and —CO—O-Z⁴-R²³, wherein
      Z⁴ and R²³ is as defined in aspect 1.
         50. A compound according to any of aspects 43 to 47, wherein
   R¹³ is heterocyclyl, or heteroaryl; each of which may be optionally substituted with a substitutent selected from the group consisting of C₁₋₆-alkyl, amino, and —CO—O-Z⁴-R²³, wherein
      Z⁴ and R²³ is as defined in aspect 1.
         51. A compound according to aspect 50, wherein
   R¹³ is C₃₋₁₀-heterocyclyl or C₅₋₁₄-heteroaryl; each of which may be optionally substituted with a substitutent selected from the group consisting of C₁₋₆-alkyl, amino, and
   —CO—O-Z⁴-R²³, wherein
      Z⁴ and R²³ is as defined in aspect 1.
         52. A compound according to any of aspects 43 to 51, wherein R²³ is C₆₋₁₃-aryl.
         53. A compound according to aspect 52, wherein R²³ is phenyl.
         54. A compound according to any of aspects 43 to 53, wherein
   R² and R³ independently of each other are C₃₋₆-alkyl, or —CO—R⁴, wherein
      R¹⁴ is as defined in aspect 43.
         55. A compound according to any of aspects 43 to 54, wherein
   R¹⁴ is C₂₋₆-alkyl, C₂₋₆-alkylene-N(R¹⁵)(R¹⁶), C(R¹⁷)(R¹⁸)—N(R¹⁹)(R²⁰), C₃₋₁₀-heterocyclyl,
   (Z²)_{f}-R²¹, C₅₋₁₄-heteroaryl, C₂₋₆-alkoxy, or —N(R⁴²) (R⁴³), wherein
      R¹⁵, R¹⁶, R¹⁷, R¹⁸, R¹⁹, R²⁰, Z², f, R²¹, R⁴² and R⁴³ are as defined in aspect 43.
         56. A compound according to any of aspects 43 to 55, wherein R¹⁵ and R¹⁶ are hydrogen.
         57. A compound according to any of aspects 43 to 56, wherein
   R¹⁴ is C₂₋₆-alkyl, C₂₋₆-alkylene-NH₂, C(R¹⁷)(R¹⁸)—N(R¹⁹)(R²⁰), C₃₋₁₀-heterocyclyl, (Z²)_{f}-R²¹, or C₅₋₁₄-heteroaryl, wherein
      R¹⁷, R¹⁸, R¹⁹, R²⁰, Z², f, and R²¹ are as defined in aspect 43.
         58. A compound according to aspect 57, wherein
   R¹⁴ is C₂₋₆-alkyl, C₂₋₆-alkylene-NH₂, C(R¹⁷)(R¹⁸)—N(R¹⁹)(R²⁰), C₅₋₆-heterocyclyl, (Z²)_{f}-R²¹, or C₅₋₆-heteroaryl, wherein
      R¹⁷, R¹⁸, R¹⁹, R²⁰, Z², f, and R²¹ are as defined in aspect 43.
         59. A compound according to aspect 58, wherein
   R¹⁴ is C₂₋₆-alkyl, C₂₋₆-alkylene-NH₂, C(R¹⁷)(R¹⁸)—N(R¹⁹)(R²⁰), piperidinyl, (Z²)_{f}-R²¹, or pyridinyl, wherein
      R¹⁷, R¹⁸, R¹⁹, R²⁰, Z², f, and R²¹ are as defined in aspect 43.
         60. A compound according to any of aspects 43 to 59, wherein
   R¹⁷ and R¹⁸ independently of each other are hydrogen, C₁₋₆-alkylene-NH₂ or (Z³)_{g}-R²²), wherein
      Z³ is —CH₂—; and
      g is 1; and
      R²² is as defined in aspect 43.
         61. A compound according to any of aspects 43 to 60, wherein
   R²² is C₃₋₁₂-cycloalkyl, C₃₋₁₀-heterocyclyl, C₆₋₁₃-aryl or C₅₋₁₄-heteroaryl.
      62. A compound according to aspect 61, wherein
   R²² is C₃₋₇-cycloalkyl, C₅₋₆-heterocyclyl, C₆₋₁₃-aryl or C₅₋₆-heteroaryl.
      63. A compound according to aspect 62, wherein
   R²² is C₅₋₆-heterocyclyl.
      64. A compound according to any of aspects 43 to 63, wherein
   R¹⁷ and R¹⁸ are hydrogen.
      65. A compound according to any of aspects 43 to 64, wherein
   R¹⁹ and R²⁰ independently of each other are hydrogen, C₂₋₆-alkylene-NH₂, C₁₋₆-alkylene-CF₃ or C₃₋₇-cycloalkyl.
      66. A compound according to aspect 65, wherein
   R¹⁹ and R²⁰ are hydrogen.
      67. A compound according to any of aspects 43 to 66, wherein
   f is 1;
   Z² is —CH₂—; and
   R²¹ is as defined in aspect 43.
      68. A compound according to any of aspects 43 to 67, wherein
   R²¹ is heterocyclyl or heteroaryl.
      69. A compound according to aspect 68, wherein
R²¹ is C₃₋₁₀-heterocyclyl or C₅₋₁₄-heteroaryl.
   70. A compound according to aspect 69, wherein
   R²¹ is C₅₋₆-heterocyclyl or C₅₋₆-heteroaryl.
      71. A compound according to aspect 70, wherein
   R²¹ is piperidinyl, morpholinyl, imidazolyl, pyrrolidinyl, or pyridinyl.
      72. A compound according to aspect 56, wherein
   R¹⁴ is hydrogen, C₁₋₆-alkyl, —N(R¹⁵)(R¹⁶), or C₁₋₆-alkoxy, wherein
      R¹⁵ and R¹⁶ are as defined in aspect 43.
         73. A compound according to any of aspects 43 to 72, wherein R¹⁵ and R¹⁶ are hydrogen.
         74. A compound according to any of aspects 43 to 73, wherein
   R² and R³ independently of each other are C₃₋₆-alkyl.
      75. A compound according to any of aspects 43 to 74, wherein a is 1.
      76. A compound according to any of aspects 1 to 75, wherein
   E is C₃₋₁₂-cycloalkyl, C₃₋₁₀-heterocyclyl, C₆₋₁₃-aryl or C₅₋₁₄-heteroaryl; each of which may be optionally substituted with one or more substituents selected from the group consisting of halogen, hydroxy, cyano, nitro, —NR⁴R⁵, —CO—R⁶, C₁₋₆-alkyl, C₁₋₆-alkoxy, trifluoromethyl, trifluoromethoxy, and -L¹-Q¹, wherein
      R⁴, R⁵, R⁶, L¹, and Q¹ are as defined in aspect 1.
         77. A compound according to any of aspects 1 to 42, wherein
   E is aryl or heteroaryl; each of which may be optionally substituted with one or more substituents selected from the group consisting of halogen, hydroxy, cyano, nitro, —NR⁴R⁵, —CO—R⁶, C₁₋₆-alkyl, C₁₋₆-alkoxy, trifluoromethyl, trifluoromethoxy, and -L¹-Q¹, wherein
      R⁴, R⁵, R⁶, L¹, and Q¹ are as defined in aspect 1.
         78. A compound according to aspect 76 or aspect 77, wherein
   E is C₆₋₁₃-aryl or C₅₋₁₄-heteroaryl; each of which may be optionally substituted with one or more substituents selected from the group consisting of halogen, hydroxy, cyano, nitro, —NR⁴R⁵, —CO—R⁶, C₁₋₆-alkyl, C₁₋₆-alkoxy, trifluoromethyl, trifluoromethoxy, and -L¹-Q¹, wherein
      R⁴, R⁵, R⁶, L¹, and Q¹ are as defined in aspect 1.
         79. A compound according to aspect 78, wherein
   E is C₆₋₁₃-aryl or C₅₋₁₄-heteroaryl; each of which may be optionally substituted with one or more substituents selected from the group consisting of halogen, —NR⁴R⁵, C₁₋₆-alkyl, C₁₋₆-alkoxy, and -L¹-Q¹, wherein
      R⁴, R⁵, L¹, and Q¹ are as defined in aspect 1.
         80. A compound according to any of aspects 1 to 79, wherein
   R⁴ and R⁵ independently of each other are hydrogen, C₁₋₆-alkyl, or aryl.
      81. A compound according to aspect 80, wherein
   R⁴ and R⁵ independently of each other are hydrogen, C₁₋₆-alkyl, or C₆₋₁₃-aryl.
      82. A compound according to aspect 81, wherein
   R⁴ and R⁵ independently of each other are hydrogen, C₁₋₆-alkyl, or phenyl.
      83. A compound according to any of aspects 1 to 82, wherein
   L¹ is a direct bond, —CH₂—, —O—, —CH₂—O—, or —O—CH₂—.
      84. A compound according to aspect 83, wherein
   L¹ is a direct bond.
      85. A compound according to aspect 83, wherein
   L¹ is —CH₂—.
      86. A compound according to aspect 83, wherein
   L¹ is —O—.
      87. A compound according to any of aspects 1 to 86, wherein
   Q is C₃₋₁₂-cycloalkyl, C₃₋₁₀-heterocyclyl, C₆₋₁₃-aryl, or C₅₋₁₄-heteroaryl; each of which may be optionally substituted with one or more substituents selected from the group consisting of halogen, hydroxy, cyano, nitro, trifluoromethyl, trifluoromethoxy, —NR², R²⁷, —CO—R²⁸, —S(O)₂—R²⁹, C₁₋₆-alkyl, C₁₋₆-alkoxy, C₃₋₇-cycloalkyl and C₃₋₇-cycloalkoxy, wherein
      R²⁶, R²⁷, R²⁸, and R²⁹ are as defined in aspect 1.
         88. A compound according to aspect 87, wherein
   Q¹ is C₃₋₇-cycloalkyl, C₅₋₆-heterocyclyl, C₆₋₁₃-aryl, or C₅₋₆-heteroaryl; each of which may be optionally substituted with one or more substituents selected from the group consisting of halogen, hydroxy, cyano, nitro, trifluoromethyl, trifluoromethoxy, —NR², R²⁷, —CO—R²⁸, —S(O)₂—R²⁹, C₁₋₆-alkyl, C₁₋₆-alkoxy, C₃₋₇-cycloalkyl and C₃₋₇-cycloalkoxy, wherein
      R²⁶, R²⁷, R²⁸, and R²⁹ are as defined in aspect 1.
         89. A compound according to aspect 88, wherein
   Q¹ is C₃₋₇-cycloalkyl, or C₆₋₁₃-aryl; each of which may be optionally substituted with one or more substituents selected from the group consisting of halogen, hydroxy, cyano, nitro, trifluoromethyl, trifluoromethoxy, —NR²⁶R²⁷, —CO—R²⁸, —S(O)₂—R²⁹, C₁₋₆-alkyl, C₁₋₆-alkoxy, C₃₋₇-cycloalkyl and C₃₋₇-cycloalkoxy, wherein
      R²⁶, R²⁷, R²⁸, and R²⁹ are as defined in aspect 1.
         90. A compound according to aspect 89, wherein
   Q¹ is C₅₋₆-cycloalkyl, or C₆₋₁₀-aryl; each of which may be optionally substituted with one or more substituents selected from the group consisting of halogen, hydroxy, cyano, nitro, trifluoromethyl, trifluoromethoxy, —NR²⁶R²⁷, —CO—R²⁸, —S(O)₂—R²⁹, C₁₋₆-alkyl, C₁₋₆-alkoxy, C₃₋₇-cycloalkyl and C₃₋₇-cycloalkoxy, wherein
      R²⁶, R²⁷, R²⁸, and R²⁹ are as defined in aspect 1.
         91. A compound according to aspect 90, wherein
   Q¹ is phenyl or cyclohexyl, which may be optionally substituted with one or more substituents selected from the group consisting of halogen, hydroxy, cyano, nitro, trifluoromethyl, trifluoromethoxy, —NR²⁶R²⁷, —CO—R²⁸, —S(O)₂—R²⁹, C₁₋₆-alkyl, C₁₋₆-alkoxy, C₃₋₇-cycloalkyl and C₃₋₇-cycloalkoxy, wherein
      R²⁶, R²⁷, R²⁸, and R²⁹ are as defined in aspect 1.
         92. A compound according to any of aspects 1 to 91, wherein
   R²⁶ and R²⁷ independently of each other are hydrogen, or C₁₋₆-alkyl.
      93. A compound according to aspect 92, wherein
   R²⁶ and R²⁷ independently of each other are hydrogen, or methyl.
      94. A compound according to any of aspects 1 to 93, wherein
   R²⁸ is methyl.
      95. A compound according to any of aspects 1 to 94, wherein
   R²⁹ is C₁₋₆-alkyl.
      96. A compound according to aspect 95, wherein
   R²⁹ is methyl.
      97. A compound according to any of aspects 1 to 86, wherein
   Q¹ is L³-R³¹, wherein
      L³ is —CH₂—, —CH₂—O—C(O)—, or —C(O)—O—CH₂—; and
      R³¹ is as defined in aspect 1.
         98. A compound according to any of aspects 1 to 97, wherein
   R³¹ is C₆₋₁₃-aryl or C₃₋₁₀-heteroaryl.
      99. A compound according to aspect 98, wherein
   R³¹ is C₆₋₁₀-aryl or C₅₋₆-heteroaryl.
      100. A compound according to aspect 99, wherein
   R³¹ is phenyl.
      101. A compound according to any of aspects 1 to 100, wherein b is 1.
      102. A compound according to any of aspects 1 to 101, wherein c is 1.
      103. A compound according to any of aspects 1 to 102, wherein d is 0.
      104. A compound according to any of aspects 1 to 103, wherein
   G² is C₃₋₁₂-cycloalkyl, C₃₋₁₀-heterocyclyl, C₆₋₁₃-aryl or C₅₋₁₄-heteroaryl; each of which may be optionally substituted with one or more substituents selected from the group consisting of halogen, hydroxy, cyano, nitro, difluoromethyl, trifluoromethyl, difluoromethoxy, trifluoromethoxy, —NR⁹R¹⁰, C₁₋₆-alkyl, C₁₋₆-alkoxy, C₃₋₇-cycloalkyl, C₃₋₇-cycloalkoxy or -L²-Q², wherein
      R⁹, R¹⁰, L², and Q² are as defined in aspect 1.
         105. A compound according to any of aspects 1 to 103, wherein
   G² is aryl or heteroaryl; each of which may be optionally substituted with one or more substituents selected from the group consisting of halogen, hydroxy, cyano, nitro, difluoromethyl, trifluoromethyl, difluoromethoxy, trifluoromethoxy, —NR⁹R¹⁰, C₁₋₆-alkyl, C₁₋₆-alkoxy, C₃₋₇-cycloalkyl, C₃₋₇-cycloalkoxy or -L²-Q², wherein
      R⁹, R¹⁰, L², and Q² are as defined in aspect 1.
         106. A compound according to aspect 104 or aspect 105, wherein
   G² is C₆₋₁₃-aryl or C₅₋₁₄-heteroaryl; each of which may be optionally substituted with one or more substituents selected from the group consisting of halogen, hydroxy, cyano, nitro, difluoromethyl, trifluoromethyl, difluoromethoxy, trifluoromethoxy, —NR⁹R¹⁰, C₁₋₆-alkyl, C₁₋₆-alkoxy, C₃₋₇-cycloalkyl, C₃₋₇-cycloalkoxy or -L²-Q², wherein
      R⁹, R¹⁰, L², and Q² are as defined in aspect 1.
         107. A compound according to aspect 106, wherein
   G² is C₆₋₁₀-aryl or C₅₋₁₀-heteroaryl; each of which may be optionally substituted with one or more substituents selected from the group consisting of halogen, hydroxy, cyano, nitro, difluoromethyl, trifluoromethyl, difluoromethoxy, trifluoromethoxy, —NR⁹R¹⁰, C₁₋₆-alkyl, C₁₋₆-alkoxy, C₃₋₇-cycloalkyl, C₃₋₇-cycloalkoxy or -L²-Q², wherein
      R⁹, R¹⁰, L², and Q² are as defined in aspect 1.
         108. A compound according to any of aspects 1 to 107, wherein
   R⁹ and R¹⁰ are independently hydrogen, C₁₋₆-alkyl, C₆₋₁₃-aryl, C₅₋₁₄-heteroaryl, —CO—R³⁴ or —SO₂—R³⁵, wherein
      R³⁴ and R³⁵ are as defined in aspect 1.
         109. A compound according to aspect 108, wherein
   R³⁴ is hydrogen, C₁₋₆-alkyl or C₁₋₆-alkoxy; and
   R³⁵ is C₁₋₆-alkyl.
      110. A compound according to aspect 109, wherein
   R⁹ and R¹⁰ are hydrogen.
      111. A compound according to any of aspects 1 to 110, wherein
   L² is a direct bond, —CH₂—, —O—, —CO—, —CH₂—O—, —O—CH₂— or —NR³⁶—, wherein
      R³⁶ is hydrogen or methyl.
         112. A compound according to any of aspects 1 to 111, wherein
   L² is a direct bond, —CH₂—, —O—, —CO—, —CH₂—O—, or —O—CH₂—.
      113. A compound according to any of aspects 1 to 112, wherein
   Q² is C₃₋₁₂-cycloalkyl, C₃₋₁₀-heterocyclyl, C₆₋₁₃-aryl or C₅₋₁₄-heteroaryl; each of which may be optionally substituted with halogen, hydroxy, cyano, nitro, trifluoromethyl, —NR³⁷R³⁸, —CO—R³⁹, —O—R⁴⁰, C₁₋₆-alkyl, C₁₋₆-hydroxyalkyl, C₃₋₇-cycloalkyl or C₃₋₇-cycloalkoxy, wherein
      R³⁷, R³⁸, R³⁹, and R⁴⁰ are as defined in aspect 1.
         114. A compound according to aspect 113, wherein
   Q² is C₃₋₁₂-cycloalkyl, C₃₋₁₀-heterocyclyl, C₆₋₁₃-aryl or C₅₋₁₄-heteroaryl; each of which may be optionally substituted with halogen, hydroxy, cyano, nitro, trifluoromethyl, —NR³⁷R³⁸, —CO—R³⁹, —O—R⁴⁰, C₁₋₆-alkyl, or C₁₋₆-hydroxyalkyl, wherein
      R³⁷, R³⁸, R³⁹, and R⁴⁰ are as defined in aspect 1.
         115. A compound according to aspect 113, wherein
   Q² is C₃₋₇-cycloalkyl, C₅₋₆-heterocyclyl, C₆₋₁₃-aryl or C₅₋₆-heteroaryl; each of which may be optionally substituted with halogen, hydroxy, cyano, nitro, trifluoromethyl, —NR³⁷R³⁸, —CO—R³⁹, —O—R⁴⁰, C₁₋₆-alkyl, C₁₋₆-hydroxyalkyl, C₃₋₇-cycloalkyl or C₃₋₇-cycloalkoxy, wherein
      R³⁷, R³⁸, R³⁹, and R⁴⁰ are as defined in aspect 1.
         116. A compound according to aspect 115, wherein
   Q² is C₃₋₇-cycloalkyl, C₅₋₆-heterocyclyl, C₆₋₁₃-aryl or C₅₋₆-heteroaryl; each of which may be optionally substituted with halogen, hydroxy, cyano, nitro, trifluoromethyl, —NR³⁷R³⁸, —CO—R³⁹, —O—R⁴⁰, C₁₋₆-alkyl, or C₁₋₆-hydroxyalkyl, wherein
      R³⁷, R³⁸, R³⁹, and R⁴⁰ are as defined in aspect 1.
         117. A compound according to any of aspects 1 to 116, wherein
   R³⁷ and R³⁸ independently of each other are hydrogen or C₁₋₆-alkyl.
      118. A compound according to aspect 117, wherein
   R³⁷ and R³⁸ independently of each other are hydrogen or methyl.
      119. A compound according to any of aspects 1 to 118, wherein
   R³⁹ is hydrogen or C₁₋₆-alkyl.
      120. A compound according to aspect 119, wherein
   R³⁹ is hydrogen or methyl.
      121. A compound according to any of aspects 1 to 120, wherein
   R⁴⁰ is trifluoromethyl.
      122. A compound according to any of aspects 1 to 121, wherein
   R¹ is hydrogen, C₁₋₆-alkyl, C₂₋₆-alkenyl, or C₂₋₆-alkynyl.
      123. A compound according to aspect 122, wherein
   R¹ is hydrogen.
      124. A compound according to aspect 1, where the compound is
(S,S)-6-(4-amino-butyl)-1-biphenyl-4-ylmethyl-3-naphthalen-2-ylmethyl-piperazine-2,5-dione,,
(S,S)-6-(4-amino-butyl)-1-biphenyl-4-ylmethyl-3-naphthalen-1-ylmethyl-piperazine-2,5-dione,
(S,S)-6-(4-amino-butyl)-3-(4-benzyloxy-benzyl)-1-biphenyl-4-ylmethyl-piperazine-2,5-dione,
(S,S)-6-(4-amino-butyl)-1,3-bis-biphenyl-4-ylmethyl-piperazine-2,5-dione,
(S,S)-6-(4-amino-butyl)-3-naphthalen-2-ylmethyl-1-(4-phenoxy-benzyl)-piperazine-2,5-dione,
(S,S)-6-(4-amino-butyl)-3-benzo[b]thiophen-3-ylmethyl-1-biphenyl-4-ylmethyl-piperazine-2,5-dione,
(S,S)-6-(4-amino-butyl)-3-(4-benzoyl-benzyl)-1-biphenyl-4-ylmethyl-piperazine-2,5-dione,
(S,S)-6-(4-amino-butyl)-1-(4′-methoxy-biphenyl-4-ylmethyl)-3-naphthalen-2-ylmethyl-piperazine-2,5-dione,
(S,S)-6-(4-amino-butyl)-3-naphthalen-2-ylmethyl-1-(4′-trifluoromethyl-biphenyl-4-ylmethyl)piperazine-2,5-dione,
(S,S)-6-(4-amino-butyl)-1-(4′-chloro-biphenyl-4-ylmethyl)-3-naphthalen-2-ylmethyl-piperazine-2,5-dione,
(S,S)-6-(4-amino-butyl)-1-(9H-fluoren-2-ylmethyl)-3-naphthalen-2-ylmethyl-piperazine-2,5-dione,
(S,S)-4′-[2-(4-amino-butyl)-5-naphthalen-2-ylmethyl-3,6-dioxo-piperazin- -ylmethyl]-biphenyl-2-carboxylic acid methyl,
(S,S)-6-(4-amino-butyl)-3-(4-benzoyl-benzyl)-1-(4-phenoxy-benzyl)-piperazine-2,5-dione,
(S,S)-6-(4-amino-butyl)-3-(4-methoxy-benzyl)-1-(4-phenoxy-benzyl)-piperazine-2,5-dione,
(S,S)-6-(4-amino-butyl)-3-(4-chloro-benzyl)-1-(4-phenoxy-benzyl)-piperazine-2,5-dione,
(S,S)-6-(4-amino-butyl)-3-(4-methyl-benzyl)-1-(4-phenoxy-benzyl)-piperazine-2,5-dione,
(S,S)-4′-[2-(4-amino-butyl)-5-naphthalen-2-ylmethyl-3,6-dioxo-piperazin-1-ylmethyl]-biphenyl-2-carbonitrile,
(S,S)-6-(4-amino-butyl)-1-(4-cyclohexyloxy-benzyl)-3-naphthalen-2-ylmethyl-piperazine-2,5-dione,
(S,S)-6-(4-amino-butyl)-3-naphthalen-2-ylmethyl-1-[4-(3-trifluoromethyl-cyclohexyloxy)-benzyl]-piperazine-2,5-dione,
(S,S)-6-(4-amino-butyl)-1-(4-cyclohexyl-benzyl)-3-naphthalen-2-ylmethyl-piperazine-2,5-dione,
(S,S)-1-biphenyl-4-ylmethyl-6-(4-dimethylamino-butyl)-3-naphthalen-2-ylmethyl-piperazine-2,5-dione,
(S,S)-1-biphenyl-4-ylmethyl-6-(4-methylamino-butyl)-3-naphthalen-2-ylmethyl-piperazine-2,5-dione,
(S,S)-6-(4-amino-butyl)-3-(4-ethoxy-benzyl)-1-(4-phenoxy-benzyl)-piperazine-2,5-dione,
(S,S)-6-(4-amino-butyl)-1-biphenyl-4-ylmethyl-3-(4-propoxy-benzyl)-piperazine-2,5-dione,
(S,S)-6-(4-amino-butyl)-1-biphenyl-4-ylmethyl-3-(4-isopropoxy-benzyl)-piperazine-2,5-dione,
(S,S)-6-(4-amino-butyl)-1-(4-phenoxy-benzyl)-3-(4-pyrrol-1-yl-benzyl)-piperazine-2,5-dione,
(S,S)-6-(4-amino-butyl)-1-biphenyl-4-ylmethyl-3-(4-cyclopropylmethoxy-benzyl)-piperazine-2,5-dione,
(S,S)-6-(4-amino-butyl)-1-biphenyl-4-ylmethyl-3-(4-cyclohexyloxy-benzyl)-piperazine-2,5-dione,
(S,S)-1-biphenyl-4-ylmethyl-6-(4-isopropylamino-butyl)-3-naphthalen-2-ylmethyl-piperazine-2,5-dione,
(S,S)-6-(4-amino-butyl)-1-biphenyl-4-ylmethyl-3-(4-phenoxy-benzyl)-piperazine-2,5-dione,
(S,S)-6-(4-amino-butyl)-1-biphenyl-4-ylmethyl-3-(4-m-tolyloxy-benzyl)-piperazine-2,5-dione,
(S,S)-6-(4-amino-butyl)-1-biphenyl-4-ylmethyl-3-[4-(4-methoxy-phenoxy)-benzyl]-piperazine-2,5-dione,
(S,S)-6-(4-amino-butyl)-1-[4-(4-dimethylamino-phenoxy)-benzyl]-3-naphthalen-2-ylmethyl-piperazine-2,5-dione,
(S,S)-6-(4-amino-butyl)-1-[4-(4-methoxy-phenoxy)-benzyl]-3-naphthalen-2-ylmethyl-piperazine-2,5-dione,
(S,S)-1-[4-(3-acetyl-phenoxy)-benzyl]-6-(4-amino-butyl)-3-naphthalen-2-ylmethyl-piperazine-2,5-dione,
(S,S)-6-(4-amino-butyl)-1-[4-(4-ethanesulfonyl-phenoxy)-benzyl]-3-naphthalen-2-ylmethyl-piperazine-2,5-dione,
(S,S)-6-(4-amino-butyl)-1-biphenyl-4-ylmethyl-3-[4-(4-chloro-phenoxy)-benzyl]-piperazine-2,5-dione,
(S,S)-3-[4-(4-acetyl-phenoxy)-benzyl]-6-(4-amino-butyl)-1-biphenyl-4-ylmethyl-piperazine-2,5-dione,
(S,S)-3-[4-(3-acetyl-phenoxy)-benzyl]-6-(4-amino-butyl)-1-biphenyl-4-ylmethyl-piperazine-2,5-dione,
(S,S)-6-(4-amino-butyl)-1-biphenyl-4-ylmethyl-3-(4-methoxy-benzyl)-piperazine-2,5-dione,
(S,S)-6-(4-amino-butyl)-1-biphenyl-4-ylmethyl-3-(4-ethoxy-benzyl)-piperazine-2,5-dione,
(S,S)-6-(4-amino-butyl)-1-biphenyl-4-ylmethyl-3-[4-(3-trifluoromethoxy-phenoxy)-benzyl]-piperazine-2,5-dione,
(S,S)-6-(4-amino-butyl)-1-biphenyl-4-ylmethyl-3-[4-(4-fluoro-phenoxy)-benzyl]-piperazine-2,5-dione,
(S,S)-6-(4-amino-butyl)-1-biphenyl-4-ylmethyl-3-[4-(3-nitro-phenoxy)-benzyl]-piperazine-2,5-dione,
(S,S)-6-(4-amino-butyl)-1-(4-phenoxy-benzyl)-3-(4-propoxy-benzyl)-piperazine-2,5-dione,
(S,S)-6-(4-amino-butyl)-1-biphenyl-4-ylmethyl-3-[4-(pyridin-3-yloxy)-benzyl]-piperazine-2,5-dione,
(S,S)-6-(4-amino-butyl)-1-biphenyl-4-ylmethyl-3-[4-(4-dimethylamino-phenoxy)-benzyl]-piperazine-2,5-dione,
(S,S)-6-(4-amino-butyl)-3-naphthalen-2-ylmethyl-1-(6-phenyl-pyridin-3-ylmethyl)-piperazine-2,5-dione,
(S,S)-3-{4-[5-(4-amino-butyl)-4-biphenyl-4-ylmethyl-3,6-dioxo-piperazin-2-ylmethyl]-phenoxy}-benzaldehyde,
(S,S)-6-(4-amino-butyl)-1-(4-bromo-benzyl)-3-naphthalen-2-ylmethyl-piperazine-2,5-dione,
(S,S)-6-(4-amino-butyl)-3-(4-isopropoxy-benzyl)-1-(4-phenoxy-benzyl)-piperazine-2,5-dione,
(S,S)-6-[4-(2-amino-ethylamino)-butyl]-1-(4-phenoxy-benzyl)-3-(4-propoxy-benzyl)-piperazine-2,5-dione,
(S,S)-3-amino-N-(1-biphenyl-4-ylmethyl-5-naphthalen-2-ylmethyl-3,6-dioxo-piperazin-2-ylmethyl)-3-methyl-N-piperidin-4-ylmethyl-butyramide,
(S,S)-3-amino-N-(1-biphenyl-4-ylmethyl-5-naphthalen-2-ylmethyl-3,6-dioxo-piperazin-2-ylmethyl)-N-pyridin-4-ylmethyl-propionamide,
(S,S)-3-amino-N-[5-(4-ethoxy-benzyl)-3,6-dioxo-1-(4-phenoxy-benzyl)-piperazin-2-ylmethyl]-3-methyl-N-piperidin-4-ylmethyl-butyramide,
(S,S)-3-amino-N-[5-(4-ethoxy-benzyl)-3,6-dioxo-1-(4-phenoxy-benzyl)-piperazin-2-ylmethyl]-N-piperidin-4-ylmethyl-propionamide,
(S,S)-6-{[bis-(3H-imidazol-4-ylmethyl)-amino]-methyl}-3-(4-ethoxy-benzyl)-1-(4-phenoxy-benzyl)-piperazine-2,5-dione,
(S,S)-3-amino-N-(2-amino-2-methyl-propyl)-N-[5-(4-ethoxy-benzyl)-3,6-dioxo-1-(4-phenoxy-benzyl)-piperazin-2-ylmethyl]-3-methyl-butyramide,
(S,S)-1-[4-(4-acetyl-phenoxy)-benzyl]-6-(4-amino-butyl)-3-naphthalen-2-ylmethyl-piperazine-2,5-dione,
(S,S)-6-(4-amino-butyl)-1-biphenyl-4-ylmethyl-3-[4-(3-hydroxymethyl-phenoxy)-benzyl]-piperazine-2,5-dione,
(S,S)-6-{4-[(1H-imidazol-2-ylmethyl)-amino]-butyl}-3-(4-methoxy-benzyl)-1-(4-phenoxy-benzyl)-piperazine-2,5-dione,
(S,S)-3-(4-methoxy-benzyl)-1-(4-phenoxy-benzyl)-6-{4-[(pyridin-2-ylmethyl)-amino]-butyl}-piperazine-2,5-dione,
R,2′S, 5′S)-2-amino-N-[5-(4-ethoxy-benzyl)-3,6-dioxo-1-(4-phenoxy-benzyl)-piperazin-2-ylmethyl]-3-(1H-imidazol-4-yl)-propionamide,
(S,S)-2-(3-amino-propylamino)-N-[1-[4-(methyl-phenyl-amino)-benzyl]-3,6-dioxo-5-(4-propoxy-benzyl)-piperazin-2-ylmethyl]-acetamide,
N-[4-((2S,5S)-1-biphenyl-4-ylmethyl-5-naphthalen-2-ylmethyl-3,6-dioxo-piperazin-2-y)-butyl]-acetamide,
S,6S)-1-biphenyl-4-ylmethyl-6-(4-dimethylamino-butyl)-3-naphthalen-2-ylmethyl-piperazine-2,5-dione,
N-[4-((2S,5S)-1-biphenyl-4-ylmethyl-5-naphthalen-2-ylmethyl-3,6-dioxo-piperazin-2-y)-butyl]-guanidine hydrochloride,
S,6S)-6-[4-(3-amino-pyridin-2-ylamino)-butyl]-3-naphthalen-2-ylmethyl-1-(4-phenoxy-benzyl)-piperazine-2,5-dione,
{4-[(2S,5S)-5-naphthalen-2-ylmethyl-3,6-dioxo-1-(4-phenoxy-benzyl)-piperazin-2-yl]-butylamino}-acetonitrile,
N-((2S,5S)-1-biphenyl-4-ylmethyl-5-naphthalen-2-ylmethyl-3,6-dioxo-piperazin-2-ylmethyl)-N-piperidin-4-ylmethyl-acetamide,
(3S,6S)-1-biphenyl-4-ylmethyl-6-[(cyclohexylmethyl-piperidin-4-ylmethyl-amino)-methyl]-3-naphthalen-2-ylmethyl-piperazine-2,5-dione,
(3S,6S)-1-biphenyl-4-ylmethyl-6-[(ethyl-piperidin-4-ylmethyl-amino)-methyl]-3-naphthalen-2-ylmethyl-piperazine-2,5-dione,
(3S,6S)-1-biphenyl-4-ylmethyl-3-naphthalen-2-ylmethyl-6-[(piperidin-4-ylmethyl-pyridin-4-ylmethyl-amino)-methyl]-piperazine-2,5-dione,
3-amino-N-((2S,5S)-1-biphenyl-4-ylmethyl-5-naphthalen-2-ylmethyl-3,6-dioxo-piperazin-2-ylmethyl)-N-piperidin-4-ylmethyl-propionamide,
4-{[((2S,5S)-1-biphenyl-4-ylmethyl-5-naphthalen-2-ylmethyl-3,6-dioxo-piperazin-2-ylmethyl)-(piperidine-4-carbonyl)-amino]-methyl}-piperidine-1-carboxylic acid benzyl ester,
4-{[((2S,5S)-1-biphenyl-4-ylmethyl-5-naphthalen-2-ylmethyl-3,6-dioxo-piperazin-2-ylmethyl)-((R,S)-piperidine-3-carbonyl)-amino]-methyl}-piperidine-1-carboxylic acid benzyl ester,
piperidine-4-carboxylic acid ((2S,5S)-1-biphenyl-4-ylmethyl-5-naphthalen-2-ylmethyl-3,6-dioxo-piperazin-2-ylmethyl)-piperidin-4-ylmethyl-amide,
(R, S)-piperidine-3-carboxylic acid ((2S,5S)-1-biphenyl-4-ylmethyl-5-naphthalen-2-ylmethyl-3,6-dioxo-piperazin-2-ylmethyl)-piperidin-4-ylmethyl-amide,
4-amino-N-((2S,5S)-1-biphenyl-4-ylmethyl-5-naphthalen-2-ylmethyl-3,6-dioxo-piperazin-2-ylmethyl)-N-piperidin-4-ylmethyl-butyramide,
(3S,6S)-6-{([(3-amino-propyl)-piperidin-4-ylmethyl-amino]-methyl}-1-biphenyl-4-ylmethyl-3-naphthalen-2-ylmethyl-piperazine-2,5-dione,
1H-imidazole-4-carboxylic acid [(2S,5S)-5-naphthalen-2-ylmethyl-3,6-dioxo-1-(4-phenoxy-benzyl)-piperazin-2-ylmethyl]-piperidin-4-ylmethyl-amide,
2-amino-N-[(2S,5S)-5-naphthalen-2-ylmethyl-3,6-dioxo-1-(4-phenoxy-benzyl)-piperazin-2-ylmethyl]-N-piperidin-4-ylmethyl-acetamide,
3-amino-N-[(2S,5S)-5-naphthalen-2-ylmethyl-3,6-dioxo-1-(4-phenoxy-benzyl)-piperazin-2-ylmethyl]-N-piperidin-4-ylmethyl-propionamide,
N-[(2S,5 S)-5-naphthalen-2-ylmethyl-3,6-dioxo-1-(4-phenoxy-benzyl)-piperazin-2-ylmethyl]-2-piperidin-4-yl-N-piperidin-4-ylmethyl-acetamide,
(R,S)-2,5-.diamino-pentanoic acid [(2S,5S)-5-naphthalen-2-ylmethyl-3,6-dioxo-1-(4-phenoxy-benzyl)-piperazin-2-ylmethyl]-piperidin-4-ylmethyl-amide,
(3S,6S)-6-{[(3-dimethylamino-propyl)-piperidin-4-ylmethyl-amino]-methyl}-3-naphthalen-2-ylmethyl-1-(4-phenoxy-benzyl)-piperazine-2,5-dione,
3-amino-N-(1-methyl-piperidin-4-ylmethyl)-N-[(2S,5S)-5-naphthalen-2-ylmethyl-3,6-dioxo-1-(4-phenoxy-benzyl)-piperazin-2-ylmethyl]-propionamide,
piperidine-3-carboxylic acid [(2S,5S)-5-naphthalen-2-ylmethyl-3,6-dioxo-1-(4-phenoxy-benzyl)-piperazin-2-ylmethyl]-piperidin-4-ylmethyl-amide,
(3S,6S)-1-biphenyl-4-ylmethyl-6-{[bis-(1-methyl-piperidin-4-ylmethyl)-amino]-methyl}-3-naphthalen-2-ylmethyl-piperazine-2,5-dione,
(3S,6S)-6-{[(3-amino-propyl)-piperidin-4-ylmethyl-amino]-methyl}-1-(4-phenoxy-benzyl)-3-(4-trifluoromethyl-benzyl)-piperazine-2,5-dione,
(3S,6S)-6-{[(3-hydroxy-propyl)-piperidin-4-ylmethyl-amino]-methyl}-1-(4-phenoxy-benzyl)-3-(4-trifluoromethyl-benzyl)-piperazine-2,5-dione,
3-amino-N-[(2S,5S)-3,6-dioxo-1-(4-phenoxy-benzyl)-5-(4-trifluoromethyl-benzyl)-piperazin-2-ylmethyl]-N-piperidin-4-ylmethyl-propionamide,
N-((2S5S)—-biphenyl-4-ylmethyl-5-naphthalen-2-ylmethyl-3,6-dioxo-piperazin-2-ylmethyl)-2-(R, S)-morpholin-2-yl-N-piperidin-4-ylmethyl-acetamide,
(3S,6S)-1-biphenyl-4-ylmethyl-3-naphthalen-2-ylmethyl-6-[(piperidin-4-ylmethyl-pyridin-3-ylmethyl-amino)-methyl]-piperazine-2,5-dione,
(3S,6S)-1-(4-phenoxy-benzyl)-6-[(piperidin-4-ylmethyl-pyridin-3-ylmethyl-amino)-methyl]-3-(4-trifluoromethyl-benzyl)-piperazine-2,5-dione,
N-((2S,5S)-1-biphenyl-4-ylmethyl-5-naphthalen-2-ylmethyl-3,6-dioxo-piperazin-2-ylmethyl)-2-cyclopropylamino-N-piperidin-4-ylmethyl-acetamide,
N-((2S,5S)-1-biphenyl-4-ylmethyl-5-naphthalen-2-ylmethyl-3,6-dioxo-piperazin-2-ylmethyl)-N-piperidin-4-ylmethyl-2-(2,2,2-trifluoro-ethylamino)-acetamide,
N-((2S,5S)-1-biphenyl-4-ylmethyl-5-naphthalen-2-ylmethyl-3,6-dioxo-piperazin-2-ylmethyl)-2-imidazol-1-yl-N-piperidin-4-ylmethyl-acetamide,
2-[((2S,5S)-1-biphenyl-4-ylmethyl-5-naphthalen-2-ylmethyl-3,6-dioxo-piperazin-2-ylmethyl)-piperidin-4-ylmethyl-amino]-acetamide,
N-((2S,5S)-1-biphenyl-4-ylmethyl-5-naphthalen-2-ylmethyl-3,6-dioxo-piperazin-2-ylmethyl)-N-piperidin-4-ylmethyl-2-pyridin-3-yl-acetamide,
N-((2S,5S)-1-biphenyl-4-ylmethyl-5-naphthalen-2-ylmethyl-3,6-dioxo-piperazin-2-ylmethyl)-N-piperidin-4-ylmethyl-nicotinamide,
N-((2S,5S)-1-biphenyl-4-ylmethyl-5-naphthalen-2-ylmethyl-3,6-dioxo-piperazin-2-ylmethyl)-N-piperidin-4-ylmethyl-2-pyrrolidin-1-yl-acetamide,
3-amino-N-((2S,5S)-1-biphenyl-4-ylmethyl-5-naphthalen-2-ylmethyl-3,6-dioxo-piperazin-2-ylmethyl)-N-pyridin-3-ylmethyl-propionamide,
(S,S)-6-(4-Amino-butyl)-3-(3-chloro-4-methoxy-benzyl)-1-(4-phenoxy-benzyl)-piperazine-2,5-dione,
(S,S)-6-(4-Amino-butyl)-1-biphenyl-4-ylmethyl-3-(1-methoxy-naphthalen-2-ylmethyl)-piperazine-2,5-dione,
(S,S)-6-(4-Amino-butyl)-1-biphenyl-4-ylmethyl-3-(6-chloro-naphthalen-2-ylmethyl)-piperazine-2,5-dione,
(S,S)-6-(4-Amino-butyl)-3-(4-amino-3,5-dibromo-benzyl)-1-(4-phenoxy-benzyl)-piperazine-2,5-dione,
(S,S)-6-(4-Amino-butyl)-3-(4-hydroxy-3,5-dibromo-benzyl)-1-(4-phenoxy-benzyl)-piperazine-2,5-dione,
(S,S)-6-(4-Amino-butyl)-3-naphthalen-2-ylmethyl-1-[4-(pyridin-4-yloxy)-benzyl]-piperazine-2,5-dione,
(S,S)-6-(4-Amino-butyl)-1-(4-phenoxy-benzyl)-3-(5,6,7,8-tetrahydro-naphthalen-2-ylmethyl)-piperazine-2,5-dione,
(S,S)-6-(4-Amino-butyl)-3-naphthalen-2-ylmethyl-1-(4-o-tolyloxy-benzyl)-piperazine-2,5-dione,
(S,S)-6-(4-Amino-butyl)-1-[4-(3-chloro-phenoxy)-benzyl]-3-naphthalen-2-ylmethyl-piperazine-2,5-dione,
(S,S)-6-(4-Amino-butyl)-1-biphenyl-4-ylmethyl-3-(3-methoxy-naphthalen-2-ylmethyl)-piperazine-2,5-dione,
(S,S)-6-(4-Amino-butyl)-1-[4-(tert-butyl-diphenyl-silanyloxy)-benzyl]-3-naphthalen-2-ylmethyl-piperazine-2,5-dione (exp 15)
(S,S)-Carbonic acid 4-[2-(4-amino-butyl)-5-naphthalen-2-ylmethyl-3,6-dioxo-piperazin-1-ylmethyl]-phenyl ester benzyl ester,
(S,S)-6-(4-Amino-butyl)-1-[4-(methyl-phenyl-amino)-benzyl]-3-naphthalen-2-ylmethyl-piperazine-2,5-dione,
(S,S)-6-(4-Amino-butyl)-1-(4-benzyl-benzyl)-3-naphthalen-2-ylmethyl-piperazine-2,5-dione,
(S,S)-6-(4-Amino-butyl)-1-(3-methyl-4-phenoxy-benzyl)-3-naphthalen-2-ylmethyl-piperazine-2,5-dione,
(S,S)-6-(4-Amino-butyl)-1-(3-methoxy-4-phenoxy-benzyl)-3-naphthalen-2-ylmethyl-piperazine-2,5-dione,
(S,S)-3-(4-Amino-butyl)-4-biphenyl-4-ylmethyl-1-methyl-6-naphthalen-2-ylmethyl-piperazine-2,5-dione,
(S,S)-6-(5-Amino-pentyl)-1-biphenyl-4-ylmethyl-3-naphthalen-2-ylmethyl-piperazine-2,5-dione,
(S,S)-6-(4-Amino-butyl)-3-naphthalen-2-ylmethyl-1-(3-phenoxy-benzyl)-piperazine-2,5-dione,
(S,S)-6-(4-Amino-butyl)-3-(4-benzyloxy-benzyl)-1-(3-phenoxy-benzyl)-piperazine-2,5-dione,
(S,S)-6-(4-Amino-butyl)-3-naphthalen-1-ylmethyl-1-(3-phenoxy-benzyl)-piperazine-2,5-dione,
(S,S)-6-(4-Amino-butyl)-3-biphenyl-4-ylmethyl-1-(3-phenoxy-benzyl)-piperazine-2,5-dione,
(S,S)-6-(5-Amino-pentyl)-3-(4-benzyloxy-benzyl)-1-biphenyl-4-ylmethyl-piperazine-2,5-dione,
(S,S)-6-(5-Amino-pentyl)-1,3-bis-(4-benzyloxy-benzyl)-piperazine-2,5-dione,
(S,S)-6-(5-Amino-pentyl)-1-(4-benzyloxy-benzyl)-3-naphthalen-1-ylmethyl-piperazine-2,5-dione,
(S,S)-6-(5-Amino-pentyl)-1-(4-benzyloxy-benzyl)-3-biphenyl-4-ylmethyl-piperazine-2,5-dione,
(S,S)-6-(4-Amino-butyl)-3-(3,4-dichloro-benzyl)-1-(4-phenoxy-benzyl)-piperazine-2,5-dione,
(S,S)-6-(4-Amino-butyl)-3-naphthalen-1-ylmethyl-1-(4-phenoxy-benzyl)-piperazine-2,5-dione,
(S,S)-6-(4-Amino-butyl)-1-(9H-fluoren-3-ylmethyl)-3-naphthalen-1-ylmethyl-piperazine-2,5-dione, or
(S,S)-6-(4-Amino-butyl)-1-(4-benzyloxy-benzyl)-3-naphthalen-1-ylmethyl-piperazine-2,5-dione.
   125. A compound according to any of aspects 1 to 124, wherein the compound is an agonist of the MC4 receptor.
   126. A compound according to aspect 125, wherein the compound is selective for the MC4 receptor.
   127. A pharmaceutical composition comprising a compound according to any of aspects 1 to 126.
   128. Use of a compound according to any of aspects 1 to 126 for the preparation of a medicament.
   129. Use of a compound according to any of aspects 1 to 126 for increasing the activity of the MC4 receptor.
   130. Use of a compound according to any of aspects 1 to 126 for the delaying or prevention of the progression from IGT to type 2 diabetes.
   131. Use of a compound according to any of aspects 1 to 126 for the preparation of a medicament for the delaying or prevention of the progression from IGT to type 2 diabetes.
   132. Use of a compound according to any of aspects 1 to 126 for the delaying or prevention of the progression from non-insulin requiring type 2 diabetes to insulin requiring type 2 diabetes.
   133. Use of a compound according to any of aspects 1 to 126 for the preparation of a medicament for the delaying or prevention of the progression from non-insulin requiring type 2 diabetes to insulin requiring type 2 diabetes.
   134. Use of a compound according to any of aspects 1 to 126 for appetite regulation.
   135. Use of a compound according to any of aspects 1 to 126 for treating a condition which is improved by the activation of the MC4 receptor.
   136. Use of a compound according to any of aspects 1 to 126 for the preparation of a medicament for treating a condition which is improved by the activation of the MC4 receptor.
   137. Use of a compound according to any of aspects 1 to 126 for the preparation of a medicament for appetite regulation.
   138. Use according to aspect 135 or aspect 136, where the condition to be treated is a disease or condition related to overweight or obesity.
   139. Use according to aspect 135 or aspect 136, where the condition to be treated is a disease or condition selected from overweight or obesity, atherosclerosis, hypertension, diabetes, type 2 diabetes, impaired glucose tolerance, dyslipidaemia, coronary heart disease, gallbladder disease, osteoarthritis, cancer, sexual dysfunction and the risk for premature death in a patient in need thereof.
   140. Use according to aspect 139, wherein the disease is overweight or obesity.
   141. Use according to aspect 139, wherein the disease is type 2 diabetes.
   142. Use according to aspect 141, wherein the patient in need thereof is obese.
   143. Use according to aspect 139, wherein the disease is dyslipidemia.
   144. Use according to aspect 143, wherein the patient in need thereof is obese.
   145. Use according to aspect 139, wherein the condition is sexual dysfunction.
   146. Use of a compound according to any of aspects 1 to 126 for reducing the weight of a subject.
   147. Use of a compound according to any of aspects 1 to 126 for the preparation of a medicament for reducing the weight of a subject.
   148. Use according to aspect 146 or aspect 147, wherein the subject is a mammal.
   149. Use according to aspect 148, wherein the subject is a human.
   150. Use of a compound according to any of aspects 1 to 126 for the suppression of appetite or for satiety induction.
   151. Use of a compound according to any of aspects 1 to 126 for the preparation of a medicament for the suppression of appetite or for satiety induction.
   152. Use of a compound according to any of aspects 1 to 126 for treatment of eating disorders such as bulimia and binge eating.
   153. Use of a compound according to any of aspects 1 to 126 for the preparation of a medicament for treatment of eating disorders such as bulimia and binge eating.
   154. A method for the treatment of a condition which is improved by the activation of the MC4 receptor, the method comprising administering to a subject in need thereof a therapeutically effective amount of a compound according to any of aspects 1 to 126.
   155. A method for the treatment of hyperglycemia, the method comprising administering to a subject in need thereof a therapeutically effective amount of a compound according to any of aspects 1 to 126.
   156. A method for the treatment of IGT, the method comprising administering to a subject in need thereof a therapeutically effective amount of a compound according to any of aspects 1 to 126.
   157. A method for the treatment of Syndrome X, the method comprising administering to a subject in need thereof a therapeutically effective amount of a compound according to any of aspects 1 to 126.
   158. A method for the treatment of type 2 diabetes, the method comprising administering to a subject in need thereof a therapeutically effective amount of a compound according to any of aspects 1 to 126.
   159. A method for the treatment of type 1 diabetes, the method comprising administering to a subject in need thereof a therapeutically effective amount of a compound according to any of aspects 1 to 126.
   160. A method for the treatment of dyslipidemia or hyperlipidemia, the method comprising administering to a subject in need thereof a therapeutically effective amount of a compound according to any of aspects 1 to 126.
   161. A method for the treatment of sexual dysfunction, the method comprising administering to a subject in need thereof a therapeutically effective amount of a compound according to any of aspects 1 to 126.
   162. A method for reducing the weight of a subject, the method comprising administering to a subject in need thereof a therapeutically effective amount of a compound according to any of aspects 1 to 126.
   163. A method for the suppression of appetite or for satiety induction, the method comprising administering to a subject in need thereof a therapeutically effective amount of a compound according to any of aspects 1 to 126.
   164. A method for treatment of eating disorders such as bulimia and binge eating, the method comprising administering to a subject in need thereof a therapeutically effective amount of a compound according to any of aspects 1 to 126.
   165. A method for treating a disease or condition related to overweight or obesity, the method comprising administering to a subject in need thereof a therapeutically effective amount of a compound according to any of aspects 1 to 126.
   166. A method for the treatment of overweight or obesity, the method comprising administering to a subject in need thereof a therapeutically effective amount of a compound according to any of aspects 1 to 126.
   167. A method according to any of aspects 155 to 166, wherein the therapeutically effective amount of the compound is in the range of from about 0.05 mg to about 2000 mg, such as from about 0.1 mg to about 1000 mg, for example from about 0.5 mg to about 500 mg per day.
   168. A method according to any of aspects 155 to 167 in a regimen which comprises treatment with a further antidiabetic agent.
   169. A method according to any of aspects 155 to 168 in a regimen which comprises treatment with a further antihyperlipidemic agent.
   170. A method according to any of aspects 155 to 169 in a regimen which comprises treatment with a further antiobesity agent.
   171. A method according to any of aspects 155 to 170 in a regimen which comprises treatment with a further antihypertensive agent.
   172. A compound according to any of aspects 1 to 123, where the compound is an agonist of the MC1 receptor.
   173. A compound according to aspect 172, wherein the compound is selective for the MC1 receptor.
   174. A pharmaceutical composition comprising a compound according to aspect 172 or aspect 173.
   175. Use of a compound according to any of aspects 1 to 123 or aspect 172 or aspect 173 for increasing the activity of the MC1 receptor.
   176. Use of a compound according to any of aspects 1 to 123 or aspect 172 or aspect 173 for treating a condition which is improved by the activation of the MC1 receptor.
   177. Use of a compound according to any of aspects 1 to 123 or aspect 172 or aspect 173 for the preparation of a medicament for treating a condition which is improved by the activation of the MC1 receptor.
   178. Use of a compound according to any of aspects 1 to 123 or aspect 172 or aspect 173 for increasing skin pigmentation, for protecting the skin against ultraviolet radiation (UVR), for inhibiting the effects of UVR, for protecting the skin against local skin irritants, for modulating the inflammatory responses in the skin, for functionally antagonising the actions of proinflammatory cytokines produced in the skin after a local irritation, for regulating the immune response, for preventing contact dermatitis, or for inhibiting chronic inflammatory responses.
   179. Use of a compound according to any of aspects 1 to 123 or aspect 172 or aspect 173 for the preparation of a medicament for increasing skin pigmentation, for protecting the skin against ultraviolet radiation (UVR), for inhibiting the effects of UVR, for protecting the skin against local skin irritants, for modulating the inflammatory responses in the skin, for functionally antagonising the actions of proinflammatory cytokines produced in the skin after a local irritation, for regulating the immune response, for preventing contact dermatitis, or for inhibiting chronic inflammatory responses.
   180. A method for the treatment of a condition which is improved by the activation of the MC1 receptor, the method comprising administering to a subject in need thereof a therapeutically effective amount of a compound according to any of aspects 1 to 123 or aspect 172 or aspect 173.
   181. A method for increasing skin pigmentation, for protecting the skin against ultraviolet radiation (UVR), for inhibiting the effects of UVR, for protecting the skin against local skin irritants, for modulating the inflammatory responses in the skin, for functionally antagonising the actions of proinflammatory cytokines produced in the skin after a local irritation, for regulating the immune response, for preventing contact dermatitis, or for inhibiting chronic inflammatory responses, the method comprising administering to a subject in need thereof a therapeutically effective amount of a compound according to any of aspects 1 to 123 or aspect 172 or aspect 173.
   182. A method according to aspect 180 or aspect 181, wherein the therapeutically effective amount of the compound is in the range of from about 0.05 mg to about 2000 mg, such as from about 0.1 mg to about 1000 mg, for example from about 0.5 mg to about 500 mg per day.
   183. A compound according to any of aspects 1 to 123, where the compound is an agonist of the MC2 receptor.
   184. A compound according to aspect 183, wherein the compound is selective for the MC2 receptor.
   185. A pharmaceutical composition comprising a compound according to aspect 183 or aspect 184.
   186. Use of a compound according to any of aspects 1 to 123 or aspect 183 or aspect 184 for increasing the activity of the MC2 receptor.
   187. Use of a compound according to any of aspects 1 to 123 or aspect 183 or aspect 184 for treating a condition which is improved by the activation of the MC2 receptor.
   188. Use of a compound according to any of aspects 1 to 123 or aspect 183 or aspect 184 for the preparation of a medicament for treating a condition which is improved by the activation of the MC2 receptor.
   189. Use of a compound according to any of aspects 1 to 123 or aspect 183 or aspect 184 for regulating glucocorticoid production.
   190. Use of a compound according to any of aspects 1 to 123 or aspect 183 or aspect 184 for the preparation of a medicament for regulating glucocorticoid production.
   191. A method for the treatment of a condition which is improved by the activation of the MC2 receptor, the method comprising administering to a subject in need thereof a therapeutically effective amount of a compound according to any of aspects 1 to 123 or aspect 183 or aspect 184.
   192. A method for regulating glucocorticoid production, the method comprising administering to a subject in need thereof a therapeutically effective amount of a compound according to any of aspects 1 to 123 or aspect 183 or aspect 184.
   193. A method according to aspect 191 or aspect 192, wherein the therapeutically effective amount of the compound is in the range of from about 0.05 mg to about 2000 mg, such as from about 0.1 mg to about 1000 mg, for example from about 0.5 mg to about 500 mg per day.
   194. A compound according to any of aspects 1 to 123, where the compound is an agonist of the MC3 receptor.
   195. A compound according to aspect 194, wherein the compound is selective for the MC3 receptor. 196. A pharmaceutical composition comprising a compound according to aspect 194 or aspect 195.
   197. Use of a compound according to any of aspects 1 to 123 or aspect 194 or aspect 195 for increasing the activity of the MC3 receptor.
   198. Use of a compound according to any of aspects 1 to 123 or aspect 194 or aspect 195 for treating a condition which is improved by the activation of the MC3 receptor.
   199. Use of a compound according to any of aspects 1 to 123 or aspect 194 or aspect 195 for the preparation of a medicament for treating a condition which is improved by the activation of the MC3 receptor.
   200. Use according to aspect 198 or aspect 199, wherein the condition to be treated is hypertension.
   201. Use according to aspect 198 or aspect 199, wherein the condition to be treated is overweight or obesity.
   202. Use according to aspect 198 or aspect 199, wherein the condition to be treated is sexual dysfunction.
   203. Use of a compound according to any of aspects 1 to 123 or aspect 194 or aspect 195 for reducing blood pressure and heart rate or for inducing natriuresis.
   204. Use of a compound according to any of aspects 1 to 123 or aspect 194 or aspect 195 for the preparation of a medicament for reducing blood pressure and heart rate or for inducing natriuresis.
   205. A method for the treatment of a condition which is improved by the activation of the MC3 receptor, the method comprising administering to a subject in need thereof a therapeutically effective amount of a compound according to any of aspects 1 to 123 or aspect 194 or aspect 195.
   206. A method for the treatment of hypertension, the method comprising administering to a subject in need thereof a therapeutically effective amount of a compound according to any of aspects 1 to 123 or aspect 194 or aspect 195.
   207. A method for the treatment of overweight or obesity, the method comprising administering to a subject in need thereof a therapeutically effective amount of a compound according to any of aspects 1 to 123 or aspect 194 or aspect 195.
   208. A method for the treatment of sexual dysfunction, the method comprising administering to a subject in need thereof a therapeutically effective amount of a compound according to any of aspects 1 to 123 or aspect 194 or aspect 195.
   209. A method according to any of aspects 205 to 208, wherein the therapeutically effective amount of the compound is in the range of from about 0.05 mg to about 2000 mg, such as from about 0.1 mg to about 1000 mg, for example from about 0.5 mg to about 500 mg per day.
   210. A compound according to any of aspects 1 to 123, where the compound is an agonist of the MC5 receptor.
   211. A compound according to aspect 210, wherein the compound is selective for the MC5 receptor.
   212. A pharmaceutical composition comprising a compound according to aspect 210 or aspect 211.
   213. Use of a compound according to any of aspects 1 to 123 or aspect 210 or aspect 211 for increasing the activity of the MC5 receptor.
   214. Use of a compound according to any of aspects 1 to 123 or aspect 210 or aspect 211 for treating a condition which is improved by the activation of the MC5 receptor.
   215. Use of a compound according to any of aspects 1 to 123 or aspect 210 or aspect 211 for the preparation of a medicament for treating a condition which is improved by the activation of the MC5 receptor.
   216. Use according to aspect 214 or aspect 215, wherein the condition to be treated is hypertension.
   217. Use of a compound according to any of aspects 1 to 123 or aspect 210 or aspect 211 for regulating exocrine gland secretion, for regulating aldosterone secretion, for suppressing stress-induced alarm substances, or for stimulating exocrine glands, cardiac and testicular functions.
   218. Use of a compound according to any of aspects 1 to 123 or aspect 210 or aspect 211 for the preparation of a medicament for regulating exocrine gland secretion, for regulating aldosterone secretion, for suppressing stress-induced alarm substances, or for stimulating exocrine glands, cardiac and testicular functions.
   219. A method for the treatment of a condition which is improved by the activation of the MC5 receptor, the method comprising administering to a subject in need thereof a therapeutically effective amount of a compound according to any of aspects 1 to 123 or aspect 210 or aspect 211.
   220. A method for treatment of hypertension, the method comprising administering to a subject in need thereof a therapeutically effective amount of a compound according to any of aspects 1 to 123 or aspect 210 or aspect 211.
   221. A method for regulating exocrine gland secretion, for regulating aldosterone secretion, for suppressing stress-induced alarm substances, or for stimulating exocrine glands, cardiac and testicular functions, the method comprising administering to a subject in need thereof a therapeutically effective amount of a compound according to any of aspects 1 to 123 or aspect 210 or aspect 211.
   222. A method according to any of aspects 219 to 221, wherein the therapeutically effective amount of the compound is in the range of from about 0.05 mg to about 2000 mg, such as from about 0.1 mg to about 1000 mg, for example from about 0.5 mg to about 500 mg per day.
   223. A compound according to any of aspects 1 to 123, where the compound is an agonist of the MC3 receptor and the MC4 receptor.
   224. A compound according to aspect 223, wherein the compound is selective for the MC3 and MC4 receptor.
   225. A pharmaceutical composition comprising a compound according to aspect 223 or aspect 224.
   226. Use of a compound according to aspect 223 or aspect 224 for increasing the activity of the MC3 receptor.
   227. Use of a compound according to aspect 223 or aspect 224 for increasing the activity of the MC4 receptor.
   228. Use according to aspect 226 or aspect 227 for increasing the activity of the MC3 receptor and increasing the activity of the MC4 receptor.
   229. Use of a compound according to aspect 223 or aspect 224 for treating a condition which is improved by the activation of the MC3 receptor.
   230. Use of a compound according to aspect 223 or aspect 224 for treating a condition which is improved by the activation of the MC4 receptor.
   231. Use of a compound according to aspect 223 or aspect 224 for treating a condition which is improved by the activation of the MC3 receptor and by the activation of the MC4 receptor.
   232. Use of a compound according to aspect 223 or aspect 224 for the preparation of a medicament for treating a condition which is improved by the activation of the MC3 receptor.
   233. Use of a compound according to aspect 223 or aspect 224 for the preparation of a medicament for treating a condition which is improved by the activation of the MC4 receptor.
   234. Use of a compound according to aspect 223 or aspect 224 for the preparation of a medicament for treating a condition which is improved by the activation of the MC3 receptor and by the activation of the MC4 receptor.
   235. Use according to any of aspects 229 to 234, wherein the condition to be treated is overweight or obesity.
   236. Use according to any of aspects 229 to 234, wherein the condition to be treated is sexual dysfunction.
   237. A method for the treatment of a condition which is improved by the activation of the MC3 receptor, the method comprising administering to a subject in need thereof a therapeutically effective amount of a compound according to aspect 223 or aspect 224.
   238. A method for the treatment of a condition which is improved by the activation of the MC4 receptor, the method comprising administering to a subject in need thereof a therapeutically effective amount of a compound according to aspect 223 or aspect 224.
   239. A method for the treatment of a condition which is improved by the activation of the MC3 receptor and by the activation of the MC4 receptor, the method comprising administering to a subject in need thereof a therapeutically effective amount of a compound according to aspect 223 or aspect 224.
   240. A method for the treatment of overweight or obesity, the method comprising administering to a subject in need thereof a therapeutically effective amount of a compound according to aspect 223 or aspect 224.
   241. A method for the treatment of sexual dysfunction, the method comprising administering to a subject in need thereof a therapeutically effective amount of a compound according to aspect 223 or aspect 224.
   242. A method according to any of aspects 237 to 241, wherein the therapeutically effective amount of the compound is in the range of from about 0.05 mg to about 2000 mg, such as from about 0.1 mg to about 1000 mg, for example from about 0.5 mg to about 500 mg per day.
   243. A compound according to any of aspects 1 to 123, where the compound is an agonist of the MC3 receptor and the MC5 receptor.
   244. A compound according to aspect 243, wherein the compound is selective for the MC3 and MC5 receptor.
   245. A pharmaceutical composition comprising a compound according to aspect 243 or aspect 244.
   246. Use of a compound according to aspect 243 or aspect 244 for increasing the activity of the MC3 receptor.
   247. Use of a compound according to aspect 243 or aspect 244 for increasing the activity of the MC5 receptor.
   248. Use according to aspect 246 or aspect 247 for increasing the activity of the MC3 receptor and increasing the activity of the MC5 receptor.
   249. Use of a compound according to aspect 243 or aspect 244 for treating a condition which is improved by the activation of the MC3 receptor.
   250. Use of a compound according to aspect 243 or aspect 244 for treating a condition which is improved by the activation of the MC5 receptor.
   251. Use of a compound according to aspect 243 or aspect 244 for treating a condition which is improved by the activation of the MC3 receptor and by the activation of the MC5 receptor.
   252. Use of a compound according to aspect 243 or aspect 244 for the preparation of a medicament for treating a condition which is improved by the activation of the MC3 receptor.
   253. Use of a compound according to aspect 243 or aspect 244 for the preparation of a medicament for treating a condition which is improved by the activation of the MC5 receptor.
   254. Use of a compound according to aspect 243 or aspect 244 for the preparation of a medicament for treating a condition which is improved by the activation of the MC3 receptor and by the activation of the MC5 receptor.
   255. Use according to any of aspects 249 to 254, wherein the condition to be treated is hypertension.
   256. A method for the treatment of a condition which is improved by the activation of the MC3 receptor, the method comprising administering to a subject in need thereof a therapeutically effective amount of a compound according to aspect 243 or aspect 244.
   257. A method for the treatment of a condition which is improved by the activation of the MC5 receptor, the method comprising administering to a subject in need thereof a therapeutically effective amount of a compound according to aspect 243 or aspect 244.
   258. A method for the treatment of a condition which is improved by the activation of the MC3 receptor and by the activation of the MC5 receptor, the method comprising administering to a subject in need thereof a therapeutically effective amount of a compound according to aspect 243 or aspect 244.
   259. A method for the treatment of hypertension, the method comprising administering to a subject in need thereof a therapeutically effective amount of a compound according to aspect 243 or aspect 244.
   260. A method according to any of aspects 256 to 259, wherein the therapeutically effective amount of the compound is in the range of from about 0.05 mg to about 2000 mg, such as from about 0.1 mg to about 1000 mg, for example from about 0.5 mg to about 500 mg per day.
   261. Use of a compound according to any of aspects 1 to 123 for increasing antipyretic activity.
   262. Use of a compound according to any of aspects 1 to 123 for the preparation of a medicament for increasing antipyretic activity.
   263. A method for increasing antipyretic activity, the method comprising administering to a subject in need thereof a therapeutically effective amount of a compound according to any of aspects 1 to 123.
   264. A method according to aspect 263, wherein the therapeutically effective amount of the compound is in the range of from about 0.05 mg to about 2000 mg, such as from about 0.1 mg to about 1000 mg, for example from about 0.5 mg to about 500 mg per day.
   265. Use of a compound according to any of aspects 1 to 123 for inducing lipolysis.
   266. Use of a compound according to any of aspects 1 to 123 for the preparation of a medicament for inducing lipolysis.
   267. A method for inducing lipolysis, the method comprising administering to a subject in need thereof a therapeutically effective amount of a compound according to any of aspects 1 to 123.
   268. A method according to aspect 267, wherein the therapeutically effective amount of the compound is in the range of from about 0.05 mg to about 2000 mg, such as from about 0.1 mg to about 1000 mg, for example from about 0.5 mg to about 500 mg per day.
   269. A method of promoting the sale and/or use of a compound according to any of aspects 1 to 123 comprising distributing information related to the use of the compound in the prevention or treatment of any condition or combination of conditions recited in any of the foregoing aspects.
   270. A pharmaceutical product comprising (a) a composition according to any of aspects 1 to 123, (b) a pharmaceutically acceptable carrier, excipient, or diluent, and (c) a notice associated with said container in form prescribed by a governmental agency regulating the manufacture, use, or sale of pharmaceuticals, which notice is reflective of approval by said agency of said pharmaceutical product for human or veterinary administration to treat at least one condition recited in any of the foregoing aspects.
   271. A compound comprising a chemical structure according to Formula (I).
   272. A compound comprising a chemical structure according to any one of aspects 1-123.
   273. Use of the compound of aspect 271 in any of the foregoing use and/or method aspects.
   274. Use of the compound of aspect 272 in any of the foregoing use and/or method aspects.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Unless explicitly indicated or clearly contradicted by context, approximate values described herein (e.g., “about 10”) can be replaced by corresponding exact values and visa versa.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out their invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A compound of the formula: 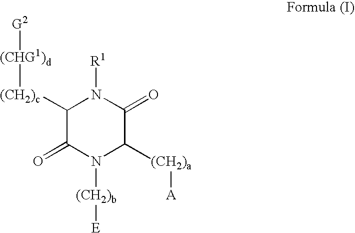
or an optical isomer, geometric isomer, diastereomer, tautomer, or a pharmaceutically acceptable salt thereof, wherein
A is —NR²R³ or guanidinyl, the last optionally substituted with C₁₋₆-alkyl, wherein
R² and R³ independently of each other are hydrogen, C₁₋₆-alkyl,
C₁₋₆-alkylene-N(R¹¹)(R¹²), C₁₋₆-alkylene-CN, C₁₋₆-alkylene-OH,
C₁₋₆-alkylene-C(O)—N(R¹¹)(R¹²), (Z¹)ₑ-R¹³, or —CO—R¹⁴, wherein
R¹¹ and R¹² independently of each other are hydrogen or C₁₋₆-alkyl;
Z¹ is C₁₋₆-alkylene;
e is an integer selected from 0 or 1;
R¹³ is cycloalkyl, heterocyclyl, aryl, or heteroaryl; each of which may be optionally substituted with a substitutent selected from the group consisting of C₁₋₆-alkyl, amino, and —CO—O-Z⁴-R²³, wherein
Z⁴ is C₁₋₁₆-alkylene; and
R²³ is aryl; and
R¹⁴ is hydrogen, C₁₋₆-alkyl, —N(R¹⁵)(R¹⁶), C₁₋₆-alkylene-N(R¹⁵)(R¹⁶),
C(R¹⁷)(R¹⁸)—N(R¹⁹)(R²⁰), heterocyclyl, (Z²)_{f}-R²¹, heteroaryl, or C₁₋₆-alkoxy, wherein
R¹⁵ and R¹⁶ independently of each other are hydrogen, or C₁₋₆-alkyl;
R¹⁷ and R¹⁸ independently of each other are hydrogen,
C₁₋₆-alkylene-NH₂ or (Z³)₉—R²²), wherein
Z³ is C₁₋₆-alkylene;
g is an integer selected from 0 or 1; and
R²² is cycloalkyl, heterocyclyl, aryl or heteroaryl;
R¹⁹ and R²⁰ independently of each other are hydrogen,
C₂₋₆-alkylene-NH₂, C₁₋₆-alkylene-CF₃ or cycloalkyl; and
Z² is C₁₋₆-alkylene;
f is an integer selected from 0 or 1; and
R²¹ is cycloalkyl, heterocyclyl, aryl or heteroaryl;
a is an integer selected from 1, 2, 3, 4, or 5;
E is cycloalkyl, heterocyclyl, aryl or heteroaryl; each of which may be optionally substituted with one or more substituents selected from the group consisting of halogen, hydroxy, cyano, nitro, —NR⁴R⁵, —CO—R⁶, C₁₋₆-alkyl, C₁₋₆-alkoxy, trifluoromethyl, trifluoromethoxy, and -L¹-Q¹, wherein
R⁴ and R⁵ independently of each other are hydrogen, C₁₋₆-alkyl, —CO—R²⁴, or aryl, wherein
R²⁴ is hydrogen, C₁₋₆-alkyl or C₁₋₆-alkoxy;
R⁶ is C₁₋₆-alkyl or C₁₋₆-alkoxy;
L¹ is a direct bond, —CH₂—, —O—, —CO—, —CH₂—O—, —O—CH₂— or —NR²⁵—, wherein
R²⁵ is hydrogen or C₁₋₆-alkyl; and
Q¹ is cycloalkyl, heterocyclyl, aryl or heteroaryl; each of which may be optionally substituted with one or more substituents selected from the group consisting of halogen, hydroxy, cyano, nitro, trifluoromethyl, trifluoromethoxy, —NR²⁶R²⁷, —CO—R²⁸, —S(O)₂—R²⁹, C₁₋₆-alkyl, C₁₋₆-alkoxy, C₃₋₇-cycloalkyl and C₃₋₇-cycloalkoxy, wherein
R²⁶ and R²⁷ independently of each other are hydrogen, C₁₋₆-alkyl, or —CO—R³⁰, wherein
R³⁰ is hydrogen, C₁₋₆-alkyl or C₁₋₆-alkoxy;
R²⁸ is C₁₋₆-alkyl or C₁₋₆-alkoxy; and
R²⁹ is C₁₋₆-alkyl, —NH—C₁₋₆-alkyl, or —N(C₁₋₆-alkyl)₂;
or
Q¹ is L³-R³¹, wherein
L³ is —CH₂—, —O—, —CO—, —CH₂—O—, —O—CH₂—, —CH₂—O—C(O)—, or —C(O)—O—CH₂—; and
R³¹ is aryl or heteroaryl;
b is an integer selected from 0, 1, or 2;
G¹ is C₁₋₆-alkyl, C₁₋₆-alkoxy, cycloalkyl, C₃₋₇-cycloalkoxy, aryl or heteroaryl; each of which may be optionally substituted with halogen, hydroxy, cyano, nitro, trifluoromethyl, trifluoromethoxy, —NR⁷R⁸, C₁₋₆-alkyl, C₁₋₆-alkoxy, C₃₋₇-cycloalkyl, C₃₋₇-cycloalkoxy, wherein
R⁷ and R⁸ independently of each other are hydrogen, C₁₋₆-alkyl, aryl, heteroaryl,
—CO—R³² or —SO₂—R³³, wherein
R³² is hydrogen, C₁₋₆-alkyl or C₁₋₆-alkoxy; and
R³³ is C₁₋₆-alkyl, —NH—C₁₋₆-alkyl, —N(C₁₋₆-alkyl)₂;
G² is cycloalkyl, heterocyclyl, aryl, or heteroaryl; each of which may be optionally substituted with one or more substituents selected from the group consisting of halogen, hydroxy, cyano, nitro, difluoromethyl, trifluoromethyl, difluoromethoxy, trifluoromethoxy, —NR⁹R¹⁰, C₁₋₆-alkyl, C₁₋₆-alkoxy, C₃₋₇-cycloalkyl, C₃₋₇-cycloalkoxy or -L²-Q², wherein
R⁹ and R¹⁰ are independently hydrogen, C₁₋₆-alkyl, aryl, heteroaryl, —CO—R³⁴ or —SO₂—R³, wherein
R³⁴ is hydrogen, C₁₋₆-alkyl or C₁₋₆-alkoxy; and
R³⁵ is C₁₋₆-alkyl, —NH—C₁₋₆-alkyl, or —N(C₁₋₆-alkyl)₂;
L² is a direct bond, —CH₂—, —O—, —CO—, —CH₂—O—, —O—CH₂— or —NR³⁶—, wherein
R³⁶ is hydrogen or C₁₋₆-alkyl; and
Q² is cycloalkyl, heterocyclyl, aryl or heteroaryl; each of which may be optionally substituted with halogen, hydroxy, cyano, nitro, trifluoromethyl, —NR³⁷R³⁸, —CO—R³⁹, —O—R⁴⁰, C₁₋₆-alkyl, C₁₋₆-hydroxyalkyl, C₃₋₇-cycloalkyl or C₃₋₇-cycloalkoxy, wherein
R³⁷ and R³⁸ independently of each other are hydrogen, C₁₋₆-alkyl or —CO—R⁴¹, wherein
R⁴¹ is hydrogen, C₁₋₆-alkyl or C₁₋₆-alkoxy;
R³⁹ is hydrogen, C₁₋₆-alkyl or C₁₋₆-alkoxy; and
R⁴⁰ is C₁₋₆-alkyl or trifluoromethyl;
c is an integer selected from 0, 1, or 2;
d is an integer selected from 0, or 1; and
R¹ is hydrogen, alkyl, alkenyl, or alkynyl.

2. The compound of claim 1, wherein A is —NR²R³.

3. The compound of claim 1, wherein R² is hydrogen, C₁₋₆-alkyl, C₁₋₆-alkylene-N(R¹¹)(R¹²), C₁₋₆-alkylene-CN, C₁₋₆-alkylene-OH, C₁₋₆-alkylene-C(O)—N(R¹¹)(R¹²), (Z¹)ₑ-R¹³, or —CO—R¹⁴; and R³ is hydrogen, C₁₋₆₆-alkyl, C₁₋₆-alkylene-N(R¹¹)(R¹²), (Z¹)ₑ-R¹³, or —CO—R¹⁴.

4. The compound of claim 1, wherein R² and R³ independently of each other are hydrogen, C₁₋₆-alkyl, C₁₋₆-alkylene-N(R¹¹)(R¹²), (Z¹)ₑ-R¹³, or —CO—R¹⁴.

5. The compound of claim 1, wherein R¹¹ and R¹² are hydrogen.

6. The compound of claim 1, wherein e is 1 and Z¹ is —CH₂—.

7. The compound of claim 1, wherein R¹³ is a cycloalkyl or aryl; either of which may be optionally substituted with C₁₋₆-alkyl, amino or —CO—O-Z⁴-R²³.

8. The compound of claim 1, wherein R²³ is C₆₋₁₃-aryl.

9. The compound of claim 1, wherein R² and R³ independently of each other are hydrogen, C₁₋₆-alkyl, or —CO—R¹⁴.

10. The compound of claim 1, wherein R¹⁴ is hydrogen, C₁₋₆-alkyl, —NR¹⁵R¹⁶, C₁₋₆-alkylene-N(R¹⁵)(R¹⁶), C(R¹⁷)(R¹⁸)—N(R¹⁹)(R²⁰), C₃₋₁₀-heterocyclyl, (Z²)_{f}-R²¹, C₅₋₁₄-heteroaryl, or C₁₋₆-alkoxy.

11. The compound of claim 1, wherein R¹⁵ and R¹⁶ are each hydrogen.

12. The compound of claim 1, wherein R¹⁷ and R¹⁸ independently of each other are hydrogen, a C₁₋₆-alkylene-NH₂, or (Z³)_{g}-R²².

13. The compound of claim 1, wherein R²² is C₃₋₁₂-cycloalkyl, C₃₋₁₀-heterocyclyl, C₆₋₁₃-aryl, or C₅₋₁₄-heteroaryl.

14. The compound of claim 1, wherein R¹⁷ and R¹³ are each hydrogen.

15. The compound of claim 1, wherein R¹⁹ and R²⁰ independently of each other are hydrogen, C₂₋₆-alkylene-NH₂, C₁₋₆-alkylene-CF₃, or C₃₋₇-cycloalkyl.

16. The compound of claim 1, wherein f is 1 and Z² is —CH₂—.

17. The compound of claim 1, wherein R²¹ is a heterocyclyl or heteroaryl.

18. The compound of claim 1, wherein A is guanidinyl optionally substituted with C₁₋₆-alkyl.

19. The compound of claim 1, wherein a is 1, 4, or 5.

20. The compound of claim 1, wherein A is —NR²R³, R² and R³ are hydrogen, and a is 4 or 5.

21. The compound of claim 1, wherein the sum of the carbon and nitrogen atoms in the —(CH₂)ₐ-A group is at least 4.

22. The compound of claim 1, wherein R² is C₃₋₆-alkyl, C₃₋₆-alkylene-N(R¹¹)(R¹²), C₃₋₆-alkylene-CN, C₃₋₆-alkylene-OH, C₃₋₆-alkylene-C(O)—N(R¹¹)(R¹²), (Z¹)ₑ-R¹³, or —CO—R¹⁴; and R³ is C₃₋₆-alkyl, C₃₋₆-alkylene-N(R¹¹)(R¹²), (Z¹)ₑ-R¹³, or —CO—R⁴; R¹⁴ is C₂₋₆-alkyl, C₂₋₆-alkylene-N(R¹⁵)(R¹⁶), C(R¹⁷)(R¹⁸)—N(R¹⁹)(R²⁰), heterocyclyl, (Z²)_{f}-R²¹, heteroaryl, C₂₋₆-alkoxy, or —N(R⁴²)(R⁴³), R¹⁵ and R¹⁶ independently of each other are hydrogen, or C₁₋₆-alkyl; R¹⁷ and R¹⁸ independently of each other are hydrogen, C₁₋₆-alkylene-NH₂ or (Z³)₉—R²²); Z³ is C₁₋₆-alkylene; g is an integer selected from 0 or 1; R²² is cycloalkyl, heterocyclyl, aryl or heteroaryl; R¹⁹ and R²⁰ independently of each other are hydrogen, C₂₋₆-alkylene-NH₂, C₁₋₆-alkylene-CF₃ or cycloalkyl; Z² is C₁₋₆-alkylene; f is an integer selected from 0 or 1; R²¹ is cycloalkyl, heterocyclyl, aryl or heteroaryl; and R⁴² and R⁴³ independently of each other are C₁₋₆-alkyl.

23. The compound of claim 1, wherein R² and R³ independently of each other are C₃₋₆-alkyl, C₃₋₆-alkylene-N(R¹¹)(R¹²), (Z¹)ₑ-R¹³, or —CO—R¹⁴; R¹⁴ is C₂₋₆-alkyl, C₂₋₆-alkylene-N(R¹⁵)(R¹⁶), C(R¹⁷)(R¹⁸)—N(R¹⁹)(R²⁰), heterocyclyl, (Z²)_{f}-R²¹, heteroaryl, C₂₋₆-alkoxy, or —N(R⁴²)(R⁴³); R¹⁵ and R¹⁶ independently of each other are hydrogen, or C₁₋₆-alkyl; R¹⁷ and R¹⁸ independently of each other are hydrogen, C₁₋₆-alkylene-NH₂ or (Z³)_{g}-R²²); Z³ is C₁₋₆-alkylene; g is an integer selected from 0 or 1; R²² is cycloalkyl, heterocyclyl, aryl or heteroaryl; R¹⁹ and R²⁰ independently of each other are hydrogen, C₂₋₆-alkylene-NH₂, C₁₋₆-alkylene-CF₃ or cycloalkyl; Z² is C₁₋₆-alkylene; f is an integer selected from 0 or 1; R²¹ is cycloalkyl, heterocyclyl, aryl or heteroaryl; and R⁴² and R⁴³ independently of each other are C₁₋₆-alkyl.

24. The compound of claim 1, wherein R² and R³ independently of each other are C₃₋₆-alkyl, C₃₋₆-alkylene-CN, C₃₋₆-alkylene-OH, C₃₋₆-alkylene-C(O)—NH₂, (Z¹)ₑ-R¹³, or —CO—R¹⁴; R¹⁴ is C₂₋₆-alkyl, C₂₋₆-alkylene-N(R¹⁵)(R¹⁶), C(R¹⁷)(R¹⁸)—N(R¹⁹)(R²⁰), heterocyclyl, (Z²)_{f}-R²¹, heteroaryl, C₂₋₆-alkoxy, or —N(R⁴²)(R⁴³); R¹⁵ and R¹⁶ independently of each other are hydrogen, or C₁₋₆-alkyl; R¹⁷ and R¹⁸ independently of each other are hydrogen, C₁₋₆-alkylene-NH₂ or (Z³)_{g}-R²²); Z³ is C₁₋₆-alkylene; g is an integer selected from 0 or 1; R²² is cycloalkyl, heterocyclyl, aryl or heteroaryl; R¹⁹ and R²⁰ independently of each other are hydrogen, C₂₋₆-alkylene-NH₂, C₁₋₆-alkylene-CF₃ or cycloalkyl; Z² is C₁₋₆-alkylene; f is an integer selected from 0 or 1; R²¹ is cycloalkyl, heterocyclyl, aryl or heteroaryl; and R⁴² and R⁴³ independently of each other are C₁₋₆-alkyl.

25. The compound of claim 1, wherein E is aryl or heteroaryl, each of which may be optionally substituted with one or more substituents selected from the group consisting of halogen, hydroxy, cyano, nitro, —NR⁴R⁵, —CO—R⁶, C₁₋₆-alkyl, C₁₋₆-alkoxy, trifluoromethyl, trifluoromethoxy, and -L¹-Q¹.

26. The compound of claim 1, wherein L¹ is a direct bond, —CH₂—, or —O.

27. The compound of claim 1, wherein Q¹ is C₃₋₁₂-cycloalkyl, C₃₋₁₀-heterocyclyl, C₆₋₁₃-aryl, or C₅₋₁₄-heteroaryl; each of which may be optionally substituted with one or more substituents selected from the group consisting of halogen, hydroxy, cyano, nitro, trifluoromethyl, trifluoromethoxy, —NR²⁶R²⁷, —CO—R²⁸, —S(O)₂—R²⁹, C₁₋₆-alkyl, C₁₋₆-alkoxy, C₃₋₇-cycloalkyl and C₃₋₇-cycloalkoxy.

28. The compound of claim 1, wherein R²⁶ and R²⁷ independently of each other are hydrogen or C₁₋₆-alkyl.

29. The compound of claim 1, wherein R²⁸ is methyl.

30. The compound of claim 1, wherein R²⁹ is C₁₋₆-alkyl.

31. The compound of claim 1, wherein R³¹ is C₆₋₁₃-aryl or C₃₋₁₀-heteroaryl.

32. The compound of claim 1, wherein (a) b is 1, (b) c is 1, and/or (c) d is 0.

33. The compound of claim 1, wherein G² is C₃₋₁₂-cycloalkyl, C₃₋₁₀-heterocyclyl, C₆₋₁₃-aryl or C₅₋₁₄-heteroaryl; each of which may be optionally substituted with one or more substituents selected from the group consisting of halogen, hydroxy, cyano, nitro, difluoromethyl, trifluoromethyl, difluoromethoxy, trifluoromethoxy, —NR⁹R¹⁰, C₁₋₆-alkyl, C₁₋₆-alkoxy, C₃₋₇-cycloalkyl, C₃₋₇-cycloalkoxy or -L²-Q².

34. The compound of claim 1, wherein R⁹ and R¹⁰ are independently hydrogen, C₁₋₆-alkyl, C₆₋₁₃-aryl, C₅₋₁₄-heteroaryl, —CO—R³⁴ or —SO₂—R³⁵.

35. The compound of claim 1, wherein L² is a direct bond, —CH₂—, —O—, —CO—, —CH₂—O—, or —O—CH₂—.

36. The compound of claim 1, wherein Q² is C₃₋₁₂-cycloalkyl, C₃₋₁₀-heterocyclyl, C₆₋₁₃-aryl or C₅₋₁₄-heteroaryl; each of which may be optionally substituted with halogen, hydroxy, cyano, nitro, trifluoromethyl, —NR³⁷R³⁸, —CO—R³⁹, —O—R⁴⁰, C₁₋₆-alkyl, C₁₋₆-hydroxyalkyl, C₃₋₇-cycloalkyl, or C₃₋₇-cycloalkoxy.

37. The compound of claim 1, wherein R³⁷ and R³⁸ independently of each other are hydrogen or C₁₋₆-alkyl.

38. The compound of claim 1, wherein R³⁹ is hydrogen or C₁₋₆-alkyl.

39. The compound of claim 1, wherein R⁴⁰ is trifluoromethyl.

40. The compound of claim 1, wherein R¹ is hydrogen, C₁₋₆-alkyl, C₂₋₆-alkenyl, or C₂₋₆-alkynyl.

41. The compound of claim 1, where the compound is selected from the group consisting of
(S,S)-6-(4-amino-butyl)-1-biphenyl-4-ylmethyl-3-naphthalen-2-ylmethyl-piperazine-2,5-dione,
(S,S)-6-(4-amino-butyl)-1-biphenyl-4-ylmethyl-3-naphthalen-1-ylmethyl-piperazine-2,5-dione,
(S,S)-6-(4-amino-butyl)-3-(4-benzyloxy-benzyl)-1-biphenyl-4-ylmethyl-piperazine-2,5-dione,
(S,S)-6-(4-amino-butyl)-1,3-bis-biphenyl-4-ylmethyl-piperazine-2,5-dione,
(S,S)-6-(4-amino-butyl)-3-naphthalen-2-ylmethyl-1-(4-phenoxy-benzyl)-piperazine-2,5-dione,
(S,S)-6-(4-amino-butyl)-3-benzo[b]thiophen-3-ylmethyl-1-biphenyl-4-ylmethyl-piperazine-2,5-dione,
(S,S)-6-(4-amino-butyl)-3-(4-benzoyl-benzyl)-1-biphenyl-4-ylmethyl-piperazine-2,5-dione,
(S,S)-6-(4-amino-butyl)-1-(4′-methoxy-biphenyl-4-ylmethyl)-3-naphthalen-2-ylmethyl-piperazine-2,5-dione,
(S,S)-6-(4-amino-butyl)-3-naphthalen-2-ylmethyl-1-(4′-trifluoromethyl-biphenyl-4-ylmethyl)-piperazine-2,5-dione,
(S,S)-6-(4-amino-butyl)-1-(4′-chloro-biphenyl-4-ylmethyl)-3-naphthalen-2-ylmethyl-piperazine-2,5-dione,
(S,S)-6-(4-amino-butyl)-1-(9H-fluoren-2-ylmethyl)-3-naphthalen-2-ylmethyl-piperazine-2,5-dione,
(S,S)-4′-[2-(4-amino-butyl)-5-naphthalen-2-ylmethyl-3,6-dioxo-piperazin-1-ylmethyl]-biphenyl-2-carboxylic acid methyl,
(S,S)-6-(4-amino-butyl)-3-(4-benzoyl-benzyl)-1-(4-phenoxy-benzyl)-piperazine-2,5-dione,
(S,S)-6-(4-amino-butyl)-3-(4-methoxy-benzyl)-1-(4-phenoxy-benzyl)-piperazine-2,5-dione,
(S,S)-6-(4-amino-butyl)-3-(4-chloro-benzyl)-1-(4-phenoxy-benzyl)-piperazine-2,5-dione,
(S,S)-6-(4-amino-butyl)-3-(4-methyl-benzyl)-1-(4-phenoxy-benzyl)-piperazine-2,5-dione,
(S,S)-4′-[2-(4-amino-butyl)-5-naphthalen-2-ylmethyl-3,6-dioxo-piperazin-1-ylmethyl]-biphenyl-2-carbonitrile,
(S,S)-6-(4-amino-butyl)-1-(4-cyclohexyloxy-benzyl)-3-naphthalen-2-ylmethyl-piperazine-2,5-dione,
(S,S)-6-(4-amino-butyl)-3-naphthalen-2-ylmethyl-1-[4-(3-trifluoromethyl-cyclohexyloxy)-benzyl]-piperazine-2,5-dione,
(S,S)-6-(4-amino-butyl)-1-(4-cyclohexyl-benzyl)-3-naphthalen-2-ylmethyl-piperazine-2,5-dione,
(S,S)-1-biphenyl-4-ylmethyl-6-(4-dimethylamino-butyl)-3-naphthalen-2-ylmethy-piperazine-2,5-dione,
(S,S)-1-biphenyl-4-ylmethyl-6-(4-methylamino-butyl)-3-naphthalen-2-ylmethyl-piperazine-2,5-dione,
(S,S)-6-(4-amino-butyl)-3-(4-ethoxy-benzyl)-1-(4-phenoxy-benzyl)-piperazine-2,5-dione,
(S,S)-6-(4-amino-butyl)-1-biphenyl-4-ylmethyl-3-(4-propoxy-benzyl)-piperazine-2,5-dione,
(S,S)-6-(4-amino-butyl)-1-biphenyl-4-ylmethyl-3-(4-isopropoxy-benzyl)-piperazine-2,5-dione,
(S,S)-6-(4-amino-butyl)-1-(4-phenoxy-benzyl)-3-(4-pyrrol-1-yl-benzyl)-piperazine-2,5-dione,
(S,S)-6-(4-amino-butyl)-1-biphenyl-4-ylmethyl-3-(4-cyclopropylmethoxy-benzyl)-piperazine-2,5-dione,
(S,S)-6-(4-amino-butyl)-1-biphenyl-4-ylmethyl-3-(4-cyclohexyloxy-benzyl)-piperazine-2,5-dione,
(S,S)-1-biphenyl-4-ylmethyl-6-(4-isopropylamino-butyl)-3-naphthalen-2-ylmethyl-piperazine-2,5-dione,
(S,S)-6-(4-amino-butyl)-1-biphenyl-4-ylmethyl-3-(4-phenoxy-benzyl)-piperazine-2,5-dione,
(S,S)-6-(4-amino-butyl)-1-biphenyl-4-ylmethyl-3-(4-m-tolyloxy-benzyl)-piperazine-2,5-dione,
(S,S)-6-(4-amino-butyl)-1-biphenyl-4-ylmethyl-3-[4-(4-methoxy-phenoxy)-benzyl]-piperazine-2,5-dione,
(S,S)-6-(4-amino-butyl)-1-[4-(4-dimethylamino-phenoxy)-benzyl]-3-naphthalen-2-ylmethyl-piperazine-2,5-dione,
(S,S)-6-(4-amino-butyl)-1-[4-(4-methoxy-phenoxy)-benzyl]-3-naphthalen-2-ylmethyl-piperazine-2,5-dione,
(S,S)-1-[4-(3-acetyl-phenoxy)-benzyl]-6-(4-amino-butyl)-3-naphthalen-2-ylmethyl-piperazine-2,5-dione,
(S,S)-6-(4-amino-butyl)-1-[4-(4-ethanesulfonyl-phenoxy)-benzyl]-3-naphthalen-2-ylmethyl-piperazine-2,5-dione,
(S,S)-6-(4-amino-butyl)-1-biphenyl-4-ylmethyl-3-[4-(4-chloro-phenoxy)-benzyl]-piperazine-2,5-dione,
(S,S)-3-[4-(4-acetyl-phenoxy)-benzyl]-6-(4-amino-butyl)-1-biphenyl-4-ylmethyl-piperazine-2,5-dione,
(S,S)-3-[4-(3-acetyl-phenoxy)-benzyl]-6-(4-amino-butyl)-1-biphenyl-4-ylmethyl-piperazine-2,5-dione,
(S,S)-6-(4-amino-butyl)-1-biphenyl-4-ylmethyl-3-(4-methoxy-benzyl)-piperazine-2,5-dione,
(S,S)-6-(4-amino-butyl)-1-biphenyl-4-ylmethyl-3-(4-ethoxy-benzyl)-piperazine-2,5-dione,
(S,S)-6-(4-amino-butyl)-1-biphenyl-4-ylmethyl-3-[4-(3-trifluoromethoxy-phenoxy)-benzyl]-piperazine-2,5-dione,
(S,S)-6-(4-amino-butyl)-1-biphenyl-4-ylmethyl-3-[4-(4-fluoro-phenoxy)-benzyl]-piperazine-2,5-dione,
(S,S)-6-(4-amino-butyl)-1-biphenyl-4-ylmethyl-3-[4-(3-nitro-phenoxy)-benzyl]-piperazine-2,5-dione,
(S,S)-6-(4-amino-butyl)-1-(4-phenoxy-benzyl)-3-(4-propoxy-benzyl)-piperazine-2,5-dione,
(S,S)-6-(4-amino-butyl)-1-biphenyl-4-ylmethyl-3-[4-(pyridin-3-yloxy)-benzyl]-piperazine-2,5-dione,
(S,S)-6-(4-amino-butyl)-1-biphenyl-4-ylmethyl-3-[4-(4-dimethylamino-phenoxy)-benzyl]-piperazine-2,5-dione,
(S,S)-6-(4-amino-butyl)-3-naphthalen-2-ylmethyl-1-(6-phenyl-pyridin-3-ylmethyl)-piperazine-2,5-dione,
(S,S)-3-{4-[5-(4-amino-butyl)-4-biphenyl-4-ylmethyl-3,6-dioxo-piperazin-2-ylmethyl]-phenoxy}-benzaldehyde,
(S,S)-6-(4-amino-butyl)-1-(4-bromo-benzyl)-3-naphthalen-2-ylmethyl-piperazine-2,5-dione,
(S,S)-6-(4-amino-butyl)-3-(4-isopropoxy-benzyl)-1-(4-phenoxy-benzyl)-piperazine-2,5-dione,
(S,S)-6-[4-(2-amino-ethylamino)-butyl]-1-(4-phenoxy-benzyl)-3-(4-propoxy-benzyl)-piperazine-2,5-dione,
(S,S)-3-amino-N-(1-biphenyl-4-ylmethyl-5-naphthalen-2-ylmethyl-3,6-dioxo-piperazin-2-ylmethyl)-3-methyl-N-piperidin-4-ylmethyl-butyramide,
(S,S)-3-amino-N-(1-biphenyl-4-ylmethyl-5-naphthalen-2-ylmethyl-3,6-dioxo-piperazin-2-ylmethyl)-N-pyridin-4-ylmethyl-propionamide,
(S,S)-3-amino-N-[5-(4-ethoxy-benzyl)-3,6-dioxo-1-(4-phenoxy-benzyl)-piperazin-2-ylmethyl]-3-methyl-N-piperidin-4-ylmethyl-butyramide,
(S,S)-3-amino-N-[5-(4-ethoxy-benzyl)-3,6-dioxo-1-(4-phenoxy-benzyl)-piperazin-2-ylmethyl]-N-piperidin-4-ylmethyl-propionamide,
(S,S)-6-{[bis-(3H-imidazol-4-ylmethyl)-amino]-methyl}-3-(4-ethoxy-benzyl)-1-(4-phenoxy-benzyl)-piperazine-2,5-dione,
(S,S)-3-amino-N-(2-amino-2-methyl-propyl)-N-[5-(4-ethoxy-benzyl)-3,6-dioxo-1-(4-phenoxy-benzyl)-piperazin-2-ylmethyl]-3-methyl-butyramide,
(S,S)-1-[4-(4-acetyl-phenoxy)-benzyl]-6-(4-amino-butyl)-3-naphthalen-2-ylmethyl-piperazine-2,5-dione,
(S,S)-6-(4-amino-butyl)-1-biphenyl-4-ylmethyl-3-[4-(3-hydroxymethyl-phenoxy)-benzyl]-piperazine-2,5-dione,
(S,S)-6-{4-[(1H-imidazol-2-ylmethyl)-amino]-butyl}-3-(4-methoxy-benzyl)-1-(4-phenoxy-benzyl)-piperazine-2,5-dione,
(S,S)-3-(4-methoxy-benzyl)-1-(4-phenoxy-benzyl)-6-{4-[(pyridin-2-ylmethyl)-amino]-butyl}-piperazine-2,5-dione,
(2R,2′S,5′S)-2-amino-N-[5-(4-ethoxy-benzyl)-3,6-dioxo-1-(4-phenoxy-benzyl)-piperazin-2-ylmethyl]-3-(1H-imidazol-4-yl)-propionamide,
(S,S)-2-(3-amino-propylamino)-N-[1-[4-(methyl-phenyl-amino)-benzyl]-3,6-dioxo-5-(4-propoxy-benzyl)-piperazin-2-ylmethyl]-acetamide,
N-[4-((2S,5S)-1-biphenyl-4-ylmethyl-5-naphthalen-2-ylmethyl-3,6-dioxo-piperazin-2-y)-butyl]-acetamide,
(3S,6 S)-1-biphenyl-4-ylmethyl-6-(4-dimethylamino-butyl)-3-naphthalen-2-ylmethyl-piperazine-2,5-dione,
N-[4-((2S,5S)-1-biphenyl-4-ylmethyl-5-naphthalen-2-ylmethyl-3,6-dioxo-piperazin-2-yl)-butyl]-guanidine hydrochloride,
(3S6S)-6-[4-(3-amino-pyridin-2-ylamino)-butyl]-3-naphthalen-2-ylmethyl-1-(4-phenoxy-benzyl)-piperazine-2,5-dione,
{4-[(2S,5S)-5-naphthalen-2-ylmethyl-3,6-dioxo-1-(4-phenoxy-benzyl)-piperazin-2-yl]-butylamino}-acetonitrile,
N-((2S,5S)-1-biphenyl-4-ylmethyl-5-naphthalen-2-ylmethyl-3,6-dioxo-piperazin-2-ylmethyl)-N-piperidin-4-ylmethyl-acetamide,
(3S,6S)-1-biphenyl-4-ylmethyl-6-[(cyclohexylmethyl-piperidin-4-ylmethyl-amino)-methyl]-3-naphthalen-2-ylmethyl-piperazine-2,5-dione,
(3S,6S)-1-biphenyl-4-ylmethyl-6-[(ethyl-piperidin-4-ylmethyl-amino)-methyl]-3-naphthalen-2-ylmethyl-piperazine-2,5-dione,
(3S,6S)-1-biphenyl-4-ylmethyl-3-naphthalen-2-ylmethyl-6-[(piperidin-4-ylmethyl-pyridin-4-ylmethyl-amino)-methyl]-piperazine-2,5-dione,
3-amino-N-((2S,5S)-1-biphenyl-4-ylmethyl-5-naphthalen-2-ylmethyl-3,6-dioxo-piperazin-2-ylmethyl)-N-piperidin-4-ylmethyl-propionamide,
4-{[((2S,5S)-1-biphenyl-4-ylmethyl-5-naphthalen-2-ylmethyl-3,6-dioxo-piperazin-2-ylmethyl)-(piperidine-4-carbonyl)-amino]-methyl}-piperidine-1-carboxylic acid benzyl ester,
4-{[((2S,5S)-1-biphenyl-4-ylmethyl-5-naphthalen-2-ylmethyl-3,6-dioxo-piperazin-2-ylmethyl)-((R,S)-piperidine-3-carbonyl)-amino]-methyl}-piperidine-1-carboxylic acid benzyl ester,
piperidine-4-carboxylic acid ((2S,5S)-1-biphenyl-4-ylmethyl-5-naphthalen-2-ylmethyl-3,6-dioxo-piperazin-2-ylmethyl)-piperidin-4-ylmethyl-amide,
(R,S)-piperidine-3-carboxylic acid ((2S,5S)-1-biphenyl-4-ylmethyl-5-naphthalen-2-ylmethyl-3,6-dioxo-piperazin-2-ylmethyl)-piperidin-4-ylmethyl-amide,
4-amino-N-((2S,5 S)-1-biphenyl-4-ylmethyl-5-naphthalen-2-ylmethyl-3,6-dioxo-piperazin-2-ylmethyl)-N-piperidin-4-ylmethyl-butyramide,
(3S,6S)-6-{[(3-amino-propyl)-piperidin-4-ylmethyl-amino]-methyl}-1-biphenyl-4-ylmethyl-3-naphthalen-2-ylmethyl-piperazine-2,5-dione,
1H-imidazole-4-carboxylic acid [(2S,5S)-5-naphthalen-2-ylmethyl-3,6-dioxo-1-(4-phenoxy-benzyl)-piperazin-2-ylmethyl]-piperidin-4-ylmethyl-amide,
2-amino-N-[(2S,5S)-5-naphthalen-2-ylmethyl-3,6-dioxo-1-(4-phenoxy-benzyl)-piperazin-2-ylmethyl]-N-piperidin-4-ylmethyl-acetamide,
3-amino-N-[(2S,5 S)-5-naphthalen-2-ylmethyl-3,6-dioxo-1-(4-phenoxy-benzyl)-piperazin-2-ylmethyl]-N-piperidin-4-ylmethyl-propionamide,
N-[(2S,5S)-5-naphthalen-2-ylmethyl-3,6-dioxo-1-(4-phenoxy-benzyl)-piperazin-2-ylmethyl]-2-piperidin-4-yl-N-piperidin-4-ylmethyl-acetamide,
(R,S)-2,5-diamino-pentanoic acid [(2S,5 S)-5-naphthalen-2-ylmethyl-3,6-dioxo-1-(4-phenoxy-benzyl)-piperazin-2-ylmethyl]-piperidin-4-ylmethyl-amide,
(3S,6S)-6-{[(3-dimethylamino-propyl)-piperidin-4-ylmethyl-amino]-methyl}-3-naphthalen-2-ylmethyl-1-(4-phenoxy-benzyl)-piperazine-2,5-dione,
3-amino-N-(1-methyl-piperidin-4-ylmethyl)-N-[(2S,5S)-5-naphthalen-2-ylmethyl-3,6-dioxo-1-(4-phenoxy-benzyl)-piperazin-2-ylmethyl]-propionamide,
piperidine-3-carboxylic acid [(2S,5S)-5-naphthalen-2-ylmethyl-3,6-dioxo-1-(4-phenoxy-benzyl)-piperazin-2-ylmethyl]-piperidin-4-ylmethyl-amide,
(3S,6S)-1-biphenyl-4-ylmethyl-6-{[bis-(1-methyl-piperidin-4-ylmethyl)-amino]-methyl}-3-naphthalen-2-ylmethyl-piperazine-2,5-dione,
(3S,6S)-6-{[(3-amino-propyl)-piperidin-4-ylmethyl-amino]-methyl}-1-(4-phenoxy-benzyl)-3-(4-trifluoromethyl-benzyl)-piperazine-2,5-dione,
(3S,6S)-6-{[(3-hydroxy-propyl)-piperidin-4-ylmethyl-amino]-methyl}-1-(4-phenoxy-benzyl)-3-(4-trifluoromethyl-benzyl)-piperazine-2,5-dione,
3-amino-N-[(2S,5S)-3,6-dioxo-1-(4-phenoxy-benzyl)-5-(4-trifluoromethyl-benzyl)-piperazin-2-ylmethyl]-N-piperidin-4-ylmethyl-propionamide,
N-((2S,5S)-1-biphenyl-4-ylmethyl-5-naphthalen-2-ylmethyl-3,6-dioxo-piperazin-2-ylmethyl)-2-(R, S)-morpholin-2-yl-N-piperidin-4-ylmethyl-acetamide,
(3S,6S)-1-biphenyl-4-ylmethyl-3-naphthalen-2-ylmethyl-6-[(piperidin-4-ylmethyl-pyridin-3-ylmethyl-amino)-methyl]-piperazine-2,5-dione,
(3S,6S)-1-(4-phenoxy-benzyl)-6-[(piperidin-4-ylmethyl-pyridin-3-ylmethyl-amino)-methyl]-3-(4-trifluoromethyl-benzyl)-piperazine-2,5-dione,
N-((2S,5S)-1-biphenyl-4-ylmethyl-5-naphthalen-2-ylmethyl-3,6-dioxo-piperazin-2-ylmethyl)-2-cyclopropylamino-N-piperidin-4-ylmethyl-acetamide,
N-((2S,5S)-1-biphenyl-4-ylmethyl-5-naphthalen-2-ylmethyl-3,6-dioxo-piperazin-2-ylmethyl)-N-piperidin-4-ylmethyl-2-(2,2,2-trifluoro-ethylamino)-acetamide,
N-((2S,5S)-1-biphenyl-4-ylmethyl-5-naphthalen-2-ylmethyl-3,6-dioxo-piperazin-2-ylmethyl)-2-imidazol-1-yl-N-piperidin-4-ylmethyl-acetamide,
2-[((2S,5 S)-1-biphenyl-4-ylmethyl-5-naphthalen-2-ylmethyl-3,6-dioxo-piperazin-2-ylmethyl)-piperidin-4-ylmethyl-amino]-acetamide,
N-((2S,5S)-1-biphenyl-4-ylmethyl-5-naphthalen-2-ylmethyl-3,6-dioxo-piperazin-2-ylmethyl)-N-piperidin-4-ylmethyl-2-pyridin-3-yl-acetamide,
N-((2S,5S)-1-biphenyl-4-ylmethyl-5-naphthalen-2-ylmethyl-3,6-dioxo-piperazin-2-ylmethyl)-N-piperidin-4-ylmethyl-nicotinamide,
N-((2S,5S)-1-biphenyl-4-ylmethyl-5-naphthalen-2-ylmethyl-3,6-dioxo-piperazin-2-ylmethyl)-N-piperidin-4-ylmethyl-2-pyrrolidin-1-yl-acetamide,
3-amino-N-((2S,5S)-1-biphenyl-4-ylmethyl-5-naphthalen-2-ylmethyl-3,6-dioxo-piperazin-2-ylmethyl)-N-pyridin-3-ylmethyl-propionamide,
(S,S)-6-(4-Amino-butyl)-3-(3-chloro-4-methoxy-benzyl)-1-(4-phenoxy-benzyl)-piperazine-2,5-dione,
(S,S)-6-(4-Amino-butyl)-1-biphenyl-4-ylmethyl-3-(1-methoxy-naphthalen-2-ylmethyl)-piperazine-2,5-dione,
(S,S)-6-(4-Amino-butyl)-1-biphenyl-4-ylmethyl-3-(6-chloro-naphthalen-2-ylmethyl)-piperazine-2,5-dione,
(S,S)-6-(4-Amino-butyl)-3-(4-amino-3,5-dibromo-benzyl)-1-(4-phenoxy-benzyl)-piperazine-2,5-dione,
(S,S)-6-(4-Amino-butyl)-3-(4-hydroxy-3,5-dibromo-benzyl)-1-(4-phenoxy-benzyl)-piperazine-2,5-dione,
(S,S)-6-(4-Amino-butyl)-3-naphthalen-2-ylmethyl-1-[4-(pyridin-4-yloxy)-benzyl]-piperazine-2,5-dione,
(S,S)-6-(4-Amino-butyl)-1-(4-phenoxy-benzyl)-3-(5,6,7,8-tetrahydro-naphthalen-2-ylmethyl)-piperazine-2,5-dione,
(S,S)-6-(4-Amino-butyl)-3-naphthalen-2-ylmethyl-1-(4-o-tolyloxy-benzyl)-piperazine-2,5-dione,
(S,S)-6-(4-Amino-butyl)-1-[4-(3-chloro-phenoxy)-benzyl]-3-naphthalen-2-ylmethyl-piperazine-2,5-dione,
(S,S)-6-(4-Amino-butyl)-1-biphenyl-4-ylmethyl-3-(3-methoxy-naphthalen-2-ylmethyl)-piperazine-2,5-dione,
(S,S)-6-(4-Amino-butyl)-1-[4-(tert-butyl-diphenyl-silanyloxy)-benzyl]-3-naphthalen-2-ylmethyl-piperazine-2,5-dione (exp 15)
(S,S)-Carbonic acid 4-[2-(4-amino-butyl)-5-naphthalen-2-ylmethyl-3,6-dioxo-piperazin-1-ylmethyl]-phenyl ester benzyl ester,
(S,S)-6-(4-Amino-butyl)-1-[4-(methyl-phenyl-amino)-benzyl]-3-naphthalen-2-ylmethyl-piperazine-2,5-dione,
(S,S)-6-(4-Amino-butyl)-1-(4-benzyl-benzyl)-3-naphthalen-2-ylmethyl-piperazine-2,5-dione,
(S,S)-6-(4-Amino-butyl)-1-(3-methyl-4-phenoxy-benzyl)-3-naphthalen-2-ylmethyl-piperazine-2,5-dione,
(S,S)-6-(4-Amino-butyl)-1-(3-methoxy-4-phenoxy-benzyl)-3-naphthalen-2-ylmethyl-piperazine-2,5-dione,
(S,S)-3-(4-Amino-butyl)-4-biphenyl-4-ylmethyl-1-methyl-6-naphthalen-2-ylmethyl-piperazine-2,5-dione,
(S,S)-6-(5-Amino-pentyl)-1-biphenyl-4-ylmethyl-3-naphthalen-2-ylmethyl-piperazine-2,5-dione,
(S,S)-6-(4-Amino-butyl)-3-naphthalen-2-ylmethyl-1-(3-phenoxy-benzyl)-piperazine-2,5-dione,
(S,S)-6-(4-Amino-butyl)-3-(4-benzyloxy-benzyl)-1-(3-phenoxy-benzyl)-piperazine-2,5-dione,
(S,S)-6-(4-Amino-butyl)-3-naphthalen-1-ylmethyl-1-(3-phenoxy-benzyl)-piperazine-2,5-dione,
(S,S)-6-(4-Amino-butyl)-3-biphenyl-4-ylmethyl-1-(3-phenoxy-benzyl)-piperazine-2,5-dione,
(S,S)-6-(5-Amino-pentyl)-3-(4-benzyloxy-benzyl)-1-biphenyl-4-ylmethyl-piperazine-2,5-dione,
(S,S)-6-(5-Amino-pentyl)-1,3-bis-(4-benzyloxy-benzyl)-piperazine-2,5-dione,
(S,S)-6-(5-Amino-pentyl)-1-(4-benzyloxy-benzyl)-3-naphthalen-1-ylmethyl-piperazine-2,5-dione,
(S,S)-6-(5-Amino-pentyl)-1-(4-benzyloxy-benzyl)-3-biphenyl-4-ylmethyl-piperazine-2,5-dione,
(S,S)-6-(4-Amino-butyl)-3-(3,4-dichloro-benzyl)-1-(4-phenoxy-benzyl)-piperazine-2,5-dione,
(S,S)-6-(4-Amino-butyl)-3-naphthalen-1-ylmethyl-1-(4-phenoxy-benzyl)-piperazine-2,5-dione,
(S,S)-6-(4-Amino-butyl)-1-(9H-fluoren-3-ylmethyl)-3-naphthalen-1-ylmethyl-piperazine-2,5-dione,
(S,S)-6-(4-Amino-butyl)-1-(4-benzyloxy-benzyl)-3-naphthalen-1-ylmethyl-piperazine-2,5-dione,
and a combination of any thereof.

42. A composition comprising the compound of claim 1 in an amount sufficient to induce a physiological response in a subject associated with the modulation of melanocortin receptor activity and a pharmaceutically acceptable carrier, diluent, or excipient.

43. A method for modulating melanocortin receptor activity in a subject comprising administering to the subject the composition of claim 42 in an amount sufficient to detectably modulate melanocortin receptor activity.

44. The method of claim 43, wherein administration of the composition detectably modulates MC4 receptor activity.

45. The method of claim 44, wherein administration of the composition also detectably modulates MC3 receptor activity.

46. The method of claim 43, wherein administration of the composition detectably modulates MC5 receptor activity.

47. The method of claim 46, wherein administration of the composition also detectably modulates MC3 receptor activity.

48. A method of regulating exocrine gland secretion, regulating aldosterone secretion, suppressing stress-induced alarm substances, stimulating an exocrine gland function, stimulating cardiac function, stimulating testicular function, or any combination thereof in a subject comprising administering a therapeutically effective amount of the composition of claim 42 to the subject.

49. A method of inducing, enhancing, and/or promoting the treatment or prevention of hypertension in a subject comprising administering a therapeutically effective amount of the composition of claim 42 to the subject.

50. A method of increasing antipyretic activity in a subject comprising administering a therapeutically effective amount of the composition of the composition of claim 42 to the subject.

51. A method for inducing, enhancing, and/or promoting lipolysis in a subject comprising administering a therapeutically effective amount of the composition of claim 42 to the subject.

52. A method for promoting the suppression of appetite, inducing satiety, or a combination thereof in a subject comprising administering a therapeutically effective amount of the composition of claim 42 to the subject.

53. A method of reducing the risk, onset, and/or severity or promoting the treatment of at least one condition associated with the activation of the MC4 receptor in a subject comprising administering a therapeutically effective amount of the composition of claim 42 to the subject.

54. The method of claim 53, wherein the at least one condition comprises hyperglycemia, impaired glucose tolerance, diabetes, dyslipidemia, hyperlipidemia, or a combination of any thereof.

55. A method of reducing the risk, onset, and/or severity or promoting the treatment of at least one condition associated with the activation of the MC1 receptor in a subject comprising administering a therapeutically effective amount of the composition of claim 42 to the subject.

56. The method of claim 55, wherein administration of the composition detectably increases the subject's skin pigmentation, modulates an inflammatory response in the skin of the subject, reduces or prevents contact dermatitis in the subject, modulates an immune response in the subject, at least partially inhibits a chronic inflammatory response in the subject, or induces, promotes, and/or enhances any combination thereof.

57. A method of reducing the risk, onset, and/or severity or promoting the treatment of at least one condition associated with the activation of the MC2 receptor in a subject comprising administering a therapeutically effective amount of the composition of claim 42 to the subject.

58. A method of regulating glucocorticoid production in a subject comprising administering a therapeutically effective amount of the composition of claim 42 to the subject.

59. A method of reducing the risk, onset, and/or severity or promoting the treatment of at least one condition associated with the activation of the MC3 receptor in a subject comprising administering a therapeutically effective amount of the composition of claim 42 to the subject.

60. The method of claim 59, wherein the condition is hypertension, obesity, sexual dysfunction, or a combination of any thereof.

61. A method of reducing blood pressure; reducing heart rate; inducing, enhancing and/or promoting natriuresis; or inducing, promoting, and/or enhancing any combination thereof in a subject comprising administering a therapeutically effective amount of the composition of claim 42 to the subject.

62. A method of reducing the risk, onset, and/or severity or promoting the treatment of at least one condition associated with the activation of the MC5 receptor in a subject comprising administering a therapeutically effective amount of the composition of claim 42 to the subject.
